(19) United States
(12) Patent Application Publication
(10) Pub. No.: US 2026/0193655 A1
(43) Pub. Date: Jul. 9, 2026

(54) RNAi Agents for Inhibiting Expression of Receptor for Advanced Glycation End-products, Compositions Thereof, and Methods of Use

(71) Applicant: Arrowhead Pharmaceuticals, Inc., Pasadena, CA (US)

(72) Inventors: Anthony Nicholas, Oregon, WI (US); Erik W. Bush, Verona, WI (US); David Itiro Kasahara, Madison, WI (US); Casi M. Schienebeck, Deerfield, WI (US)

(21) Appl. No.: 19/394,336

(22) Filed: Nov. 19, 2025

Related U.S. Application Data
(63) Continuation of application No. 17/715,444, filed on Apr. 7, 2022, now Pat. No. 12,486,502.
(60) Provisional application No. 63/172,301, filed on Apr. 8, 2021.
(60) Provisional application No. 63/322,603, filed on Mar. 22, 2022.

(51) Int. Cl. C12N 15/113; A61K 9/00; A61P 11/00

(52) U.S. Cl. CPC C12N 15/113; A61K 9/0073; A61P 11/00; C12N 2310/11; C12N 2310/14

(57) ABSTRACT

Described are RNAi agents, compositions that include RNAi agents, and methods for inhibition of a Receptor for Advanced Glycation End-products (AGER or RAGE) gene. The RAGE RNAi agents and RNAi agent conjugates disclosed herein inhibit the expression of an AGER gene. Pharmaceutical compositions that include one or more RAGE RNAi agents, optionally with one or more additional therapeutics, are also described. Delivery of the described RAGE RNAi agents to pulmonary cells, in vivo, provides for inhibition of AGER gene expression and a reduction in membrane RAGE activity, which can provide a therapeutic benefit to subjects, including human subjects, for the treatment of various diseases including pulmonary inflammation diseases such as severe asthma.

### CROSS REFERENCE TO RELATED APPLICATIONS

This application is a continuation of U.S. patent application Ser. No. 17/715,444, filed on Apr. 7, 2022, which claims the benefit of priority from U.S. Provisional Patent Application Ser. No. 63/172,301, filed on Apr. 8, 2021, and U.S. Provisional Patent Application Ser. No. 63/322,603, filed on Mar. 22, 2022, the contents of each of which are incorporated herein by reference in their entirety.

### SEQUENCE LISTING

This application contains a Sequence Listing (in compliance with Standard ST26), which has been submitted in xml format and is hereby incorporated by reference in its entirety. The xml sequence listing file is named 30691-US2_SeqListing.xml, created Nov. 19, 2025, and is 3,666,807 bytes in size.

### FIELD OF THE INVENTION

The present disclosure relates to RNA interference (RNAi) agents, e.g., double stranded RNAi agents, for inhibition of Receptor for Advanced Glycation End-products (“RAGE” or “AGER”) gene expression, compositions that include RAGE RNAi agents, and methods of use thereof.

### BACKGROUND

The Receptor for Advanced Glycation End-products (“RAGE” or “AGER”) is a 35 kilodalton transmembrane protein of the immunoglobulin superfamily which functions as a pro-inflammatory pattern recognition receptor. In its full-length, membrane-bound form, the receptor has three functional domains: an extracellular ligand-binding domain, a hydrophobic transmembrane domain, and a cytoplasmic domain that mediates ligand-dependent signal transduction. A second, non-membrane bound soluble form of the receptor (sRAGE) contains only the extracellular ligand-binding domain; formed by proteolytic cleavage of full-length membrane-bound RAGE (or by alternative splicing), sRAGE antagonizes RAGE function since it binds ligands but lacks a cytoplasmic signaling domain.

RAGE is expressed at constitutively high levels in the lung, primarily localized to type 1 alveolar epithelial cells. Other tissues in the body normally express RAGE at low levels, but expression is upregulated in the presence of RAGE ligands and chronic inflammation. As a pattern recognition receptor, RAGE binds a wide variety of endogenous ligands, including advanced glycation end-products (sugar-modified proteins or lipids), high mobility group box 1 (HMGB1) and S100 proteins. Different intermediate signaling pathways can be activated by different RAGE ligands (e.g. ERK1/2, p38 and JAK/STAT) culminating in the production of reactive oxygen species, sustained activation of NF-κB and the transcription of pro-inflammatory genes (e.g. interleukins, interferon, TNF alpha). Transcription of the gene encoding RAGE itself is promoted by NF-κB, creating a positive feedback loop that perpetuates chronic inflammation.

RAGE has been linked to the chronic, pathological inflammation that contributes to many diseases, including: pulmonary disease (asthma, acute respiratory distress syndrome, idiopathic pulmonary fibrosis, chronic obstructive pulmonary disease, cystic fibrosis, pneumonia, lung cancer, bronchopulmonary dysplasia), cardiovascular disease (atherosclerosis, myocardial infarction, heart failure, peripheral vascular disease), cancer, diabetes, chronic kidney disease, neurodegenerative disease, rheumatoid arthritis, non-alcoholic steatohepatitis, injury caused by certain viral infections including SARS-CoV-2, certain ocular inflammatory conditions, and skeletal muscle wasting.

In the pulmonary disease space, RAGE knockout (KO) mice are completely protected, physiologically and histologically, from allergic asthma produced by challenge with house dust mite allergen or ovalbumin. Similarly, RAGE knockout mice are protected from hyperoxia or lipopolysaccharide-induced acute lung injury and inflammation. (See, e.g., Oczypok et al., Paediatr Respir Rev., 23:40-49 (2017); Wang et al., Shock, 50:472-482 (2018)). Genome-wide association studies (GWAS) have linked a variant gain-of-function RAGE allele (G82S) to increased inflammation, decreased pulmonary function, and risk of asthma (see, e.g., Hancock et al., Nat Genet., 42:45-52 (2010); Repapi et al., Nat Genet., 42:36-44 (2010)).

Despite its potential attractiveness as a drug target, development of potent and selective RAGE inhibitors has proven extremely challenging. Rather than binding to a discrete domain, a wide range of RAGE ligands interact with multiple binding sites within the antibody-like extracellular domain (see, e.g., Rojas et al., Current Drug Targets, 20:340-346 (2019)). While certain RNAi agents capable of inhibiting the expression of a RAGE in vitro have been previously identified and reported in various studies, or are otherwise commercially available, the known RNAi agent constructs are neither sufficiently potent nor sufficiently specific to be viable as a therapeutic drug candidate. Thus, there exists a need for RAGE RNAi agents suitable for use as a therapeutic in the treatment of RAGE-associated diseases and disorders.

### SUMMARY

There continues to exist a need for novel RNA interference (RNAi) agents (termed RNAi agents, RNAi triggers, or triggers), e.g., double stranded RNAi agents, that are able to selectively and efficiently inhibit the expression of a RAGE (AGER) gene, including for use as a therapeutic or medicament. Further, there exists a need for compositions of novel RAGE-specific RNAi agents for the treatment of diseases or disorders associated with pathological inflammation and/or disorders that can be mediated at least in part by a reduction in AGER gene expression and/or RAGE receptor levels.

The nucleotide sequences and chemical modifications of the RAGE RNAi agents disclosed herein, as well as their combination with certain specific targeting ligands suitable for selectively and efficiently delivering the RAGE RNAi agents in vivo, differ from those previously disclosed or known in the art. As shown in, for example, the various Examples herein, the disclosed RAGE RNAi agents provide for highly potent and efficient inhibition of the expression of an AGER (RAGE) gene.

In general, the present disclosure features RAGE gene-specific RNAi agents, compositions that include RAGE RNAi agents, and methods for inhibiting expression of an AGER (RAGE) gene in vitro and/or in vivo using the RAGE RNAi agents and compositions that include RAGE RNAi agents described herein. The RAGE RNAi agents described herein are able to selectively and efficiently decrease or inhibit expression of an AGER gene, and thereby reduce the expression of the RAGE receptor and decrease activation of RAGE receptor signaling, including NF-κB, which ultimately results in reduced inflammation.

The described RAGE RNAi agents can be used in methods for therapeutic treatment (including preventative or prophylactic treatment) of symptoms and diseases including, but not limited to various pulmonary disease (asthma, acute respiratory distress syndrome, idiopathic pulmonary fibrosis, chronic obstructive pulmonary disease, cystic fibrosis, pneumonia, lung cancer, bronchopulmonary dysplasia), cardiovascular disease (atherosclerosis, myocardial infarction, heart failure, peripheral vascular disease), cancer, diabetes, chronic kidney disease, neurodegenerative disease, rheumatoid arthritis, non-alcoholic steatohepatitis, the inflammatory injury caused by certain viral infections including SARS-CoV-2, certain ocular inflammatory conditions, and skeletal muscle wasting.

In one aspect, the disclosure features RNAi agents for inhibiting expression of a RAGE (AGER) gene, wherein the RNAi agent includes a sense strand (also referred to as a passenger strand) and an antisense strand (also referred to as a guide strand). The sense strand and the antisense strand can be partially, substantially, or fully complementary to each other. The length of the RNAi agent sense strands described herein each can be 15 to 49 nucleotides in length. The length of the RNAi agent antisense strands described herein each can be 18 to 49 nucleotides in length. In some embodiments, the sense and antisense strands are independently 18 to 26 nucleotides in length. The sense and antisense strands can be either the same length or different lengths. In some embodiments, the sense and antisense strands are independently 21 to 26 nucleotides in length. In some embodiments, the sense and antisense strands are independently 21 to 24 nucleotides in length. In some embodiments, both the sense strand and the antisense strand are 21 nucleotides in length. In some embodiments, the antisense strands are independently 18, 19, 20, 21, 22, 23, 24, 25, 26, 27, 28, 29, or 30 nucleotides in length. In some embodiments, the sense strands are independently 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides in length. The RNAi agents described herein, upon delivery to a cell expressing RAGE such as a pulmonary cell (including, more specifically, type 1 alveolar epithelial cell), inhibit the expression of one or more AGER gene variants in vivo and/or in vitro.

The RAGE RNAi agents disclosed herein target a human AGER gene (see, e.g., SEQ ID NO: 1). In some embodiments, the RAGE RNAi agents disclosed herein target a portion of an AGER gene having the sequence of any of the sequences disclosed in Table 1.

In another aspect, the disclosure features compositions, including pharmaceutical compositions, that include one or more of the disclosed RAGE RNAi agents that are able to selectively and efficiently decrease expression of an AGER gene. The compositions that include one or more RAGE RNAi agents described herein can be administered to a subject, such as a human or animal subject, for the treatment (including prophylactic treatment or inhibition) of symptoms and diseases associated with RAGE receptor activity.

Examples of RAGE RNAi agent sense strands and antisense strands that can be used in a RAGE RNAi agent are provided in Tables 3, 4, 5, and 6. Examples of RAGE RNAi agent duplexes are provided in Tables 7A, 7B, 8, 9A, 9B, and 10. Examples of 19-nucleotide core stretch sequences that may consist of or may be included in the sense strands and antisense strands of certain RAGE RNAi agents disclosed herein, are provided in Table 2.

In another aspect, the disclosure features methods for delivering RAGE RNAi agents to pulmonary epithelial cells in a subject, such as a mammal, in vivo. Also described herein are compositions for use in such methods. In some embodiments, disclosed herein are methods for delivering RAGE RNAi agents to pulmonary cells (including epithelial cells, macrophages, smooth muscle, endothelial cells, and preferably type 1 alveolar epithelial cells) to a subject in vivo. In some embodiments, the subject is a human subject.

The methods disclosed herein include the administration of one or more RAGE RNAi agents to a subject, e.g., a human or animal subject, by any suitable means known in the art. The pharmaceutical compositions disclosed herein that include one or more RAGE RNAi agents can be administered in a number of ways depending upon whether local or systemic treatment is desired. Administration can be, but is not limited to, for example, intravenous, intraarterial, subcutaneous, intraperitoneal, subdermal (e.g., via an implanted device), and intraparenchymal administration. In some embodiments, the pharmaceutical compositions described herein are administered by inhalation (such as dry powder inhalation or aerosol inhalation), intranasal administration, intratracheal administration, or oropharyngeal aspiration administration.

In some embodiments, it is desired that the RAGE RNAi agents described herein inhibit the expression of an AGER gene in the pulmonary epithelium, for which the administration is by inhalation (e.g., by an inhaler device, such as a metered-dose inhaler, or a nebulizer such as a jet or vibrating mesh nebulizer, or a soft mist inhaler).

The one or more RAGE RNAi agents can be delivered to target cells or tissues using any oligonucleotide delivery technology known in the art. In some embodiments, a RAGE RNAi agent is delivered to cells or tissues by covalently linking the RNAi agent to a targeting group. In some embodiments, the targeting group can include a cell receptor ligand, such as an integrin targeting ligand. Integrins are a family of transmembrane receptors that facilitate cell-extracellular matrix (ECM) adhesion. In particular, integrin alpha-v-beta-6 (αvβ6) is an epithelial-specific integrin that is known to be a receptor for ECM proteins and the TGF-beta latency-associated peptide (LAP), and is expressed in various cells and tissues. Integrin αvβ6 is known to be highly upregulated in injured pulmonary epithelium. In some embodiments, the RAGE RNAi agents described herein are linked to an integrin targeting ligand that has affinity for integrin αvβ6. As referred to herein, an “αvβ6 integrin targeting ligand” is a compound that has affinity for integrin αvβ6, which can be utilized as a ligand to facilitate the targeting and delivery of an RNAi agent to which it is attached to the desired cells and/or tissues (i.e., to cells expressing integrin αvβ6). In some embodiments, multiple αvβ6 integrin targeting ligands or clusters of αvβ6 integrin targeting ligands are linked to a RAGE RNAi agent. In some embodiments, the RAGE RNAi agent-αvβ6 integrin targeting ligand conjugates are selectively internalized by lung epithelial cells, either through receptor-mediated endocytosis or by other means.

Examples of targeting groups useful for delivering RAGE RNAi agents that include αvβ6 integrin targeting ligands are disclosed, for example, in International Patent Application Publication No. WO 2018/085415 and International Patent Application Publication No. WO 2019/089765, the contents of each of which are incorporated by reference herein in their entirety.

A targeting group can be linked to the 3′ or 5′ end of a sense strand or an antisense strand of a RAGE RNAi agent. In some embodiments, a targeting group is linked to the 3′ or 5′ end of the sense strand. In some embodiments, a targeting group is linked to the 5′ end of the sense strand. In some embodiments, a targeting group is linked internally to a nucleotide on the sense strand and/or the antisense strand of the RNAi agent. In some embodiments, a targeting group is linked to the RNAi agent via a linker.

In another aspect, the disclosure features compositions that include one or more RAGE RNAi agents that have the duplex structures disclosed in Tables 7A, 7B, 8, 9A, 9B, and 10.

The use of RAGE RNAi agents provides methods for therapeutic (including prophylactic) treatment of diseases or disorders for which a reduction in RAGE receptor activity can provide a therapeutic benefit. The RAGE RNAi agents disclosed herein can be used to treat various respiratory diseases, including pulmonary disease (asthma, acute respiratory distress syndrome, idiopathic pulmonary fibrosis, chronic obstructive pulmonary disease, cystic fibrosis, pneumonia, lung cancer, bronchopulmonary dysplasia), cardiovascular disease (atherosclerosis, myocardial infarction, heart failure, peripheral vascular disease), cancer, diabetes, chronic kidney disease, neurodegenerative disease, rheumatoid arthritis, non-alcoholic steatohepatitis, injury caused by certain viral infections including SARS-CoV-2, certain ocular inflammatory conditions, and skeletal muscle wasting. In some embodiments, the RAGE RNAi agents disclosed herein can be used to treat a pulmonary inflammatory disease or condition. RAGE RNAi agents can further be used to treat, for example, various ocular inflammatory diseases and disorders. Such methods of treatment include administration of a RAGE RNAi agent to a human being or animal having elevated or enhanced RAGE receptor levels or RAGE receptor activity beyond desirable levels.

One aspect described herein is an RNAi agent for inhibiting expression of a receptor for advanced glycation end-products gene, comprising:
(i) an antisense strand comprising at least 17 contiguous nucleotides differing by 0 or 1 nucleotides from any one of the sequences provided in Table 3;
(ii) a sense strand comprising a nucleotide sequence that is at least partially complementary to the antisense strand; and
(iii) one or more targeting ligands.

In another aspect described is an RNAi agent capable of inhibiting expression of a receptor for advanced glycation end-products gene comprising:
(i) an antisense strand that is between 18 and 49 nucleotides in length that is at least partially complementary to a receptor for advanced glycation end-products gene (SEQ ID NO:1);
(ii) a sense strand that is at least partially complementary to the antisense strand; and
(iii) a targeting ligand linked to the sense strand.

In some embodiments, a RAGE RNAi agent disclosed herein includes an antisense strand that consists of, consists essentially of, or comprises a nucleobase sequence differing by 0 or 1 nucleobases from the nucleotide sequence (5′→3′) UUGUGUUCAGUUUCCAUUCCG (SEQ ID NO: 7). In some embodiments, a RAGE RNAi agent disclosed herein includes an antisense strand that consists of, consists essentially of, or comprises a nucleotide sequence differing by no more than 1 nucleotide from the nucleotide sequence (5′→3′) UUGUGUUCAGUUUCCAUUCCG (SEQ ID NO: 7), wherein all or substantially all of the nucleotides are modified nucleotides. In some embodiments, a RAGE RNAi agent disclosed herein includes an antisense strand that consists of, consists essentially of, or comprises a nucleobase sequence differing by 0 or 1 nucleobases from the nucleotide sequence (5′→3′) UUGUGUUCAGUUUCCAUUCCG (SEQ ID NO: 7), wherein SEQ ID NO: 7 is located at positions 1-21 (5′→3′) of the antisense strand.

In some embodiments, a RAGE RNAi agent disclosed herein includes an antisense strand that consists of, consists essentially of, or comprises a modified nucleotide sequence differing by no more than 1 nucleotide from the nucleotide sequence (5′→3′) usUfsgsUfgUfuCfaGfuUfuCfcAfuUfcCfsg (SEQ ID NO: 2), wherein a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; and s represents a phosphorothioate linkage, and wherein the sense strand is at least substantially complementary to the antisense strand. As the person of ordinary skill in the art would clearly understand, the inclusion of a phosphorothioate linkage as shown in the modified nucleotide sequences disclosed herein replaces the phosphodiester linkage typically present in oligonucleotides (see, e.g., FIGS. 11A through 11J showing all internucleoside linkages). In some embodiments, a RAGE RNAi agent disclosed herein includes an antisense strand that consists of, consists essentially of, or comprises the nucleotide sequence (5′→3′) usUfsgsUfgUfuCfaGfuUfuCfcAfuUfcCfsg (SEQ ID NO: 2), wherein a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; and s represents a phosphorothioate linkage, and wherein the sense strand is at least substantially complementary to the antisense strand.

In some embodiments, a RAGE RNAi agent disclosed herein includes an antisense strand that consists of, consists essentially of, or comprises a modified nucleotide sequence differing by no more than 1 nucleotide from the nucleotide sequence (5′→3′) cPrpusUfsgsUfgUfuCfaGfuUfuCfcAfuUfcCfsg (SEQ ID NO: 3), wherein a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; cPrpu represents a 5′-cyclopropyl phosphonate-2′-O-methyluridine; and s represents a phosphorothioate linkage, and wherein the sense strand is at least substantially complementary to the antisense strand. In some embodiments, a RAGE RNAi agent disclosed herein includes an antisense strand that consists of, consists essentially of, or comprises the nucleotide sequence (5′→3′) cPrpusUfsgsUfgUfuCfaGfuUfuCfcAfuUfcCfsg (SEQ ID NO: 3), wherein a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; cPrpu represents a 5′-cyclopropyl phosphonate-2′-O-methyluridine; and s represents a phosphorothioate linkage, and wherein the sense strand is at least substantially complementary to the antisense strand.

In some embodiments, a RAGE RNAi agent disclosed herein includes an antisense strand that consists of, consists essentially of, or comprises a nucleobase sequence differing by 0 or 1 nucleobases from the nucleotide sequence (5′→3′) UUCCAUUCCUGUUCAUUGCCU (SEQ ID NO: 8). In some embodiments, a RAGE RNAi agent disclosed herein includes an antisense strand that consists of, consists essentially of, or comprises a nucleotide sequence differing by no more than 1 nucleotide from the nucleotide sequence (5′→3′) UUCCAUUCCUGUUCAUUGCCU (SEQ ID NO: 8), wherein all or substantially all of the nucleotides are modified nucleotides. In some embodiments, a RAGE RNAi agent disclosed herein includes an antisense strand that consists of, consists essentially of, or comprises a nucleobase sequence differing by 0 or 1 nucleobases from the nucleotide sequence (5′→3′) UUCCAUUCCUGUUCAUUGCCU (SEQ ID NO: 8), wherein SEQ ID NO: 8 is located at positions 1-21 (5′→3′) of the antisense strand.

In some embodiments, a RAGE RNAi agent disclosed herein includes an antisense strand that consists of, consists essentially of, or comprises a modified nucleotide sequence differing by no more than 1 nucleotide from the nucleotide sequence (5′→3′) usUfscsCfaUfuCfcUfgUfuCfaUfuGfcCfsu (SEQ ID NO: 4), wherein a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; and s represents a phosphorothioate linkage, and wherein the sense strand is at least substantially complementary to the antisense strand. As the person of ordinary skill in the art would clearly understand, the inclusion of a phosphorothioate linkage as shown in the modified nucleotide sequences disclosed herein replaces the phosphodiester linkage typically present in oligonucleotides (see, e.g., FIGS. 11A through 11J showing all internucleoside linkages). In some embodiments, a RAGE RNAi agent disclosed herein includes an antisense strand that consists of, consists essentially of, or comprises the nucleotide sequence (5′→3′) usUfscsCfaUfuCfcUfgUfuCfaUfuGfcCfsu (SEQ ID NO: 4), wherein a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; and s represents a phosphorothioate linkage, and wherein the sense strand is at least substantially complementary to the antisense strand.

In some embodiments, a RAGE RNAi agent disclosed herein includes an antisense strand that consists of, consists essentially of, or comprises a nucleobase sequence differing by 0 or 1 nucleobases from the nucleotide sequence (5′ 3′) UGAUGUUUUGAGCACCUACUC (SEQ ID NO: 9). In some embodiments, a RAGE RNAi agent disclosed herein includes an antisense strand that consists of, consists essentially of, or comprises a nucleotide sequence differing by no more than 1 nucleotide from the nucleotide sequence (5′→3′) UGAUGUUUUGAGCACCUACUC (SEQ ID NO: 9), wherein all or substantially all of the nucleotides are modified nucleotides. In some embodiments, a RAGE RNAi agent disclosed herein includes an antisense strand that consists of, consists essentially of, or comprises a nucleobase sequence differing by 0 or 1 nucleobases from the nucleotide sequence (5′→3′) UGAUGUUUUGAGCACCUACUC (SEQ ID NO: 9), wherein SEQ ID NO: 7 is located at positions 1-21 (5′→3′) of the antisense strand.

In some embodiments, a RAGE RNAi agent disclosed herein includes an antisense strand that consists of, consists essentially of, or comprises a modified nucleotide sequence differing by no more than 1 nucleotide from the nucleotide sequence (5′→3′) usGfsasuguuuugaGfcAfcCfuacusc (SEQ ID NO: 5), wherein a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; and s represents a phosphorothioate linkage, and wherein the sense strand is at least substantially complementary to the antisense strand. As the person of ordinary skill in the art would clearly understand, the inclusion of a phosphorothioate linkage as shown in the modified nucleotide sequences disclosed herein replaces the phosphodiester linkage typically present in oligonucleotides (see, e.g., FIGS. 11A through 11J showing all internucleoside linkages). In some embodiments, a RAGE RNAi agent disclosed herein includes an antisense strand that consists of, consists essentially of, or comprises the nucleotide sequence (5′ 3′) usGfsasuguuuugaGfcAfcCfuacusc (SEQ ID NO: 5), wherein a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; and s represents a phosphorothioate linkage, and wherein the sense strand is at least substantially complementary to the antisense strand.

In some embodiments, a RAGE RNAi agent disclosed herein includes an antisense strand that consists of, consists essentially of, or comprises a modified nucleotide sequence differing by no more than 1 nucleotide from the nucleotide sequence (5′→3′) cPrpusGfsasuguuuugaGfcAfcCfuacusc (SEQ ID NO: 6), wherein a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; cPrpu represents a 5′-cyclopropyl phosphonate-2′-O-methyluridine; and s represents a phosphorothioate linkage, and wherein the sense strand is at least substantially complementary to the antisense strand. In some embodiments, a RAGE RNAi agent disclosed herein includes an antisense strand that consists of, consists essentially of, or comprises the nucleotide sequence (5′→3′) cPrpusGfsasuguuuugaGfcAfcCfuacusc (SEQ ID NO: 6), wherein a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; cPrpu represents a 5′-cyclopropyl phosphonate-2′-O-methyluridine; and s represents a phosphorothioate linkage, and wherein the sense strand is at least substantially complementary to the antisense strand.

In some embodiments, a RAGE RNAi agent disclosed herein includes an antisense strand that consists of, consists essentially of, or comprises a nucleotide sequence that differs by 0 or 1 nucleotides from one of the following nucleotide sequences (5′→3′):

(SEQ ID NO: 7)
UUGUGUUCAGUUUCCAUUCCG;
(SEQ ID NO: 8)
UUCCAUUCCUGUUCAUUGCCU;
or
(SEQ ID NO: 9)
UGAUGUUUUGAGCACCUACUC;

wherein the RAGE RNAi agent further includes a sense strand that is at least partially complementary to the antisense strand; and wherein all or substantially all of the nucleotides on both the antisense strand and the sense strand are modified nucleotides.

In some embodiments, a RAGE RNAi agent disclosed herein includes an antisense strand that consists of, consists essentially of, or comprises a nucleotide sequence that differs by 0 or 1 nucleotides from one of the following nucleotide sequences (5′→3′):

(SEQ ID NO: 7)
UUGUGUUCAGUUUCCAUUCCG;
(SEQ ID NO: 8)
UUCCAUUCCUGUUCAUUGCCU;
or
(SEQ ID NO: 9)
UGAUGUUUUGAGCACCUACUC;

wherein the RAGE RNAi agent further includes a sense strand that is at least partially complementary to the antisense strand; wherein all or substantially all of the nucleotides on both the antisense strand and the sense strand are modified nucleotides; and wherein the sense strand further includes inverted abasic residues at the 3′ terminal end and at the 5′ end of the nucleotide sequence, and the sense strand also includes a targeting ligand that is covalently linked to the 5′ terminal end, wherein the targeting ligand includes a compound having affinity for an integrin receptor.

In some embodiments, a RAGE RNAi agent disclosed herein includes an antisense strand that consists of, consists essentially of, or comprises a nucleotide sequence that differs by 0 or 1 nucleotides from one of the following nucleotide sequences (5′→3′):

(SEQ ID NO: 7)
UUGUGUUCAGUUUCCAUUCCG;
(SEQ ID NO: 8)
UUCCAUUCCUGUUCAUUGCCU;
or
(SEQ ID NO: 9)
UGAUGUUUUGAGCACCUACUC;

wherein the RAGE RNAi agent further includes a sense strand that is at least partially complementary to the antisense strand; wherein all or substantially all of the nucleotides on both the antisense strand and the sense strand are modified nucleotides; and wherein the sense strand further includes inverted abasic residues at the 3′ terminal end and at the 5′ end of the nucleotide sequence, and the sense strand also includes a targeting ligand that is covalently linked to the 5′ terminal end, wherein the targeting ligand includes a compound having affinity for an integrin receptor; and wherein the respective antisense strand sequence is located at positions 1-21 of the antisense strand.

In some embodiments, a RAGE RNAi agent disclosed herein includes an antisense strand and a sense strand, wherein the antisense strand and the sense strand consist of, consist essentially of, or comprise nucleotide sequences that differ by 0 or 1 nucleotides from one of the following nucleotide sequence (5′→3′) pairs:

(SEQ ID NO: 7)
UUGUGUUCAGUUUCCAUUCCG
and
(SEQ ID NO: 19)
CGGAAUGGAAACUGAACACAA;
(SEQ ID NO: 8)
UUCCAUUCCUGUUCAUUGCCU
and
(SEQ ID NO: 21)
AGGCAAUGAACAGGAAUIGAA;
or
(SEQ ID NO: 9)
UGAUGUUUUGAGCACCUACUC
and
(SEQ ID NO: 20)
GAGUAGGUGCUCAAAACAUCA;

wherein all or substantially all of the nucleotides on both the antisense strand and the sense strand are modified nucleotides.

In some embodiments, a RAGE RNAi agent disclosed herein includes an antisense strand and a sense strand, wherein the antisense strand and the sense strand consist of, consist essentially of, or comprise nucleotide sequences that differ by 0 or 1 nucleotides from one of the following nucleotide sequences (5′→3′) pairs:

(SEQ ID NO: 7)
UUGUGUUCAGUUUCCAUUCCG
and
(SEQ ID NO: 19)
CGGAAUGGAAACUGAACACAA;
(SEQ ID NO: 8)
UUCCAUUCCUGUUCAUUGCCU
and
(SEQ ID NO: 21)
AGGCAAUGAACAGGAAUIGAA;
or
(SEQ ID NO: 9)
UGAUGUUUUGAGCACCUACUC
and
(SEQ ID NO: 20)
GAGUAGGUGCUCAAAACAUCA;

wherein all or substantially all of the nucleotides on both the antisense strand and the sense strand are modified nucleotides; and wherein the sense strand further includes inverted abasic residues at the 3′ terminal end and at the 5′ end of the nucleotide sequence, and the sense strand also includes a targeting ligand that is covalently linked to the 5′ terminal end, wherein the targeting ligand includes a compound with affinity for an integrin receptor.

In some embodiments, a RAGE RNAi agent disclosed herein includes an antisense strand that consists of, consists essentially of, or comprises a modified nucleotide sequence that differs by 0 or 1 nucleotides from one of the following nucleotide sequences (5′→3′):

(SEQ ID NO: 2)
usUfsgsUfgUfuCfaGfuUfuCfcAfuUfcCfsg;
(SEQ ID NO: 3)
cPrpusUfsgsUfgUfuCfaGfuUfuCfcAfuUfcCfsg;
(SEQ ID NO: 5)
usGfsasuguuuugaGfcAfcCfuacusc;
(SEQ ID NO: 6)
cPrpusGfsasuguuuugaGfcAfcCfuacusc;
(SEQ ID NO: 4)
usUfscsCfaUfuCfcUfgUfuCfaUfuGfcCfsu;

wherein a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; cPrpu represents a 5′-cyclopropyl phosphonate-2′-O-methyluridine; s represents a phosphorothioate linkage; and wherein the RAGE RNAi agent further includes the sense strand that is at least partially complementary to the antisense strand; and wherein all or substantially all of the nucleotides of the sense strand are modified nucleotides.

In some embodiments, a RAGE RNAi agent disclosed herein includes an antisense strand that consists of, consists essentially of, or comprises a modified nucleotide sequence that differs by 0 or 1 nucleotides from one of the following nucleotide sequences (5′→3′):

(SEQ ID NO: 2)
usUfsgsUfgUfuCfaGfuUfuCfcAfuUfcCfsg;
(SEQ ID NO: 3)
cPrpusUfsgsUfgUfuCfaGfuUfuCfcAfuUfcCfsg;
(SEQ ID NO: 5)
usGfsasuguuuugaGfcAfcCfuacusc;
(SEQ ID NO: 6)
cPrpusGfsasuguuuugaGfcAfcCfuacusc;
(SEQ ID NO: 4)
usUfscsCfaUfuCfcUfgUfuCfaUfuGfcCfsu;

wherein the RAGE RNAi agent further includes the sense strand that is at least partially complementary to the antisense strand; wherein all or substantially all of the nucleotides of the sense strand are modified nucleotides; wherein all or substantially all of the nucleotides on both the antisense strand and the sense strand are modified nucleotides; and wherein the sense strand further includes inverted abasic residues at the 3′ terminal end and at the 5′ end of the nucleotide sequence, and the sense strand also includes a targeting ligand that is covalently linked to the 5′ terminal end, wherein the targeting ligand includes a compound with affinity for an integrin receptor.

In some embodiments, a RAGE RNAi agent disclosed herein includes an antisense strand and a sense strand that consists of, consists essentially of, or comprises one of the following nucleotide sequence pairs (5′→3′):

(SEQ ID NO: 2)
usUfsgsUfgUfuCfaGfuUfuCfcAfuUfcCfsg
and
(SEQ ID NO: 13)
csggaauggAfAfAfcugaacacaa;
(SEQ ID NO: 3)
cPrpusUfsgsUfgUfuCfaGfuUfuCfcAfuUfcCfsg
and
(SEQ ID NO: 13)
csggaauggAfAfAfcugaacacaa;
(SEQ ID NO: 5)
usGfsasuguuuugaGfcAfcCfuacusc
and
(SEQ ID NO: 14)
gsaguagGfuGfcUfcaaaacauca;
(SEQ ID NO: 6)
cPrpusGfsasuguuuugaGfcAfcCfuacusc
and
(SEQ ID NO: 14)
gsaguagGfuGfcUfcaaaacauca;
and
(SEQ ID NO: 4)
usUfscsCfaUfuCfcUfgUfuCfaUfuGfcCfsu
and
(SEQ ID NO: 15)
asggcaaugAfAfCfaggaauigaa;

wherein a, c, g, i, and u represent 2′-O-methyl adenosine, cytidine, guanosine, inosine, and uridine, respectively; Af, Cf, Gf, and Uf represent 2′-fluoro adenosine, cytidine, guanosine, and uridine, respectively; cPrpu represents a 5′-cyclopropyl phosphonate-2′-O-methyluridine; Tri-SM6.1-αvβ6-(TA14) represents the tridentate αvβ6 epithelial cell targeting ligand with the chemical structure as shown in FIG. 1; and s represents a phosphorothioate linkage; and wherein the sense strand also includes a targeting ligand having affinity for an integrin receptor, wherein the targeting ligand is optionally linked at the 5′-end of the sense strand.

In some embodiments, a RAGE RNAi agent disclosed herein includes an antisense strand and a sense strand that consists of, consists essentially of, or comprises modified nucleotide sequences that differs by 0 or 1 nucleotides from one of the following sequence pairs (5′→3′):

(SEQ ID NO: 2)
usUfsgsUfgUfuCfaGfuUfuCfcAfuUfcCfsg
and
(SEQ ID NO: 10)
Tri-SM6.1-αvβ6-(TA14)csggaauggAf
AfAfcugaacacaas(invAb);
(SEQ ID NO: 3)
cPrpusUfsgsUfgUfuCfaGfuUfuCfcAfuUfcCfsg
and
(SEQ ID NO: 10)
Tri-SM6.1-αvβ6-(TA14)csggaauggAfAfAf
cugaacacaas(invAb);
(SEQ ID NO: 5)
usGfsasuguuuugaGfcAfcCfuacusc
and
(SEQ ID NO: 11)
Tri-SM6.1-αvβ6-(TA14)gsaguagGfuG
fcUfcaaaacaucas(invAb);
(SEQ ID NO: 6)
cPrpusGfsasuguuuugaGfcAfcCfuacusc
and
(SEQ ID NO: 11)
Tri-SM6.1-αvβ6-(TA14)gsaguagGfuGf
cUfcaaaacaucas(invAb);
and
(SEQ ID NO: 4)
usUfscsCfaUfuCfcUfgUfuCfaUfuGfcCfsu
and
(SEQ ID NO: 12)
Tri-SM6.1-αvβ6-(TA14)asggcaaugAfAfC
faggaauigaas(invAb);

wherein a, c, g, i, and u represent 2′-O-methyl adenosine, cytidine, guanosine, inosine, and uridine, respectively; Af, Cf, Gf, and Uf represent 2′-fluoro adenosine, cytidine, guanosine, and uridine, respectively; cPrpu represents a 5′-cyclopropyl phosphonate-2′-O-methyluridine; Tri-SM6.1-αvβ6-(TA14) represents the tridentate αvβ6 epithelial cell targeting ligand with the chemical structure as shown in FIG. 1; (invAb) represents an inverted abasic deoxyribonucleotide (see also Table 11), and s represents a phosphorothioate linkage.

In some embodiments, a RAGE RNAi agent disclosed herein includes an antisense strand that includes a nucleobase sequence that differs by 0 or 1 nucleobases from the nucleotide sequences selected from the group consisting of (5′→3′):

(SEQ ID NO: 55)
UUGUGUUCAGUUUCCAUUC;
(SEQ ID NO: 69)
UUCCAUUCCUGUUCAUUGC;
and
(SEQ ID NO: 65)
UGAUGUUUUGAGCACCUAC.

In some embodiments, a RAGE RNAi agent disclosed herein includes an antisense strand that includes a nucleobase sequence that differs by 0 or 1 nucleobases from the nucleotide sequences selected from the group consisting of (5′→3′):

(SEQ ID NO: 55)
UUGUGUUCAGUUUCCAUUC;
(SEQ ID NO: 69)
UUCCAUUCCUGUUCAUUGC;
and
(SEQ ID NO: 65)
UGAUGUUUUGAGCACCUAC.

wherein all or substantially all of the nucleotides are modified nucleotides.

In some embodiments, a RAGE RNAi agent disclosed herein includes an antisense strand that includes a nucleobase sequence that differs by 0 or 1 nucleobases from the nucleotide sequences selected from the group consisting of (5′→3′):

(SEQ ID NO: 55)
UUGUGUUCAGUUUCCAUUC;
(SEQ ID NO: 69)
UUCCAUUCCUGUUCAUUGC;
and
(SEQ ID NO: 65)
UGAUGUUUUGAGCACCUAC.

wherein all or substantially all of the nucleotides are modified nucleotides, and wherein SEQ ID NO: 55, SEQ ID NO: 69 and SEQ ID NO: 65, respectively, is located at nucleotide positions 1-19 (5′→3′) of the antisense strand.

In some embodiments, a RAGE RNAi agent disclosed herein includes an antisense strand and a sense strand that each include a nucleobase sequences that differs by 0 or 1 nucleobases from the nucleotide sequence pairs selected from the group consisting of (5′→3′):

(SEQ ID NO: 55)
UUGUGUUCAGUUUCCAUUC
and
(SEQ ID NO: 298)
GAAUGGAAACUGAACACAA;
(SEQ ID NO: 69)
UUCCAUUCCUGUUCAUUGC;
and
(SEQ ID NO: 316)
GCAAUGAACAGGAAUIGAA;
or
(SEQ ID NO: 65)
UGAUGUUUUGAGCACCUAC
and
(SEQ ID NO: 308)
GUAGGUGCUCAAAACAUCA.

In some embodiments, a RAGE RNAi agent disclosed herein includes an antisense strand and a sense strand that each include a nucleobase sequences that differs by 0 or 1 nucleobases from the nucleotide sequence pairs selected from the group consisting of (5′→3′):

(SEQ ID NO: 55)
UUGUGUUCAGUUUCCAUUC
and
(SEQ ID NO: 298)
GAAUGGAAACUGAACACAA;
(SEQ ID NO: 69)
UUCCAUUCCUGUUCAUUGC;
and
(SEQ ID NO: 316)
GCAAUGAACAGGAAUIGAA;
or
(SEQ ID NO: 65)
UGAUGUUUUGAGCACCUAC
and
(SEQ ID NO: 308)
GUAGGUGCUCAAAACAUCA

### Definitions

As used herein, the terms “oligonucleotide” and “polynucleotide” mean a polymer of linked nucleosides each of which can be independently modified or unmodified.

As used herein, an “RNAi agent” (also referred to as an “RNAi trigger”) means a composition that contains an RNA or RNA-like (e.g., chemically modified RNA) oligonucleotide molecule that is capable of degrading or inhibiting (e.g., degrades or inhibits under appropriate conditions) translation of messenger RNA (mRNA) transcripts of a target gene in a sequence specific manner. As used herein, RNAi agents may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents disclosed herein are comprised of a sense strand and an antisense strand, and include, but are not limited to: short (or small) interfering RNAs (siRNAs), double stranded RNAs (dsRNA), micro RNAs (miRNAs), short hairpin RNAs (shRNA), and dicer substrates. The antisense strand of the RNAi agents described herein is at least partially complementary to the mRNA being targeted (i.e., AGER mRNA). RNAi agents can include one or more modified nucleotides and/or one or more non-phosphodiester linkages.

As used herein, the terms “silence,” “reduce,” “inhibit,” “down-regulate,” or “knockdown” when referring to expression of a given gene, mean that the expression of the gene, as measured by the level of RNA transcribed from the gene or the level of polypeptide, protein, or protein subunit translated from the mRNA in a cell, group of cells, tissue, organ, or subject in which the gene is transcribed, is reduced when the cell, group of cells, tissue, organ, or subject is treated with the RNAi agents described herein as compared to a second cell, group of cells, tissue, organ, or subject that has not or have not been so treated.

As used herein, the terms “sequence” and “nucleotide sequence” mean a succession or order of nucleobases or nucleotides, described with a succession of letters using standard nomenclature.

As used herein, a “base,” “nucleotide base,” or “nucleobase,” is a heterocyclic pyrimidine or purine compound that is a component of a nucleotide, and includes the primary purine bases adenine and guanine, and the primary pyrimidine bases cytosine, thymine, and uracil. A nucleobase may further be modified to include, without limitation, universal bases, hydrophobic bases, promiscuous bases, size-expanded bases, and fluorinated bases. (See, e.g., Modified Nucleosides in Biochemistry, Biotechnology and Medicine, Herdewijn, P. ed. Wiley-VCH, 2008). The synthesis of such modified nucleobases (including phosphoramidite compounds that include modified nucleobases) is known in the art.

As used herein, and unless otherwise indicated, the term “complementary,” when used to describe a first nucleobase or nucleotide sequence (e.g., RNAi agent sense strand or targeted mRNA) in relation to a second nucleobase or nucleotide sequence (e.g., RNAi agent antisense strand or a single-stranded antisense oligonucleotide), means the ability of an oligonucleotide or polynucleotide including the first nucleotide sequence to hybridize (form base pair hydrogen bonds under mammalian physiological conditions (or otherwise suitable in vivo or in vitro conditions)) and form a duplex or double helical structure under certain standard conditions with an oligonucleotide that includes the second nucleotide sequence. The person of ordinary skill in the art would be able to select the set of conditions most appropriate for a hybridization test. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification. For example, a and Af, as defined herein, are complementary to U (or T) and identical to A for the purposes of determining identity or complementarity.

As used herein, “perfectly complementary” or “fully complementary” means that in a hybridized pair of nucleobase or nucleotide sequence molecules, all (100%) of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “partially complementary” means that in a hybridized pair of nucleobase or nucleotide sequence molecules, at least 70%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “substantially complementary” means that in a hybridized pair of nucleobase or nucleotide sequence molecules, at least 85%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, the terms “complementary,” “fully complementary,” “partially complementary,” and “substantially complementary” are used with respect to the nucleobase or nucleotide matching between the sense strand and the antisense strand of an RNAi agent, or between the antisense strand of an RNAi agent and a sequence of an AGER mRNA.

As used herein, the term “substantially identical” or “substantial identity,” as applied to a nucleic acid sequence means the nucleotide sequence (or a portion of a nucleotide sequence) has at least about 85% sequence identity or more, e.g., at least 90%, at least 95%, or at least 99% identity, compared to a reference sequence. Percentage of sequence identity is determined by comparing two optimally aligned sequences over a comparison window. The percentage is calculated by determining the number of positions at which the same type of nucleic acid base occurs in both sequences to yield the number of matched positions, dividing the number of matched positions by the total number of positions in the window of comparison and multiplying the result by 100 to yield the percentage of sequence identity. The inventions disclosed herein encompass nucleotide sequences substantially identical to those disclosed herein.

As used herein, the terms “treat,” “treatment,” and the like, mean the methods or steps taken to provide relief from or alleviation of the number, severity, and/or frequency of one or more symptoms of a disease in a subject. As used herein, “treat” and “treatment” may include the prevention, management, prophylactic treatment, and/or inhibition or reduction of the number, severity, and/or frequency of one or more symptoms of a disease in a subject.

As used herein, the phrase “introducing into a cell,” when referring to an RNAi agent, means functionally delivering the RNAi agent into a cell. The phrase “functional delivery,” means delivering the RNAi agent to the cell in a manner that enables the RNAi agent to have the expected biological activity, e.g., sequence-specific inhibition of gene expression.

Unless stated otherwise, use of the symbol [symbol] as used herein means that any group or groups may be linked thereto that is in accordance with the scope of the inventions described herein.

As used herein, the term “isomers” refers to compounds that have identical molecular formulae, but that differ in the nature or the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images are termed “enantiomers,” or sometimes optical isomers. A carbon atom bonded to four non-identical substituents is termed a “chiral center.”

As used herein, unless specifically identified in a structure as having a particular conformation, for each structure in which asymmetric centers are present and thus give rise to enantiomers, diastereomers, or other stereoisomeric configurations, each structure disclosed herein is intended to represent all such possible isomers, including their optically pure and racemic forms. For example, the structures disclosed herein are intended to cover mixtures of diastereomers as well as single stereoisomers.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

The person of ordinary skill in the art would readily understand and appreciate that the compounds and compositions disclosed herein may have certain atoms (e.g., N, O, or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the environment (such as pH), as would be readily understood by the person of ordinary skill in the art. Correspondingly, compounds described herein with labile protons or basic atoms should also be understood to represent salt forms of the corresponding compound. Compounds described herein may be in a free acid, free base, or salt form. Pharmaceutically acceptable salts of the compounds described herein should be understood to be within the scope of the invention.

As used herein, the term “linked” or “conjugated” when referring to the connection between two compounds or molecules means that two compounds or molecules are joined by a covalent bond. Unless stated, the terms “linked” and “conjugated” as used herein may refer to the connection between a first compound and a second compound either with or without any intervening atoms or groups of atoms.

As used herein, the term “including” is used to herein mean, and is used interchangeably with, the phrase “including but not limited to.” The term “or” is used herein to mean, and is used interchangeably with, the term “and/or,” unless the context clearly indicates otherwise.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other objects, features, aspects, and advantages of the invention will be apparent from the following detailed description, accompanying figures, and from the claims.

### BRIEF DESCRIPTION OF THE DRAWINGS

FIG. 1. Chemical structure representation of the tridentate αvβ6 epithelial cell targeting ligand referred to herein as Tri-SM6.1-αvβ6-(TA14).

FIG. 2. Chemical structure representation of the peptide αvβ6 epithelial cell targeting ligand referred to herein as αvβ6-pep1.

FIG. 3A to 3E. Chemical structure representation of RAGE RNAi agent conjugate AC000292 (AM10309-AS (SEQ ID NO: 3), CS000363 (SEQ ID NO: 10)) shown as a free acid.

FIG. 4A to 4E. Chemical structure representation of RAGE RNAi agent conjugate AC000292 (AM10309-AS (SEQ ID NO: 3), CS000363 (SEQ ID NO: 10)) shown as a sodium salt.

FIG. 5A to 5E. Chemical structure representation of RAGE RNAi agent conjugate AC001266 (AM11897-AS (SEQ ID NO: 5), CS001579 (SEQ ID NO: 11)) shown as a free acid.

FIG. 6A to 6E. Chemical structure representation of RAGE RNAi agent conjugate AC001266 (AM11897-AS (SEQ ID NO: 5), CS001579 (SEQ ID NO: 11)) shown as a sodium salt.

FIG. 7A to 7E. Chemical structure representation of RAGE RNAi agent conjugate AC001267 (AM11898-AS (SEQ ID NO: 6), CS001579 (SEQ ID NO: 11)) shown as a free acid.

FIG. 8A to 8E. Chemical structure representation of RAGE RNAi agent conjugate AC001267 (AM11898-AS (SEQ ID NO: 6), CS001579 (SEQ ID NO: 11)) shown as a sodium salt.

FIG. 9A to 9E. Chemical structure representation of RAGE RNAi agent conjugate AC001268 (AM10754-AS (SEQ ID NO: 4), CS001582 (SEQ ID NO: 12)) shown as a free acid.

FIG. 10A to 10E. Chemical structure representation of RAGE RNAi agent conjugate AC001268 (AM10754-AS (SEQ ID NO: 4), CS001582 (SEQ ID NO: 12)) shown as a sodium salt.

FIG. 11A. Schematic diagram of the modified sense and antisense strands of the RAGE RNAi agent conjugate having the structure of AC000286 (AM10308-AS (SEQ ID NO: 2), CS000363 (SEQ ID NO: 10)) (see, e.g., Tables 8 and 10), having a tridentate αvβ6 epithelial cell targeting ligand linked at the 5′ end of the sense strand.

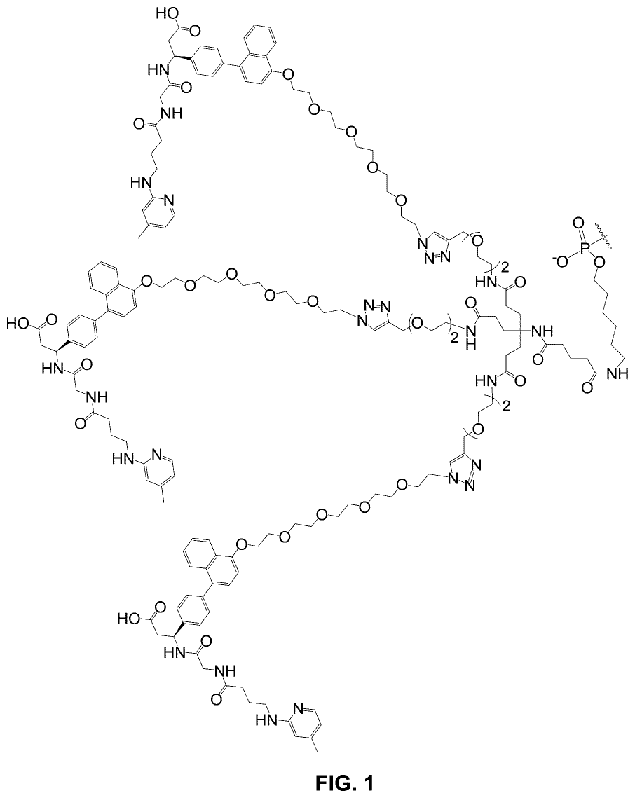

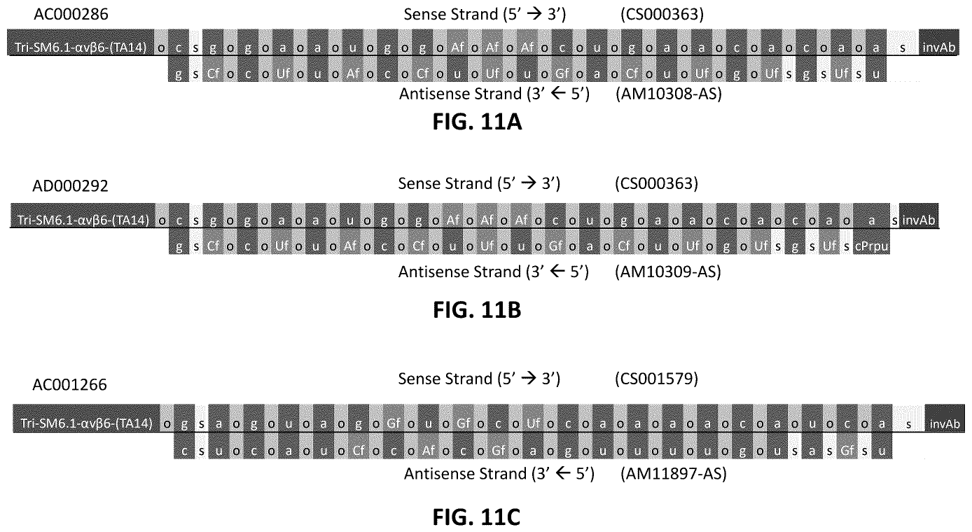

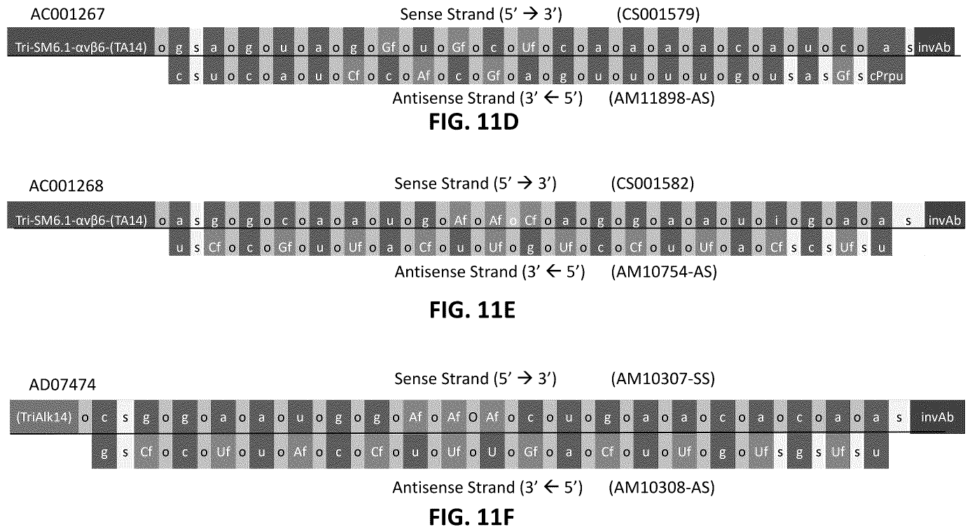

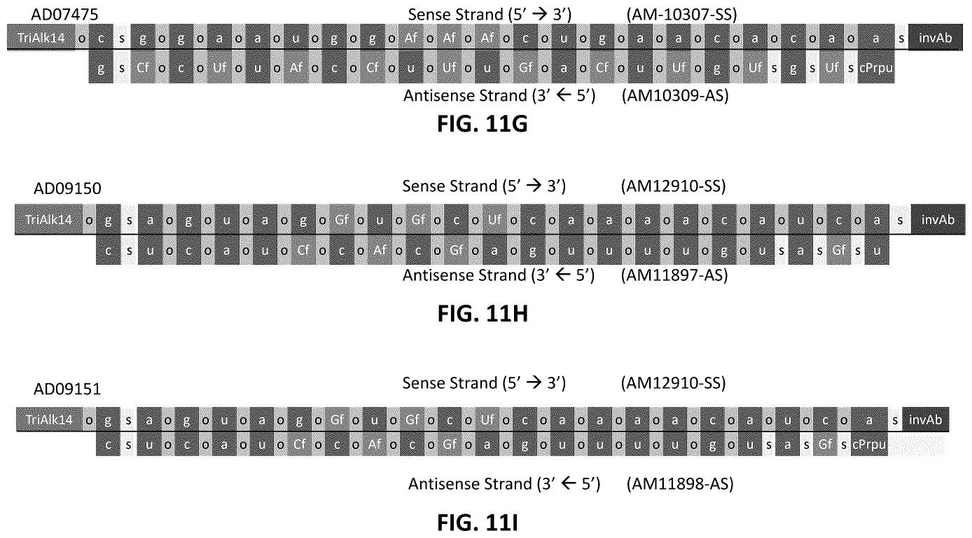

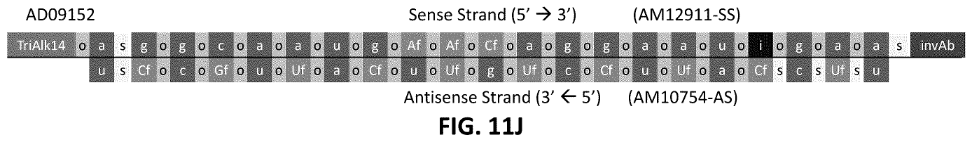

The following abbreviations are used in FIGS. 11A to 11J: a, c, g, i, and u are 2′-O-methyl modified nucleotides; Af, Cf, Gf, and Uf are 2′-fluoro modified nucleotides; o is a phosphodiester linkage; s is a phosphorothioate linkage; invAb is an inverted abasic residue (see, e.g., Table 11); cPrpu is a 5′-cyclopropyl phosphonate-2′-O-methyluridine modified nucleotide (see, e.g., Table 11); Tri-SM6.1-αvβ6-(TA14) is the tridentate αvβ6 epithelial cell targeting ligand having the structure shown in FIG. 1; and (TriAlk14) is the linking group as shown in Table 11, which is suitable for subsequent coupling to targeting ligands (See also, Example 1 herein).

FIG. 11B. Schematic diagram of the modified sense and antisense strands of the RAGE RNAi agent conjugate having the structure of AC000292 (AM10309-AS (SEQ ID NO: 3), CS000363 (SEQ ID NO: 10)) (see, e.g., Tables 8 and 10), having a tridentate αvβ6 epithelial cell targeting ligand linked at the 5′ end of the sense strand.

FIG. 11C. Schematic diagram of the modified sense and antisense strands of the RAGE RNAi agent conjugate having the structure of AC001266 (AM11897-AS (SEQ ID NO: 5), CS001579 (SEQ ID NO: 11)) (see, e.g., Tables 8 and 10), having a tridentate αvβ6 epithelial cell targeting ligand linked at the 5′ end of the sense strand.

FIG. 11D. Schematic diagram of the modified sense and antisense strands of the RAGE RNAi agent conjugate having the structure of AC001267 (AM11898-AS (SEQ ID NO: 6), CS001579 (SEQ ID NO: 11)) (see, e.g., Tables 8 and 10), having a tridentate αvβ6 epithelial cell targeting ligand linked at the 5′ end of the sense strand.

FIG. 11E. Schematic diagram of the modified sense and antisense strands of the RAGE RNAi agent conjugate having the structure of AC001268 (AM10754-AS (SEQ ID NO: 4), CS001582 (SEQ ID NO: 12)) (see, e.g., Tables 8 and 10), having a tridentate αvβ6 epithelial cell targeting ligand linked at the 5′ end of the sense strand.

FIG. 11F. Schematic diagram of the modified sense and antisense strands of the RAGE RNAi agent duplex having the structure of AD07474 (AM10308-AS (SEQ ID NO: 2), AM10307-SS (SEQ ID NO: 16)) (see, e.g., Table 7B), having a (TriAlk14) linker at the 5′ end of the sense strand.

FIG. 11G. Schematic diagram of the modified sense and antisense strands of the RAGE RNAi agent duplex having the structure of AD07475 (AM10309-AS (SEQ ID NO: 3), AM10307-SS (SEQ ID NO: 16)) (see, e.g., Table 7B), having a (TriAlk14) linker at the 5′ end of the sense strand.

FIG. 11H. Schematic diagram of the modified sense and antisense strands of the RAGE RNAi agent duplex having the structure of AD09150 (AM11897-AS (SEQ ID NO: 5), AM12910-SS (SEQ ID NO: 17)) (see, e.g., Table 7B), having a (TriAlk14) linker at the 5′ end of the sense strand.

FIG. 11I. Schematic diagram of the modified sense and antisense strands of the RAGE RNAi agent duplex having the structure of AD09151 (AM11898-AS (SEQ ID NO: 6), AM12910-SS (SEQ ID NO: 17)) (see, e.g., Table 7B), having a (TriAlk14) linker at the 5′ end of the sense strand.

FIG. 11J. Schematic diagram of the modified sense and antisense strands of the RAGE RNAi agent duplex having the structure of AD09152 (AM10754-AS (SEQ ID NO: 4), AM12911-SS (SEQ ID NO: 18)) (see, e.g., Table 7B), having a (TriAlk14) linker at the 5′ end of the sense strand.

### DETAILED DESCRIPTION

### RNAi Agents

Described herein are RNAi agents for inhibiting expression of the AGER (or RAGE) gene (referred to herein as RAGE RNAi agents or RAGE RNAi triggers). Each RAGE RNAi agent disclosed herein comprises a sense strand and an antisense strand. The length of the RNAi agent sense strands described herein each can be 15 to 49 nucleotides in length. The length of the RNAi agent antisense strands described herein each can be 18 to 49 nucleotides in length. In some embodiments, the sense and antisense strands are independently 18 to 26 nucleotides in length. The sense and antisense strands can be either the same length or different lengths. In some embodiments, the sense and antisense strands are independently 21 to 26 nucleotides in length. In some embodiments, the sense and antisense strands are independently 21 to 24 nucleotides in length. In some embodiments, both the sense strand and the antisense strand are 21 nucleotides in length. In some embodiments, the antisense strands are independently 18, 19, 20, 21, 22, 23, 24, 25, 26, 27, 28, 29, or 30 nucleotides in length. In some embodiments, the sense strands are independently 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides in length. In some embodiments, a double-stranded RNAi agent has a duplex length of about 16, 17, 18, 19, 20, 21, 22, 23 or 24 nucleotides.

Examples of nucleotide sequences used in forming RAGE RNAi agents are provided in Tables 2, 3, 4, 5, 6, and 10. Examples of RNAi agent duplexes, that include the sense strand and antisense strand sequences in Tables 2, 3, 4, 5, 6, are shown in Tables 7A, 7B, 8, 9A, 9B, and 10.

In some embodiments, the region of perfect, substantial, or partial complementarity between the sense strand and the antisense strand is 15-26 (e.g., 15, 16, 17, 18, 19, 20, 21, 22, 23, 24, 25, or 26) nucleotides in length and occurs at or near the 5′ end of the antisense strand (e.g., this region may be separated from the 5′ end of the antisense strand by 0, 1, 2, 3, or 4 nucleotides that are not perfectly, substantially, or partially complementary).

A sense strand of the RAGE RNAi agents described herein includes at least 15 consecutive nucleotides that have at least 85% identity to a core stretch sequence (also referred to herein as a “core stretch” or “core sequence”) of the same number of nucleotides in an AGER mRNA. In some embodiments, a sense strand core stretch sequence is 100% (perfectly) complementary or at least about 85% (substantially) complementary to a core stretch sequence in the antisense strand, and thus the sense strand core stretch sequence is typically perfectly identical or at least about 85% identical to a nucleotide sequence of the same length (sometimes referred to, e.g., as a target sequence) present in the AGER mRNA target. In some embodiments, this sense strand core stretch is 15, 16, 17, 18, 19, 20, 21, 22, or 23 nucleotides in length. In some embodiments, this sense strand core stretch is 17 nucleotides in length. In some embodiments, this sense strand core stretch is 19 nucleotides in length.

An antisense strand of a RAGE RNAi agent described herein includes at least 15 consecutive nucleotides that have at least 85% complementarity to a core stretch of the same number of nucleotides in an AGER mRNA and to a core stretch of the same number of nucleotides in the corresponding sense strand. In some embodiments, an antisense strand core stretch is 100% (perfectly) complementary or at least about 85% (substantially) complementary to a nucleotide sequence (e.g., target sequence) of the same length present in the AGER mRNA target. In some embodiments, this antisense strand core stretch is 15, 16, 17, 18, 19, 20, 21, 22, 23, 24, 25, or 26 nucleotides in length. In some embodiments, this antisense strand core stretch is 19 nucleotides in length. In some embodiments, this antisense strand core stretch is 17 nucleotides in length. A sense strand core stretch sequence can be the same length as a corresponding antisense core sequence or it can be a different length.

The RAGE RNAi agent sense and antisense strands anneal to form a duplex. A sense strand and an antisense strand of a RAGE RNAi agent can be partially, substantially, or fully complementary to each other. Within the complementary duplex region, the sense strand core stretch sequence is at least 85% complementary or 100% complementary to the antisense core stretch sequence. In some embodiments, the sense strand core stretch sequence contains a sequence of at least 15, at least 16, at least 17, at least 18, at least 19, at least 20, at least 21, at least 22, or at least 23 nucleotides that is at least 85% or 100% complementary to a corresponding 15, 16, 17, 18, 19, 20, 21, 22, or 23 nucleotide sequence of the antisense strand core stretch sequence (i.e., the sense and antisense core stretch sequences of a RAGE RNAi agent have a region of at least 15, at least 16, at least 17, at least 18, at least 19, at least 20, at least 21, at least 22, or at least 23 nucleotides that is at least 85% base paired or 100% base paired.)

In some embodiments, the antisense strand of a RAGE RNAi agent disclosed herein differs by 0, 1, 2, or 3 nucleotides from any of the antisense strand sequences in Table 2 or Table 3. In some embodiments, the sense strand of a RAGE RNAi agent disclosed herein differs by 0, 1, 2, or 3 nucleotides from any of the sense strand sequences in Table 2, Table 4, Table 5, Table 6, or Table 10.

In some embodiments, the sense strand and/or the antisense strand can optionally and independently contain an additional 1, 2, 3, 4, 5, or 6 nucleotides (extension) at the 3′ end, the 5′ end, or both the 3′ and 5′ ends of the core stretch sequences. The antisense strand additional nucleotides, if present, may or may not be complementary to the corresponding sequence in the AGER mRNA. The sense strand additional nucleotides, if present, may or may not be identical to the corresponding sequence in the AGER mRNA. The antisense strand additional nucleotides, if present, may or may not be complementary to the corresponding sense strand's additional nucleotides, if present.

As used herein, an extension comprises 1, 2, 3, 4, 5, or 6 nucleotides at the 5′ and/or 3′ end of the sense strand core stretch sequence and/or antisense strand core stretch sequence. The extension nucleotides on a sense strand may or may not be complementary to nucleotides, either core stretch sequence nucleotides or extension nucleotides, in the corresponding antisense strand. Conversely, the extension nucleotides on an antisense strand may or may not be complementary to nucleotides, either core stretch nucleotides or extension nucleotides, in the corresponding sense strand. In some embodiments, both the sense strand and the antisense strand of an RNAi agent contain 3′ and 5′ extensions. In some embodiments, one or more of the 3′ extension nucleotides of one strand base pairs with one or more 5′ extension nucleotides of the other strand. In other embodiments, one or more of 3′ extension nucleotides of one strand do not base pair with one or more 5′ extension nucleotides of the other strand. In some embodiments, a RAGE RNAi agent has an antisense strand having a 3′ extension and a sense strand having a 5′ extension. In some embodiments, the extension nucleotide(s) are unpaired and form an overhang. As used herein, an “overhang” refers to a stretch of one or more unpaired nucleotides located at a terminal end of either the sense strand or the antisense strand that does not form part of the hybridized or duplexed portion of an RNAi agent disclosed herein.

In some embodiments, a RAGE RNAi agent comprises an antisense strand having a 3′ extension of 1, 2, 3, 4, 5, or 6 nucleotides in length. In other embodiments, a RAGE RNAi agent comprises an antisense strand having a 3′ extension of 1, 2, or 3 nucleotides in length. In some embodiments, one or more of the antisense strand extension nucleotides comprise nucleotides that are complementary to the corresponding AGER mRNA sequence. In some embodiments, one or more of the antisense strand extension nucleotides comprise nucleotides that are not complementary to the corresponding AGER mRNA sequence.

In some embodiments, a RAGE RNAi agent comprises a sense strand having a 3′ extension of 1, 2, 3, 4, or 5 nucleotides in length. In some embodiments, one or more of the sense strand extension nucleotides comprises adenosine, uracil, or thymidine nucleotides, AT dinucleotide, or nucleotides that correspond to or are the identical to nucleotides in the AGER mRNA sequence. In some embodiments, the 3′ sense strand extension includes or consists of one of the following sequences, but is not limited to: T, UT, TT, UU, UUT, TTT, or TTTT (each listed 5′ to 3′).

A sense strand can have a 3′ extension and/or a 5′ extension. In some embodiments, a RAGE RNAi agent comprises a sense strand having a 5′ extension of 1, 2, 3, 4, 5, or 6 nucleotides in length. In some embodiments, one or more of the sense strand extension nucleotides comprise nucleotides that correspond to or are identical to nucleotides in the AGER mRNA sequence.

Examples of sequences used in forming RAGE RNAi agents are provided in Tables 2, 3, 4, 5, 6, and 10. In some embodiments, a RAGE RNAi agent antisense strand includes a sequence of any of the sequences in Tables 2, 3, or 10. In certain embodiments, a RAGE RNAi agent antisense strand comprises or consists of any one of the modified sequences in Table 3. In some embodiments, a RAGE RNAi agent antisense strand includes the sequence of nucleotides (from 5′ end→3′ end) 1-17, 2-15, 2-17, 1-18, 2-18, 1-19, 2-19, 1-20, 2-20, 1-21, or 2-21, of any of the sequences in Tables 2 or 3. In some embodiments, a RAGE RNAi agent sense strand includes the sequence of any of the sequences in Tables 2, 4, 5, or 6. In some embodiments, a RAGE RNAi agent sense strand includes the sequence of nucleotides (from 5′ end→3′ end) 1-18, 1-19, 1-20, 1-21, 2-19, 2-20, 2-21, 3-20, 3-21, or 4-21 of any of the sequences in Tables 2, 4, 5, or 6. In certain embodiments, a RAGE RNAi agent sense strand comprises or consists of a modified sequence of any one of the modified sequences in Table 4, 5, 6, or 10.

In some embodiments, the sense and antisense strands of the RNAi agents described herein contain the same number of nucleotides. In some embodiments, the sense and antisense strands of the RNAi agents described herein contain different numbers of nucleotides. In some embodiments, the sense strand 5′ end and the antisense strand 3′ end of an RNAi agent form a blunt end. In some embodiments, the sense strand 3′ end and the antisense strand 5′ end of an RNAi agent form a blunt end. In some embodiments, both ends of an RNAi agent form blunt ends. In some embodiments, neither end of an RNAi agent is blunt-ended. As used herein a “blunt end” refers to an end of a double stranded RNAi agent in which the terminal nucleotides of the two annealed strands are complementary (form a complementary base-pair).

In some embodiments, the sense strand 5′ end and the antisense strand 3′ end of an RNAi agent form a frayed end. In some embodiments, the sense strand 3′ end and the antisense strand 5′ end of an RNAi agent form a frayed end. In some embodiments, both ends of an RNAi agent form a frayed end. In some embodiments, neither end of an RNAi agent is a frayed end. As used herein a frayed end refers to an end of a double stranded RNAi agent in which the terminal nucleotides of the two annealed strands form a pair (i.e., do not form an overhang) but are not complementary (i.e. form a non-complementary pair). In some embodiments, one or more unpaired nucleotides at the end of one strand of a double stranded RNAi agent form an overhang. The unpaired nucleotides may be on the sense strand or the antisense strand, creating either 3′ or 5′ overhangs. In some embodiments, the RNAi agent contains: a blunt end and a frayed end, a blunt end and 5′ overhang end, a blunt end and a 3′ overhang end, a frayed end and a 5′ overhang end, a frayed end and a 3′ overhang end, two 5′ overhang ends, two 3′ overhang ends, a 5′ overhang end and a 3′ overhang end, two frayed ends, or two blunt ends. Typically, when present, overhangs are located at the 3′ terminal ends of the sense strand, the antisense strand, or both the sense strand and the antisense strand.

The RAGE RNAi agents disclosed herein may also be comprised of one or more modified nucleotides. In some embodiments, substantially all of the nucleotides of the sense strand and substantially all of the nucleotides of the antisense strand of the RAGE RNAi agent are modified nucleotides. The RAGE RNAi agents disclosed herein may further be comprised of one or more modified internucleoside linkages, e.g., one or more phosphorothioate linkages. In some embodiments, a RAGE RNAi agent contains one or more modified nucleotides and one or more modified internucleoside linkages. In some embodiments, a 2′-modified nucleotide is combined with modified internucleoside linkage.

In some embodiments, a RAGE RNAi agent is prepared or provided as a salt, mixed salt, or a free-acid. In some embodiments, a RAGE RNAi agent is prepared as a pharmaceutically acceptable salt. In some embodiments, a RAGE RNAi agent is prepared as a pharmaceutically acceptable sodium salt. Such forms that are well known in the art are within the scope of the inventions disclosed herein.

### Modified Nucleotides

Modified nucleotides, when used in various oligonucleotide constructs, can preserve activity of the compound in cells while at the same time increasing the serum stability of these compounds, and can also minimize the possibility of activating interferon activity in humans upon administration of the oligonucleotide construct.

In some embodiments, a RAGE RNAi agent contains one or more modified nucleotides. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides can include, but are not limited to, deoxyribonucleotides, nucleotide mimics, abasic nucleotides, 2′-modified nucleotides, inverted nucleotides, modified nucleobase-comprising nucleotides, bridged nucleotides, peptide nucleic acids (PNAs), 2′,3′-seco nucleotide mimics (unlocked nucleobase analogues), locked nucleotides, 3′-O-methoxy (2′ internucleoside linked) nucleotides, 2′-F-Arabino nucleotides, 5′-Me, 2′-fluoro nucleotide, morpholino nucleotides, vinyl phosphonate deoxyribonucleotides, vinyl phosphonate containing nucleotides, and cyclopropyl phosphonate containing nucleotides. 2′-modified nucleotides (i.e., a nucleotide with a group other than a hydroxyl group at the 2′ position of the five-membered sugar ring) include, but are not limited to, 2′-O-methyl nucleotides (also referred to as 2′-methoxy nucleotides), 2′-fluoro nucleotides (also referred to herein and in the art as 2′-deoxy-2′-fluoro nucleotides), 2′-deoxy nucleotides, 2′-methoxyethyl(2′-O-2-methoxylethyl) nucleotides (also referred to as 2′-MOE), 2′-amino nucleotides, and 2′-alkyl nucleotides. It is not necessary for all positions in a given compound to be uniformly modified. Conversely, more than one modification can be incorporated in a single RAGE RNAi agent or even in a single nucleotide thereof. The RAGE RNAi agent sense strands and antisense strands can be synthesized and/or modified by methods known in the art. Modification at one nucleotide is independent of modification at another nucleotide.

Modified nucleobases include synthetic and natural nucleobases, such as 5-substituted pyrimidines, 6-azapyrimidines and N-2, N-6 and 0-6 substituted purines, (e.g., 2-aminopropyladenine, 5-propynyluracil, or 5-propynylcytosine), 5-methylcytosine (5-me-C), 5-hydroxymethyl cytosine, inosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, the 5′ and/or 3′ end of the antisense strand can include abasic residues (Ab), which can also be referred to as an “abasic site” or “abasic nucleotide.” An abasic residue (Ab) is a nucleotide or nucleoside that lacks a nucleobase at the 1′ position of the sugar moiety. (See, e.g., U.S. Pat. No. 5,998,203). In some embodiments, an abasic residue can be placed internally in a nucleotide sequence. In some embodiments, Ab or AbAb can be added to the 3′ end of the antisense strand. In some embodiments, the 5′ end of the sense strand can include one or more additional abasic residues (e.g., (Ab) or (AbAb)). In some embodiments, UUAb, UAb, or Ab are added to the 3′ end of the sense strand. In some embodiments, an abasic (deoxyribose) residue can be replaced with a ribitol (abasic ribose) residue.

In some embodiments, all or substantially all of the nucleotides of an RNAi agent are modified nucleotides. As used herein, an RNAi agent wherein substantially all of the nucleotides present are modified nucleotides is an RNAi agent having four or fewer (i.e., 0, 1, 2, 3, or 4) nucleotides in both the sense strand and the antisense strand being ribonucleotides (i.e., unmodified). As used herein, a sense strand wherein substantially all of the nucleotides present are modified nucleotides is a sense strand having two or fewer (i.e., 0, 1, or 2) nucleotides in the sense strand being unmodified ribonucleotides. As used herein, an antisense sense strand wherein substantially all of the nucleotides present are modified nucleotides is an antisense strand having two or fewer (i.e., 0, 1, or 2) nucleotides in the sense strand being unmodified ribonucleotides. In some embodiments, one or more nucleotides of an RNAi agent is an unmodified ribonucleotide. Chemical structures for certain modified nucleotides are set forth in Table 11 herein.

### Modified Internucleoside Linkages

In some embodiments, one or more nucleotides of a RAGE RNAi agent are linked by non-standard linkages or backbones (i.e., modified internucleoside linkages or modified backbones). Modified internucleoside linkages or backbones include, but are not limited to, phosphorothioate groups (represented herein as a lower case “s”), chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, a sense strand of a RAGE RNAi agent can contain 1, 2, 3, 4, 5, or 6 phosphorothioate linkages, an antisense strand of a RAGE RNAi agent can contain 1, 2, 3, 4, 5, or 6 phosphorothioate linkages, or both the sense strand and the antisense strand independently can contain 1, 2, 3, 4, 5, or 6 phosphorothioate linkages. In some embodiments, a sense strand of a RAGE RNAi agent can contain 1, 2, 3, or 4 phosphorothioate linkages, an antisense strand of a RAGE RNAi agent can contain 1, 2, 3, or 4 phosphorothioate linkages, or both the sense strand and the antisense strand independently can contain 1, 2, 3, or 4 phosphorothioate linkages.

In some embodiments, a RAGE RNAi agent sense strand contains at least two phosphorothioate internucleoside linkages. In some embodiments, the phosphorothioate internucleoside linkages are between the nucleotides at positions 1-3 from the 3′ end of the sense strand. In some embodiments, one phosphorothioate internucleoside linkage is at the 5′ end of the sense strand nucleotide sequence, and another phosphorothioate linkage is at the 3′ end of the sense strand nucleotide sequence. In some embodiments, two phosphorothioate internucleoside linkage are located at the 5′ end of the sense strand, and another phosphorothioate linkage is at the 3′ end of the sense strand. In some embodiments, the sense strand does not include any phosphorothioate internucleoside linkages between the nucleotides, but contains one, two, or three phosphorothioate linkages between the terminal nucleotides on both the 5′ and 3′ ends and the optionally present inverted abasic residue terminal caps. In some embodiments, the targeting ligand is linked to the sense strand via a phosphorothioate linkage.

In some embodiments, a RAGE RNAi agent antisense strand contains four phosphorothioate internucleoside linkages. In some embodiments, the four phosphorothioate internucleoside linkages are between the nucleotides at positions 1-3 from the 5′ end of the antisense strand and between the nucleotides at positions 19-21, 20-22, 21-23, 22-24, 23-25, or 24-26 from the 5′ end. In some embodiments, three phosphorothioate internucleoside linkages are located between positions 1~4 from the 5′ end of the antisense strand, and a fourth phosphorothioate internucleoside linkage is located between positions 20-21 from the 5′ end of the antisense strand. In some embodiments, a RAGE RNAi agent contains at least three or four phosphorothioate internucleoside linkages in the antisense strand.

### Capping Residues or Moieties

In some embodiments, the sense strand may include one or more capping residues or moieties, sometimes referred to in the art as a “cap,” a “terminal cap,” or a “capping residue.” As used herein, a “capping residue” is a non-nucleotide compound or other moiety that can be incorporated at one or more termini of a nucleotide sequence of an RNAi agent disclosed herein. A capping residue can provide the RNAi agent, in some instances, with certain beneficial properties, such as, for example, protection against exonuclease degradation. In some embodiments, inverted abasic residues (invAb) (also referred to in the art as “inverted abasic sites”) are added as capping residues (see Table 11). (See, e.g., F. Czauderna, Nucleic Acids Res., 2003, 31(11), 2705-16). Capping residues are generally known in the art, and include, for example, inverted abasic residues as well as carbon chains such as a terminal C₃H₇ (propyl), C₆H₁₃ (hexyl), or C₁₂H₂₅ (dodecyl) groups. In some embodiments, a capping residue is present at either the 5′ terminal end, the 3′ terminal end, or both the 5′ and 3′ terminal ends of the sense strand. In some embodiments, the 5′ end and/or the 3′ end of the sense strand may include more than one inverted abasic deoxyribose moiety as a capping residue.

In some embodiments, one or more inverted abasic residues (invAb) are added to the 3′ end of the sense strand. In some embodiments, one or more inverted abasic residues (invAb) are added to the 5′ end of the sense strand. In some embodiments, one or more inverted abasic residues or inverted abasic sites are inserted between the targeting ligand and the nucleotide sequence of the sense strand of the RNAi agent. In some embodiments, the inclusion of one or more inverted abasic residues or inverted abasic sites at or near the terminal end or terminal ends of the sense strand of an RNAi agent allows for enhanced activity or other desired properties of an RNAi agent.

In some embodiments, one or more inverted abasic residues (invAb) are added to the 5′ end of the sense strand. In some embodiments, one or more inverted abasic residues can be inserted between the targeting ligand and the nucleotide sequence of the sense strand of the RNAi agent. The inverted abasic residues may be linked via phosphate, phosphorothioate (e.g., shown herein as (invAb) s)), or other internucleoside linkages. In some embodiments, the inclusion of one or more inverted abasic residues at or near the terminal end or terminal ends of the sense strand of an RNAi agent may allow for enhanced activity or other desired properties of an RNAi agent. In some embodiments, an inverted abasic (deoxyribose) residue can be replaced with an inverted ribitol (abasic ribose) residue. In some embodiments, the 3′ end of the antisense strand core stretch sequence, or the 3′ end of the antisense strand sequence, may include an inverted abasic residue. The chemical structures for inverted abasic deoxyribose residues are shown in Table 11 below.

### RAGE RNAi Agents

The RAGE RNAi agents disclosed herein are designed to target specific positions on an AGER (RAGE) gene (e.g., SEQ ID NO:1 (NM_001136.5)). As defined herein, an antisense strand sequence is designed to target an AGER gene at a given position on the gene when the 5′ terminal nucleobase of the antisense strand is aligned with a position that is 21 nucleotides downstream (towards the 3′ end) from the position on the gene when base pairing to the gene. For example, as illustrated in Tables 1 and 2 herein, an antisense strand sequence designed to target an AGER gene at position 177 requires that when base pairing to the gene, the 5′ terminal nucleobase of the antisense strand is aligned with position 197 of an AGER gene.

As provided herein, a RAGE RNAi agent does not require that the nucleobase at position 1 (5′→3′) of the antisense strand be complementary to the gene, provided that there is at least 85% complementarity (e.g., at least 85, 86, 87, 88, 89, 90, 91, 92, 93, 94, 95, 96, 97, 98, 99, or 100% complementarity) of the antisense strand and the gene across a core stretch sequence of at least 16 consecutive nucleotides. For example, for a RAGE RNAi agent disclosed herein that is designed to target position 177 of an AGER gene, the 5′ terminal nucleobase of the antisense strand of the of the RAGE RNAi agent must be aligned with position 197 of the gene; however, the 5′ terminal nucleobase of the antisense strand may be, but is not required to be, complementary to position 197 of an AGER gene, provided that there is at least 85% complementarity (e.g., at least 85, 86, 87, 88, 89, 90, 91, 92, 93, 94, 95, 96, 97, 98, 99, or 100% complementarity) of the antisense strand and the gene across a core stretch sequence of at least 16 consecutive nucleotides. As shown by, among other things, the various examples disclosed herein, the specific site of binding of the gene by the antisense strand of the RAGE RNAi agent (e.g., whether the RAGE RNAi agent is designed to target an AGER gene at position 177, at position 90, at position 330, or at some other position) is an important factor to the level of inhibition achieved by the RAGE RNAi agent. (See, e.g., Kamola et al., The siRNA Non-seed Region and Its Target Sequences are Auxiliary Determinants of Off-Target Effects, PLOS Computational Biology, 11(12), FIG. 1 (2015)).

In some embodiments, the RAGE RNAi agents disclosed herein target an AGER gene at or near the positions of the AGER sequence shown in Table 1. In some embodiments, the antisense strand of a RAGE RNAi agent disclosed herein includes a core stretch sequence that is fully, substantially, or at least partially complementary to a target RAGE 19-mer sequence disclosed in Table 1.

TABLE 1
AGER (RAGE) 19-mer mRNA Target Sequences (taken
from homo sapiens advanced glycosylation end-
product specific receptor (AGER), transcript
variant 1, GenBank NM_001136.5 (SEQ ID NO: 1))

| | | Corre- | |
|---|---|---|---|
| | | sponding | Targeted |
| | | Positions | Gene |
| | | of | Position |
| SEQ | AGER (RAGE) 19-mer | Sequence | (as |
| ID | Target Sequences | on SEQ ID | referred |
| No. | (5′→3′) | NO: 1 | to herein) |
| 22 | GAAUGGAAACUGAACACAG | 179-197 | 177 |
| 23 | GUAGGUGCUCAAAACAUCA | 92-110 | 90 |
| 24 | GCAAUGAACAGGAAUGGAA | 332-350 | 330 |
| 25 | AAUGGAAACUGAACACAGG | 180-198 | 178 |
| 26 | CAGAUUCCUGGGAAGCCAG | 386-404 | 384 |
| 27 | CUGGGAAGCCAGAAAUUGU | 393-411 | 391 |
| 28 | CACUGGUGCUGAAGUGUAA | 129-147 | 127 |
| 29 | GACAGAAGCUUGGAAGGUC | 202-220 | 200 |
| 30 | GGAUGAGGGGAUUUUCCGG | 307-325 | 305 |
| 31 | AUUCCUGGGAAGCCAGAAA | 389-407 | 387 |
| 32 | AUUCUGCCUCUGAACUCAC | 414-432 | 412 |
| 33 | CCCUGCAGGGACUCUUAGC | 481-499 | 479 |
| 34 | CCUGCAGGGACUCUUAGCU | 482-500 | 480 |
| 35 | CCACCUUCUCCUGUAGCUU | 642-660 | 640 |
| 36 | CUUCUCCUGUAGCUUCAGC | 646-664 | 644 |
| 37 | UGCUGGUCCUCAGUCUGUG | 63-81 | 61 |
| 38 | GCUGGUCCUCAGUCUGUGG | 64-82 | 62 |
| 39 | UCCGUGUCUACCAGAUUCC | 375-393 | 373 |
| 40 | CGUGUCUACCAGAUUCCUG | 377-395 | 375 |
| 41 | CACCUUCUCCUGUAGCUUC | 643-661 | 641 |
| 42 | CCUCAAAUCCACUGGAUGA | 830-848 | 828 |
| 43 | UAGAUUCUGCCUCUGAACU | 411-429 | 409 |
| 44 | GAUUCUGCCUCUGAACUCA | 413-431 | 411 |
| 45 | CUGGUGUUCCCAAUAAGGU | 435-453 | 433 |
| 46 | GGUGUUCCCAAUAAGGUGG | 437-455 | 435 |
| 47 | UUAGCUGGCACUUGGAUGG | 495-513 | 493 |
| 48 | UAAUGAGAAGGGAGUAUCU | 529-547 | 527 |
| 49 | GAGAAGGGAGUAUCUGUGA | 533-551 | 531 |
| 50 | GCAUCAGCAUCAUCGAACC | 981-999 | 979 |
| 51 | UGAACAGGAAUGGAAAGGA | 336-354 | 334 |
| 52 | CUACCGAGUCCGUGUCUAC | 367-385 | 365 |
| 53 | UGGGAAGCCAGAAAUUGUA | 394-412 | 392 |
| 54 | CCUAAUGAGAAGGGAGUAU | 527-545 | 525 |

Homo sapiens advanced glycosylation end-product specific receptor (AGER), transcript variant 1, GenBank NM_001136.5 (SEQ ID NO:1), gene transcript (1420 bases):

| | |
|---|---|
| 1 | agacagagcc aggaccctgg aaggaagcag gatggctgcc ggaacagcag ttggagcctg |
| 61 | ggtgctggtc ctcagtctgt ggggggcagt agtaggtgct caaaacatca cagcccggat |
| 121 | tggcgagcca ctggtgctga agtgtaaggg ggcccccaag aaaccacccc agcggctgga |
| 181 | atggaaactg aacacaggcc ggacagaagc ttggaaggtc ctgtctcccc agggaggagg |
| 241 | cccctgggac agtgtggctc gtgtccttcc caacggctcc ctcttccttc cggctgtcgg |
| 301 | gatccaggat gaggggattt tccggtgcca ggcaatgaac aggaatggaa aggagaccaa |
| 361 | gtccaactac cgagtccgtg tctaccagat tcctgggaag ccagaaattg tagattctgc |
| 421 | ctctgaactc acggctggtg ttcccaataa ggtggggaca tgtgtgtcag agggaagcta |
| 481 | ccctgcaggg actcttagct ggcacttgga tgggaagccc ctggtgccta atgagaaggg |
| 541 | agtatctgtg aaggaacaga ccaggagaca ccctgagaca gggctcttca cactgcagtc |
| 601 | ggagctaatg gtgaccccag cccggggagg agatccccgt cccaccttct cctgtagctt |
| 661 | cagcccaggc cttccccgac accgggcctt gcgcacagcc cccatccagc cccgtgtctg |
| 721 | ggagcctgtg cctctggagg aggtccaatt ggtggtggag ccagaaggtg gagcagtagc |
| 781 | tcctggtgga accgtaaccc tgacctgtga agtccctgcc cagccctctc ctcaaatcca |
| 841 | ctggatgaag gatggtgtgc ccttgcccct tccccccagc cctgtgctga tcctccctga |
| 901 | gatagggcct caggaccagg gaacctacag ctgtgtggcc acccattcca gccacgggcc |
| 961 | ccaggaaagc cgtgctgtca gcatcagcat catcgaacca ggcgaggagg ggccaactgc |
| 1021 | aggctctgtg ggaggatcag ggctgggaac tctagccctg gccctgggga tcctgggagg |
| 1081 | cctggggaca gccgccctgc tcattggggt catcttgtgg caaaggcggc aacgccgagg |
| 1141 | agaggagagg aaggccccag aaaaccagga ggaagaggag gagcgtgcag aactgaatca |
| 1201 | gtcggaggaa cctgaggcag gcgagagtag tactggaggg ccttgagggg cccacagaca |
| 1261 | gatcccatcc atcagctccc ttttcttttt cccttgaact gttctggcct cagaccaact |
| 1321 | ctctcctgta taatctctct cctgtataac cccaccttgc caagctttct tctacaacca |
| 1381 | gagcccccca caatgatgat taaacacctg acacatcttg |

In some embodiments, a RAGE RNAi agent includes an antisense strand wherein position 19 of the antisense strand (5′→3′) is capable of forming a base pair with position 1 of a 19-mer target sequence disclosed in Table 1. In some embodiments, a RAGE RNAi agent includes an antisense strand wherein position 1 of the antisense strand (5′→3′) is capable of forming a base pair with position 19 of a 19-mer target sequence disclosed in Table 1.

In some embodiments, a RAGE RNAi agent includes an antisense strand wherein position 2 of the antisense strand (5′→3′) is capable of forming a base pair with position 18 of a 19-mer target sequence disclosed in Table 1. In some embodiments, a RAGE RNAi agent includes an antisense strand wherein positions 2 through 18 of the antisense strand (5′→3′) are capable of forming base pairs with each of the respective complementary bases located at positions 18 through 2 of the 19-mer target sequence disclosed in Table 1.

For the RNAi agents disclosed herein, the nucleotide at position 1 of the antisense strand (from 5′ end→3′ end) can be perfectly complementary to an AGER gene, or can be non-complementary to an AGER gene. In some embodiments, the nucleotide at position 1 of the antisense strand (from 5′ end→3′ end) is a U, A, or dT. In some embodiments, the nucleotide at position 1 of the antisense strand (from 5′ end→3′ end) forms an A:U or U:A base pair with the sense strand.

In some embodiments, a RAGE RNAi agent antisense strand comprises the sequence of nucleotides (from 5′ end→3′ end) 2-18 or 2-19 of any of the antisense strand sequences in Table 2 or Table 3. In some embodiments, a RAGE RNAi sense strand comprises the sequence of nucleotides (from 5′ end→3′ end) 1-17, 1-18, or 2-18 of any of the sense strand sequences in Table 2, Table 4, Table 5, or Table 6.

In some embodiments, a RAGE RNAi agent is comprised of (i) an antisense strand comprising the sequence of nucleotides (from 5′ end→3′ end) 2-18 or 2-19 of any of the antisense strand sequences in Table 2 or Table 3, and (ii) a sense strand comprising the sequence of nucleotides (from 5′ end→3′ end) 1-17 or 1-18 of any of the sense strand sequences in Table 2, Table 4, Table 5, or Table 6.

In some embodiments, the RAGE RNAi agents include core 19-mer nucleotide sequences shown in the following Table 2.

TABLE 2
RAGE RNAi Agent Antisense Strand and Sense Strand Core Stretch Base
Sequences (N = any nucleobase; I = inosine (hypoxanthine) nucleobase)

| | | | | Corre- | |
|---|---|---|---|---|---|
| | | | | sponding | |
| | | | | Positions | |
| | | | | of | |
| | Antisense Strand | | Sense Strand | Identified | |
| | Base Sequence | | Base Sequence | Sequence | |
| SEQ | (5′→3′) (Shown | SEQ | (5′→3′) (Shown | on | Targeted |
| ID | as an Unmodified | ID | as an Unmodified | SEQ ID | Gene |
| NO:. | Nucleotide Sequence) | NO:. | Nucleotide Sequence) | NO: 1 | Position |
| 55 | UUGUGUUCAGUUUCCAUUC | 298 | GAAUGGAAACUGAACACAA | 179-197 | 177 |
| 56 | AUGUGUUCAGUUUCCAUUC | 299 | GAAUGGAAACUGAACACAU | 179-197 | 177 |
| 57 | CUGUGUUCAGUUUCCAUUC | 300 | GAAUGGAAACUGAACACAG | 179-197 | 177 |
| 58 | NUGUGUUCAGUUUCCAUUC | 301 | GAAUGGAAACUGAACACAN | 179-197 | 177 |
| 59 | NUGUGUUCAGUUUCCAUUN | 302 | NAAUGGAAACUGAACACAN | 179-197 | 177 |
| 60 | UUGUGUUCAGUUUCCAUUC | 303 | GAAUGGAAACUIAACACAA | 179-197 | 177 |
| 61 | AUGUGUUCAGUUUCCAUUC | 304 | GAAUGGAAACUIAACACAU | 179-197 | 177 |
| 62 | CUGUGUUCAGUUUCCAUUC | 305 | GAAUGGAAACUIAACACAG | 179-197 | 177 |
| 63 | NUGUGUUCAGUUUCCAUUC | 306 | GAAUGGAAACUIAACACAN | 179-197 | 177 |
| 64 | NUGUGUUCAGUUUCCAUUN | 307 | NAAUGGAAACUIAACACAN | 179-197 | 177 |
| 65 | UGAUGUUUUGAGCACCUAC | 308 | GUAGGUGCUCAAAACAUCA | 92-110 | 90 |
| 66 | AGAUGUUUUGAGCACCUAC | 309 | GUAGGUGCUCAAAACAUCU | 92-110 | 90 |
| 67 | NGAUGUUUUGAGCACCUAC | 310 | GUAGGUGCUCAAAACAUCN | 92-110 | 90 |
| 68 | NGAUGUUUUGAGCACCUAN | 311 | NUAGGUGCUCAAAACAUCN | 92-110 | 90 |
| 69 | UUCCAUUCCUGUUCAUUGC | 312 | GCAAUGAACAGGAAUGGAA | 332-350 | 330 |
| 70 | AUCCAUUCCUGUUCAUUGC | 313 | GCAAUGAACAGGAAUGGAU | 332-350 | 330 |
| 71 | NUCCAUUCCUGUUCAUUGC | 314 | GCAAUGAACAGGAAUGGAN | 332-350 | 330 |
| 72 | NUCCAUUCCUGUUCAUUGN | 315 | NCAAUGAACAGGAAUGGAN | 332-350 | 330 |
| 73 | UUCCAUUCCUGUUCAUUGC | 316 | GCAAUGAACAGGAAUIGAA | 332-350 | 330 |
| 74 | AUCCAUUCCUGUUCAUUGC | 317 | GCAAUGAACAGGAAUIGAU | 332-350 | 330 |
| 75 | NUCCAUUCCUGUUCAUUGC | 318 | GCAAUGAACAGGAAUIGAN | 332-350 | 330 |
| 76 | NUCCAUUCCUGUUCAUUGN | 319 | NCAAUGAACAGGAAUIGAN | 332-350 | 330 |
| 77 | UCUGUGUUCAGUUUCCAUU | 320 | AAUGGAAACUGAACACAGA | 180-198 | 178 |
| 78 | ACUGUGUUCAGUUUCCAUU | 321 | AAUGGAAACUGAACACAGU | 180-198 | 178 |
| 79 | CCUGUGUUCAGUUUCCAUU | 322 | AAUGGAAACUGAACACAGG | 180-198 | 178 |
| 80 | NCUGUGUUCAGUUUCCAUU | 323 | AAUGGAAACUGAACACAGN | 180-198 | 178 |
| 81 | NCUGUGUUCAGUUUCCAUN | 324 | NAUGGAAACUGAACACAGN | 180-198 | 178 |
| 82 | UCUGUGUUCAGUUUCCAUU | 325 | AAUGGAAACUGAACACAIA | 180-198 | 178 |
| 83 | ACUGUGUUCAGUUUCCAUU | 326 | AAUGGAAACUGAACACAIU | 180-198 | 178 |
| 84 | CCUGUGUUCAGUUUCCAUU | 327 | AAUGGAAACUGAACACAIG | 180-198 | 178 |
| 85 | NCUGUGUUCAGUUUCCAUU | 328 | AAUGGAAACUGAACACAIN | 180-198 | 178 |
| 86 | NCUGUGUUCAGUUUCCAUN | 329 | NAUGGAAACUGAACACAIN | 180-198 | 178 |
| 87 | UUGGCUUCCCAGGAAUCUG | 330 | CAGAUUCCUGGGAAGCCAA | 386-404 | 384 |
| 88 | AUGGCUUCCCAGGAAUCUG | 331 | CAGAUUCCUGGGAAGCCAU | 386-404 | 384 |
| 89 | CUGGCUUCCCAGGAAUCUG | 332 | CAGAUUCCUGGGAAGCCAG | 386-404 | 384 |
| 90 | NUGGCUUCCCAGGAAUCUG | 333 | CAGAUUCCUGGGAAGCCAN | 386-404 | 384 |
| 91 | NUGGCUUCCCAGGAAUCUN | 334 | NAGAUUCCUGGGAAGCCAN | 386-404 | 384 |
| 92 | UUGGCUUCCCAGGAAUCUG | 335 | CAGAUUCCUGGGAAICCAA | 386-404 | 384 |
| 93 | AUGGCUUCCCAGGAAUCUG | 336 | CAGAUUCCUGGGAAICCAU | 386-404 | 384 |
| 94 | CUGGCUUCCCAGGAAUCUG | 337 | CAGAUUCCUGGGAAICCAG | 386-404 | 384 |
| 95 | NUGGCUUCCCAGGAAUCUG | 338 | CAGAUUCCUGGGAAICCAN | 386-404 | 384 |
| 96 | NUGGCUUCCCAGGAAUCUN | 339 | NAGAUUCCUGGGAAICCAN | 386-404 | 384 |
| 97 | ACAAUUUCUGGCUUCCCAG | 340 | CUGGGAAGCCAGAAAUUGU | 393-411 | 391 |
| 98 | UCAAUUUCUGGCUUCCCAG | 341 | CUGGGAAGCCAGAAAUUGA | 393-411 | 391 |
| 99 | NCAAUUUCUGGCUUCCCAG | 342 | CUGGGAAGCCAGAAAUUGN | 393-411 | 391 |
| 100 | NCAAUUUCUGGCUUCCCAN | 343 | NUGGGAAGCCAGAAAUUGN | 393-411 | 391 |
| 101 | UUACACUUCAGCACCAGUG | 344 | CACUGGUGCUGAAGUGUAA | 129-147 | 127 |
| 102 | AUACACUUCAGCACCAGUG | 345 | CACUGGUGCUGAAGUGUAU | 129-147 | 127 |
| 103 | NUACACUUCAGCACCAGUG | 346 | CACUGGUGCUGAAGUGUAN | 129-147 | 127 |
| 104 | NUACACUUCAGCACCAGUN | 347 | NACUGGUGCUGAAGUGUAN | 129-147 | 127 |
| 105 | UACCUUCCAAGCUUCUGUC | 348 | GACAGAAGCUUGGAAGGUA | 202-220 | 200 |
| 106 | GACCUUCCAAGCUUCUGUC | 349 | GACAGAAGCUUGGAAGGUC | 202-220 | 200 |
| 107 | AACCUUCCAAGCUUCUGUC | 350 | GACAGAAGCUUGGAAGGUU | 202-220 | 200 |
| 108 | NACCUUCCAAGCUUCUGUC | 351 | GACAGAAGCUUGGAAGGUN | 202-220 | 200 |
| 109 | NACCUUCCAAGCUUCUGUN | 352 | NACAGAAGCUUGGAAGGUN | 202-220 | 200 |
| 110 | UACCUUCCAAGCUUCUGUC | 353 | GACAGAAGCUUGGAAGIUA | 202-220 | 200 |
| 111 | GACCUUCCAAGCUUCUGUC | 354 | GACAGAAGCUUGGAAGIUC | 202-220 | 200 |
| 112 | AACCUUCCAAGCUUCUGUC | 355 | GACAGAAGCUUGGAAGIUU | 202-220 | 200 |
| 113 | NACCUUCCAAGCUUCUGUC | 356 | GACAGAAGCUUGGAAGIUN | 202-220 | 200 |
| 114 | NACCUUCCAAGCUUCUGUN | 357 | NACAGAAGCUUGGAAGIUN | 202-220 | 200 |
| 115 | UCGGAAAAUCCCCUCAUCC | 358 | GGAUGAGGGGAUUUUCCGA | 307-325 | 305 |
| 116 | CCGGAAAAUCCCCUCAUCC | 359 | GGAUGAGGGGAUUUUCCGG | 307-325 | 305 |
| 117 | ACGGAAAAUCCCCUCAUCC | 360 | GGAUGAGGGGAUUUUCCGU | 307-325 | 305 |
| 118 | NCGGAAAAUCCCCUCAUCC | 361 | GGAUGAGGGGAUUUUCCGN | 307-325 | 305 |
| 119 | NCGGAAAAUCCCCUCAUCN | 362 | NGAUGAGGGGAUUUUCCGN | 307-325 | 305 |
| 120 | UCGGAAAAUCCCCUCAUCC | 363 | GGAUGAGGGGAUUUUCCIA | 307-325 | 305 |
| 121 | CCGGAAAAUCCCCUCAUCC | 364 | GGAUGAGGGGAUUUUCCIG | 307-325 | 305 |
| 122 | ACGGAAAAUCCCCUCAUCC | 365 | GGAUGAGGGGAUUUUCCIU | 307-325 | 305 |
| 123 | NCGGAAAAUCCCCUCAUCC | 366 | GGAUGAGGGGAUUUUCCIN | 307-325 | 305 |
| 124 | NCGGAAAAUCCCCUCAUCN | 367 | NGAUGAGGGGAUUUUCCIN | 307-325 | 305 |
| 125 | UUUCUGGCUUCCCAGGAAU | 368 | AUUCCUGGGAAGCUAGAAA | 389-407 | 387 |
| 126 | AUUCUGGCUUCCCAGGAAU | 369 | AUUCCUGGGAAGCUAGAAU | 389-407 | 387 |
| 127 | NUUCUGGCUUCCCAGGAAU | 370 | AUUCCUGGGAAGCUAGAAN | 389-407 | 387 |
| 128 | NUUCUGGCUUCCCAGGAAN | 371 | NUUCCUGGGAAGCUAGAAN | 389-407 | 387 |
| 129 | UUGAGUUCAGAGGCAGAAU | 372 | AUUCUGCCUCUGAACUCAC | 414-432 | 412 |
| 130 | GUGAGUUCAGAGGCAGAAU | 373 | AUUCUGCCUCUGAACUCAC | 414-432 | 412 |
| 131 | AUGAGUUCAGAGGCAGAAU | 374 | AUUCUGCCUCUGAACUCAU | 414-432 | 412 |
| 132 | NUGAGUUCAGAGGCAGAAU | 375 | AUUCUGCCUCUGAACUCAN | 414-432 | 412 |
| 133 | NUGAGUUCAGAGGCAGAAN | 376 | NUUCUGCCUCUGAACUCAN | 414-432 | 412 |
| 134 | UCUAAGAGUCCCUGCAGGG | 377 | CCCUGCAGGGACUCUUAGA | 481-499 | 479 |
| 135 | ACUAAGAGUCCCUGCAGGG | 378 | CCCUGCAGGGACUCUUAGU | 481-499 | 479 |
| 136 | GCUAAGAGUCCCUGCAGGG | 379 | CCCUGCAGGGACUCUUAGC | 481-499 | 479 |
| 137 | NCUAAGAGUCCCUGCAGGG | 380 | CCCUGCAGGGACUCUUAGN | 481-499 | 479 |
| 138 | NCUAAGAGUCCCUGCAGGN | 381 | NCCUGCAGGGACUCUUAGN | 481-499 | 479 |
| 139 | AGCUAAGAGUCCCUGCAGG | 382 | CCUGCAGGGACUCUUAGCU | 482-500 | 480 |
| 140 | UGCUAAGAGUCCCUGCAGG | 383 | CCUGCAGGGACUCUUAGCA | 482-500 | 480 |
| 141 | NGCUAAGAGUCCCUGCAGG | 384 | CCUGCAGGGACUCUUAGCN | 482-500 | 480 |
| 142 | NGCUAAGAGUCCCUGCAGN | 385 | NCUGCAGGGACUCUUAGCN | 482-500 | 480 |
| 143 | AGCUAAGAGUCCCUGCAGG | 386 | CCUGCAGGGACUCUUAICU | 482-500 | 480 |
| 144 | UGCUAAGAGUCCCUGCAGG | 387 | CCUGCAGGGACUCUUAICA | 482-500 | 480 |
| 145 | NGCUAAGAGUCCCUGCAGG | 388 | CCUGCAGGGACUCUUAICN | 482-500 | 480 |
| 146 | NGCUAAGAGUCCCUGCAGN | 389 | NCUGCAGGGACUCUUAICN | 482-500 | 480 |
| 147 | AAGCUACAGGAGAAGGUGG | 390 | CCACCUUCUCCUGUAGCUU | 642-660 | 640 |
| 148 | UAGCUACAGGAGAAGGUGG | 391 | CCACCUUCUCCUGUAGCUA | 642-660 | 640 |
| 149 | NAGCUACAGGAGAAGGUGG | 392 | CCACCUUCUCCUGUAGCUN | 642-660 | 640 |
| 150 | NAGCUACAGGAGAAGGUGN | 393 | NCACCUUCUCCUGUAGCUN | 642-660 | 640 |
| 151 | AAGCUACAGGAGAAGGUGG | 394 | CCACCUUCUCCUGUAICUU | 642-660 | 640 |
| 152 | UAGCUACAGGAGAAGGUGG | 395 | CCACCUUCUCCUGUAICUA | 642-660 | 640 |
| 153 | NAGCUACAGGAGAAGGUGG | 396 | CCACCUUCUCCUGUAICUN | 642-660 | 640 |
| 154 | NAGCUACAGGAGAAGGUGN | 397 | NCACCUUCUCCUGUAICUN | 642-660 | 640 |
| 155 | UCUGAAGCUACAGGAGAAG | 398 | CUUCUCCUGUAGCUUCAGA | 646-664 | 644 |
| 156 | ACUGAAGCUACAGGAGAAG | 399 | CUUCUCCUGUAGCUUCAGU | 646-664 | 644 |
| 157 | GCUGAAGCUACAGGAGAAG | 400 | CUUCUCCUGUAGCUUCAGC | 646-664 | 644 |
| 158 | NCUGAAGCUACAGGAGAAG | 401 | CUUCUCCUGUAGCUUCAGN | 646-664 | 644 |
| 159 | NCUGAAGCUACAGGAGAAN | 402 | NUUCUCCUGUAGCUUCAGN | 646-664 | 644 |
| 160 | UCUGAAGCUACAGGAGAAG | 403 | CUUCUCCUGUAGCUUCAIA | 646-664 | 644 |
| 161 | ACUGAAGCUACAGGAGAAG | 404 | CUUCUCCUGUAGCUUCAIU | 646-664 | 644 |
| 162 | GCUGAAGCUACAGGAGAAG | 405 | CUUCUCCUGUAGCUUCAIC | 646-664 | 644 |
| 163 | NCUGAAGCUACAGGAGAAG | 406 | CUUCUCCUGUAGCUUCAIN | 646-664 | 644 |
| 164 | NCUGAAGCUACAGGAGAAN | 407 | NUUCUCCUGUAGCUUCAIN | 646-664 | 644 |
| 165 | UACAGACUGAGGACCAGCA | 408 | UGCUGGUCCUCAGUCUGUA | 63-81 | 61 |
| 166 | AACAGACUGAGGACCAGCA | 409 | UGCUGGUCCUCAGUCUGUU | 63-81 | 61 |
| 167 | CACAGACUGAGGACCAGCA | 410 | UGCUGGUCCUCAGUCUGUG | 63-81 | 61 |
| 168 | NACAGACUGAGGACCAGCA | 411 | UGCUGGUCCUCAGUCUGUN | 63-81 | 61 |
| 169 | NACAGACUGAGGACCAGCN | 412 | UGCUGGUCCUCAGUCUGUN | 63-81 | 61 |
| 170 | UACAGACUGAGGACCAGCA | 413 | UGCUGGUCCUCAGUCUIUA | 63-81 | 61 |
| 171 | AACAGACUGAGGACCAGCA | 414 | UGCUGGUCCUCAGUCUIUU | 63-81 | 61 |
| 172 | CACAGACUGAGGACCAGCA | 415 | UGCUGGUCCUCAGUCUIUG | 63-81 | 61 |
| 173 | NACAGACUGAGGACCAGCA | 416 | UGCUGGUCCUCAGUCUIUN | 63-81 | 61 |
| 174 | NACAGACUGAGGACCAGCN | 417 | UGCUGGUCCUCAGUCUIUN | 63-81 | 61 |
| 175 | UCACAGACUGAGGACCAGC | 418 | GCUGGUCCUCAGUCUGUGA | 64-82 | 62 |
| 176 | ACACAGACUGAGGACCAGC | 419 | GCUGGUCCUCAGUCUGUGU | 64-82 | 62 |
| 177 | NCACAGACUGAGGACCAGC | 420 | GCUGGUCCUCAGUCUGUGN | 64-82 | 62 |
| 178 | NCACAGACUGAGGACCAGN | 421 | NCUGGUCCUCAGUCUGUGN | 64-82 | 62 |
| 179 | UCACAGACUGAGGACCAGC | 422 | GCUGGUCCUCAGUCUGUIA | 64-82 | 62 |
| 180 | ACACAGACUGAGGACCAGC | 423 | GCUGGUCCUCAGUCUGUIU | 64-82 | 62 |
| 181 | NCACAGACUGAGGACCAGC | 424 | GCUGGUCCUCAGUCUGUIN | 64-82 | 62 |
| 182 | NCACAGACUGAGGACCAGN | 425 | NCUGGUCCUCAGUCUGUIN | 64-82 | 62 |
| 183 | UCACAGACUGAGGACCAGC | 426 | GCUGGUCCUCAGUCUIUGA | 64-82 | 62 |
| 184 | ACACAGACUGAGGACCAGC | 427 | GCUGGUCCUCAGUCUIUGU | 64-82 | 62 |
| 185 | NCACAGACUGAGGACCAGC | 428 | GCUGGUCCUCAGUCUIUGN | 64-82 | 62 |
| 186 | NCACAGACUGAGGACCAGN | 429 | NCUGGUCCUCAGUCUIUGN | 64-82 | 62 |
| 187 | UGAAUCUGGUAGACACGGA | 430 | UCCGUGUCUACCAGAUUCA | 375-393 | 373 |
| 188 | AGAAUCUGGUAGACACGGA | 431 | UCCGUGUCUACCAGAUUCU | 375-393 | 373 |
| 189 | GGAAUCUGGUAGACACGGA | 432 | UCCGUGUCUACCAGAUUCC | 375-393 | 373 |
| 190 | NGAAUCUGGUAGACACGGA | 433 | UCCGUGUCUACCAGAUUCN | 375-393 | 373 |
| 191 | NGAAUCUGGUAGACACGGN | 434 | NCCGUGUCUACCAGAUUCN | 375-393 | 373 |
| 192 | UGAAUCUGGUAGACACGGA | 435 | UCCGUGUCUACCAIAUUCA | 375-393 | 373 |
| 193 | AGAAUCUGGUAGACACGGA | 436 | UCCGUGUCUACCAIAUUCU | 375-393 | 373 |
| 194 | GGAAUCUGGUAGACACGGA | 437 | UCCGUGUCUACCAIAUUCC | 375-393 | 373 |
| 195 | NGAAUCUGGUAGACACGGA | 438 | UCCGUGUCUACCAIAUUCN | 375-393 | 373 |
| 196 | NGAAUCUGGUAGACACGGN | 439 | NCCGUGUCUACCAIAUUCN | 375-393 | 373 |
| 197 | UAGGAAUCUGGUAGACACG | 440 | CGUGUCUACCAGAUUCCUA | 377-395 | 375 |
| 198 | AAGGAAUCUGGUAGACACG | 441 | CGUGUCUACCAGAUUCCUU | 377-395 | 375 |
| 199 | CAGGAAUCUGGUAGACACG | 442 | CGUGUCUACCAGAUUCCUG | 377-395 | 375 |
| 200 | NAGGAAUCUGGUAGACACG | 443 | CGUGUCUACCAGAUUCCUN | 377-395 | 375 |
| 201 | NAGGAAUCUGGUAGACACN | 444 | NGUGUCUACCAGAUUCCUN | 377-395 | 375 |
| 202 | UAAGCUACAGGAGAAGGUG | 445 | CACCUUCUCCUGUAGCUUA | 643-661 | 641 |
| 203 | AAAGCUACAGGAGAAGGUG | 446 | CACCUUCUCCUGUAGCUUU | 643-661 | 641 |
| 204 | GAAGCUACAGGAGAAGGUG | 447 | CACCUUCUCCUGUAGCUUC | 643-661 | 641 |
| 205 | NAAGCUACAGGAGAAGGUG | 448 | CACCUUCUCCUGUAGCUUN | 643-661 | 641 |
| 206 | NAAGCUACAGGAGAAGGUN | 449 | NACCUUCUCCUGUAGCUUN | 643-661 | 641 |
| 207 | UAAGCUACAGGAGAAGGUG | 450 | CACCUUCUCCUGUAICUUA | 643-661 | 641 |
| 208 | AAAGCUACAGGAGAAGGUG | 451 | CACCUUCUCCUGUAICUUU | 643-661 | 641 |
| 209 | GAAGCUACAGGAGAAGGUG | 452 | CACCUUCUCCUGUAICUUC | 643-661 | 641 |
| 210 | NAAGCUACAGGAGAAGGUG | 453 | CACCUUCUCCUGUAICUUN | 643-661 | 641 |
| 211 | NAAGCUACAGGAGAAGGUN | 454 | NACCUUCUCCUGUAICUUN | 643-661 | 641 |
| 212 | UCAUCCAGUGGAUUUGAGG | 455 | CCUCAAAUCCACUGGAUGA | 830-848 | 828 |
| 213 | ACAUCCAGUGGAUUUGAGG | 456 | CCUCAAAUCCACUGGAUGU | 830-848 | 828 |
| 214 | NCAUCCAGUGGAUUUGAGG | 457 | CCUCAAAUCCACUGGAUGN | 830-848 | 828 |
| 215 | NCAUCCAGUGGAUUUGAGN | 458 | NCUCAAAUCCACUGGAUGN | 830-848 | 828 |
| 216 | UCAUCCAGUGGAUUUGAGG | 459 | CCUCAAAUCCACUIGAUGA | 830-848 | 828 |
| 217 | ACAUCCAGUGGAUUUGAGG | 460 | CCUCAAAUCCACUIGAUGU | 830-848 | 828 |
| 218 | NCAUCCAGUGGAUUUGAGG | 461 | CCUCAAAUCCACUIGAUGN | 830-848 | 828 |
| 219 | NCAUCCAGUGGAUUUGAGN | 462 | NCUCAAAUCCACUIGAUGN | 830-848 | 828 |
| 220 | AGUUCAGAGGCAGAAUCUA | 463 | UAGAUUCUGCCUCUGAACU | 411-429 | 409 |
| 221 | UGUUCAGAGGCAGAAUCUA | 464 | UAGAUUCUGCCUCUGAACA | 411-429 | 409 |
| 222 | NGUUCAGAGGCAGAAUCUA | 465 | UAGAUUCUGCCUCUGAACN | 411-429 | 409 |
| 223 | NGUUCAGAGGCAGAAUCUN | 466 | NAGAUUCUGCCUCUGAACN | 411-429 | 409 |
| 224 | AGUUCAGAGGCAGAAUCUA | 467 | UAGAUUCUGCCUCUIAACU | 411-429 | 409 |
| 225 | UGUUCAGAGGCAGAAUCUA | 468 | UAGAUUCUGCCUCUIAACA | 411-429 | 409 |
| 226 | NGUUCAGAGGCAGAAUCUA | 469 | UAGAUUCUGCCUCUIAACN | 411-429 | 409 |
| 227 | NGUUCAGAGGCAGAAUCUN | 470 | NAGAUUCUGCCUCUIAACN | 411-429 | 409 |
| 228 | UGAGUUCAGAGGCAGAAUC | 471 | GAUUCUGCCUCUGAACUCA | 413-431 | 411 |
| 229 | AGAGUUCAGAGGCAGAAUC | 472 | GAUUCUGCCUCUGAACUCU | 413-431 | 411 |
| 230 | NGAGUUCAGAGGCAGAAUN | 473 | GAUUCUGCCUCUGAACUCN | 413-431 | 411 |
| 231 | NGAGUUCAGAGGCAGAAUN | 474 | NAUUCUGCCUCUGAACUCN | 413-431 | 411 |
| 232 | ACCUUAUUGGGAACACCAG | 475 | CUGGUGUUCCCAAUAAGGU | 435-453 | 433 |
| 233 | UCCUUAUUGGGAACACCAG | 476 | CUGGUGUUCCCAAUAAGGA | 435-453 | 433 |
| 234 | NCCUUAUUGGGAACACCAG | 477 | CUGGUGUUCCCAAUAAGGN | 435-453 | 433 |
| 235 | NCCUUAUUGGGAACACCAN | 478 | NUGGUGUUCCCAAUAAGGN | 435-453 | 433 |
| 236 | UCACCUUAUUGGGAACACC | 479 | GGUGUUCCCAAUAAGGUGA | 437-455 | 435 |
| 237 | ACACCUUAUUGGGAACACC | 480 | GGUGUUCCCAAUAAGGUGU | 437-455 | 435 |
| 238 | CCACCUUAUUGGGAACACC | 481 | GGUGUUCCCAAUAAGGUGG | 437-455 | 435 |
| 239 | NCACCUUAUUGGGAACACC | 482 | GGUGUUCCCAAUAAGGUGN | 437-455 | 435 |
| 240 | NCACCUUAUUGGGAACACN | 483 | NGUGUUCCCAAUAAGGUGN | 437-455 | 435 |
| 241 | UCACCUUAUUGGGAACACC | 484 | GGUGUUCCCAAUAAIGUGA | 437-455 | 435 |
| 242 | ACACCUUAUUGGGAACACC | 485 | GGUGUUCCCAAUAAIGUGU | 437-455 | 435 |
| 243 | CCACCUUAUUGGGAACACC | 486 | GGUGUUCCCAAUAAIGUGG | 437-455 | 435 |
| 244 | NCACCUUAUUGGGAACACC | 487 | GGUGUUCCCAAUAAIGUGN | 437-455 | 435 |
| 245 | NCACCUUAUUGGGAACACN | 488 | NGUGUUCCCAAUAAIGUGN | 437-455 | 435 |
| 246 | UCAUCCAAGUGCCAGCUAA | 489 | UUAGCUGGCACUUGGAUGA | 495-513 | 493 |
| 247 | ACAUCCAAGUGCCAGCUAA | 490 | UUAGCUGGCACUUGGAUGU | 495-513 | 493 |
| 248 | CCAUCCAAGUGCCAGCUAA | 491 | UUAGCUGGCACUUGGAUGG | 495-513 | 493 |
| 249 | NCAUCCAAGUGCCAGCUAA | 492 | UUAGCUGGCACUUGGAUGN | 495-513 | 493 |
| 250 | NCAUCCAAGUGCCAGCUAN | 493 | NUAGCUGGCACUUGGAUGN | 495-513 | 493 |
| 251 | UCAUCCAAGUGCCAGCUAA | 494 | UUAGCUGGCACUUIGAUGA | 495-513 | 493 |
| 252 | ACAUCCAAGUGCCAGCUAA | 495 | UUAGCUGGCACUUIGAUGU | 495-513 | 493 |
| 253 | CCAUCCAAGUGCCAGCUAA | 496 | UUAGCUGGCACUUIGAUGG | 495-513 | 493 |
| 254 | NCAUCCAAGUGCCAGCUAA | 497 | UUAGCUGGCACUUIGAUGN | 495-513 | 493 |
| 255 | NCAUCCAAGUGCCAGCUAN | 498 | NUAGCUGGCACUUIGAUGN | 495-513 | 493 |
| 256 | AGAUACUCCCUUCUCAUUA | 499 | UAAUGAGAAGGGAGUAUCU | 529-547 | 527 |
| 257 | UGAUACUCCCUUCUCAUUA | 500 | UAAUGAGAAGGGAGUAUCA | 529-547 | 527 |
| 258 | NGAUACUCCCUUCUCAUUA | 501 | UAAUGAGAAGGGAGUAUCN | 529-547 | 527 |
| 259 | NGAUACUCCCUUCUCAUUN | 502 | NAAUGAGAAGGGAGUAUCN | 529-547 | 527 |
| 260 | AGAUACUCCCUUCUCAUUA | 503 | UAAUGAGAAGGGAIUAUCU | 529-547 | 527 |
| 261 | UGAUACUCCCUUCUCAUUA | 504 | UAAUGAGAAGGGAIUAUCA | 529-547 | 527 |
| 262 | NGAUACUCCCUUCUCAUUA | 505 | UAAUGAGAAGGGAIUAUCN | 529-547 | 527 |
| 263 | NGAUACUCCCUUCUCAUUN | 506 | NAAUGAGAAGGGAIUAUCN | 529-547 | 527 |
| 264 | UCACAGAUACUCCCUUCUC | 507 | GAGAAGGGAGUAUCUGUGA | 533-551 | 531 |
| 265 | ACACAGAUACUCCCUUCUC | 508 | GAGAAGGGAGUAUCUGUGU | 533-551 | 531 |
| 266 | NCACAGAUACUCCCUUCUC | 509 | GAGAAGGGAGUAUCUGUGN | 533-551 | 531 |
| 267 | NCACAGAUACUCCCUUCUN | 510 | NAGAAGGGAGUAUCUGUGN | 533-551 | 531 |
| 268 | UCACAGAUACUCCCUUCUC | 511 | GAGAAGGGAGUAUCUIUGA | 533-551 | 531 |
| 269 | ACACAGAUACUCCCUUCUC | 512 | GAGAAGGGAGUAUCUIUGU | 533-551 | 531 |
| 270 | NCACAGAUACUCCCUUCUC | 513 | GAGAAGGGAGUAUCUIUGN | 533-551 | 531 |
| 271 | NCACAGAUACUCCCUUCUN | 514 | NAGAAGGGAGUAUCUIUGN | 533-551 | 531 |
| 272 | UGUUCGAUGAUGCUGAUGC | 515 | GCAUCAGCAUCAUCGAACA | 981-999 | 979 |
| 273 | AGUUCGAUGAUGCUGAUGC | 516 | GCAUCAGCAUCAUCGAACU | 981-999 | 979 |
| 274 | GGUUCGAUGAUGCUGAUGC | 517 | GCAUCAGCAUCAUCGAACC | 981-999 | 979 |
| 275 | NGUUCGAUGAUGCUGAUGC | 518 | GCAUCAGCAUCAUCGAACN | 981-999 | 979 |
| 276 | NGUUCGAUGAUGCUGAUGN | 519 | NCAUCAGCAUCAUCGAACN | 981-999 | 979 |
| 277 | UGUUCGAUGAUGCUGAUGC | 520 | GCAUCAGCAUCAUCIAACA | 981-999 | 979 |
| 278 | AGUUCGAUGAUGCUGAUGC | 521 | GCAUCAGCAUCAUCIAACU | 981-999 | 979 |
| 279 | GGUUCGAUGAUGCUGAUGC | 522 | GCAUCAGCAUCAUCIAACC | 981-999 | 979 |
| 280 | NGUUCGAUGAUGCUGAUGC | 523 | GCAUCAGCAUCAUCIAACN | 981-999 | 979 |
| 281 | NGUUCGAUGAUGCUGAUGN | 524 | NCAUCAGCAUCAUCIAACN | 981-999 | 979 |
| 282 | ACCUUUCCAUUCCUGUUCA | 525 | UGAACAGGAAUGGAAAGGU | 336-354 | 334 |
| 283 | UCCUUUCCAUUCCUGUUCA | 526 | UGAACAGGAAUGGAAAGGA | 336-354 | 334 |
| 284 | NCCUUUCCAUUCCUGUUCA | 527 | UGAACAGGAAUGGAAAGGN | 336-354 | 334 |
| 285 | NCCUUUCCAUUCCUGUUCN | 528 | NGAACAGGAAUGGAAAGGN | 336-354 | 334 |
| 286 | AUAGACACGGACUCGGUAG | 529 | CUACCGAGUCCGUGUCUAA | 367-385 | 365 |
| 287 | UUAGACACGGACUCGGUAG | 530 | CUACCGAGUCCGUGUCUAU | 367-385 | 365 |
| 288 | NUAGACACGGACUCGGUAG | 531 | CUACCGAGUCCGUGUCUAN | 367-385 | 365 |
| 289 | NUAGACACGGACUCGGUAN | 532 | NUACCGAGUCCGUGUCUAN | 367-385 | 365 |
| 290 | AACAAUUUCUGGCUUCCCA | 533 | UGGGAAGCCAGAAAUUGUA | 394-412 | 392 |
| 291 | UACAAUUUCUGGCUUCCCA | 534 | UGGGAAGCCAGAAAUUGUU | 394-412 | 392 |
| 292 | NACAAUUUCUGGCUUCCCA | 535 | UGGGAAGCCAGAAAUUGUN | 394-412 | 392 |
| 293 | NACAAUUUCUGGCUUCCCN | 536 | NGGGAAGCCAGAAAUUGUN | 394-412 | 392 |
| 294 | AUACUCCCUUCUCAUUAGG | 537 | CCUAAUGAGAAGGGAGUAA | 527-545 | 525 |
| 295 | UUACUCCCUUCUCAUUAGG | 538 | CCUAAUGAGAAGGGAGUAU | 527-545 | 525 |
| 296 | NUACUCCCUUCUCAUUAGG | 539 | CCUAAUGAGAAGGGAGUAN | 527-545 | 525 |
| 297 | NUACUCCCUUCUCAUUAGN | 540 | NCUAAUGAGAAGGGAGUAN | 527-545 | 525 |

The RAGE RNAi agent sense strands and antisense strands that comprise or consist of the nucleotide sequences in Table 2 can be modified nucleotides or unmodified nucleotides. In some embodiments, the RAGE RNAi agents having the sense and antisense strand sequences that comprise or consist of any of the nucleotide sequences in Table 2 are all or substantially all modified nucleotides.

In some embodiments, the antisense strand of a RAGE RNAi agent disclosed herein differs by 0, 1, 2, or 3 nucleotides from any of the antisense strand sequences in Table 2. In some embodiments, the sense strand of a RAGE RNAi agent disclosed herein differs by 0, 1, 2, or 3 nucleotides from any of the sense strand sequences in Table 2.

In some embodiments, the antisense strand of a RAGE RNAi agent disclosed comprises at least 15 contiguous nucleotides differing by 0, 1, 2, or 3 nucleotides from any of the antisense strand sequences in Table 2. In some embodiments, the sense strand of a RAGE RNAi agent disclosed herein comprises at least 15 contiguous nucleotides differing by 0, 1, 2, or 3 nucleotides from any of the sense strand sequences in Table 2.

As used herein, each N listed in a sequence disclosed in Table 2 may be independently selected from any and all nucleobases (including those found on both modified and unmodified nucleotides). In some embodiments, an N nucleotide listed in a sequence disclosed in Table 2 has a nucleobase that is complementary to the N nucleotide at the corresponding position on the other strand. In some embodiments, an N nucleotide listed in a sequence disclosed in Table 2 has a nucleobase that is not complementary to the N nucleotide at the corresponding position on the other strand. In some embodiments, an N nucleotide listed in a sequence disclosed in Table 2 has a nucleobase that is the same as the N nucleotide at the corresponding position on the other strand. In some embodiments, an N nucleotide listed in a sequence disclosed in Table 2 has a nucleobase that is different from the N nucleotide at the corresponding position on the other strand.

Certain modified RAGE RNAi agent sense and antisense strands are provided in Table 3, Table 4, Table 5, Table 6, and Table 10. Certain modified RAGE RNAi agent antisense strands, as well as their underlying unmodified nucleobase sequences, are provided in Table 3. Certain modified RAGE RNAi agent sense strands, as well as their underlying unmodified nucleobase sequences, are provided in Tables 4, 5, and 6. In forming RAGE RNAi agents, each of the nucleotides in each of the underlying base sequences listed in Tables 3, 4, 5, and 6, as well as in Table 2, above, can be a modified nucleotide.

The RAGE RNAi agents described herein are formed by annealing an antisense strand with a sense strand. A sense strand containing a sequence listed in Table 2, Table 4, Table 5, or Table 6 can be hybridized to any antisense strand containing a sequence listed in Table 2 or Table 3, provided the two sequences have a region of at least 85% complementarity over a contiguous 16, 17, 18, 19, 20, or 21 nucleotide sequence.

In some embodiments, a RAGE RNAi agent antisense strand comprises a nucleotide sequence of any of the sequences in Table 2 or Table 3.

In some embodiments, a RAGE RNAi agent comprises or consists of a duplex having the nucleobase sequences of the sense strand and the antisense strand of any of the sequences in Table 2, Table 3, Table 4, Table 5, Table 6, or Table 10.

Examples of antisense strands containing modified nucleotides are provided in Table 3. Examples of sense strands containing modified nucleotides are provided in Tables 4, 5 and 6.

As used in Tables 3, 4, 5, 6, and 10, the following notations are used to indicate modified nucleotides, targeting groups, and linking groups:
A=adenosine-3′-phosphate
C=cytidine-3′-phosphate
G=guanosine-3′-phosphate
U=uridine-3′-phosphate
I=inosine-3′-phosphate
a=2′-O-methyladenosine-3′-phosphate
as=2′-O-methyladenosine-3′-phosphorothioate
c=2′-O-methylcytidine-3′-phosphate
cs=2′-O-methylcytidine-3′-phosphorothioate
g=2′-O-methylguanosine-3′-phosphate
gs=2′-O-methylguanosine-3′-phosphorothioate
i=2′-O-methylinosine-3′-phosphate
is==2′-O-methylinosine-3′-phosphorothioate
t=2′-O-methyl-5-methyluridine-3′-phosphate
ts=2′-O-methyl-5-methyluridine-3′-phosphorothioate
u=2′-O-methyluridine-3′-phosphate
us=2′-O-methyluridine-3′-phosphorothioate
Af=2′-fluoroadenosine-3′-phosphate
Afs=2′-fluoroadenosine-3′-phosporothioate
Cf=2′-fluorocytidine-3′-phosphate
Cfs=2′-fluorocytidine-3′-phosphorothioate
Gf=2′-fluoroguanosine-3′-phosphate
Gfs=2′-fluoroguanosine-3′-phosphorothioate
Tf=2′-fluoro-5′-methyluridine-3′-phosphate
Tfs=2′-fluoro-5′-methyluridine-3′-phosphorothioate
Uf=2′-fluorouridine-3′-phosphate
Ufs=2′-fluorouridine-3′-phosphorothioate
dT=2′-deoxythymidine-3′-phosphate
A_{UNA}=2′,3′-seco-adenosine-3′-phosphate
A_{UNA}s=2′,3′-seco-adenosine-3′-phosphorothioate
C_{UNA}=2′,3′-seco-cytidine-3′-phosphate
C_{UNA}s=2′,3′-seco-cytidine-3′-phosphorothioate
G_{UNA}=2′,3′-seco-guanosine-3′-phosphate
G_{UNA}s=2′,3′-seco-guanosine-3′-phosphorothioate
U_{UNA}=2′,3′-seco-uridine-3′-phosphate
U_{UNA}s=2′,3′-seco-uridine-3′-phosphorothioate
a_2N=see Table 11
a_2Ns=see Table 11
(invAb)=inverted abasic deoxyribonucleotide-5′-phosphate, see Table 11
(invAb)s=inverted abasic deoxyribonucleotide-5′-phosphorothioate, see Table 11
s=phosphorothioate linkage
p=terminal phosphate (as synthesized)
vpdN=vinyl phosphonate deoxyribonucleotide
cPrpa=5′-cyclopropyl phosphonate-2′-O-methyladenosine-3′-phosphate (see Table 11)
cPrpas=5′-cyclopropyl phosphonate-2′-O-methyladenosine-3′-phosphorothioate (see Table 11)
cPrpu=5′-cyclopropyl phosphonate-2′-O-methyluridine-3′-phosphate (see Table 11)
cPrpus=5′-cyclopropyl phosphonate-2′-O-methyluridine-3′-phosphorothioate (see Table 11)
(Alk-SS-C6)=see Table 11
(C6-SS-Alk)=see Table 11
(C6-SS-C6)=see Table 11
(6-SS-6)=see Table 11
(C6-SS-Alk-Me)=see Table 11
(NH2-C6)=see Table 11
(TriAlk14)=see Table 11
(TriAlk14)s=see Table 11
-C6-=see Table 11
-C6s-=see Table 11
-L6-C6-=see Table 11
-L6-C6s-=see Table 11
-Alk-cyHex-=see Table 11
-Alk-cyHexs-=see Table 11
(TA14)=see Table 11 (structure of (TriAlk14) s after conjugation)
(TA14)s=see Table 11 (structure of (TriAlk14) s after conjugation)

As the person of ordinary skill in the art would readily understand, unless otherwise indicated by the sequence (such as, for example, by a phosphorothioate linkage “s”), when present in an oligonucleotide, the nucleotide monomers are mutually linked by 5′-3′-phosphodiester bonds. As the person of ordinary skill in the art would clearly understand, the inclusion of a phosphorothioate linkage as shown in the modified nucleotide sequences disclosed herein replaces the phosphodiester linkage typically present in oligonucleotides. Further, the person of ordinary skill in the art would readily understand that the terminal nucleotide at the 3′ end of a given oligonucleotide sequence would typically have a hydroxyl (—OH) group at the respective 3′ position of the given monomer instead of a phosphate moiety ex vivo. Additionally, for the embodiments disclosed herein, when viewing the respective strand 5′→3′, the inverted abasic residues are inserted such that the 3′ position of the deoxyribose is linked at the 3′ end of the preceding monomer on the respective strand (see, e.g., Table 11). Moreover, as the person of ordinary skill would readily understand and appreciate, while the phosphorothioate chemical structures depicted herein typically show the anion on the sulfur atom, the inventions disclosed herein encompass all phosphorothioate tautomers (e.g., where the sulfur atom has a double-bond and the anion is on an oxygen atom). Unless expressly indicated otherwise herein, such understandings of the person of ordinary skill in the art are used when describing the RAGE RNAi agents and compositions of RAGE RNAi agents disclosed herein.

Certain examples of targeting groups and linking groups used with the RAGE RNAi agents disclosed herein are included in the chemical structures provided below in Table 11. Each sense strand and/or antisense strand can have any targeting groups or linking groups listed herein, as well as other targeting or linking groups, conjugated to the 5′ and/or 3′ end of the sequence.

TABLE 3
RAGE RNAi Agent Antisense Strand Sequences

| | | | Underlying Base Sequence | |
|---|---|---|---|---|
| | | SEQ | (5′→3′) (Shown as an | SEQ |
| AS | | ID | Unmodified Nucleotide | ID |
| Strand ID | Modified Antisense Strand (5′→3′) | NO. | Sequence) | NO. |
| AM10308-AS | usUfsgsUfgUfuCfaGfuUfuCfcAfuUfeCfsg | 2 | UUGUGUUCAGUUUCCAUUCCG | 7 |
| AM10309-AS | cPrpusUfsgsUfgUfuCfaGfuUfuCfcAfuUfcCfsg | 3 | UUGUGUUCAGUUUCCAUUCCG | 7 |
| AMI0311-AS | usCfsusGfuGfuUfcAfgUfuUfcCfaUfuCfsc | 543 | UCUGUGUUCAGUUUCCAUUCC | 801 |
| AM10312-AS | cPrpusCfsusGfuGfuUfcAfgUfuUfcCfaUfuCfsc | 544 | UCUGUGUUCAGUUUCCAUUCC | 801 |
| AM10314-AS | usUfsgsGfcUfuCfcCfaGfgAfaUfcUfgGfsu | 545 | UUGGCUUCCCAGGAAUCUGGU | 802 |
| AM10315-AS | cPrpusUfsgsGfcUfuCfcCfaGfgAfaUfcUfgGfsu | 546 | UUGGCUUCCCAGGAAUCUGGU | 802 |
| AM10317-AS | asCfsasAfuUfuCfuGfgCfuUfcCfcAfgGfsa | 547 | ACAAUUUCUGGCUUCCCAGGA | 803 |
| AM10318-AS | cPrpasCfsasAfuUfuCfuGfgCfuUfcCfcAfgGfsa | 548 | ACAAUUUCUGGCUUCCCAGGA | 803 |
| AM10467-AS | usUfsasCfaCfuUfcAfgCfaCfcAfgUfgGfsc | 549 | UUACACUUCAGCACCAGUGGC | 804 |
| AM10469-AS | usAfscsCfuUfcCfaAfgCfuUfcUfgUfcCfsg | 550 | UACCUUCCAAGCUUCUGUCCG | 805 |
| AM10471-AS | usCfsgsGfaAfaAfuCfcCfcUfcAfuCfcUfsg | 551 | UCGGAAAAUCCCCUCAUCCUG | 806 |
| AM10473-AS | usUfsusCfuGfgCfuUfcCfcAfgGfaAfuCfsu | 552 | UUUCUGGCUUCCCAGGAAUCU | 807 |
| AM10475-AS | usUfsgsAfgUfuCfaGfaGfgCfaGfaAfuCfsu | 553 | UUGAGUUCAGAGGCAGAAUCU | 808 |
| AM10477-AS | usCfsusAfaGfaGfuCfcCfuGfcAfgGfgUfsa | 554 | UCUAAGAGUCCCUGCAGGGUA | 809 |
| AM10479-AS | asGfscsUfaAfgAfgUfcCfcUfgCfaGfgGfsu | 555 | AGCUAAGAGUCCCUGCAGGGU | 810 |
| AM10481-AS | asAfsgsCfuAfcAfgGfaGfaAfgGfuGfgGfsa | 556 | AAGCUACAGGAGAAGGUGGGA | 811 |
| AM10483-AS | usCfsusGfaAfgCfuAfcAfgGfaGfaAfgGfsu | 557 | UCUGAAGCUACAGGAGAAGGU | 812 |
| AM10571-AS | usAfscsAfgAfcUfgAfgGfaCfcAfgCfaCfsc | 558 | UACAGACUGAGGACCAGCACC | 813 |
| AM10573-AS | usAfscsAfgAfC_{UNA}UfgAfgGfaCfcAfgCfaCfsc | 559 | UACAGACUGAGGACCAGCACC | 813 |
| AM10575-AS | usCfsasCfaGfaCfuGfaGfgAfcCfaGfcAfsc | 560 | UCACAGACUGAGGACCAGCAC | 814 |
| AM10717-AS | usUfsgsUfgUfuCfaGfuUfuCfcAfuUfcCfsc | 561 | UUGUGUUCAGUUUCCAUUCCG | 815 |
| AM10720-AS | usUfsgsUfgUfU_{UNA}CfaGfuUfuCfcAfuUfcCfsg | 562 | UUGUGUUCAGUUUCCAUUCCG | 7 |
| AM10722-AS | usUfsgsUfgUfucaguUfuCfcAfuUfcCfsg | 563 | UUGUGUUCAGUUUCCAUUCCG | 7 |
| AM10723-AS | usUfsgsuguucaguUfuCfcAfuuccsg | 564 | UUGUGUUCAGUUUCCAUUCCG | 7 |
| AM10724-AS | usUfsgsuguucaGfuUfuCfcAfuuccsg | 565 | UUGUGUUCAGUUUCCAUUCCG | 7 |
| AM10752-AS | usGfsasUfgUfuUfuGfaGfcAfcCfuAfcUfsc | 566 | UGAUGUUUUGAGCACCUACUC | 9 |
| AM10754-AS | usUfscsCfaUfuCfcUfgUfuCfaUfuGfcCfsu | 4 | UUCCAUUCCUGUUCAUUGCCU | 8 |
| AM10756-AS | usGfsasAfuCfuGfgUfaGfaCfaCfgGfaCfsu | 568 | UGAAUCUGGUAGACACGGACU | 818 |
| AM10758-AS | usAfsgsGfaAfuCfuGfgUfaGfaCfaCfgGfsa | 569 | UAGGAAUCUGGUAGACACGGA | 819 |
| AM10760-AS | usAfsasGfcUfaCfaGfgAfgAfaGfgUfgGfsg | 570 | UAAGCUACAGGAGAAGGUGGG | 820 |
| AM10762-AS | usCfsasUfcCfaGfuGfgAfuUfuGfaGfgAfsg | 571 | UCAUCCAGUGGAUUUGAGGAG | 821 |
| AM10774-AS | asGfsusUfcAfgAfgGfcAfgAfaUfcUfaCfsc | 572 | AGUUCAGAGGCAGAAUCUACC | 822 |
| AM10776-AS | usGfsasGfuUfC_{UNA}AfgAfgGfcAfgAfaUfcUfsa | 573 | UGAGUUCAGAGGCAGAAUCUA | 823 |
| AM10778-AS | asCfscsUfuAfuUfgGfgAfaCfaCfcAfgCfsc | 574 | ACCUUAUUGGGAACACCAGCC | 824 |
| AM10780-AS | usCfsasCfcUfuAfuUfgGfgAfaCfaCfcAfsg | 575 | UCACCUUAUUGGGAACACCAG | 825 |
| AM10782-AS | usCfsasUfcCfaAfgUfgCfcAfgCfuAfaGfsc | 576 | UCAUCCAAGUGCCAGCUAAGC | 826 |
| AM10784-AS | asGfsasUfaCfuCfcCfuUfcUfcAfuUfaGfsg | 577 | AGAUACUCCCUUCUCAUUAGG | 827 |
| AM10786-AS | usCfsasCfaGfaUfaCfuCfcCfuUfcUfcAfsc | 578 | UCACAGAUACUCCCUUCUCAC | 828 |
| AM10788-AS | usGfsusUfcGfaUfgAfuGfcUfgAfuGfcUfsg | 579 | UGUUCGAUGAUGCUGAUGCUG | 829 |
| AM11103-AS | cPrpusUfgUfgUfuCfaGfuUfuCfcAfuUfcCfsg | 580 | UUGUGUUCAGUUUCCAUUCCG | 7 |
| AM11104-AS | cPrpuUfgUfgUfuCfaGfuUfuCfcAfuUfcCfsg | 581 | UUGUGUUCAGUUUCCAUUCCG | 7 |
| AM11188-AS | cPrpusUfsgsuguucaguUfuCfcAfuuccsg | 582 | UUGUGUUCAGUUUCCAUUCCG | 7 |
| AM11190-AS | usUfsgsuguuC_{UNA}aguUfuCfcAfuuccsg | 583 | UUGUGUUCAGUUUCCAUUCCG | 7 |
| AMI1191-AS | usUfsgsuguU_{UNA}caguUfuCfcAfuuccsg | 584 | UUGUGUUCAGUUUCCAUUCCG | 7 |
| AM11192-AS | usUfsgsugU_{UNA}UcaguUfuCfcAfuuccsg | 585 | UUGUGUUCAGUUUCCAUUCCG | 7 |
| AM11194-AS | usUfsgsuguucaguUfuCfcAfuuccsc | 586 | UUGUGUUCAGUUUCCAUUCCG | 815 |
| AM11196-AS | usUfsgsuguucaguUfuCfcAfuuccsa | 587 | UUGUGUUCAGUUUCCAUUCCA | 830 |
| AM11757-AS | cPrpuUfguguucaguUfuCfcAfuuccsg | 588 | UUGUGUUCAGUUUCCAUUCCG | 7 |
| AM11758-AS | cPrpuUfguguU_{UNA}caguUfuCfcAfuuccsg | 589 | UUGUGUUCAGUUUCCAUUCCG | 7 |
| AM11759-AS | cPrpuUfguguucaguUfuCfcAfuuccsc | 590 | UUGUGUUCAGUUUCCAUUCCG | 815 |
| AM11760-AS | cPrpuUfguguU_{UNA}caguUfuCfcAfuuccsc | 591 | UUGUGUUCAGUUUCCAUUCCG | 815 |
| AM11761-AS | usUfsgsuguU_{UNA}caguUfuCfcAfuuccsc | 592 | UUGUGUUCAGUUUCCAUUCCG | 815 |
| AM11762-AS | cPrpusUfsgsuguU_{UNA}caguUfuCfcAfuuccsg | 593 | UUGUGUUCAGUUUCCAUUCCG | 7 |
| AM11763-AS | cPrpusUfsgsuguucaguUfuCfcAfuuccsc | 594 | UUGUGUUCAGUUUCCAUUCCC | 815 |
| AM11764-AS | cPrpusUfsgsuguU_{UNA}caguUfuCfcAfuuccsc | 595 | UUGUGUUCAGUUUCCAUUCCG | 815 |
| AM11889-AS | usCfscsUfuUfcCfaUfuCfcUfgUfuCfaUfsc | 596 | UCCUUUCCAUUCCUGUUCAUC | 831 |
| AMI1892-AS | usUfsasGfaCfaCfgGfaCfuCfgGfuAfgUfsc | 597 | UUAGACACGGACUCGGUAGUC | 832 |
| AM11894-AS | asUfsasCfuCfcCfuUfcUfcAfuUfaGfgCfsa | 598 | AUACUCCCUUCUCAUUAGGCA | 833 |
| AM11895-AS | cPrpusGfsasUfgUfuUfuGfaGfcAfcCfuAfcUfsc | 599 | UGAUGUUUUGAGCACCUACUC | 9 |
| AM11897-AS | usGfsasuguuuugaGfcAfcCfuacusc | 5 | UGAUGUUUUGAGCACCUACUC | 9 |
| AM11898-AS | cPrpusGfsasuguuuugaGfcAfcCfuacusc | 6 | UGAUGUUUUGAGCACCUACUC | 9 |
| AM12234-AS | cPrpusUfscsCfaUfuCfcUfgUfuCfaUfuGfcCfsu | 602 | UUCCAUUCCUGUUCAUUGCCU | 8 |
| AM12236-AS | usUfscscauuccugUfuCfaUfugccsu | 603 | UUCCAUUCCUGUUCAUUGCCU | 8 |
| AM12237-AS | cPrpusUfscscauuccugUfuCfaUfugccsu | 604 | UUCCAUUCCUGUUCAUUGCCU | 8 |
| AM12240-AS | usUfscscauU_{UNA}CCugUfuCfaUfugccsu | 605 | UUCCAUUCCUGUUCAUUGCCU | 8 |
| AM12241-AS | usUfscscaU_{UNA}UccugUfuCfaUfugccsu | 606 | UUCCAUUCCUGUUCAUUGCCU | 8 |
| AM12245-AS | usUfscscauuccugUfuCfaUfugccsc | 607 | UUCCAUUCCUGUUCAUUGCCC | 834 |
| AM12593-AS | usGfsasuguuuugaGfcAfcCfuacusg | 608 | UGAUGUUUUGAGCACCUACUG | 835 |
| AM12594-AS | usGfsasuguU_{UNA}UugaGfcAfcCfuacusc | 609 | UGAUGUUUUGAGCACCUACUC | 9 |
| AM12596-AS | usGfsasuguuuugaGfcAfcCfuacusa | 610 | UGAUGUUUUGAGCACCUACUA | 836 |
| AM12755-AS | usUfscsCfaUfuccugUfuCfaUfuGfccsu | 611 | UUCCAUUCCUGUUCAUUGCCU | 8 |
| AM12756-AS | cPrpusUfscsCfaUfuccugUfuCfaUfuGfccsu | 612 | UUCCAUUCCUGUUCAUUGCCU | 8 |
| AM12757-AS | cPrpuUfcCfaUfuccugUfuCfaUfuGfccsu | 613 | UUCCAUUCCUGUUCAUUGCCU | 8 |
| AM14090-AS | usUfsgsUfguucaguUfuCfcAfuuccsg | 614 | UUGUGUUCAGUUUCCAUUCCG | 7 |
| AM14091-AS | usUfsgsuguUfcaguUfuCfcAfuuccsg | 615 | UUGUGUUCAGUUUCCAUUCCG | 7 |
| AM14093-AS | usGfsasUfguuuugaGfcAfcCfuacusc | 616 | UGAUGUUUUGAGCACCUACUC | 9 |
| AM14094-AS | usGfsasuguuuugaGfcAfcCfuAfcusc | 617 | UGAUGUUUUGAGCACCUACUC | 9 |
| AM14095-AS | usGfsasuguUfuugaGfcAfcCfuacusc | 618 | UGAUGUUUUGAGCACCUACUC | 9 |
| AMI5021-AS | cPrpusUfsgsuguucaguUfuCfcAfuuccsa | 619 | UUGUGUUCAGUUUCCAUUCCA | 830 |
| AM15767-AS | cPrpusAfscsAfaUfuucugGfcUfuCfcCfagsg | 620 | UACAAUUUCUGGCUUCCCAGG | 837 |
| AM15770-AS | cPrpusCfsusGfuGfuucagUfuUfcCfaUfucsc | 621 | UCUGUGUUCAGUUUCCAUUCC | 801 |

TABLE 4
RAGE RNAi Agent Sense Strand Sequences (Shown Without Linkers, Conjugated Targeting Ligands,
or Capping Moieties)

| | | SEQ | Underlying Base Sequence (5′→3′) | SEQ |
|---|---|---|---|---|
| | Modified Sense Strand | ID | (Shown as an Unmodified | ID |
| Strand ID | (5′→3′) | NO. | Nucleotide Sequence) | NO. |
| AM10307-SS-NL | csggaauggAfAfAfcugaacacaa | 13 | CGGAAUGGAAACUGAACACAA | 19 |
| AM10310-SS-NL | gsgaauggaAfAfCfugaacacaia | 623 | GGAAUGGAAACUGAACACAIA | 839 |
| AM10313-SS-NL | asccagauuCfCfUfgggaaiccaa | 624 | ACCAGAUUCCUGGGAAICCAA | 840 |
| AM10316-SS-NL | usccugggaAfGfCfcagaaauugu | 625 | UCCUGGGAAGCCAGAAAUUGU | 841 |
| AM10466-SS-NL | gsccacuggUfGfCfugaaguguaa | 626 | GCCACUGGUGCUGAAGUGUAA | 842 |
| AM10468-SS-NL | csggacagaAfGfCfuuggaagiua | 627 | CGGACAGAAGCUUGGAAGIUA | 843 |
| AM10470-SS-NL | csaggaugaGfGfGfgauuuuccia | 628 | CAGGAUGAGGGGAUUUUCCIA | 844 |
| AM10472-SS-NL | asgauuccuGfGfGfaagcuagaaa | 629 | AGAUUCCUGGGAAGCUAGAAA | 845 |
| AM10474-SS-NL | a_2NsgauucugCfCfUfcugaacucaa | 630 | (A^{2N})GAUUCUGCCUCUGAACUCAA | 846 |
| AM10476-SS-NL | usacccugcAfGfGfgacucuuaga | 631 | UACCCUGCAGGGACUCUUAGA | 847 |
| AM10478-SS-NL | ascccugcaGfGfGfacucuuaicu | 632 | ACCCUGCAGGGACUCUUAICU | 848 |
| AM10480-SS-NL | uscccaccuUfCfUfccuguaicuu | 633 | UCCCACCUUCUCCUGUAICUU | 849 |
| AM10482-SS-NL | asccuucucCfUfGfuagcuucaia | 634 | ACCUUCUCCUGUAGCUUCAIA | 850 |
| AM10570-SS-NL | gsgugcuggUfCfCfucagucuiua | 635 | GGUGCUGGUCCUCAGUCUIUA | 851 |
| AM10572-SS-NL | gsgugcuggUfCfCfucagucugua | 636 | GGUGCUGGUCCUCAGUCUGUA | 852 |
| AM10574-SS-NL | gsugcugguCfCfUfcagucuguia | 637 | GUGCUGGUCCUCAGUCUGUIA | 853 |
| AM10576-SS-NL | gsugcugguCfCfUfcagucuiuga | 638 | GUGCUGGUCCUCAGUCUIUGA | 854 |
| AM10644-SS-NL | csggaauggAfAfAfcugaacacaa | 13 | CGGAAUGGAAACUGAACACAA | 19 |
| AM10716-SS-NL | gsggaauggAfAfAfcugaacacaa | 640 | GGGAAUGGAAACUGAACACAA | 855 |
| AM10718-SS-NL | csggaauggAfAfAfcuiaacacaa | 641 | CGGAAUGGAAACUIAACACAA | 856 |
| AM10719-SS-NL | csggaauggAfa_2NAfcuiaacacaa | 642 | CGGAAUGGA(A^{2N})ACUIAACACAA | 857 |
| AM10721-SS-NL | csggaauggAfAfAfcugaauacaa | 643 | CGGAAUGGAAACUGAACACAA | 858 |
| AM10725-SS-NL | csggaauGfgAfaAfcugaacacaa | 644 | CGGAAUGGAAACUGAACACAA | 19 |
| AM10737-SS-NL | usccugggaAfGfCfcagaaauugu | 645 | UCCUGGGAAGCCAGAAAUUGU | 841 |
| AM10751-SS-NL | gsaguagguGfCfUfcaaaacauca | 646 | GAGUAGGUGCUCAAAACAUCA | 20 |
| AM10753-SS-NL | asggcaaugAfAfCfaggaauigaa | 15 | AGGCAAUGAACAGGAAUIGAA | 21 |
| AM10755-SS-NL | asguccgugUfCfUfaccaiauuca | 648 | AGUCCGUGUCUACCAIAUUCA | 861 |
| AM10757-SS-NL | usccgugucUfAfCfcagauuccua | 649 | UCCGUGUCUACCAGAUUCCUA | 862 |
| AM10759-SS-NL | csccaccuuCfUfCfcuguaicuua | 650 | CCCACCUUCUCCUGUAICUUA | 863 |
| AM10761-SS-NL | csuccucaaAfUfCfcacuigauga | 651 | CUCCUCAAAUCCACUIGAUGA | 864 |
| AM10773-SS-NL | gsguagauuCfUfGfccucuiaacu | 652 | GGUAGAUUCUGCCUCUIAACU | 865 |
| AM10775-SS-NL | usagauucuGfCfCfucugaacuca | 653 | UAGAUUCUGCCUCUGAACUCA | 866 |
| AM10777-SS-NL | gsgcuggugUfUfCfccaauaaggu | 654 | GGCUGGUGUUCCCAAUAAGGU | 867 |
| AM10779-SS-NL | csugguguuCfCfCfaauaagiuga | 655 | CUGGUGUUCCCAAUAAGIUGA | 868 |
| AM10781-SS-NL | gscuuagcuGfGfCfacuuigauga | 656 | GCUUAGCUGGCACUUIGAUGA | 869 |
| AM10783-SS-NL | cscuaaugaGfAfAfgggaiuaucu | 657 | CCUAAUGAGAAGGGAIUAUCU | 870 |
| AM10785-SS-NL | gsugagaagGfGfAfguaucuiuga | 658 | GUGAGAAGGGAGUAUCUIUGA | 871 |
| AM10787-SS-NL | csagcaucaGfCfAfucauciaaca | 659 | CAGCAUCAGCAUCAUCIAACA | 872 |
| AM11105-SS-NL | cggaauggAfAfAfcugaacacaa | 660 | CGGAAUGGAAACUGAACACAA | 19 |
| AM11106-SS-NL | csggaauggAfAfAfcugaacacaa | 13 | CGGAAUGGAAACUGAACACAA | 19 |
| AM11107-SS-NL | cggaauggAfAfAfcugaacacaa | 662 | CGGAAUGGAAACUGAACACAA | 19 |
| AM11189-SS-NL | csggaauGfgAfaAfcugaauacaa | 663 | CGGAAUGGAAACUGAACACAA | 858 |
| AM11193-SS-NL | gsggaauGfgAfaAfcugaacacaa | 664 | GGGAACGGAAACCGAACACAA | 855 |
| AM11195-SS_NL | usggaauGfgAfaAfcugaacacaa | 665 | CGGAAUGGAAACUGAACACAA | 873 |
| AM11197-SS-NL | csggaauGfgAfa_2NAfcugaacacaa | 666 | CGGAAUGGA(A^{2N})ACUGAACACAA | 874 |
| AM11512-SS-NL | cggaauggAfAfAfcugaacacaa | 667 | CGGAAUGGAAACUGAACACAA | 19 |
| AM11513-SS-NL | csggaauggAfAfAfcugaacacaa | 13 | CGGAAUGGAAACUGAACACAA | 19 |
| AM11514-SS-NL | csggaauggAfAfAfcugaacacaa | 13 | CGGAAUGGAAACUGAACACAA | 19 |
| AM11515-SS-NL | cggaauggAfAfAfcugaacacaa | 670 | CGGAAUGGAAACUGAACACAA | 19 |
| AM11516-SS-NL | csggaauggAfAfAfcugaacacaa | 13 | CGGAAUGGAAACUGAACACAA | 19 |
| AM11517-SS-NL | cggaauggAfAfAfcugaacacaa | 672 | CGGAAUGGAAACUGAACACAA | 19 |
| AM11888-SS-NL | gsaugaacaGfGfAfauggaaagga | 673 | GAUGAACAGGAAUGGAAAGGA | 875 |
| AM11890-SS-NL | gsaugaacaGfGfAfauggaaagia | 674 | GAUGAACAGGAAUGGAAAGIA | 876 |
| AM11891-SS-NL | gsacuaccgAfGfUfccgugucuaa | 675 | GACUACCGAGUCCGUGUCUAA | 877 |
| AM11893-SS-NL | usgccuaauGfAfGfaagggaguau | 676 | UGCCUAAUGAGAAGGGAGUAU | 878 |
| AM11896-SS-NL | gsaguagguGfcUfcAfaaacauca | 677 | GAGUAGGUGCUCAAAACAUCA | 20 |
| AM11899-SS-NL | gsaguagiuGfcUfcAfaaacauca | 678 | GAGUAGIUGCUCAAAACAUCA | 879 |
| AM11900-SS-NL | gsaguagGfuGfcUfcaaaacauca | 14 | GAGUAGGUGCUCAAAACAUCA | 20 |
| AM11901-SS-NL | gsaguagguGfcUfcaaaacauca | 680 | GAGUAGGUGCUCAAAACAUCA | 20 |
| AM12235-SS-NL | asggcaaUfgAfaCfaggaauigaa | 681 | AGGCAAUGAACAGGAAUIGAA | 21 |
| AM12238-SS-NL | asggcaaUfgAfaCfaggaauggaa | 682 | AGGCAAUGAACAGGAAUGGAA | 880 |
| AM12239-SS-NL | asggcaaUfgAfaCfaggaaugiaa | 683 | AGGCAAUGAACAGGAAUGIAA | 881 |
| AM12242-SS-NL | asggcaaUfgAfaCfagiaauggaa | 684 | AGGCAAUGAACAGIAAUGGAA | 882 |
| AM12243-SS-NL | asggcaaUfgAfaCfaigaauggaa | 685 | AGGCAAUGAACAIGAAUGGAA | 883 |
| AM12244-SS-NL | gsggcaaUfgAfaCfaggaauigaa | 686 | GGGCAAUGAACAGGAAUIGAA | 884 |
| AM12592-SS-NL | csaguagGfuGfcUfcaaaacauca | 687 | GAGUAGGUGCUCAAAACAUCA | 885 |
| AM12595-SS-NL | usa_2NguagGfuGfcUfcaaaacauca | 688 | U(A^{2N})GUAGGUGCUCAAAACAUCA | 886 |
| AM12597-SS-NL | gsaguagguGfcUfCfaaaacauca | 689 | GAGUAGGUGCUCAAAACAUCA | 20 |
| AM12754-SS-NL | asggcaaugAfAfCfaggaauggaa | 690 | AGGCAAUGAACAGGAAUGGAA | 880 |
| AM12910-SS-NL | gsaguagGfuGfcUfcaaaacauca | 14 | GAGUAGGUGCUCAAAACAUCA | 20 |
| AM12911-SS-NL | asggcaaugAfAfCfaggaauigaa | 15 | AGGCAAUGAACAGGAAUIGAA | 21 |
| AM13987-SS-NL | csggaauggAfAfAfcugaacacaa | 13 | CGGAAUGGAAACUGAACACAA | 19 |
| AM14092-SS-NL | csggaauggAfaAfcUfgaacacaa | 694 | CGGAAUGGAAACUGAACACAA | 19 |
| AM15766-SS-NL | cscugggaaGfCfCfagaaauugua | 695 | CCUGGGAAGCCAGAAAUUGUA | 887 |
| AM16133-SS-NL | csggaauggAfAfAfcugaacacaa | 13 | CGGAAUGGAAACUGAACACAA | 19 |

(A^{2N}) = 2-aminoadenine-containing nucleotide; 1 = hypoxanthine (inosine) nucleotide}

TABLE 5
RAGE RNAi Agent Sense Strand Sequences (Shown With TriAlk14 Linker (see Table 11 for
structure information)).

| | | | Underlying Base Sequence TriAlkl4 | |
|---|---|---|---|---|
| | | SEQ ID | (Shown as an Unmodified Nucleotide | SEQ ID |
| Strand ID | Modified Sense Strand (5′→3′) | NO. | Sequence) | NO. |
| AM10307-SS | (TriAlk14)csggaauggAfAfAfcugaacacaas(invAb) | 16 | CGGAAUGGAAACUGAACACAA | 19 |
| AM10310-SS | (TriAlk14)gsgaauggaAfAfCfugaacacaias(invAb) | 698 | GGAAUGGAAACUGAACACAIA | 839 |
| AM10313-SS | (TriAlk14)asccagauuCfCfUfgggaaiccaas(invAb) | 699 | ACCAGAUUCCUGGGAAICCAA | 840 |
| AM10316-SS | (TriAlk14)usccugggaAfGfCfcagaaauugus(invAb) | 700 | UCCUGGGAAGCCAGAAAUUGU | 841 |
| AM10466-SS | (TriAlk14)gsccacuggUfGfCfugaaguguaas(invAb) | 701 | GCCACUGGUGCUGAAGUGUAA | 842 |
| AM10468-SS | (TriAlk14)csggacagaAfGfCfuuggaagiuas(invAb) | 702 | CGGACAGAAGCUUGGAAGIUA | 843 |
| AM10470-SS | (TriAlk14)csaggaugaGfGfGfgauuuuccias(invAb) | 703 | CAGGAUGAGGGGAUUUUCCIA | 844 |
| AM10472-SS | (TriAlk14)asgauuccuGfGfGfaagcuagaaas(invAb) | 704 | AGAUUCCUGGGAAGCUAGAAA | 845 |
| AM10474-SS | (TriAlk14)a_2NsgauucugCfCfUfcugaacucaas(invAb) | 705 | (A^{2N})GAUUCUGCCUCUGAACUCAA | 846 |
| AM10476-SS | (TriAlk14)usacccugcAfGfGfgacucuuagas(invAb) | 706 | UACCCUGCAGGGACUCUUAGA | 847 |
| AM10478-SS | (TriAlk14)ascccugcaGfGfGfacucuuaicus(invAb) | 707 | ACCCUGCAGGGACUCUUAICU | 848 |
| AM10480-SS | (TriAlk14)uscccaccuUfCfUfccuguaicuus(invAb) | 708 | UCCCACCUUCUCCUGUAICUU | 849 |
| AM10482-SS | (TriAlk14)asccuucucCfUfGfuagcuucaias(invAb) | 709 | ACCUUCUCCUGUAGCUUCAIA | 850 |
| AM10570-SS | (TriAlk14)gsgugcuggUfCfCfucagucuiuas(invAb) | 710 | GGUGCUGGUCCUCAGUCUIUA | 851 |
| AM10572-SS | (TriAlk14)gsgugcuggUfCfCfucagucuguas(invAb) | 711 | GGUGCUGGUCCUCAGUCUGUA | 852 |
| AM10574-SS | (TriAlk14)gsugcugguCfCfUfcagucuguias(invAb) | 712 | GUGCUGGUCCUCAGUCUGUIA | 853 |
| AM10576-SS | (TriAlk14)gsugcugguCfCfUfcagucuiugas(invAb) | 713 | GUGCUGGUCCUCAGUCUIUGA | 854 |
| AM10644-SS | (TriAlk14)csggaauggAfAfAfcugaacacaas(invAb) | 16 | CGGAAUGGAAACUGAACACAA | 19 |
| AM10716-SS | (TriAlk14)gsggaauggAfAfAfcugaacacaas(invAb) | 715 | GGGAAUGGAAACUGAACACAA | 855 |
| AM10718-SS | (TriAlk14)csggaauggAfAfAfcuiaacacaas(invAb) | 716 | CGGAAUGGAAACUIAACACAA | 856 |
| AM10719-SS | (TriAlk14)csggaauggAfa_2NAfcuiaacacaas(invAb) | 717 | CGGAAUGGA(A^{2N})ACUIAACACAA | 857 |
| AM10721-SS | (TriAlk14)csggaauggAfAfAfcugaauacaas(invAb) | 718 | CGGAAUGGAAACUGAAUACAA | 858 |
| AM10725-SS | (TriAlk14)csggaauGfgAfaAfcugaacacaas(invAb) | 719 | CGGAAUGGAAACUGAACACAA | 19 |
| AM10737-SS | (TriAlk14)usccugggaAfGfCfcagaaauugus(invAb) | 720 | UCCUGGGAAGCCAGAAAUUGU | 841 |
| AM10751-SS | (TriAlk14)gsaguagguGfCfUfcaaaacaucas(invAb) | 721 | GAGUAGGUGCUCAAAACAUCA | 20 |
| AM10753-SS | (TriAlk14)asggcaaugAfAfCfaggaauigaas(invAb) | 18 | AGGCAAUGAACAGGAAUIGAA | 21 |
| AM10755-SS | (TriAlk14)asguccgugUfCfUfaccaiauucas(invAb) | 723 | AGUCCGUGUCUACCAIAUUCA | 861 |
| AM10757-SS | (TriAlk14)usccgugucUfAfCfcagauuccuas(invAb) | 724 | UCCGUGUCUACCAGAUUCCUA | 862 |
| AM10759-SS | (TriAlk14)csccaccuuCfUfCfcuguaicuuas(invAb) | 725 | CCCACCUUCUCCUGUAICUUA | 863 |
| AM10761-SS | (TriAlk14)csuccucaaAfUfCfcacuigaugas(invAb) | 726 | CUCCUCAAAUCCACUIGAUGA | 864 |
| AM10773-SS | (TriAlk14)gsguagauuCfUfGfccucuiaacus(invAb) | 727 | GGUAGAUUCUGCCUCUIAACU | 865 |
| AM10775-SS | (TriAlk14)usagauucuGfCfCfucugaacucas(invAb) | 728 | UAGAUUCUGCCUCUGAACUCA | 866 |
| AM10777-SS | (TriAlk14)gsgcuggugUfUfCfccaauaaggus(invAb) | 729 | GGCUGGUGUUCCCAAUAAGGU | 867 |
| AM10779-SS | (TriAlk14)csugguguuCfCfCfaauaagiugas(invAb) | 730 | CUGGUGUUCCCAAUAAGIUGA | 868 |
| AM10781-SS | (TriAlk14)gscuuagcuGfGfCfacuuigaugas(invAb) | 731 | GCUUAGCUGGCACUUIGAUGA | 869 |
| AM10783-SS | (TriAlk14)cscuaaugaGfAfAfgggaiuaucus(invAb) | 732 | CCUAAUGAGAAGGGAIUAUCU | 870 |
| AM10785-SS | (TriAlk14)gsugagaagGfGfAfguaucuiugas(invAb) | 733 | GUGAGAAGGGAGUAUCUIUGA | 871 |
| AM10787-SS | (TriAlk14)csagcaucaGfCfAfucauciaacas(invAb) | 734 | CAGCAUCAGCAUCAUCIAACA | 872 |
| AM11105-SS | (TriAlk14)cggaauggAfAfAfcugaacacaas(invAb) | 735 | CGGAAUGGAAACUGAACACAA | 19 |
| AM11106-SS | (TriAlk14)csggaauggAfAfAfcugaacacaa(invAb) | 736 | CGGAAUGGAAACUGAACACAA | 19 |
| AM11107-SS | (TriAlk14)cggaauggAfAfAfcugaacacaa(invAb) | 737 | CGGAAUGGAAACUGAACACAA | 19 |
| AM11189-SS | (TriAlk14)csggaauGfgAfaAfcugaauacaas(invAb) | 738 | CGGAAUGGAAACUGAAUACAA | 858 |
| AM11193-SS | (TriAlk14)gsggaauGfgAfaAfcugaacacaas(invAb) | 739 | CGGAAUGGAAACUGAACACAA | 855 |
| AM11195-SS | (TriAlk14)usggaauGfgAfaAfcugaacacaas(invAb) | 740 | CGGAAUGGAAACUGAACACAA | 873 |
| AM11197-SS | (TriAlk14)csggaauGfgAfa_2NAfcugaacacaas(invAb) | 741 | CGGAAUGGA(A^{2N})ACUGAACACAA | 874 |
| AM11512-SS | (TriAlk14)cggaauggAfAfAfcugaacacaas(invAb) | 742 | CGGAAUGGAAACUGAACACAA | 19 |
| AM11513-SS | (TriAlk14)csggaauggAfAfAfcugaacacaas(invAb) | 16 | CGGAAUGGAAACUGAACACAA | 19 |
| AM11514-SS | (TriAlk14)csggaauggAfAfAfcugaacacaas(invAb) | 16 | CGGAAUGGAAACUGAACACAA | 19 |
| AM11515-SS | (TriAlk14)cggaauggAfAfAfcugaacacaas(invAb) | 745 | CGGAAUGGAAACUGAACACAA | 19 |
| AM11516-SS | (TriAlk14)csggaauggAfAfAfcugaacacaas(invAb) | 16 | CGGAAUGGAAACUGAACACAA | 19 |
| AM11517-SS | (TriAlk14)cggaauggAfAfAfcugaacacaas(invAb) | 747 | CGGAAUGGAAACUGAACACAA | 19 |
| AM11888-SS | (TriAlk14)gsaugaacaGfGfAfauggaaaggas(invAb) | 748 | GAUGAACAGGAAUGGAAAGGA | 875 |
| AM1189O-SS | (TriAlk14)gsaugaacaGfGfAfauggaaagias(invAb) | 749 | GAUGAACAGGAAUGGAAAGIA | 876 |
| AM11891-SS | (TriAlk14)gsacuaccgAfGfUfccgugucuaas(invAb) | 750 | GACUACCGAGUCCGUGUCUAA | 877 |
| AM11893-SS | (TriAlk14)usgccuaauGfAfGfaagggaguaus(invAb) | 751 | UGCCUAAUGAGAAGGGAGUAU | 878 |
| AM11896-SS | (TriAlk14)gsaguagguGfcUfcAfaaacaucas(invAb) | 752 | GAGUAGGUGCUCAAAACAUCA | 20 |
| AM11899-SS | (TriAlk14)gsaguagiuGfcUfcAfaaacaucas(invAb) | 753 | GAGUAGIUGCUCAAAACAUCA | 879 |
| AM11900-SS | (TriAlk14)gsaguagGfuGfcUfcaaaacaucas(invAb) | 17 | GAGUAGGUGCUCAAAACAUCA | 20 |
| AM11901-SS | (TriAlk14)gsaguagguGfcUfcaaaacaucas(invAb) | 755 | GAGUAGGUGCUCAAAACAUCA | 20 |
| AM12235-SS | (TriAlk14)asggcaaUfgAfaCfaggaauigaas(invAb) | 756 | AGGCAAUGAACAGGAAUIGAA | 21 |
| AM12238-SS | (TriAlk14)asggcaaUfgAfaCfaggaauggaas(invAb) | 757 | AGGCAAUGAACAGGAAUGGAA | 880 |
| AM12239-SS | (TriAlk14)asggcaaUfgAfaCfaggaaugiaas(invAb) | 758 | AGGCAAUGAACAGGAAUGIAA | 881 |
| AM12242-SS | (TriAlk14)asggcaaUfgAfaCfagiaauggaas(invAb) | 759 | AGGCAAUGAACAGIAAUGGAA | 882 |
| AM12243-SS | (TriAlk14)asggcaaUfgAfaCfaigaauggaas(invAb) | 760 | AGGCAAUGAACAIGAAUGGAA | 883 |
| AM12244-SS | (TriAlk14)gsggcaaUfgAfaCfaggaauigaas(invAb) | 761 | GGGCAAUGAACAGGAAUIGAA | 884 |
| AM12592-SS | (TriAlk14)csaguagGfuGfcUfcaaaacaucas(invAb) | 762 | GAGUAGGUGCUCAAAACAUCA | 885 |
| AM12595-SS | (TriAlk14)usa_2NguagGfuGfcUfcaaaacaucas(invAb) | 763 | U(A^{2N})GUAGGUGCUCAAAACAUCA | 886 |
| AM12597-SS | (TriAlk14)gsaguagguGfcUfCfaaaacaucas(invAb) | 764 | GAGUAGGUGCUCAAAACAUCA | 20 |
| AM12754-SS | (TriAlk14)asggcaaugAfAfCfaggaauggaas(invAb) | 765 | AGGCAAUGAACAGGAAUGGAA | 880 |
| AM12910-SS | (TriAlk14)gsaguagGfuGfcUfcaaaacaucas(invAb) | 17 | GAGUAGGUGCUCAAAACAUCA | 20 |
| AM12911-SS | (TriAlk14)asggcaaugAfAfCfaggaauigaas(invAb) | 18 | AGGCAAUGAACAGGAAUIGAA | 21 |
| AM13987-SS | (TriAlk14)csggaauggAfAfAfcugaacacaas(invAb) | 16 | CGGAAUGGAAACUGAACACAA | 19 |
| AM14092-SS | (TriAlk14)csggaauggAfaAfcUfgaacacaas(invAb) | 769 | CGGAAUGGAAACUGAACACAA | 19 |
| AM15766-SS | (TriAlk14)cscugggaaGfCfCfagaaauuguas(invAb) | 770 | CCUGGGAAGCCAGAAAUUGUA | 887 |
| AM16133-SS | (TriAlk14)scsggaauggAfAfAfcugaacacaas(invAb) | 771 | CGGAAUGGAAACUGAACACAA | 19 |

(A^{2N}) = 2-aminoadenine-containing nucleotide; 1 = hypoxanthine (inosine) nucleotide}

TABLE 6
RAGE RNAi Agent Sense Strand Sequences (Shown with Targeting Ligand Conjugate. The structure of
avβ6-SM6.1 is shown in Table 11, and the structure of Tri-SM6.1-avβ6-(TA14) is shown in FIG. 1.)

| | | | Corresponding |
|---|---|---|---|
| | | | Sense Strand |
| | | | AM Number |
| | | SEQ | Without Linker |
| Strand | | ID | or Conjugate |
| ID | Modified Sense Strand (5′→3′) | NO. | (See Table 4) |
| CS000363 | Tri-SM6.1-avβ6-(TA14)csggaauggAfAfAfcugaacacaas(invAb) | 10 | AM10307-SS-NL |
| CS000368 | Tri-SM6.1-avβ6-(TA14)cggaauggAfAfAfcugaacacaas(invAb) | 773 | AM11105-SS-NL |
| CS000369 | Tri-SM6.1-avβ6-(TA14)csggaauggAfAfAfcugaacacaa(invAb) | 774 | AM11106-SS-NL |
| CS000386 | Tri-SM6.1-avβ6-(TA14)cggaauggAfAfAfcugaacacaa(invAb) | 775 | AM11107-SS-NL |
| CS000497 | Tri-SM6.1-avβ6-(TA14)csggaauGfgAfaAfcugaauacaas(invAb) | 776 | AM11189-SS-NL |
| CS000499 | Tri-SM6.1-avβ6-(TA14)csggaauGfgAfaAfcugaacacaas(invAb) | 777 | AM10725-SS-NL |
| CS000503 | Tri-SM6.1-avβ6-(TA14)gsggaauGfgAfaAfcugaacacaas(invAb) | 778 | AM11193-SS-NL |
| CS000505 | Tri-SM6.1-avβ6-(TA14)usggaauGfgAfaAfcugaacacaas(invAb) | 779 | AM11195-SS-NL |
| CS000507 | Tri-SM6.1-avβ6-(TA14)csggaauGfgAfa_2NAfcugaacacaas(invAb) | 780 | AM11197-SS-NL |
| CS000531 | Tri-SM6.1-avβ6-(TA14)csggaauggAfAfAfcugaauacaas(invAb) | 781 | AM10721-SS-NL |
| CS000672 | avβ6-SM6.1-L6-C6-csggaauggAfAfAfcugaacacaas(invAb) | 782 | AM11514-SS-NL |
| CS000673 | avβ6-SM6.1-L6-C6s-(invAb)scggaauggAfAfAfcugaacacaas(invAb) | 783 | AM11515-SS-NL |
| CS000674 | avβ6-SM6.1-Alk-cyHex-csggaauggAfAfAfcugaacacaas(invAb) | 784 | AM11516-SS-NL |
| CS000675 | avβ6-SM6.1-Alk-cyHexs-(invAb)scggaauggAfAfAfcugaacacaas | 785 | AM11517-SS-NL |
| | (invAb) | | |
| CS000690 | avβ6-pep1-C6-csggaauggAfAfAfcugaacacaas(invAb) | 786 | AM11514-SS-NL |
| CS000691 | avβ6-pep1-C6s-(invAb)scggaauggAfAfAfcugaacacaas(invAb) | 787 | AM11515-SS-NL |
| CS000986 | Tri-SM6.1-avβ6-(TA14)gsggaauggAfAfAfcugaacacaas(invAb) | 788 | AM10716-SS-NL |
| CS000988 | Tri-SM6.1-avβ6-(TA14)csggaauggAfAfAfcuiaacacaas(invAb) | 789 | AM10718-SS-NL |
| CS000989 | Tri-SM6.l-avβ6-(TA14)csggaauggAfa_2NAfcuiaacacaas(invAb) | 790 | AM10719-SS-NL |
| CS001021 | Tri-SM6.1-avβ6-(TA14)gsgaauggaAfAfCfugaacacaias(invAb) | 791 | AM10310-SS-NL |
| CS001024 | Tri-SM6.1-avβ6-(TA14)asccagauuCfCfUfgggaaiccaas(invAb) | 792 | AM10313-SS-NL |
| CS001027 | Tri-SM6.1-avβ6-(TA14)usccugggaAfGfCfcagaaauugus(invAb) | 793 | AM10316-SS-NL |
| CS001579 | Tri-SM6.1-avβ6-(TA14)gsaguagGfuGfcUfcaaaacaucas(invAb) | 11 | AM12910-SS-NL |
| CS001582 | Tri-SM6.1-avβ6-(TA14)asggcaaugAfAfCfaggaauigaas(invAb) | 12 | AM12911-SS-NL |
| CS002138 | Tri-SM6.1-avβ6-(TA14)csggaauggAfaAfcUfgaacacaas(invAb) | 796 | AM14092-SS-NL |
| CS002399 | Tri-SM6.1-avβ6-(TA14)gsaguagGfuGfcUfcaaaacauca(invAb) | 797 | AM12910-SS-NL |
| CS002976 | Tri-SM6.1-avβ6-(TA14)cscugggaaGfCfCfagaaauuguas(invAb) | 798 | AM15766-SS-NL |
| CS003048 | Tri-SM6.1-avβ6-(TA14)scsggaauggAfAfAfcugaacacaas(invAb) | 799 | AM10307-SS-NL |

The RAGE RNAi agents disclosed herein are formed by annealing an antisense strand with a sense strand. A sense strand containing a sequence listed in Table 2, Table 4, Table 5, or Table 6 can be hybridized to any antisense strand containing a sequence listed in Table 2 or Table 3, provided the two sequences have a region of at least 85% complementarity over a contiguous 15, 16, 17, 18, 19, 20, or 21 nucleotide sequence.

As shown in Table 5 above, certain of the example RAGE RNAi agent nucleotide sequences are shown to further include reactive linking groups at one or both of the 5′ terminal end and the 3′ terminal end of the sense strand. For example, many of the RAGE RNAi agent sense strand sequences shown in Table 5 above have a (TriAlk14) linking group at the 5′ end of the nucleotide sequence. Other linking groups, such as an (NH2-C6) linking group or a (6-SS-6) or (C6-SS-C6) linking group, may be present as well or alternatively in certain embodiments. Such reactive linking groups are positioned to facilitate the linking of targeting ligands, targeting groups, and/or PK/PD modulators to the RAGE RNAi agents disclosed herein. Linking or conjugation reactions are well known in the art and provide for formation of covalent linkages between two molecules or reactants. Suitable conjugation reactions for use in the scope of the inventions herein include, but are not limited to, amide coupling reaction, Michael addition reaction, hydrazone formation reaction, inverse-demand Diels-Alder cycloaddition reaction, oxime ligation, and Copper (I)-catalyzed or strain-promoted azide-alkyne cycloaddition reaction cycloaddition reaction.

In some embodiments, targeting ligands, such as the integrin targeting ligands shown in the examples and figures disclosed herein, can be synthesized as activated esters, such as tetrafluorophenyl (TFP) esters, which can be displaced by a reactive amino group (e.g., NH₂-C₆) to attach the targeting ligand to the RAGE RNAi agents disclosed herein. In some embodiments, targeting ligands are synthesized as azides, which can be conjugated to a propargyl (e.g., TriAlk14) or DBCO group, for example, via Copper (I)-catalyzed or strain-promoted azide-alkyne cycloaddition reaction.

Additionally, certain of the nucleotide sequences can be synthesized with a dT nucleotide at the 3′ terminal end of the sense strand, followed by (3′→5′) a linker (e.g., C6-SS-C6). The linker can, in some embodiments, facilitate the linkage to additional components, such as, for example, a PK/PD modulator or one or more targeting ligands. As described herein, the disulfide bond of C6-SS-C6 is first reduced, removing the dT from the molecule, which can then facilitate the conjugation of the desired PK/PD modulator. The terminal dT nucleotide therefore is not a part of the fully conjugated construct.

In some embodiments, the antisense strand of a RAGE RNAi agent disclosed herein differs by 0, 1, 2, or 3 nucleotides from any of the antisense strand sequences in Table 3 or Table 10. In some embodiments, the sense strand of a RAGE RNAi agent disclosed herein differs by 0, 1, 2, or 3 nucleotides from any of the sense strand sequences in Table 4, Table 5, Table 6, or Table 10.

In some embodiments, a RAGE RNAi agent antisense strand comprises a nucleotide sequence of any of the sequences in Table 2 or Table 3. In some embodiments, a RAGE RNAi agent antisense strand comprises the sequence of nucleotides (from 5′ end→3′ end) 1-17, 2-17, 1-18, 2-18, 1-19, 2-19, 1-20, 2-20, 1-21, 2-21, 1-22, 2-22, 1-23, 2-23, 1-24, or 2-24 of any of the sequences in Table 2, Table 3, or Table 10. In certain embodiments, a RAGE RNAi agent antisense strand comprises or consists of a modified sequence of any one of the modified sequences in Table 3 or Table 10.

In some embodiments, a RAGE RNAi agent sense strand comprises the nucleotide sequence of any of the sequences in Table 2 or Table 4. In some embodiments, a RAGE RNAi agent sense strand comprises the sequence of nucleotides (from 5′ end→3′ end) 1-17, 2-17, 3-17, 4-17, 1-18, 2-18, 3-18, 4-18, 1-19, 2-19, 3-19, 4-19, 1-20, 2-20, 3-20, 4-20, 1-21, 2-21, 3-21, 4-21, 1-22, 2-22, 3-22, 4-22, 1-23, 2-23, 3-23, 4-23, 1-24, 2-24, 3-24, or 4-24, of any of the sequences in Table 2, Table 4, Table 5, Table 6, or Table 10. In certain embodiments, a RAGE RNAi agent sense strand comprises or consists of a modified sequence of any one of the modified sequences in Table 3 or Table 10.

For the RNAi agents disclosed herein, the nucleotide at position 1 of the antisense strand (from 5′ end→3′ end) can be perfectly complementary to an AGER gene, or can be non-complementary to an AGER gene. In some embodiments, the nucleotide at position 1 of the antisense strand (from 5′ end→3′ end) is a U, A, or dT (or a modified version of U, A or dT). In some embodiments, the nucleotide at position 1 of the antisense strand (from 5′ end→3′ end) forms an A:U or U:A base pair with the sense strand.

In some embodiments, a RAGE RNAi agent antisense strand comprises the sequence of nucleotides (from 5′ end→3′ end) 2-18 or 2-19 of any of the antisense strand sequences in Table 2, Table 3, or Table 10. In some embodiments, a RAGE RNAi sense strand comprises the sequence of nucleotides (from 5′ end→3′ end) 1-17 or 1-18 of any of the sense strand sequences in Table 2, Table 4, Table 5, Table 6, or Table 10.

In some embodiments, a RAGE RNAi agent includes (i) an antisense strand comprising the sequence of nucleotides (from 5′ end→3′ end) 2-18 or 2-19 of any of the antisense strand sequences in Table 2, Table 3, or Table 10, and (ii) a sense strand comprising the sequence of nucleotides (from 5′ end→3′ end) 1-17 or 1-18 of any of the sense strand sequences in Table 2, Table 4, Table 5, Table 6, or Table 10.

A sense strand containing a sequence listed in Table 2 or Table 4 can be hybridized to any antisense strand containing a sequence listed in Table 2 or Table 3 provided the two sequences have a region of at least 85% complementarity over a contiguous 16, 17, 18, 19, 20, or 21 nucleotide sequence. In some embodiments, the RAGE RNAi agent has a sense strand consisting of the modified sequence of any of the modified sequences in Table 4, Table 5, Table 6, or Table 10, and an antisense strand consisting of the modified sequence of any of the modified sequences in Table 3 or Table 10. Certain representative sequence pairings are exemplified by the Duplex ID Nos. shown in Tables 7A, 7B, 8, 9A and 9B.

In some embodiments, a RAGE RNAi agent comprises, consists of, or consists essentially of a duplex represented by any one of the Duplex ID Nos. presented herein. In some embodiments, a RAGE RNAi agent consists of any of the Duplex ID Nos. presented herein. In some embodiments, a RAGE RNAi agent comprises the sense strand and antisense strand nucleotide sequences of any of the Duplex ID Nos. presented herein. In some embodiments, a RAGE RNAi agent comprises the sense strand and antisense strand nucleotide sequences of any of the Duplex ID Nos. presented herein and a targeting group, linking group, and/or other non-nucleotide group wherein the targeting group, linking group, and/or other non-nucleotide group is covalently linked (i.e., conjugated) to the sense strand or the antisense strand. In some embodiments, a RAGE RNAi agent includes the sense strand and antisense strand modified nucleotide sequences of any of the Duplex ID Nos. presented herein. In some embodiments, a RAGE RNAi agent comprises the sense strand and antisense strand modified nucleotide sequences of any of the Duplex ID Nos. presented herein and a targeting group, linking group, and/or other non-nucleotide group, wherein the targeting group, linking group, and/or other non-nucleotide group is covalently linked to the sense strand or the antisense strand.

In some embodiments, a RAGE RNAi agent comprises an antisense strand and a sense strand having the nucleotide sequences of any of the antisense strand/sense strand duplexes of Tables 2, 7A, 7B, 8, 9A, 9B, or 10, and comprises a targeting group. In some embodiments, a RAGE RNAi agent comprises an antisense strand and a sense strand having the nucleotide sequences of any of the antisense strand/sense strand duplexes of Tables 2, 7A, 7B, 8, 9A, 9B, or 10, and comprises one or more αvβ6 integrin targeting ligands.

In some embodiments, a RAGE RNAi agent comprises an antisense strand and a sense strand having the nucleotide sequences of any of the antisense strand/sense strand duplexes of Tables 2, 7A, 7B, 8, 9A, 9B, or 10, and comprises a targeting group that is an integrin targeting ligand. In some embodiments, a RAGE RNAi agent comprises an antisense strand and a sense strand having the nucleotide sequences of any of the antisense strand/sense strand duplexes of Tables 2, 7A, 7B, 8, 9A, 9B, or 10, and comprises one or more αvβ6 integrin targeting ligands or clusters of αvβ6 integrin targeting ligands (e.g., a tridentate αvβ6 integrin targeting ligand).

In some embodiments, a RAGE RNAi agent comprises an antisense strand and a sense strand having the modified nucleotide sequences of any of the antisense strand/sense strand duplexes of Tables 7A, 7B, 8, 9A, 9B, and 10.

In some embodiments, a RAGE RNAi agent comprises an antisense strand and a sense strand having the modified nucleotide sequences of any of the antisense strand/sense strand duplexes of Tables 7A, 7B, 8, 9A, 9B, and 10, and comprises an integrin targeting ligand.

In some embodiments, a RAGE RNAi agent comprises, consists of, or consists essentially of any of the duplexes of Tables 7A, 7B, 8, 9A, 9B, and 10.

TABLE 7A
AGE RNAi Agent Duplexes with Corresponding Sense and Antisense
Strand ID Numbers and Sequence ID numbers for the modified and unmodified
nucleotide sequences. (Shown without Linking Agents or Conjugates)

| | | AS | AS | | SS | SS |
|---|---|---|---|---|---|---|
| | | modified | unmodified | | modified | unmodified |
| | | SEQID | SEQ ID | | SEQ ID | SEQ ID |
| Duplex | AS ID | NO: | NO: | SS ID | NO: | NO: |
| AD07474 | AM10308-AS | 2 | 7 | AM10307-SS-NL | 13 | 19 |
| AD07475 | AM10309-AS | 3 | 7 | AM10307-SS-NL | 13 | 19 |
| AD07476 | AM10311-AS | 543 | 801 | AM10310-SS-NL | 623 | 839 |
| AD07477 | AM10312-AS | 544 | 801 | AM10310-SS-NL | 623 | 839 |
| AD07478 | AM10314-AS | 545 | 802 | AM10313-SS-NL | 624 | 840 |
| AD07479 | AM10315-AS | 546 | 802 | AM10313-SS-NL | 624 | 840 |
| AD07480 | AM10317-AS | 547 | 803 | AM10316-SS-NL | 625 | 841 |
| AD07481 | AM10318-AS | 548 | 803 | AM10316-SS-NL | 625 | 841 |
| AD07559 | AM10467-AS | 549 | 804 | AM10466-SS-NL | 626 | 842 |
| AD07560 | AM10469-AS | 550 | 805 | AM10468-SS-NL | 627 | 843 |
| AD07561 | AM10471-AS | 551 | 806 | AM10470-SS-NL | 628 | 844 |
| AD07562 | AM10473-AS | 552 | 807 | AM10472-SS-NL | 629 | 845 |
| AD07563 | AM10475-AS | 553 | 808 | AM10474-SS-NL | 630 | 846 |
| AD07564 | AM10477-AS | 554 | 809 | AM10476-SS-NL | 631 | 847 |
| AD07565 | AM10479-AS | 555 | 810 | AM10478-SS-NL | 632 | 848 |
| AD07566 | AM10481-AS | 556 | 811 | AM10480-SS-NL | 633 | 849 |
| AD07567 | AM10483-AS | 557 | 812 | AM10482-SS-NL | 634 | 850 |
| AD07621 | AM10571-AS | 558 | 813 | AM10570-SS-NL | 635 | 851 |
| AD07622 | AM10573-AS | 559 | 813 | AM10572-SS-NL | 636 | 852 |
| AD07623 | AM10575-AS | 560 | 814 | AM10574-SS-NL | 637 | 853 |
| AD07624 | AM10575-AS | 560 | 814 | AM10576-SS-NL | 638 | 854 |
| AD07661 | AM10308-AS | 2 | 7 | AM10644-SS-NL | 13 | 19 |
| AD07700 | AM10717-AS | 561 | 815 | AM10716-SS-NL | 640 | 855 |
| AD07701 | AM10308-AS | 2 | 7 | AM10718-SS-NL | 641 | 856 |
| AD07702 | AM10308-AS | 2 | 7 | AM10719-SS-NL | 642 | 857 |
| AD07703 | AM10720-AS | 562 | 7 | AM10307-SS-NL | 13 | 19 |
| AD07704 | AM10308-AS | 2 | 7 | AM10721-SS-NL | 643 | 858 |
| AD07705 | AM10722-AS | 563 | 7 | AM10307-SS-NL | 13 | 19 |
| AD07706 | AM10723-AS | 564 | 7 | AM10307-SS-NL | 13 | 19 |
| AD07707 | AM10724-AS | 565 | 7 | AM10307-SS-NL | 13 | 19 |
| AD07708 | AM10723-AS | 564 | 7 | AM10725-SS-NL | 644 | 19 |
| AD07715 | AM10317-AS | 547 | 803 | AM10737-SS-NL | 645 | 841 |
| AD07725 | AM10752-AS | 566 | 9 | AM10751-SS-NL | 646 | 20 |
| AD07726 | AM10754-AS | 4 | 8 | AM10753-SS-NL | 15 | 21 |
| AD07727 | AM10756-AS | 568 | 818 | AM10755-SS-NL | 648 | 861 |
| AD07728 | AM10758-AS | 569 | 819 | AM10757-SS-NL | 649 | 862 |
| AD07729 | AM10760-AS | 570 | 820 | AM10759-SS-NL | 650 | 863 |
| AD07730 | AM10762-AS | 571 | 821 | AM10761-SS-NL | 651 | 864 |
| AD07736 | AM10774-AS | 572 | 822 | AM10773-SS-NL | 652 | 865 |
| AD07737 | AM10776-AS | 573 | 823 | AM10775-SS-NL | 653 | 866 |
| AD07738 | AM10778-AS | 574 | 824 | AM10777-SS-NL | 654 | 867 |
| AD07739 | AM10780-AS | 575 | 825 | AM10779-SS-NL | 655 | 868 |
| AD07740 | AM10782-AS | 576 | 826 | AM10781-SS-NL | 656 | 869 |
| AD07741 | AM10784-AS | 577 | 827 | AM10783-SS-NL | 657 | 870 |
| AD07742 | AM10786-AS | 578 | 828 | AM10785-SS-NL | 658 | 871 |
| AD07743 | AM10788-AS | 579 | 829 | AM10787-SS-NL | 659 | 872 |
| AD07972 | AM11103-AS | 580 | 7 | AM10307-SS-NL | 13 | 19 |
| AD07973 | AM11104-AS | 581 | 7 | AM10307-SS-NL | 13 | 19 |
| AD07974 | AM11104-AS | 581 | 7 | AM11105-SS-NL | 660 | 19 |
| AD07975 | AM11104-AS | 581 | 7 | AM11106-SS-NL | 13 | 19 |
| AD07976 | AM11104-AS | 581 | 7 | AM11107-SS-NL | 662 | 19 |
| AD08030 | AM10309-AS | 3 | 7 | AM10721-SS-NL | 643 | 858 |
| AD08031 | AM11188-AS | 582 | 7 | AM10725-SS-NL | 644 | 19 |
| AD08032 | AM10723-AS | 564 | 7 | AM11189-SS-NL | 663 | 858 |
| AD08033 | AM11188-AS | 582 | 7 | AM11189-SS-NL | 663 | 858 |
| AD08034 | AM11190-AS | 583 | 7 | AM10725-SS-NL | 644 | 19 |
| AD08035 | AM11191-AS | 584 | 7 | AM10725-SS-NL | 644 | 19 |
| AD08036 | AM11192-AS | 585 | 7 | AM10725-SS-NL | 644 | 19 |
| AD08037 | AM11194-AS | 586 | 815 | AM11193-SS-NL | 664 | 855 |
| AD08038 | AM11196-AS | 587 | 830 | AM11195-SS-NL | 665 | 873 |
| AD08039 | AM10723-AS | 564 | 7 | AM11197-SS-NL | 666 | 874 |
| AD08258 | AM10309-AS | 3 | 7 | AM11512-SS-NL | 667 | 19 |
| AD08259 | AM10309-AS | 3 | 7 | AM11513-SS-NL | 13 | 19 |
| AD08260 | AM10309-AS | 3 | 7 | AM11514-SS-NL | 13 | 19 |
| AD08261 | AM10309-AS | 3 | 7 | AM11515-SS-NL | 670 | 19 |
| AD08262 | AM10309-AS | 3 | 7 | AM11516-SS-NL | 13 | 19 |
| AD08263 | AM10309-AS | 3 | 7 | AM11517-SS-NL | 672 | 19 |
| AD08432 | AM11757-AS | 588 | 7 | AM10725-SS-NL | 644 | 19 |
| AD08433 | AM11758-AS | 589 | 7 | AM10725-SS-NL | 644 | 19 |
| AD08434 | AM11759-AS | 590 | 815 | AM11193-SS-NL | 664 | 855 |
| AD08435 | AM11760-AS | 591 | 815 | AM11193-SS-NL | 664 | 855 |
| AD08436 | AM11761-AS | 592 | 815 | AM11193-SS-NL | 664 | 855 |
| AD08437 | AM11762-AS | 593 | 7 | AM10725-SS-NL | 644 | 19 |
| AD08438 | AM11763-AS | 594 | 815 | AM11193-SS-NL | 664 | 855 |
| AD08439 | AM11764-AS | 595 | 815 | AM11193-SS-NL | 664 | 855 |
| AD08510 | AM11889-AS | 596 | 831 | AM11888-SS-NL | 673 | 875 |
| AD08511 | AM11889-AS | 596 | 831 | AM11890-SS-NL | 674 | 876 |
| AD08512 | AM11892-AS | 597 | 832 | AM11891-SS-NL | 675 | 877 |
| AD08513 | AM11894-AS | 598 | 833 | AM11893-SS-NL | 676 | 878 |
| AD08514 | AM11895-AS | 599 | 9 | AM10751-SS-NL | 646 | 20 |
| AD08515 | AM11897-AS | 5 | 9 | AM11896-SS-NL | 677 | 20 |
| AD08516 | AM11898-AS | 6 | 9 | AM11896-SS-NL | 677 | 20 |
| AD08517 | AM11898-AS | 6 | 9 | AM11899-SS-NL | 678 | 879 |
| AD08518 | AM11897-AS | 5 | 9 | AM11900-SS-NL | 14 | 20 |
| AD08519 | AM11898-AS | 6 | 9 | AM11900-SS-NL | 14 | 20 |
| AD08520 | AM11897-AS | 5 | 9 | AM11901-SS-NL | 680 | 20 |
| AD08521 | AM11898-AS | 6 | 9 | AM11901-SS-NL | 680 | 20 |
| AD08711 | AM12234-AS | 602 | 8 | AM10753-SS-NL | 15 | 21 |
| AD08712 | AM12236-AS | 603 | 8 | AM12235-SS-NL | 681 | 21 |
| AD08713 | AM12237-AS | 604 | 8 | AM12235-SS-NL | 681 | 21 |
| AD08714 | AM12236-AS | 603 | 8 | AM12238-SS-NL | 682 | 880 |
| AD08715 | AM12236-AS | 603 | 8 | AM12239-SS-NL | 683 | 881 |
| AD08716 | AM12240-AS | 605 | 8 | AM12238-SS-NL | 682 | 880 |
| AD08717 | AM12241-AS | 606 | 8 | AM12238-SS-NL | 682 | 880 |
| AD08718 | AM12236-AS | 603 | 8 | AM12242-SS-NL | 684 | 882 |
| AD08719 | AM12236-AS | 603 | 8 | AM12243-SS-NL | 685 | 883 |
| AD08720 | AM12245-AS | 607 | 834 | AM12244-SS-NL | 686 | 884 |
| AD08898 | AM10309-AS | 3 | 7 | AM10644-SS-NL | 13 | 19 |
| AD08944 | AM12593-AS | 608 | 835 | AM12592-SS-NL | 687 | 885 |
| AD08945 | AM12594-AS | 609 | 9 | AM11900-SS-NL | 14 | 20 |
| AD08946 | AM12596-AS | 610 | 836 | AM12595-SS-NL | 688 | 886 |
| AD08947 | AM11897-AS | 5 | 9 | AM12597-SS-NL | 689 | 20 |
| AD09051 | AM10754-AS | 4 | 8 | AM12754-SS-NL | 690 | 880 |
| AD09052 | AM12234-AS | 602 | 8 | AM12754-SS-NL | 690 | 880 |
| AD09053 | AM12236-AS | 603 | 8 | AM10753-SS-NL | 15 | 21 |
| AD09054 | AM10754-AS | 4 | 8 | AM12235-SS-NL | 681 | 21 |
| AD09055 | AM12755-AS | 611 | 8 | AM12235-SS-NL | 681 | 21 |
| AD09056 | AM12756-AS | 612 | 8 | AM12235-SS-NL | 681 | 21 |
| AD09057 | AM12757-AS | 613 | 8 | AM12235-SS-NL | 681 | 21 |
| AD09150 | AM11897-AS | 5 | 9 | AM12910-SS-NL | 14 | 20 |
| AD09151 | AM11898-AS | 6 | 9 | AM12910-SS-NL | 14 | 20 |
| AD09152 | AM10754-AS | 4 | 8 | AM12911-SS-NL | 15 | 21 |
| AD09797 | AM10309-AS | 3 | 7 | AM13987-SS-NL | 13 | 19 |
| AD09868 | AM10308-AS | 2 | 7 | AM13987-SS-NL | 13 | 19 |
| AD09870 | AM14090-AS | 614 | 7 | AM10725-SS-NL | 644 | 19 |
| AD09871 | AM14091-AS | 615 | 7 | AM10725-SS-NL | 644 | 19 |
| AD09872 | AM14091-AS | 615 | 7 | AM14092-SS-NL | 694 | 19 |
| AD09873 | AM14093-AS | 616 | 9 | AM11900-SS-NL | 14 | 20 |
| AD09874 | AM14094-AS | 617 | 9 | AM11900-SS-NL | 14 | 20 |
| AD09875 | AM14095-AS | 618 | 9 | AM11900-SS-NL | 14 | 20 |
| AD09876 | AM14095-AS | 618 | 9 | AM12597-SS-NL | 689 | 20 |
| AD09877 | AM14095-AS | 618 | 9 | AM11896-SS-NL | 677 | 20 |
| AD10543 | AM15021-AS | 619 | 830 | AM11195-SS-NL | 665 | 873 |
| AD11078 | AM15767-AS | 620 | 837 | AM15766-SS-NL | 695 | 887 |
| AD11080 | AM15770-AS | 621 | 801 | AM10310-SS-NL | 623 | 839 |
| AD11353 | AM10309-AS | 3 | 7 | AM16133-SS-NL | 13 | 19 |

TABLE 7B
RAGE RNAi Agent Duplexes with Corresponding Sense and Antisense
Strand ID Numbers and Sequence ID numbers for the modified and unmodified
nucleotide sequences.

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AD07474 | AM10308-AS | 2 | 7 | AM10307-SS | 16 | 19 |
| AD07475 | AM10309-AS | 3 | 7 | AM10307-SS | 16 | 19 |
| AD07476 | AM10311-AS | 543 | 801 | AM10310-SS | 698 | 839 |
| AD07477 | AM10312-AS | 544 | 801 | AM10310-SS | 698 | 839 |
| AD07478 | AM10314-AS | 545 | 802 | AM10313-SS | 699 | 840 |
| AD07479 | AM10315-AS | 546 | 802 | AM10313-SS | 699 | 840 |
| AD07480 | AM10317-AS | 547 | 803 | AM10316-SS | 700 | 841 |
| AD07481 | AM10318-AS | 548 | 803 | AM10316-SS | 700 | 841 |
| AD07559 | AM10467-AS | 549 | 804 | AM10466-SS | 701 | 842 |
| AD07560 | AM10469-AS | 550 | 805 | AM10468-SS | 702 | 843 |
| AD07561 | AM10471-AS | 551 | 806 | AM10470-SS | 703 | 844 |
| AD07562 | AM10473-AS | 552 | 807 | AM10472-SS | 704 | 845 |
| AD07563 | AM10475-AS | 553 | 808 | AM10474-SS | 705 | 846 |
| AD07564 | AM10477-AS | 554 | 809 | AM10476-SS | 706 | 847 |
| AD07565 | AM10479-AS | 555 | 810 | AM10478-SS | 707 | 848 |
| AD07566 | AM10481-AS | 556 | 811 | AM10480-SS | 708 | 849 |
| AD07567 | AM10483-AS | 557 | 812 | AM10482-SS | 709 | 850 |
| AD07621 | AM10571-AS | 558 | 813 | AM10570-SS | 710 | 851 |
| AD07622 | AM10573-AS | 559 | 813 | AM10572-SS | 711 | 852 |
| AD07623 | AM10575-AS | 560 | 814 | AM10574-SS | 712 | 853 |
| AD07624 | AM10575-AS | 560 | 814 | AM10576-SS | 713 | 854 |
| AD07661 | AM10308-AS | 2 | 7 | AM10644-SS | 16 | 19 |
| AD07700 | AM10717-AS | 561 | 815 | AM10716-SS | 715 | 855 |
| AD07701 | AM10308-AS | 2 | 7 | AM10718-SS | 716 | 856 |
| AD07702 | AM10308-AS | 2 | 7 | AM10719-SS | 717 | 857 |
| AD07703 | AM10720-AS | 562 | 7 | AM10307-SS | 16 | 19 |
| AD07704 | AM10308-AS | 2 | 7 | AM10721-SS | 718 | 858 |
| AD07705 | AM10722-AS | 563 | 7 | AM10307-SS | 16 | 19 |
| AD07706 | AM10723-AS | 564 | 7 | AM10307-SS | 16 | 19 |
| AD07707 | AM10724-AS | 565 | 7 | AM10307-SS | 16 | 19 |
| AD07708 | AM10723-AS | 564 | 7 | AM10725-SS | 719 | 19 |
| AD07715 | AM10317-AS | 547 | 803 | AM10737-SS | 720 | 841 |
| AD07725 | AM10752-AS | 566 | 9 | AM10751-SS | 721 | 20 |
| AD07726 | AM10754-AS | 4 | 8 | AM10753-SS | 18 | 21 |
| AD07727 | AM10756-AS | 568 | 818 | AM10755-SS | 723 | 861 |
| AD07728 | AM10758-AS | 569 | 819 | AM10757-SS | 724 | 862 |
| AD07729 | AM10760-AS | 570 | 820 | AM10759-SS | 725 | 863 |
| AD07730 | AM10762-AS | 571 | 821 | AM10761-SS | 726 | 864 |
| AD07736 | AM10774-AS | 572 | 822 | AM10773-SS | 727 | 865 |
| AD07737 | AM10776-AS | 573 | 823 | AM10775-SS | 728 | 866 |
| AD07738 | AM10778-AS | 574 | 824 | AM10777-SS | 729 | 867 |
| AD07739 | AM10780-AS | 575 | 825 | AM10779-SS | 730 | 868 |
| AD07740 | AM10782-AS | 576 | 826 | AM10781-SS | 731 | 869 |
| AD07741 | AM10784-AS | 577 | 827 | AM10783-SS | 732 | 870 |
| AD07742 | AM10786-AS | 578 | 828 | AM10785-SS | 733 | 871 |
| AD07743 | AM10788-AS | 579 | 829 | AM10787-SS | 734 | 872 |
| AD07972 | AM11103-AS | 580 | 7 | AM10307-SS | 16 | 19 |
| AD07973 | AM11104-AS | 581 | 7 | AM10307-SS | 16 | 19 |
| AD07974 | AM11104-AS | 581 | 7 | AM11105-SS | 735 | 19 |
| AD07975 | AM11104-AS | 581 | 7 | AM11106-SS | 736 | 19 |
| AD07976 | AM11104-AS | 581 | 7 | AM11107-SS | 737 | 19 |
| AD08030 | AM10309-AS | 3 | 7 | AM10721-SS | 718 | 858 |
| AD08031 | AM11188-AS | 582 | 7 | AM10725-SS | 719 | 19 |
| AD08032 | AM10723-AS | 564 | 7 | AM11189-SS | 738 | 858 |
| AD08033 | AM11188-AS | 582 | 7 | AM11189-SS | 738 | 858 |
| AD08034 | AM11190-AS | 583 | 7 | AM10725-SS | 719 | 19 |
| AD08035 | AM11191-AS | 584 | 7 | AM10725-SS | 719 | 19 |
| AD08036 | AM11192-AS | 585 | 7 | AM10725-SS | 719 | 19 |
| AD08037 | AM11194-AS | 586 | 815 | AM11193-SS | 739 | 855 |
| AD08038 | AM11196-AS | 587 | 830 | AM11195-SS | 740 | 873 |
| AD08039 | AM10723-AS | 564 | 7 | AM11197-SS | 741 | 874 |
| AD08258 | AM10309-AS | 3 | 7 | AM11512-SS | 742 | 19 |
| AD08259 | AM10309-AS | 3 | 7 | AM11513-SS | 16 | 19 |
| AD08260 | AM10309-AS | 3 | 7 | AM11514-SS | 16 | 19 |
| AD08261 | AM10309-AS | 3 | 7 | AM11515-SS | 745 | 19 |
| AD08262 | AM10309-AS | 3 | 7 | AM11516-SS | 16 | 19 |
| AD08263 | AM10309-AS | 3 | 7 | AM11517-SS | 747 | 19 |
| AD08432 | AM11757-AS | 588 | 7 | AM10725-SS | 719 | 19 |
| AD08433 | AM11758-AS | 589 | 7 | AM10725-SS | 719 | 19 |
| AD08434 | AM11759-AS | 590 | 815 | AM11193-SS | 739 | 855 |
| AD08435 | AM11760-AS | 591 | 815 | AM11193-SS | 739 | 855 |
| AD08436 | AM11761-AS | 592 | 815 | AM11193-SS | 739 | 855 |
| AD08437 | AM11762-AS | 593 | 7 | AM10725-SS | 719 | 19 |
| AD08438 | AM11763-AS | 594 | 815 | AM11193-SS | 739 | 855 |
| AD08439 | AM11764-AS | 595 | 815 | AM11193-SS | 739 | 855 |
| AD08510 | AM11889-AS | 596 | 831 | AM11888-SS | 748 | 875 |
| AD08511 | AM11889-AS | 596 | 831 | AM11890-SS | 749 | 876 |
| AD08512 | AM11892-AS | 597 | 832 | AM11891-SS | 750 | 877 |
| AD08513 | AM11894-AS | 598 | 833 | AM11893-SS | 751 | 878 |
| AD08514 | AM11895-AS | 599 | 9 | AM10751-SS | 721 | 20 |
| AD08515 | AM11897-AS | 5 | 9 | AM11896-SS | 752 | 20 |
| AD08516 | AM11898-AS | 6 | 9 | AM11896-SS | 752 | 20 |
| AD08517 | AM11898-AS | 6 | 9 | AM11899-SS | 753 | 879 |
| AD08518 | AM11897-AS | 5 | 9 | AM11900-SS | 17 | 20 |
| AD08519 | AM11898-AS | 6 | 9 | AM11900-SS | 17 | 20 |
| AD08520 | AM11897-AS | 5 | 9 | AM11901-SS | 755 | 20 |
| AD08521 | AM11898-AS | 6 | 9 | AM11901-SS | 755 | 20 |
| AD08711 | AM12234-AS | 602 | 8 | AM10753-SS | 18 | 21 |
| AD08712 | AM12236-AS | 603 | 8 | AM12235-SS | 756 | 21 |
| AD08713 | AM12237-AS | 604 | 8 | AM12235-SS | 756 | 21 |
| AD08714 | AM12236-AS | 603 | 8 | AM12238-SS | 757 | 880 |
| AD08715 | AM12236-AS | 603 | 8 | AM12239-SS | 758 | 881 |
| AD08716 | AM12240-AS | 605 | 8 | AM12238-SS | 757 | 880 |
| AD08717 | AM12241-AS | 606 | 8 | AM12238-SS | 757 | 880 |
| AD08718 | AM12236-AS | 603 | 8 | AM12242-SS | 759 | 882 |
| AD08719 | AM12236-AS | 603 | 8 | AM12243-SS | 760 | 883 |
| AD08720 | AM12245-AS | 607 | 834 | AM12244-SS | 761 | 884 |
| AD08898 | AM10309-AS | 3 | 7 | AM10644-SS | 16 | 19 |
| AD08944 | AM12593-AS | 608 | 835 | AM12592-SS | 762 | 885 |
| AD08945 | AM12594-AS | 609 | 9 | AM11900-SS | 17 | 20 |
| AD08946 | AM12596-AS | 610 | 836 | AM12595-SS | 763 | 886 |
| AD08947 | AM11897-AS | 5 | 9 | AM12597-SS | 764 | 20 |
| AD09051 | AM10754-AS | 4 | 8 | AM12754-SS | 765 | 880 |
| AD09052 | AM12234-AS | 602 | 8 | AM12754-SS | 765 | 880 |
| AD09053 | AM12236-AS | 603 | 8 | AM10753-SS | 18 | 21 |
| AD09054 | AM10754-AS | 4 | 8 | AM12235-SS | 756 | 21 |
| AD09055 | AM12755-AS | 611 | 8 | AM12235-SS | 756 | 21 |
| AD09056 | AM12756-AS | 612 | 8 | AM12235-SS | 756 | 21 |
| AD09057 | AM12757-AS | 613 | 8 | AM12235-SS | 756 | 21 |
| AD09150 | AM11897-AS | 5 | 9 | AM12910-SS | 17 | 20 |
| AD09151 | AM11898-AS | 6 | 9 | AM12910-SS | 17 | 20 |
| AD09152 | AM10754-AS | 4 | 8 | AM12911-SS | 18 | 21 |
| AD09797 | AM10309-AS | 3 | 7 | AM13987-SS | 16 | 19 |
| AD09868 | AM10308-AS | 2 | 7 | AM13987-SS | 16 | 19 |
| AD09870 | AM14090-AS | 614 | 7 | AM10725-SS | 719 | 19 |
| AD09871 | AM14091-AS | 615 | 7 | AM10725-SS | 719 | 19 |
| AD09872 | AM14091-AS | 615 | 7 | AM14092-SS | 769 | 19 |
| AD09873 | AM14093-AS | 616 | 9 | AM11900-SS | 17 | 20 |
| AD09874 | AM14094-AS | 617 | 9 | AM11900-SS | 17 | 20 |
| AD09875 | AM14095-AS | 618 | 9 | AM11900-SS | 17 | 20 |
| AD09876 | AM14095-AS | 618 | 9 | AM12597-SS | 764 | 20 |
| AD09877 | AM14095-AS | 618 | 9 | AM11896-SS | 752 | 20 |
| AD10543 | AM15021-AS | 619 | 830 | AM11195-SS | 740 | 873 |
| AD11078 | AM15767-AS | 620 | 837 | AM15766-SS | 770 | 887 |
| AD11080 | AM15770-AS | 621 | 801 | AM10310-SS | 698 | 839 |
| AD11353 | AM10309-AS | 3 | 7 | AM16133-SS | 771 | 19 |

TABLE 8
RAGE RNAi Agent Duplexes with Corresponding Sense and Antisense Strand
ID Numbers and Sequence ID numbers for the modified and unmodified nucleotide
sequences. (Shown with Targeting Ligand Conjugates)

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AC000286 | AM10308-AS | 2 | 7 | CS000363 | 10 | 19 |
| AC000287 | AM10309-AS | 3 | 7 | CS000363 | 10 | 19 |
| AC000288 | AM11103-AS | 580 | 7 | CS000363 | 10 | 19 |
| AC000289 | AM11104-AS | 581 | 7 | CS000363 | 10 | 19 |
| AC000290 | AM11104-AS | 581 | 7 | CS000368 | 773 | 19 |
| AC000291 | AM11104-AS | 581 | 7 | CS000369 | 774 | 19 |
| AC000292 | AM10309-AS | 3 | 7 | CS000363 | 10 | 19 |
| AC000293 | AM11103-AS | 580 | 7 | CS000363 | 10 | 19 |
| AC000294 | AM11104-AS | 581 | 7 | CS000363 | 10 | 19 |
| AC000312 | AM11104-AS | 581 | 7 | CS000386 | 775 | 19 |
| AC000414 | AM11188-AS | 582 | 7 | CS000497 | 776 | 858 |
| AC000415 | AM11190-AS | 583 | 7 | CS000499 | 777 | 19 |
| AC000416 | AM11191-AS | 584 | 7 | CS000499 | 777 | 19 |
| AC000417 | AM11192-AS | 585 | 7 | CS000499 | 777 | 19 |
| AC000418 | AM11194-AS | 586 | 815 | CS000503 | 778 | 855 |
| AC000419 | AM11196-AS | 587 | 830 | CS000505 | 779 | 873 |
| AC000420 | AM10723-AS | 564 | 7 | CS000507 | 780 | 874 |
| AC000438 | AM10308-AS | 2 | 7 | CS000531 | 781 | 858 |
| AC000439 | AM10723-AS | 564 | 7 | CS000499 | 777 | 19 |
| AC000440 | AM10309-AS | 3 | 7 | CS000531 | 781 | 858 |
| AC000441 | AM11188-AS | 582 | 7 | CS000499 | 777 | 19 |
| AC000442 | AM10723-AS | 564 | 7 | CS000497 | 776 | 858 |
| AC000549 | AM10309-AS | 3 | 7 | CS000672 | 782 | 19 |
| AC000550 | AM10309-AS | 3 | 7 | CS000673 | 783 | 19 |
| AC000551 | AM10309-AS | 3 | 7 | CS000674 | 784 | 19 |
| AC000552 | AM10309-AS | 3 | 7 | CS000675 | 785 | 19 |
| AC000567 | AM10309-AS | 3 | 7 | CS000690 | 786 | 19 |
| AC000568 | AM10309-AS | 3 | 7 | CS000691 | 787 | 19 |
| AC000790 | AM10717-AS | 561 | 815 | CS000986 | 788 | 855 |
| AC000791 | AM10308-AS | 2 | 7 | CS000988 | 789 | 856 |
| AC000792 | AM10308-AS | 2 | 7 | CS000989 | 790 | 857 |
| AC000793 | AM10720-AS | 562 | 7 | CS000363 | 10 | 19 |
| AC000794 | AM10722-AS | 563 | 7 | CS000363 | 10 | 19 |
| AC000795 | AM10723-AS | 564 | 7 | CS000363 | 10 | 19 |
| AC000796 | AM10724-AS | 565 | 7 | CS000363 | 10 | 19 |
| AC000818 | AM10311-AS | 543 | 801 | CS001021 | 791 | 839 |
| AC000819 | AM10312-AS | 544 | 801 | CS001021 | 791 | 839 |
| AC000820 | AM10314-AS | 545 | 802 | CS001024 | 792 | 840 |
| AC000821 | AM10315-AS | 546 | 802 | CS001024 | 792 | 840 |
| AC000822 | AM10317-AS | 547 | 803 | CS001027 | 793 | 841 |
| AC000823 | AM10318-AS | 548 | 803 | CS001027 | 793 | 841 |
| AC001134 | AM11762-AS | 593 | 7 | CS000499 | 777 | 19 |
| AC001266 | AM11897-AS | 5 | 9 | CS001579 | 11 | 20 |
| AC001267 | AM11898-AS | 6 | 9 | CS001579 | 11 | 20 |
| AC001268 | AM10754-AS | 4 | 8 | CS001582 | 12 | 21 |
| AC001274 | AM11757-AS | 588 | 7 | CS000499 | 777 | 19 |
| AC001653 | AM14090-AS | 614 | 7 | CS000499 | 777 | 19 |
| AC001654 | AM14091-AS | 615 | 7 | CS000499 | 777 | 19 |
| AC001655 | AM14091-AS | 615 | 7 | CS002138 | 796 | 19 |
| AC001877 | AM11897-AS | 5 | 9 | CS002399 | 797 | 20 |
| AC002047 | AM15021-AS | 619 | 830 | CS000505 | 779 | 873 |
| AC002345 | AM15767-AS | 620 | 837 | CS002976 | 798 | 887 |
| AC002347 | AM15770-AS | 621 | 801 | CS001021 | 791 | 839 |
| AC002399 | AM10309-AS | 3 | 7 | CS003048 | 799 | 19 |

TABLE 9A
Conjugate Duplex ID Numbers Referencing Position
Targeted On AGER (RAGE) Gene

| | | | Targeted AGER Gene |
|---|---|---|---|
| | | | Position |
| Duplex | AS ID | SS ID | (Of SEQ ID NO: 1) |
| AC000286 | AM10308-AS | CS000363 | 177 |
| AC000287 | AM10309-AS | CS000363 | 177 |
| AC000288 | AM11103-AS | CS000363 | 177 |
| AC000289 | AM11104-AS | CS000363 | 177 |
| AC000290 | AM11104-AS | CS000368 | 177 |
| AC000291 | AM11104-AS | CS000369 | 177 |
| AC000292 | AM10309-AS | CS000363 | 177 |
| AC000293 | AM11103-AS | CS000363 | 177 |
| AC000294 | AM11104-AS | CS000363 | 177 |
| AC000312 | AM11104-AS | CS000386 | 177 |
| AC000414 | AM11188-AS | CS000497 | 177 |
| AC000415 | AM11190-AS | CS000499 | 177 |
| AC000416 | AM11191-AS | CS000499 | 177 |
| AC000417 | AM11192-AS | CS000499 | 177 |
| AC000418 | AM11194-AS | CS000503 | 177 |
| AC000419 | AM11196-AS | CS000505 | 177 |
| AC000420 | AM10723-AS | CS000507 | 177 |
| AC000438 | AM10308-AS | CS000531 | 177 |
| AC000439 | AM10723-AS | CS000499 | 177 |
| AC000440 | AM10309-AS | CS000531 | 177 |
| AC000441 | AM11188-AS | CS000499 | 177 |
| AC000442 | AM10723-AS | CS000497 | 177 |
| AC000549 | AM10309-AS | CS000672 | 177 |
| AC000550 | AM10309-AS | CS000673 | 177 |
| AC000551 | AM10309-AS | CS000674 | 177 |
| AC000552 | AM10309-AS | CS000675 | 177 |
| AC000567 | AM10309-AS | CS000690 | 177 |
| AC000568 | AM10309-AS | CS000691 | 177 |
| AC000790 | AM10717-AS | CS000986 | 177 |
| AC000791 | AM10308-AS | CS000988 | 177 |
| AC000792 | AM10308-AS | CS000989 | 177 |
| AC000793 | AM10720-AS | CS000363 | 177 |
| AC000794 | AM10722-AS | CS000363 | 177 |
| AC000795 | AM10723-AS | CS000363 | 177 |
| AC000796 | AM10724-AS | CS000363 | 177 |
| AC000818 | AM10311-AS | CS001021 | 178 |
| AC000819 | AM10312-AS | CS001021 | 178 |
| AC000820 | AM10314-AS | CS001024 | 384 |
| AC000821 | AM10315-AS | CS001024 | 384 |
| AC000822 | AM10317-AS | CS001027 | 391 |
| AC000823 | AM10318-AS | CS001027 | 391 |
| AC001134 | AM11762-AS | CS000499 | 177 |
| AC001266 | AM11897-AS | CS001579 | 90 |
| AC001267 | AM11898-AS | CS001579 | 90 |
| AC001268 | AM10754-AS | CS001582 | 330 |
| AC001274 | AM11757-AS | CS000499 | 177 |
| AC001653 | AM14090-AS | CS000499 | 177 |
| AC001654 | AM14091-AS | CS000499 | 177 |
| AC001655 | AM14091-AS | CS002138 | 177 |
| AC001877 | AM11897-AS | CS002399 | 90 |
| AC002047 | AM15021-AS | CS000505 | 177 |
| AC002345 | AM15767-AS | CS002976 | 392 |
| AC002347 | AM15770-AS | CS001021 | 178 |
| AC002399 | AM10309-AS | CS003048 | 177 |

TABLE 9B
Conjugate ID Numbers and Corresponding AD Duplex Numbers,
Referencing Position Targeted On RAGE (AGER) Gene

| | Corresponding | Targeted AGER |
|---|---|---|
| AC Duplex | AD Duplex | Gene Position |
| Number | Number | (Of SEQ ID NO: 1) |
| AC000286 | AD07474 | 177 |
| AC000287 | AD07475 | 177 |
| AC000288 | AD07972 | 177 |
| AC000289 | AD07973 | 177 |
| AC000290 | AD07974 | 177 |
| AC000291 | AD07975 | 177 |
| AC000292 | AD07475 | 177 |
| AC000293 | AD07972 | 177 |
| AC000294 | AD07973 | 177 |
| AC000312 | AD07976 | 177 |
| AC000414 | AD08033 | 177 |
| AC000415 | AD08034 | 177 |
| AC000416 | AD08035 | 177 |
| AC000417 | AD08036 | 177 |
| AC000418 | AD08037 | 177 |
| AC000419 | AD08038 | 177 |
| AC000420 | AD08039 | 177 |
| AC000438 | AD07704 | 177 |
| AC000439 | AD07708 | 177 |
| AC000440 | AD08030 | 177 |
| AC000441 | AD08031 | 177 |
| AC000442 | AD08032 | 177 |
| AC000549 | AD08260 | 177 |
| AC000550 | AD08261 | 177 |
| AC000551 | AD08262 | 177 |
| AC000552 | AD08263 | 177 |
| AC000567 | AD08260 | 177 |
| AC000568 | AD08261 | 177 |
| AC000790 | AD07700 | 177 |
| AC000791 | AD07701 | 177 |
| AC000792 | AD07702 | 177 |
| AC000793 | AD07703 | 177 |
| AC000794 | AD07705 | 177 |
| AC000795 | AD07706 | 177 |
| AC000796 | AD07707 | 177 |
| AC000818 | AD07476 | 178 |
| AC000819 | AD07477 | 178 |
| AC000820 | AD07478 | 384 |
| AC000821 | AD07479 | 384 |
| AC000822 | AD07480 | 391 |
| AC000823 | AD07481 | 391 |
| AC001134 | AD08437 | 177 |
| AC001266 | AD09150 | 90 |
| AC001267 | AD09151 | 90 |
| AC001268 | AD09152 | 330 |
| AC001274 | AD08432 | 177 |
| AC001653 | AD09870 | 177 |
| AC001654 | AD09871 | 177 |
| AC001655 | AD09872 | 177 |
| AC001877 | AD10075 | 90 |
| AC002047 | AD10543 | 177 |
| AC002345 | AD11078 | 392 |
| AC002347 | AD11080 | 178 |
| AC002399 | AD11353 | 177 |

TABLE 10
Conjugate ID Numbers With Chemically Modified Antisense and Sense Strands (including Linkers and Conjugates)

| | | SEQ | | SEQ |
|---|---|---|---|---|
| AC ID | Sense Strand (Fully Modified | ID | | ID |
| Number | with Conjugated Targeting Ligand) (5′→3′) | NO: | Antisense Strand (5′→3′) | NO: |
| AC000286 | Tri-SM6.1-avβ6-(TA14)csggaauggAfAfAfcugaacacaas(invAb) | 10 | usUfsgsUfgUfuCfaGfuUfuCfcAfuUfcCfsg | 2 |
| AC000287 | Tri-SM6.1-avβ6-(TA14)csggaauggAfAfAfcugaacacaas(invAb) | 10 | cPrpusUfsgsUfgUfuCfaGfuUfuCfcAfuUfcCfsg | 3 |
| AC000288 | Tri-SM6.1-avβ6-(TA14)csggaauggAfAfAfcugaacacaas(invAb) | 10 | cPrpusUfgUfgUfuCfaGfuUfuCfcAfuUfcCfsg | 580 |
| AC000289 | Tri-SM6.1-avβ6-(TA14)csggaauggAfAfAfcugaacacaas(invAb) | 10 | cPrpuUfgUfgUfuCfaGfuUfuCfcAfuUfcCfsg | 581 |
| AC000290 | Tri-SM6.1-avβ6-(TA14)cggaauggAfAfAfcugaacacaas(invAb) | 773 | cPrpuUfgUfgUfuCfaGfuUfuCfcAfuUfcCfsg | 581 |
| AC000291 | Tri-SM6.1-avβ6-(TA14)csggaauggAfAfAfcugaacacaa(invAb) | 774 | cPrpuUfgUfgUfuCfaGfuUfuCfcAfuUfcCfsg | 581 |
| AC000292 | Tri-SM6.1-avβ6-(TA14)csggaauggAfAfAfcugaacacaas(invAb) | 10 | cPrpusUfsgsUfgUfuCfaGfuUfuCfcAfuUfcCfsg | 3 |
| AC000293 | Tri-SM6.1-avβ6-(TA14)csggaauggAfAfAfcugaacacaas(invAb) | 10 | cPrpusUfgUfgUfuCfaGfuUfuCfcAfuUfcCfsg | 580 |
| AC000294 | Tri-SM6.1-avβ6-(TA14)csggaauggAfAfAfcugaacacaas(invAb) | 10 | cPrpuUfgUfgUfuCfaGfuUfuCfcAfuUfcCfsg | 581 |
| AC000312 | Tri-SM6.1-avβ6-(TA14)cggaauggAfAfAfcugaacacaa(invAb) | 775 | cPrpuUfgUfgUfuCfaGfuUfuCfcAfuUfcCfsg | 581 |
| AC000414 | Tri-SM6.1-avβ6-(TA14)csggaauGfgAfaAfcugaauacaas(invAb) | 776 | cPrpusUfsgsuguucaguUfuCfcAfuuccsg | 582 |
| AC000415 | Tri-SM6.1-avβ6-(TA14)csggaauGfgAfaAfcugaacacaas(invAb) | 777 | usUfsgsuguuC_{UNA}aguUfuCfcAfuuccsg | 583 |
| AC000416 | Tri-SM6.1-avβ6-(TA14)csggaauGfgAfaAfcugaacacaas(invAb) | 777 | usUfsgsuguU_{UNA}caguUfuCfcAfuuccsg | 584 |
| AC000417 | Tri-SM6.1-avβ6-(TA14)csggaauGfgAfaAfcugaacacaas(invAb) | 777 | usUfsgsugU_{UNA}UcaguUfuCfcAfuuccsg | 585 |
| AC000418 | Tri-SM6.1-avβ6-(TA14)gsggaauGfgAfaAfcugaacacaas(invAb) | 778 | usUfsgsuguucaguUfuCfcAfuuccsc | 586 |
| AC000419 | Tri-SM6.1-avβ6-(TA14)usggaauGfgAfaAfcugaacacaas(invAb) | 779 | usUfsgsuguucaguUfuCfcAfuuccsa | 587 |
| AC000420 | Tri-SM6.1-avβ6-(TA14)csggaauGfgAfa_2NAfcugaacacaas(invAb) | 780 | usUfsgsuguucaguUfuCfcAfuuccsg | 564 |
| AC000438 | Tri-SM6.1-avβ6-(TA14)csggaauggAfAfAfcugaauacaas(invAb) | 781 | usUfsgsUfgUfuCfaGfuUfuCfcAfuUfcCfsg | 2 |
| AC000439 | Tri-SM6.1-avβ6-(TA14)csggaauGfgAfaAfcugaacacaas(invAb) | 777 | usUfsgsuguucaguUfuCfcAfuuccsg | 564 |
| AC000440 | Tri-SM6.1-avβ6-(TA14)csggaauggAfAfAfcugaauacaas(invAb) | 781 | cPrpusUfsgsUfgUfuCfaGfuUfuCfcAfuUfcCfsg | 3 |
| AC000441 | Tri-SM6.1-avβ6-(TA14)csggaauGfgAfaAfcugaacacaas(invAb) | 777 | cPrpusUfsgsuguucaguUfuCfcAfuuccsg | 582 |
| AC000442 | Tri-SM6.1-avβ6-(TA14)csggaauGfgAfaAfcugaauacaas(invAb) | 776 | usUfsgsuguucaguUfuCfcAfuuccsg | 564 |
| AC000549 | avβ6-SM6.1-L6-C6-csggaauggAfAfAfcugaacacaas(invAb) | 782 | cPrpusUfsgsUfgUfuCfaGfuUfuCfcAfuUfcCfsg | 3 |
| AC000550 | avβ6-SM6.1-L6-C6s-(invAb)scggaauggAfAfAfcugaacacaas(invAb) | 783 | cPrpusUfsgsUfgUfuCfaGfuUfuCfcAfuUfcCfsg | 3 |
| AC000551 | avβ6-SM6.1-Alk-cyHex-csggaauggAfAfAfcugaacacaas(invAb) | 784 | cPrpusUfsgsUfgUfuCfaGfuUfuCfcAfuUfcCfsg | 3 |
| AC000552 | avβ6-SM6.1-Alk-cyHexs-(invAb)scggaauggAfAfAfcugaacacaas(invAb) | 785 | cPrpusUfsgsUfgUfuCfaGfuUfuCfcAfuUfcCfsg | 3 |
| AC000567 | av6-pep1-C6-csggaauggAfAfAfcugaacacaas(invAb) | 786 | cPrpusUfsgsUfgUfuCfaGfuUfuCfcAfuUfcCfsg | 3 |
| AC000568 | avβ6-pepl-C6s-(invAb)scggaauggAfAfAfcugaacacaas(invAb) | 787 | cPrpusUfsgsUfgUfuCfaGfuUfuCfcAfuUfcCfsg | 3 |
| AC000790 | Tri-SM6.1-avβ6-(TA14)gsggaauggAfAfAfcugaacacaas(invAb) | 788 | usUfsgsUfgUfuCfaGfuUfuCfcAfuUfcCfsc | 561 |
| AC000791 | Tri-SM6.1-avβ6-(TA14)csggaauggAfAfAfcuiaacacaas(invAb) | 789 | usUfsgsUfgUfuCfaGfuUfuCfcAfuUfcCfsg | 2 |
| AC000792 | Tri-SM6.1-avβ6-(TA14)csggaauggAfa_2NAfcuiaacacaas(invAb) | 790 | usUfsgsUfgUfuCfaGfuUfuCfcAfuUfcCfsg | 2 |
| AC000793 | Tri-SM6.1-avβ6-(TA14)csggaauggAfAfAfcugaacacaas(invAb) | 10 | usUfsgsUfgUfU_{UNA}CfaGfuUfuCfcAfuUfcCfsg | 562 |
| AC000794 | Tri-SM6.1-avβ6-(TA14)csggaauggAfAfAfcugaacacaas(invAb) | 10 | usUfsgsUfgUfucaguUfuCfcAfuUfcCfsg | 563 |
| AC000795 | Tri-SM6.1-avβ6-(TA14)csggaauggAfAfAfcugaacacaas(invAb) | 10 | usUfsgsuguucaguUfuCfcAfuuccsg | 564 |
| AC000796 | Tri-SM6.1-avβ6-(TA14)csggaauggAfAfAfcugaacacaas(invAb) | 10 | usUfsgsuguucaGfuUfuCfcAfuuccsg | 565 |
| AC000818 | Tri-SM6.1-avβ6-(TA14)gsgaauggaAfAfCfugaacacaias(invAb) | 791 | usCfsusGfuGfuUfcAfgUfuUfcCfaUfuCfsc | 543 |
| AC000819 | Tri-SM6.1-avβ6-(TA14)gsgaauggaAfAfCfugaacacaias(invAb) | 791 | cPrpusCfsusGfuGfuUfcAfgUfuUfcCfaUfuCfsc | 544 |
| AC000820 | Tri-SM6.1-avβ6-(TA14)asccagauuCfCfUfgggaaiccaas(invAb) | 792 | usUfsgsGfcUfuCfcCfaGfgAfaUfcUfgGfsu | 545 |
| AC000821 | Tri-SM6.1-avβ6-(TA14)asccagauuCfCfUfgggaaiccaas(invAb) | 792 | cPrpusUfsgsGfcUfuCfcCfaGfgAfaUfcUfgGfsu | 546 |
| AC000822 | Tri-SM6.1-avβ6-(TA14)usccugggaAfGfCfcagaaauugus(invAb) | 793 | asCfsasAfuUfuCfuGfgCfuUfcCfcAfgGfsa | 547 |
| AC000823 | Tri-SM6.1-av6-(TA14)usccugggaAfGfCfcagaaauugus(invAb) | 793 | cPrpasCfsasAfuUfuCfuGfgCfuUfcCfcAfgGfsa | 548 |
| AC001134 | Tri-SM6.1-avβ6-(TA14)csggaauGfgAfaAfcugaacacaas(invAb) | 777 | cPrpusUfsgsuguU_{UNA}caguUfuCfcAfuuccsg | 593 |
| AC001266 | Tri-SM6.1-avβ6-(TA14)gsaguagGfuGfcUfcaaaacaucas(invAb) | 11 | usGfsasuguuuugaGfcAfcCfuacusc | 5 |
| AC001267 | Tri-SM6.1-avβ6-(TA14)gsaguagGfuGfcUfcaaaacaucas(invAb) | 11 | cPrpusGfsasuguuuugaGfcAfcCfuacusc | 6 |
| AC001268 | Tri-SM6.1-avβ6-(TA14)asggcaaugAfAfCfaggaauigaas(invAb) | 12 | usUfscsCfaUfuCfcUfgUfuCfaUfuGfcCfsu | 4 |
| AC001274 | Tri-SM6.1-avβ6-(TA14)csggaauGfgAfaAfcugaacacaas(invAb) | 777 | cPrpuUfguguucaguUfuCfcAfuuccsg | 588 |
| AC001653 | Tri-SM6.1-avβ6-(TA14)csggaauGfgAfaAfcugaacacaas(invAb) | 777 | usUfsgsUfguucaguUfuCfcAfuuccsg | 614 |
| AC001654 | Tri-SM6.1-avβ6-(TA14)csggaauGfgAfaAfcugaacacaas(invAb) | 777 | usUfsgsuguUfcaguUfuCfcAfuuccsg | 615 |
| AC001655 | Tri-SM6.1-avβ6-(TA14)csggaauggAfaAfcUfgaacacaas(invAb) | 796 | usUfsgsuguUfcaguUfuCfcAfuuccsg | 615 |
| AC001877 | Tri-SM6.1-avβ6-(TA14)gsaguagGfuGfcUfcaaaacauca(invAb) | 797 | usGfsasuguuuugaGfcAfcCfuacusc | 5 |
| AC002047 | Tri-SM6.1-avβ6-(TA14)usggaauGfgAfaAfcugaacacaas(invAb) | 779 | cPrpusUfsgsuguucaguUfuCfcAfuuccsa | 619 |
| AC002345 | Tri-SM6.1-avβ6-(TA14)cscugggaaGfCfCfagaaauuguas(invAb) | 798 | cPrpusAfscsAfaUfuucugGfcUfuCfcCfagsg | 620 |
| AC002347 | Tri-SM6.1-avβ6-(TA14)gsgaauggaAfAfCfugaacacaias(invAb) | 791 | cPrpusCfsusGfuGfuucagUfuUfcCfaUfucsc | 621 |
| AC002399 | Tri-SM6.1-avβ6-(TA14)scsggaauggAfAfAfcugaacacaas(invAb) | 799 | cPrpusUfsgsUfgUfuCfaGfuUfuCfcAfuUfcCfsg | 3 |

In some embodiments, a RAGE RNAi agent is prepared or provided as a salt, mixed salt, or a free-acid. In some embodiments, a RAGE RNAi agent is prepared or provided as a pharmaceutically acceptable salt. In some embodiments, a RAGE RNAi agent is prepared or provided as a pharmaceutically acceptable sodium or potassium salt The RNAi agents described herein, upon delivery to a cell expressing an AGER gene, inhibit or knockdown expression of one or more AGER genes in vivo and/or in vitro.

### Targeting Groups, Linking Groups, Pharmacokinetic/Pharmacodynamic (PK/PD) Modulators, and Delivery Vehicles

In some embodiments, a RAGE RNAi agent contains or is conjugated to one or more non-nucleotide groups including, but not limited to, a targeting group, a linking group, a pharmacokinetic/pharmacodynamic (PK/PD) modulator, a delivery polymer, or a delivery vehicle. The non-nucleotide group can enhance targeting, delivery, or attachment of the RNAi agent. The non-nucleotide group can be covalently linked to the 3′ and/or 5′ end of either the sense strand and/or the antisense strand. In some embodiments, a RAGE RNAi agent contains a non-nucleotide group linked to the 3′ and/or 5′ end of the sense strand. In some embodiments, a non-nucleotide group is linked to the 5′ end of a RAGE RNAi agent sense strand. A non-nucleotide group can be linked directly or indirectly to the RNAi agent via a linker/linking group. In some embodiments, a non-nucleotide group is linked to the RNAi agent via a labile, cleavable, or reversible bond or linker.

In some embodiments, a non-nucleotide group enhances the pharmacokinetic or biodistribution properties of an RNAi agent or conjugate to which it is attached to improve cell- or tissue-specific distribution and cell-specific uptake of the conjugate. In some embodiments, a non-nucleotide group enhances endocytosis of the RNAi agent.

Targeting groups or targeting moieties enhance the pharmacokinetic or biodistribution properties of a conjugate or RNAi agent to which they are attached to improve cell-specific (including, in some cases, organ specific) distribution and cell-specific (or organ specific) uptake of the conjugate or RNAi agent. A targeting group can be monovalent, divalent, trivalent, tetravalent, or have higher valency for the target to which it is directed. Representative targeting groups include, without limitation, compounds with affinity to cell surface molecule, cell receptor ligands, hapten, antibodies, monoclonal antibodies, antibody fragments, and antibody mimics with affinity to cell surface molecules. In some embodiments, a targeting group is linked to an RNAi agent using a linker, such as a PEG linker or one, two, or three abasic and/or ribitol (abasic ribose) residues, which in some instances can serve as linkers.

A targeting group, with or without a linker, can be attached to the 5′ or 3′ end of any of the sense and/or antisense strands disclosed in Tables 2, 3, 4, 5, 6, and 10. A linker, with or without a targeting group, can be attached to the 5′ or 3′ end of any of the sense and/or antisense strands disclosed in Tables 2, 3, 4, 5, 6, and 10.

The RAGE RNAi agents described herein can be synthesized having a reactive group, such as an amino group (also referred to herein as an amine), at the 5′-terminus and/or the 3′-terminus. The reactive group can be used subsequently to attach a targeting moiety using methods typical in the art.

For example, in some embodiments, the RAGE RNAi agents disclosed herein are synthesized having an NH₂-C₆ group at the 5′-terminus of the sense strand of the RNAi agent. The terminal amino group subsequently can be reacted to form a conjugate with, for example, a group that includes an αvβ6 integrin targeting ligand. In some embodiments, the RAGE RNAi agents disclosed herein are synthesized having one or more alkyne groups at the 5′-terminus of the sense strand of the RNAi agent. The terminal alkyne group(s) can subsequently be reacted to form a conjugate with, for example, a group that includes an αvβ6 integrin targeting ligand.

In some embodiments, a targeting group comprises an integrin targeting ligand. In some embodiments, an integrin targeting ligand is an αvβ6 integrin targeting ligand. The use of an αvβ6 integrin targeting ligand facilitates cell-specific targeting to cells having αvβ6 on its respective surface, and binding of the integrin targeting ligand can facilitate entry of the therapeutic agent, such as an RNAi agent, to which it is linked, into cells such as epithelial cells, including pulmonary epithelial cells and renal epithelial cells. Integrin targeting ligands can be monomeric or monovalent (e.g., having a single integrin targeting moiety) or multimeric or multivalent (e.g., having multiple integrin targeting moieties). The targeting group can be attached to the 3′ and/or 5′ end of the RNAi oligonucleotide using methods known in the art. The preparation of targeting groups, such as αvβ6 integrin targeting ligands, is described, for example, in International Patent Application Publication No. WO 2018/085415 and in International Patent Application Publication No. WO 2019/089765, the contents of each of which are incorporated herein in its entirety.

In some embodiments, targeting groups are linked to the RAGE RNAi agents without the use of an additional linker. In some embodiments, the targeting group is designed having a linker readily present to facilitate the linkage to a RAGE RNAi agent. In some embodiments, when two or more RNAi agents are included in a composition, the two or more RNAi agents can be linked to their respective targeting groups using the same linkers. In some embodiments, when two or more RNAi agents are included in a composition, the two or more RNAi agents are linked to their respective targeting groups using different linkers.

In some embodiments, a linking group is conjugated to the RNAi agent. The linking group facilitates covalent linkage of the agent to a targeting group, pharmacokinetic modulator, delivery polymer, or delivery vehicle. The linking group can be linked to the 3′ and/or the 5′ end of the RNAi agent sense strand or antisense strand. In some embodiments, the linking group is linked to the RNAi agent sense strand. In some embodiments, the linking group is conjugated to the 5′ or 3′ end of an RNAi agent sense strand. In some embodiments, a linking group is conjugated to the 5′ end of an RNAi agent sense strand. Examples of linking groups, include but are not limited to: C6-SS-C6, 6-SS-6, reactive groups such a primary amines (e.g., NH2-C6) and alkynes, alkyl groups, abasic residues/nucleotides, amino acids, tri-alkyne functionalized groups, ribitol, and/or PEG groups. Examples of certain linking groups are provided in Table 11.

A linker or linking group is a connection between two atoms that links one chemical group (such as an RNAi agent) or segment of interest to another chemical group (such as a targeting group, pharmacokinetic modulator, or delivery polymer) or segment of interest via one or more covalent bonds. A labile linkage contains a labile bond. A linkage can optionally include a spacer that increases the distance between the two joined atoms. A spacer may further add flexibility and/or length to the linkage. Spacers include, but are not limited to, alkyl groups, alkenyl groups, alkynyl groups, aryl groups, aralkyl groups, aralkenyl groups, and aralkynyl groups; each of which can contain one or more heteroatoms, heterocycles, amino acids, nucleotides, and saccharides. Spacer groups are well known in the art and the preceding list is not meant to limit the scope of the description. In some embodiments, a RAGE RNAi agent is conjugated to a polyethylene glycol (PEG) moiety, or to a hydrophobic group having 12 or more carbon atoms, such as a cholesterol or palmitoyl group.

In some embodiments, a RAGE RNAi agent is linked to one or more pharmacokinetic/pharmacodynamic (PK/PD) modulators. PK/PD modulators can increase circulation time of the conjugated drug and/or increase the activity of the RNAi agent through improved cell receptor binding, improved cellular uptake, and/or other means. Various PK/PD modulators suitable for use with RNAi agents are known in the art. In some embodiments, the PK/PD modulatory can be cholesterol or cholesteryl derivatives, or in some circumstances a PK/PD modulator can be comprised of alkyl groups, alkenyl groups, alkynyl groups, aryl groups, aralkyl groups, aralkenyl groups, or aralkynyl groups, each of which may be linear, branched, cyclic, and/or substituted or unsubstituted. In some embodiments, the location of attachment for these moieties is at the 5′ or 3′ end of the sense strand, at the 2′ position of the ribose ring of any given nucleotide of the sense strand, and/or attached to the phosphate or phosphorothioate backbone at any position of the sense strand.

Any of the RAGE RNAi agent nucleotide sequences listed in Tables 2, 3, 4, 5, 6, and 10, whether modified or unmodified, can contain 3′ and/or 5′ targeting group(s), linking group(s), and/or PK/PD modulator(s). Any of the RAGE RNAi agent sequences listed in Tables 3, 4, 5, 6, and 10, or are otherwise described herein, which contain a 3′ or 5′ targeting group, linking group, and/or PK/PD modulator can alternatively contain no 3′ or 5′ targeting group, linking group, or PK/PD modulator, or can contain a different 3′ or 5′ targeting group, linking group, or pharmacokinetic modulator including, but not limited to, those depicted in Table 11. Any of the RAGE RNAi agent duplexes listed in Tables 7A, 7B, 8, 9A, 9B, and 10, whether modified or unmodified, can further comprise a targeting group or linking group, including, but not limited to, those depicted in Table 11, and the targeting group or linking group can be attached to the 3′ or 5′ terminus of either the sense strand or the antisense strand of the RAGE RNAi agent duplex.

Examples of certain modified nucleotides, capping moieties, and linking groups are provided in Table 11.

TABLE 11
Structures Representing Various Modified Nucleotides, Capping Moieties, Targeting
Ligands and Targeting and Linking Groups (wherein [symbol] indicates the point of connection)

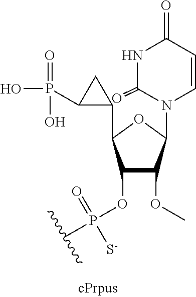

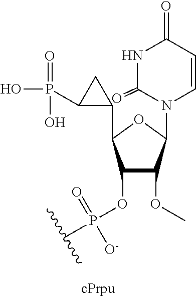

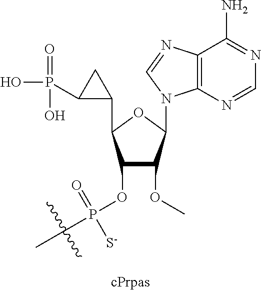

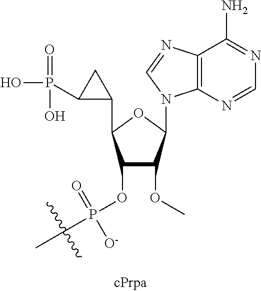

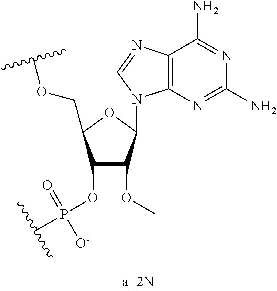

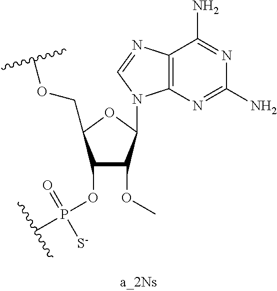

When positioned internally:

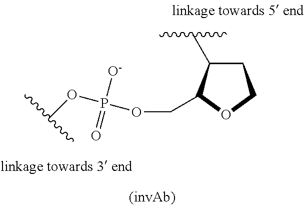

When positioned internally:

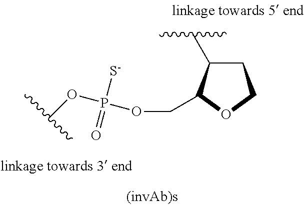

When positioned at the 3′ terminal end:

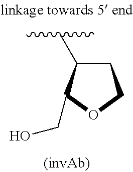

When positioned at the 3′ terminal end:

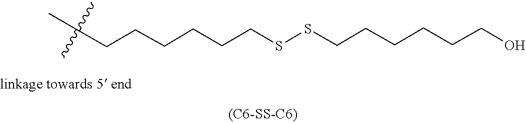 [SMILES: *CCCCCCSSCCCCCCO]

When positioned internally:

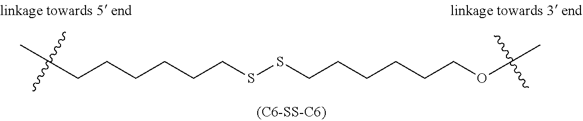

When positioned at the 3′ terminal end:

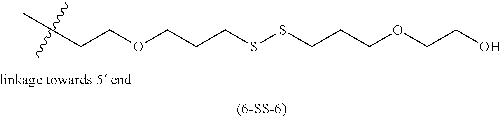 [SMILES: *CCOCCCSSCCCOCCO]

When positioned internally:

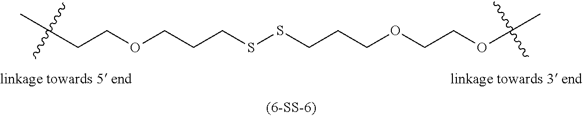

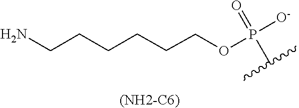 [SMILES: *P(=O)([O-])OCCCCCCN ; "(NH2-C6)"]

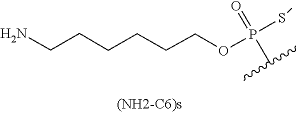 [SMILES: *P(=O)(OCCCCCCN)SC]

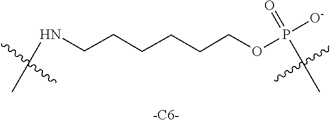 [SMILES: *NCCCCCCOP(*)(=O)[O-]]

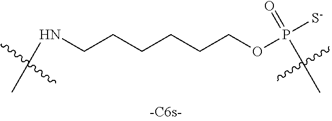 [SMILES: *NCCCCCCOP(*)(=O)[S-]]

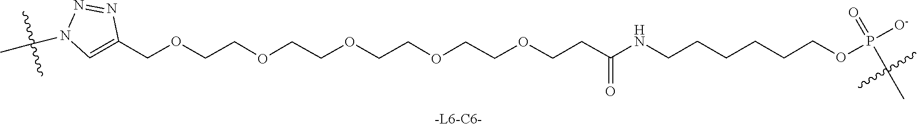 [SMILES: *n1cc(COCCOCCOCCOCCOCCC(=O)NCCCCCCOP(*)(=O)[O-])nn1]

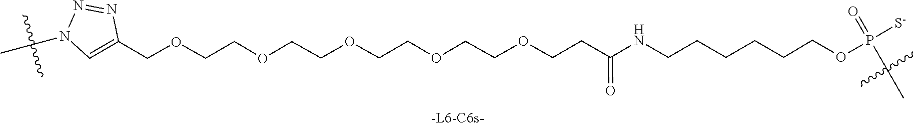 [SMILES: *n1cc(COCCOCCOCCOCCOCCC(=O)NCCCCCCOP(*)(=O)[S-])nn1]

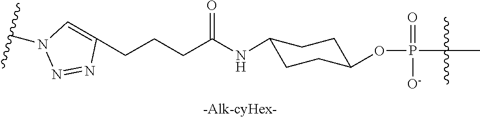 [SMILES: *n1cc(CCCC(=O)NC2CCC(OP(*)(=O)[O-])CC2)nn1]

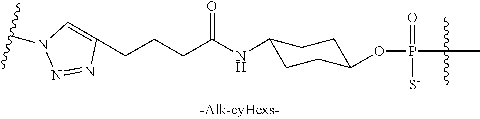 [SMILES: *n1cc(CCCC(=O)NC2CCC(OP(*)(=O)[S-])CC2)nn1]

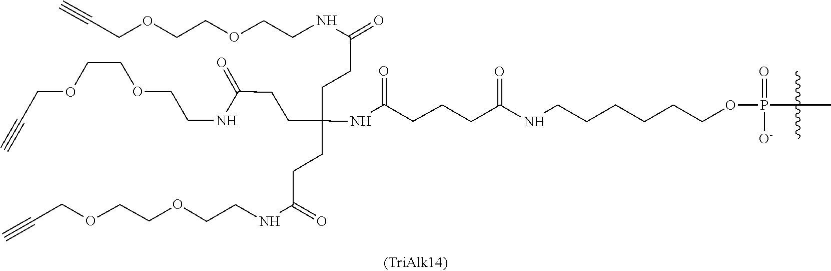 [SMILES: *P(=O)([O-])OCCCCCCNC(=O)CCCC(=O)NC(CCC(=O)NCCOCCOCC#C)(CCC(=O)NCCOCCOCC#C)CCC(=O)NCCOCCOCC#C]

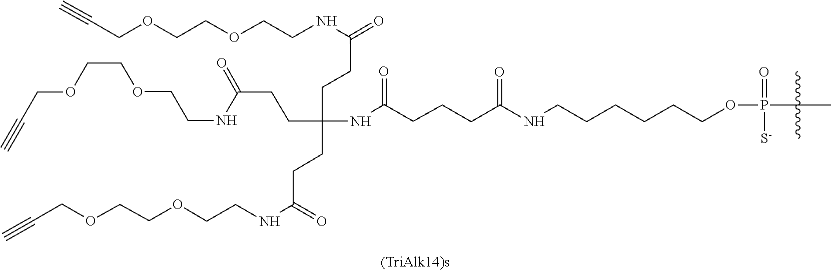 [SMILES: *P(=O)([S-])OCCCCCCNC(=O)CCCC(=O)NC(CCC(=O)NCCOCCOCC#C)(CCC(=O)NCCOCCOCC#C)CCC(=O)NCCOCCOCC#C]

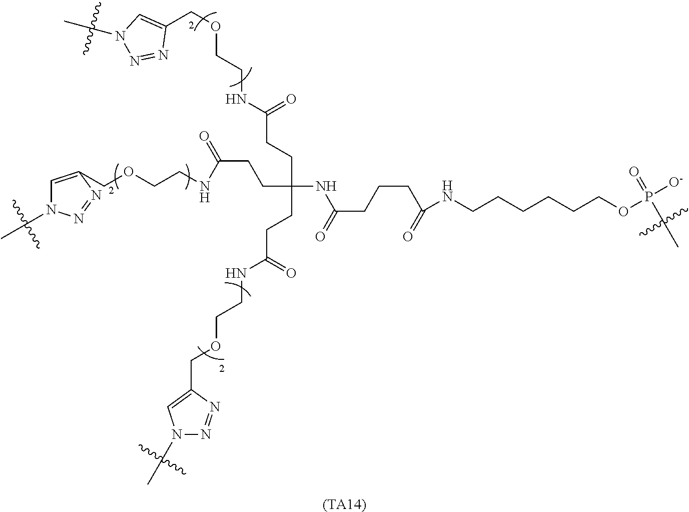

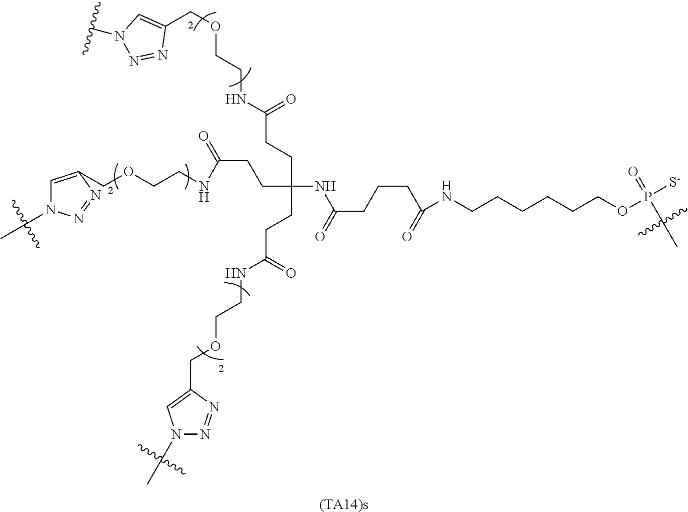

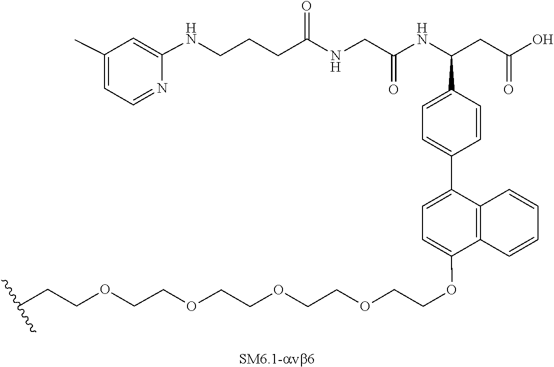 [SMILES: *CCOCCOCCOCCOCCOc1ccc(-c2ccc([C@H](CC(=O)O)NC(=O)CNC(=O)CCCNc3cc(C)ccn3)cc2)c2ccccc12]

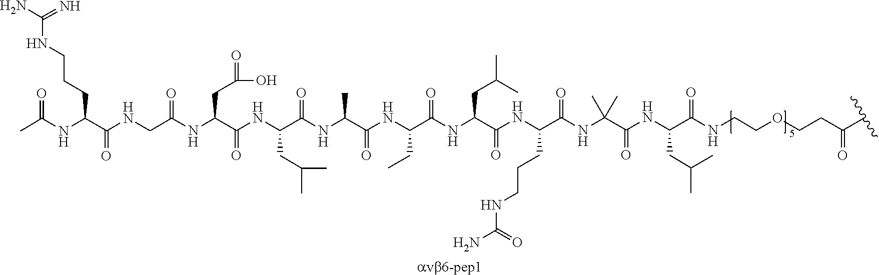

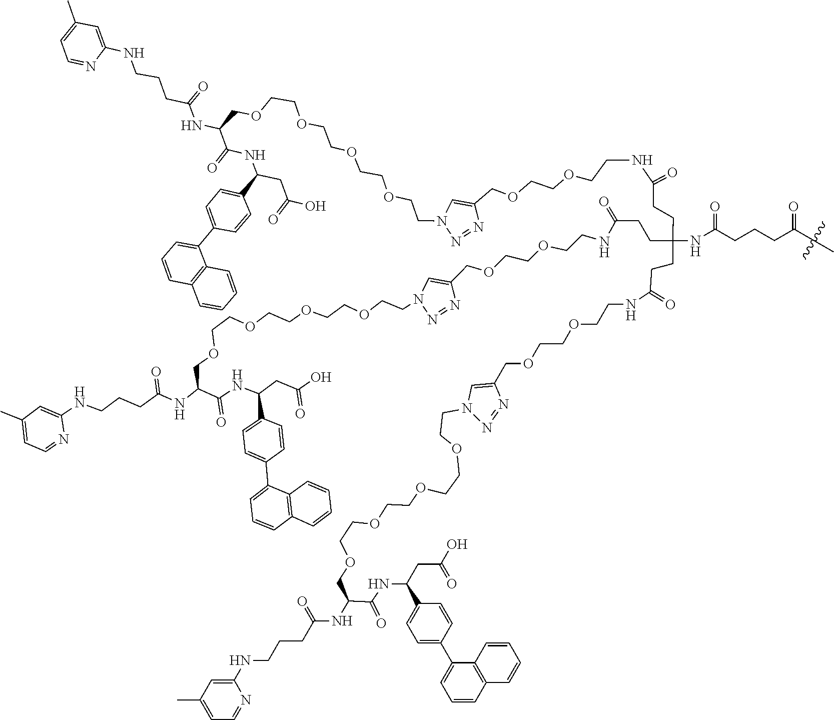 [SMILES: *C(=O)CCCC(=O)NC(CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1)(CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1)CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1]

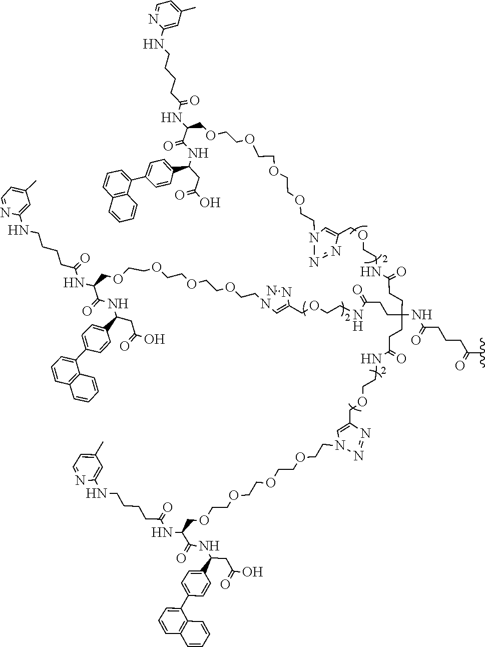

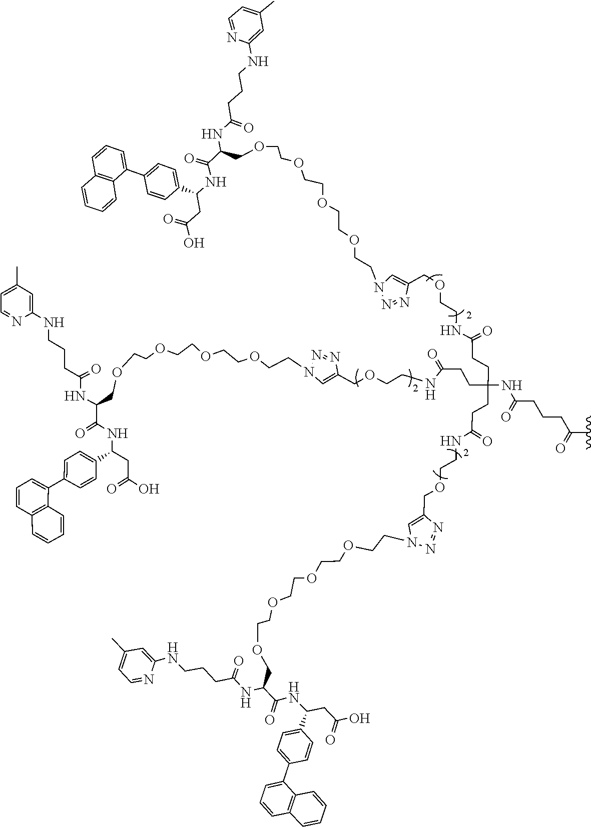

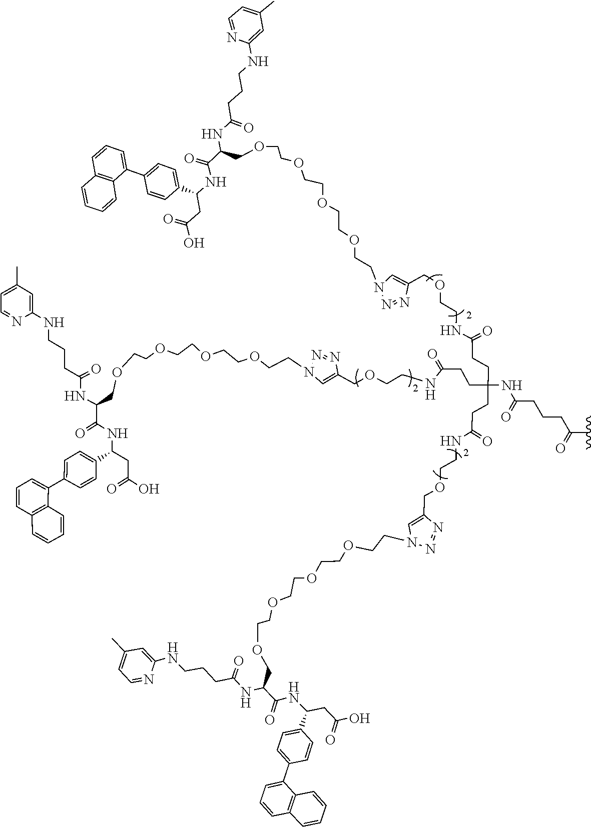

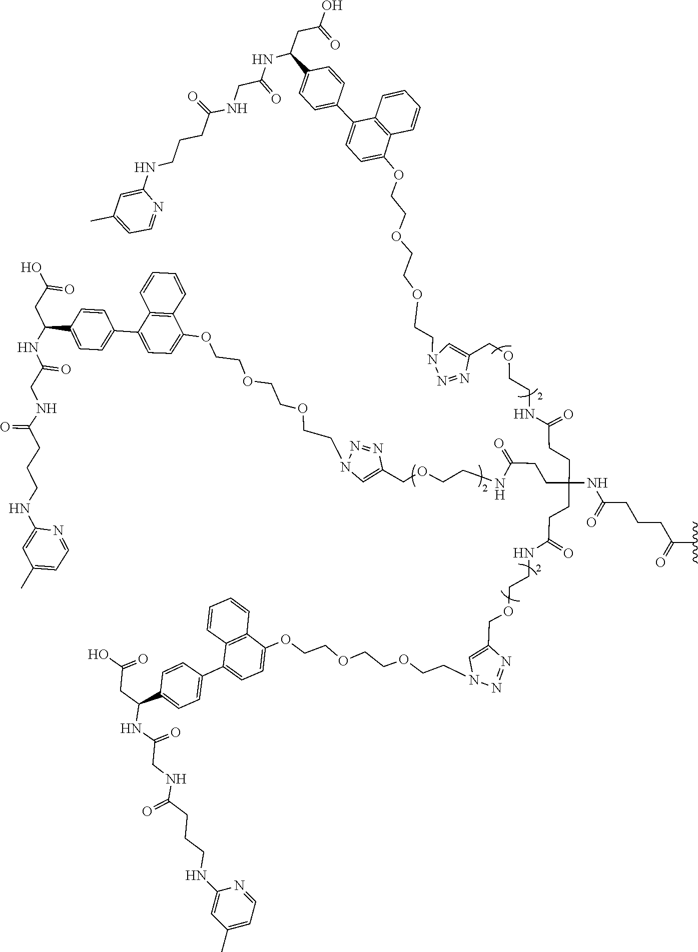

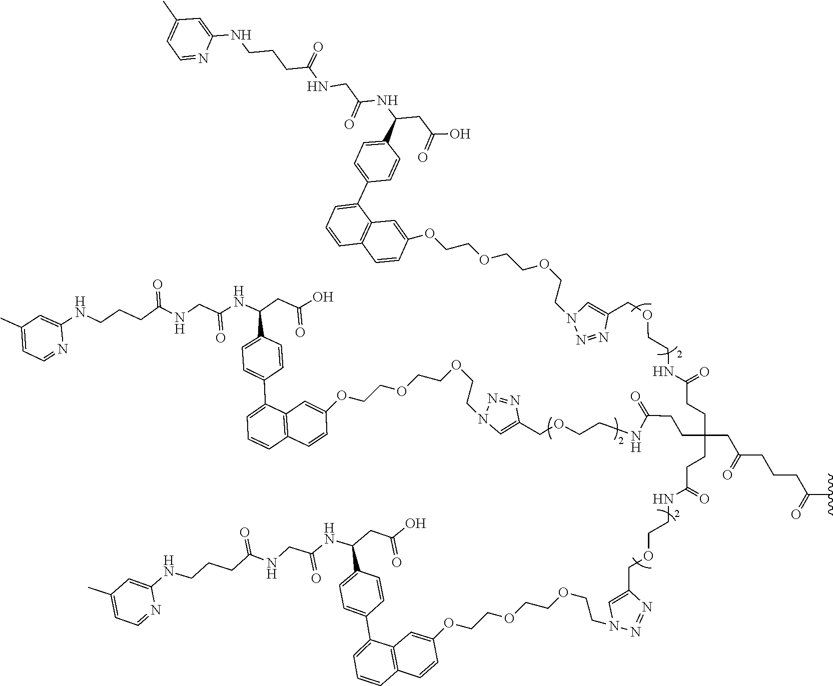

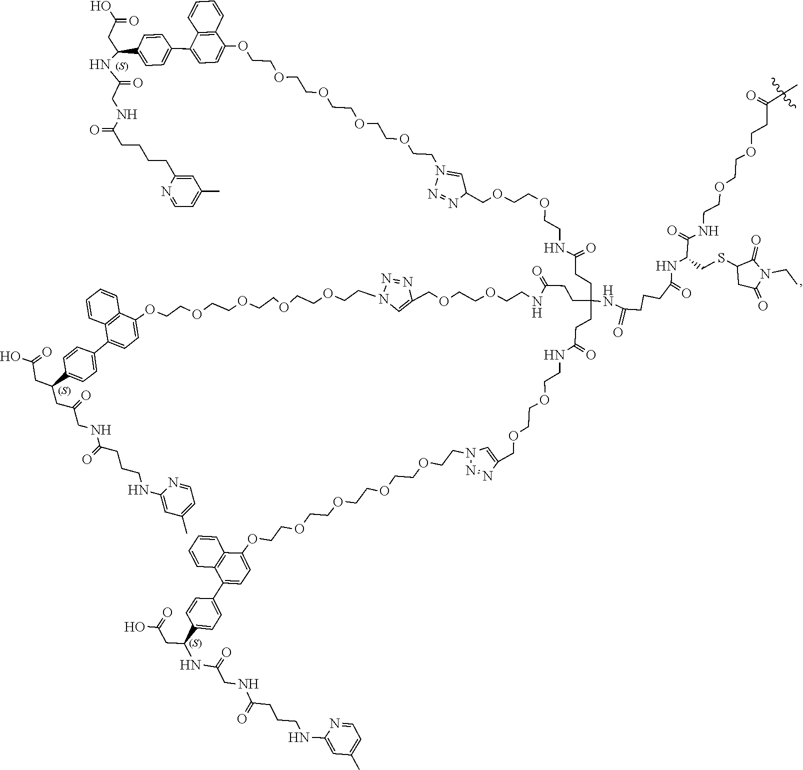

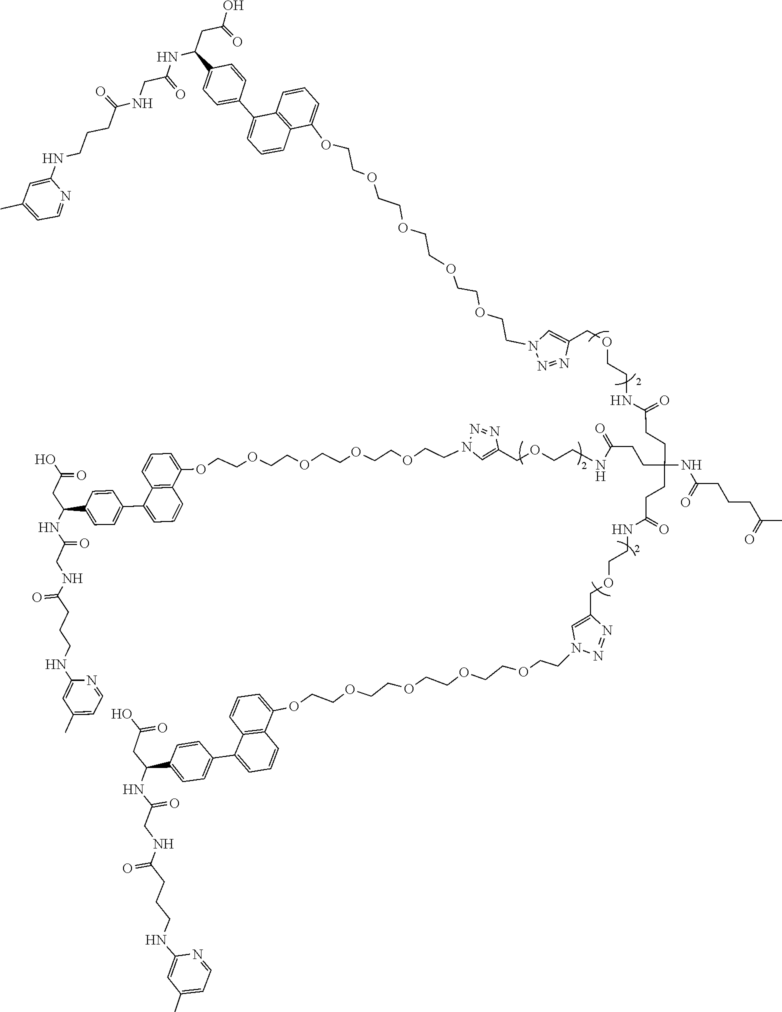

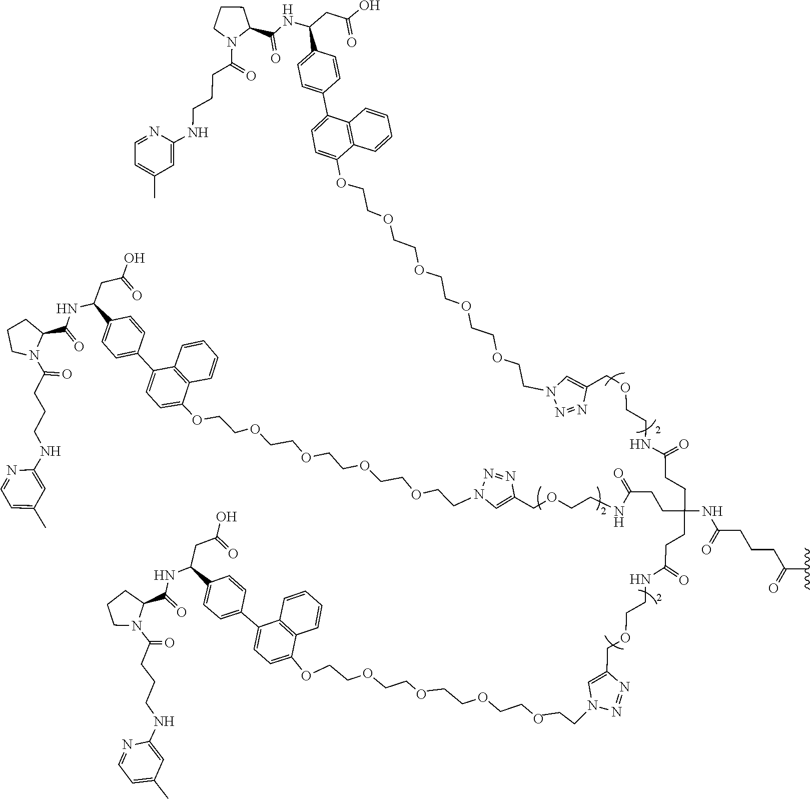

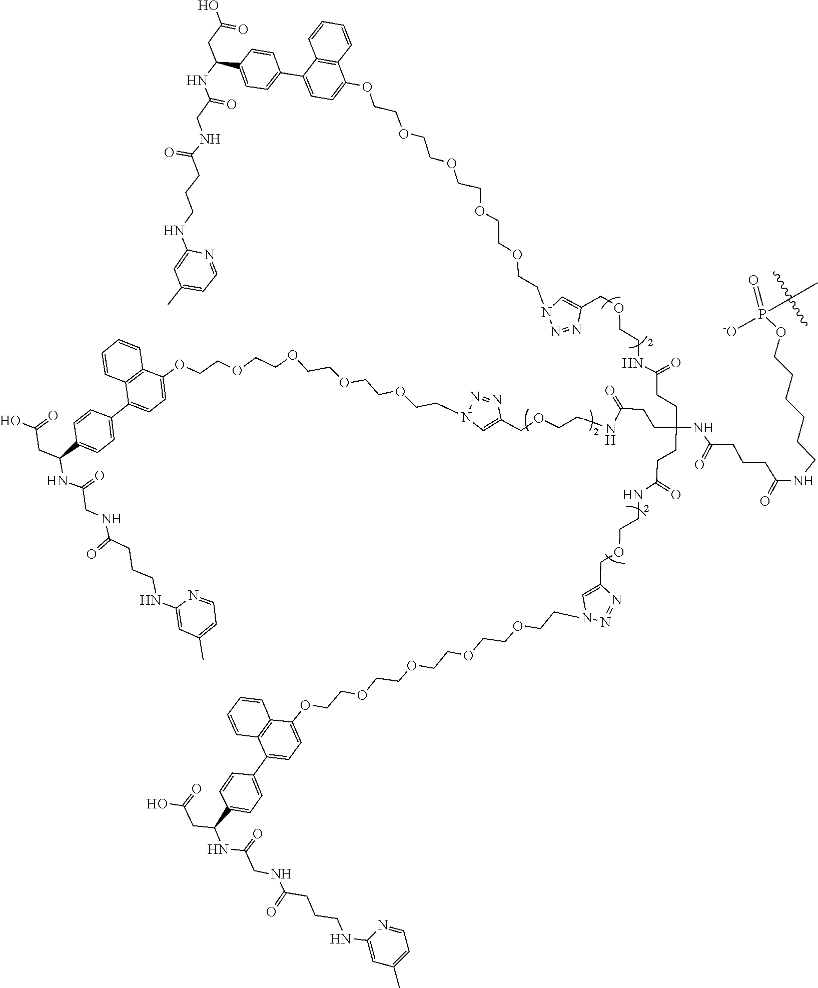

Alternatively, other linking groups known in the art may be used. In many instances, linking groups can be commercially acquired or alternatively, are incorporated into commercially available nucleotide phosphoramidites. (See, e.g., International Patent Application Publication No. WO 2019/161213, which is incorporated herein by reference in its entirety).

In some embodiments, a RAGE RNAi agent is delivered without being conjugated to a targeting ligand or pharmacokinetic/pharmacodynamic (PK/PD) modulator (referred to as being “naked” or a “naked RNAi agent”).

In some embodiments, a RAGE RNAi agent is conjugated to a targeting group, a linking group, a PK modulator, and/or another non-nucleotide group to facilitate delivery of the RAGE RNAi agent to the cell or tissue of choice, for example, to an epithelial cell in vivo. In some embodiments, a RAGE RNAi agent is conjugated to a targeting group wherein the targeting group includes an integrin targeting ligand. In some embodiments, the integrin targeting ligand is an αvβ6 integrin targeting ligand. In some embodiments, a targeting group includes one or more αvβ6 integrin targeting ligands.

In some embodiments, a delivery vehicle may be used to deliver an RNAi agent to a cell or tissue. A delivery vehicle is a compound that improves delivery of the RNAi agent to a cell or tissue. A delivery vehicle can include, or consist of, but is not limited to: a polymer, such as an amphipathic polymer, a membrane active polymer, a peptide, a melittin peptide, a melittin-like peptide (MLP), a lipid, a reversibly modified polymer or peptide, or a reversibly modified membrane active polyamine.

In some embodiments, the RNAi agents can be combined with lipids, nanoparticles, polymers, liposomes, micelles, DPCs or other delivery systems available in the art for nucleic acid delivery. The RNAi agents can also be chemically conjugated to targeting groups, lipids (including, but not limited to cholesteryl and cholesteryl derivatives), encapsulating in nanoparticles, liposomes, micelles, conjugating to polymers or DPCs (see, for example WO 2000/053722, WO 2008/022309, WO 2011/104169, and WO 2012/083185, WO 2013/032829, WO 2013/158141, each of which is incorporated herein by reference), by iontophoresis, or by incorporation into other delivery vehicles or systems available in the art such as hydrogels, cyclodextrins, biodegradable nanocapsules, bioadhesive microspheres, or proteinaceous vectors. In some embodiments the RNAi agents can be conjugated to antibodies having affinity for pulmonary epithelial cells. In some embodiments, the RNAi agents can be linked to targeting ligands that have affinity for pulmonary epithelial cells or receptors present on pulmonary epithelial cells.

### Pharmaceutical Compositions and Formulations

The RAGE RNAi agents disclosed herein can be prepared as pharmaceutical compositions or formulations (also referred to herein as “medicaments”). In some embodiments, pharmaceutical compositions include at least one RAGE RNAi agent. These pharmaceutical compositions are particularly useful in the inhibition of the expression of AGER mRNA in a target cell, a group of cells, a tissue, or an organism. The pharmaceutical compositions can be used to treat a subject having a disease, disorder, or condition that would benefit from reduction in the level of the target mRNA, or inhibition in expression of the target gene. The pharmaceutical compositions can be used to treat a subject at risk of developing a disease or disorder that would benefit from reduction of the level of the target mRNA or an inhibition in expression the target gene. In one embodiment, the method includes administering a RAGE RNAi agent linked to a targeting ligand as described herein, to a subject to be treated. In some embodiments, one or more pharmaceutically acceptable excipients (including vehicles, carriers, diluents, and/or delivery polymers) are added to the pharmaceutical compositions that include a RAGE RNAi agent, thereby forming a pharmaceutical formulation or medicament suitable for in vivo delivery to a subject, including a human.

The pharmaceutical compositions that include a RAGE RNAi agent and methods disclosed herein decrease the level of the target mRNA in a cell, group of cells, group of cells, tissue, organ, or subject, including by administering to the subject a therapeutically effective amount of a herein described RAGE RNAi agent, thereby inhibiting the expression of AGER mRNA in the subject. In some embodiments, the subject has been previously identified or diagnosed as having a disease or disorder that can be mediated at least in part by a reduction in RAGE expression. In some embodiments, the subject has been previously diagnosed with having one or more pulmonary diseases such as asthma (including severe asthma), acute respiratory distress syndrome, idiopathic pulmonary fibrosis, chronic obstructive pulmonary disease (COPD), cystic fibrosis, pneumonia, lung cancer, or bronchopulmonary dysplasia. In some embodiments the pulmonary diseases is severe asthma.

In some embodiments the subject has been previously diagnosed with having cardiovascular disease (atherosclerosis, myocardial infarction, heart failure, peripheral vascular disease), cancer diabetes, chronic kidney disease, neurodegenerative disease, rheumatoid arthritis, non-alcoholic steatohepatitis, injury caused by certain viral infections including SARS-CoV-2, certain ocular inflammatory conditions, or skeletal muscle wasting.

In some embodiments, the subject has been previously diagnosed with having one or more ocular diseases related to ocular inflammation.

Embodiments of the present disclosure include pharmaceutical compositions for delivering a RAGE RNAi agent to a pulmonary epithelial cell in vivo. Such pharmaceutical compositions can include, for example, a RAGE RNAi agent conjugated to a targeting group that comprises an integrin targeting ligand. In some embodiments, the integrin targeting ligand is comprised of an αvβ6 integrin ligand.

In some embodiments, the described pharmaceutical compositions including a RAGE RNAi agent are used for treating or managing clinical presentations in a subject that would benefit from the inhibition of expression of RAGE. In some embodiments, a therapeutically or prophylactically effective amount of one or more of pharmaceutical compositions is administered to a subject in need of such treatment. In some embodiments, administration of any of the disclosed RAGE RNAi agents can be used to decrease the number, severity, and/or frequency of symptoms of a disease in a subject.

In some embodiments, the described RAGE RNAi agents are optionally combined with one or more additional (i.e., second, third, etc.) therapeutics. A second therapeutic can be another RAGE RNAi agent (e.g., a RAGE RNAi agent that targets a different sequence within an AGER (RAGE) gene). In some embodiments, a second therapeutic can be an RNAi agent that targets the AGER gene. An additional therapeutic can also be a small molecule drug, antibody, antibody fragment, and/or aptamer. The RAGE RNAi agents, with or without the one or more additional therapeutics, can be combined with one or more excipients to form pharmaceutical compositions.

The described pharmaceutical compositions that include a RAGE RNAi agent can be used to treat at least one symptom in a subject having a disease or disorder that would benefit from reduction or inhibition in expression of AGER mRNA. In some embodiments, the subject is administered a therapeutically effective amount of one or more pharmaceutical compositions that include a RAGE RNAi agent thereby treating the symptom. In other embodiments, the subject is administered a prophylactically effective amount of one or more RAGE RNAi agents, thereby preventing or inhibiting the at least one symptom.

In some embodiments, one or more of the described RAGE RNAi agents are administered to a mammal in a pharmaceutically acceptable carrier or diluent. In some embodiments, the mammal is a human.

The route of administration is the path by which a RAGE RNAi agent is brought into contact with the body. In general, methods of administering drugs, oligonucleotides, and nucleic acids, for treatment of a mammal are well known in the art and can be applied to administration of the compositions described herein. The RAGE RNAi agents disclosed herein can be administered via any suitable route in a preparation appropriately tailored to the particular route. Thus, in some embodiments, the herein described pharmaceutical compositions are administered via inhalation, intranasal administration, intratracheal administration, or oropharyngeal aspiration administration. In some embodiments, the pharmaceutical compositions can be administered by injection, for example, intravenously, intramuscularly, intracutaneously, subcutaneously, intraarticularly, intraocularly, or intraperitoneally, or topically.

The pharmaceutical compositions including a RAGE RNAi agent described herein can be delivered to a cell, group of cells, tissue, or subject using oligonucleotide delivery technologies known in the art. In general, any suitable method recognized in the art for delivering a nucleic acid molecule (in vitro or in vivo) can be adapted for use with the compositions described herein. For example, delivery can be by local administration, (e.g., direct injection, implantation, or topical administering), systemic administration, or subcutaneous, intravenous, intraperitoneal, or parenteral routes, including intracranial (e.g., intraventricular, intraparenchymal and intrathecal), intramuscular, transdermal, airway (aerosol), nasal, oral, rectal, or topical (including buccal and sublingual) administration. In some embodiments, the compositions are administered via inhalation, intranasal administration, oropharyngeal aspiration administration, or intratracheal administration. For example, in some embodiments, it is desired that the RAGE RNAi agents described herein inhibit the expression of an AGER gene in the pulmonary epithelium, for which administration via inhalation (e.g., by an inhaler device, such as a metered-dose inhaler, or a nebulizer such as a jet or vibrating mesh nebulizer, or a soft mist inhaler) is particularly suitable and advantageous

In some embodiments, the pharmaceutical compositions described herein comprise one or more pharmaceutically acceptable excipients. The pharmaceutical compositions described herein are formulated for administration to a subject.

As used herein, a pharmaceutical composition or medicament includes a pharmacologically effective amount of at least one of the described therapeutic compounds and one or more pharmaceutically acceptable excipients. Pharmaceutically acceptable excipients (excipients) are substances other than the Active Pharmaceutical Ingredient (API, therapeutic product, e.g., RAGE RNAi agent) that are intentionally included in the drug delivery system. Excipients do not exert or are not intended to exert a therapeutic effect at the intended dosage. Excipients can act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, detergents, dextran, dextrose, diluents, disintegrants, emulsifiers, extenders, fillers, flavors, glidants, humectants, lubricants, oils, polymers, preservatives, saline, salts, solvents, sugars, surfactants, suspending agents, sustained release matrices, sweeteners, thickening agents, tonicity agents, vehicles, water-repelling agents, and wetting agents.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor® ELTM (BASF, Parsippany, NJ) or phosphate buffered saline (PBS). It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of the drug that can be in microcrystalline form, for example, in the form of an aqueous microcrystalline suspension. Liposomal formulations or biodegradable polymer systems can also be used to present the drug for both intra-articular and ophthalmic administration.

Formulations suitable for inhalation administration can be prepared by incorporating the active compound in the desired amount in an appropriate solvent, followed by sterile filtration. In general, formulations for inhalation administration are sterile solutions at physiological pH and have low viscosity (<5 cP). Salts may be added to the formulation to balance tonicity. In some cases, surfactants or co-solvents can be added to increase active compound solubility and improve aerosol characteristics. In some cases, excipients can be added to control viscosity in order to ensure size and distribution of nebulized droplets.

In some embodiments, pharmaceutical formulations that include the RAGE RNAi agents disclosed herein suitable for inhalation administration can be prepared in water for injection (sterile water), or an aqueous sodium phosphate buffer (for example, the RAGE RNAi agent formulated in 0.5 mM sodium phosphate monobasic, 0.5 mM sodium phosphate dibasic, in water).

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

The RAGE RNAi agents can be formulated in compositions in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the therapeutic effect to be achieved, and the limitations inherent in the art of compounding such an active compound for the treatment of individuals.

A pharmaceutical composition can contain other additional components commonly found in pharmaceutical compositions. Such additional components include, but are not limited to: anti-pruritics, astringents, local anesthetics, or anti-inflammatory agents (e.g., antihistamine, diphenhydramine, etc.). It is also envisioned that cells, tissues, or isolated organs that express or comprise the herein defined RNAi agents may be used as “pharmaceutical compositions.” As used herein, “pharmacologically effective amount,” “therapeutically effective amount,” or simply “effective amount” refers to that amount of an RNAi agent to produce a pharmacological, therapeutic, or preventive result.

In some embodiments, the methods disclosed herein further comprise the step of administering a second therapeutic or treatment in addition to administering an RNAi agent disclosed herein. In some embodiments, the second therapeutic is another RAGE RNAi agent (e.g., a RAGE RNAi agent that targets a different sequence within the RAGE target). In other embodiments, the second therapeutic can be a small molecule drug, an antibody, an antibody fragment, and/or an aptamer.

In some embodiments, described herein are compositions that include a combination or cocktail of at least two RAGE RNAi agents having different sequences. In some embodiments, the two or more RAGE RNAi agents are each separately and independently linked to targeting groups. In some embodiments, the two or more RAGE RNAi agents are each linked to targeting groups that include or consist of integrin targeting ligands. In some embodiments, the two or more RAGE RNAi agents are each linked to targeting groups that include or consist of αvβ6 integrin targeting ligands.

Described herein are compositions for delivery of RAGE RNAi agents to pulmonary epithelial cells. Furthermore, compositions for delivery of RAGE RNAi agents to cells, including renal epithelial cells and/or epithelial cells in the GI or reproductive tract and/or and ocular surface epithelial cells in the eye, in vivo, are generally described herein.

Generally, an effective amount of a RAGE RNAi agent disclosed herein will be in the range of from about 0.0001 to about 20 mg/kg of body weight/deposited dose, e.g., from about 0.001 to about 5 mg/kg of body weight/deposited dose. In some embodiments, an effective amount of a RAGE RNAi agent will be in the range of from about 0.01 mg/kg to about 3.0 mg/kg of body weight per deposited dose. In some embodiments, an effective amount of a RAGE RNAi agent will be in the range of from about 0.03 mg/kg to about 2.0 mg/kg of body weight per deposited dose. In some embodiments, an effective amount of a RAGE RNAi agent will be in the range of from about 0.01 to about 1.0 mg/kg of deposited dose per body weight. In some embodiments, an effective amount of a RAGE RNAi agent will be in the range of from about 0.50 to about 1.0 mg/kg of deposited dose per body weight. The amount administered will also likely depend on such variables as the overall health status of the patient, the relative biological efficacy of the compound delivered, the formulation of the drug, the presence and types of excipients in the formulation, and the route of administration. Also, it is to be understood that the initial dosage administered can be increased beyond the above upper level to rapidly achieve the desired blood-level or tissue level, or the initial dosage can be smaller than the optimum. In some embodiments, a dose is administered daily. In some embodiments, a dose is administered weekly. In further embodiments, a dose is administered bi-weekly, tri-weekly, once monthly, or once quarterly (i.e., once every three months).

For treatment of disease or for formation of a medicament or composition for treatment of a disease, the pharmaceutical compositions described herein including a RAGE RNAi agent can be combined with an excipient or with a second therapeutic agent or treatment including, but not limited to: a second or other RNAi agent, a small molecule drug, an antibody, an antibody fragment, peptide, and/or an aptamer.

The described RAGE RNAi agents, when added to pharmaceutically acceptable excipients or adjuvants, can be packaged into kits, containers, packs, or dispensers. The pharmaceutical compositions described herein can be packaged in dry powder or aerosol inhalers, other metered-dose inhalers, nebulizers, pre-filled syringes, or vials.

### Methods of Treatment and Inhibition of RAGE Expression

The RAGE RNAi agents disclosed herein can be used to treat a subject (e.g., a human or other mammal) having a disease or disorder that would benefit from administration of the RNAi agent. In some embodiments, the RNAi agents disclosed herein can be used to treat a subject (e.g., a human) that would benefit from a reduction and/or inhibition in expression of AGER mRNA and/or a reduction in RAGE receptor levels.

In some embodiments, the RNAi agents disclosed herein can be used to treat a subject (e.g., a human) having a disease or disorder for which the subject would benefit from reduction in RAGE receptors, including but not limited to, pulmonary diseases such as asthma (including severe asthma), acute respiratory distress syndrome, idiopathic pulmonary fibrosis, lung cancer, bronchopulmonary dysplasia, chronic obstructive pulmonary disease (COPD), or cystic fibrosis. In some embodiments the pulmonary diseases is severe asthma. In some embodiments the subject has been previously diagnosed with having cardiovascular disease (atherosclerosis, myocardial infarction, heart failure, peripheral vascular disease), cancer, diabetes, chronic kidney disease, neurodegenerative disease, rheumatoid arthritis, non-alcoholic steatohepatitis, injury caused by certain viral infections including SARS-CoV-2, certain ocular inflammatory conditions, or skeletal muscle wasting. Treatment of a subject can include therapeutic and/or prophylactic treatment. The subject is administered a therapeutically effective amount of any one or more RAGE RNAi agents described herein. The subject can be a human, patient, or human patient. The subject may be an adult, adolescent, child, or infant. Administration of a pharmaceutical composition described herein can be to a human being or animal.

Increased membrane RAGE activity is known to promote inflammation in tissues. In some embodiments, the described RAGE RNAi agents are used to treat at least one symptom mediated at least in part by a reduction in RAGE levels, in a subject. The subject is administered a therapeutically effective amount of any one or more of the described RAGE RNAi agents. In some embodiments, the subject is administered a prophylactically effective amount of any one or more of the described RNAi agents, thereby treating the subject by preventing or inhibiting the at least one symptom.

In certain embodiments, the present disclosure provides methods for treatment of diseases, disorders, conditions, or pathological states mediated at least in part by AGER gene expression, in a patient in need thereof, wherein the methods include administering to the patient any of the RAGE RNAi agents described herein.

In some embodiments, the RAGE RNAi agents are used to treat or manage a clinical presentation or pathological state in a subject, wherein the clinical presentation or pathological state is mediated at least in part by a reduction in RAGE expression. The subject is administered a therapeutically effective amount of one or more of the RAGE RNAi agents or RAGE RNAi agent-containing compositions described herein. In some embodiments, the method comprises administering a composition comprising a RAGE RNAi agent described herein to a subject to be treated.

In a further aspect, the disclosure features methods of treatment (including prophylactic or preventative treatment) of diseases or symptoms that may be addressed by a reduction in RAGE receptor levels, the methods comprising administering to a subject in need thereof a RAGE RNAi agent that includes an antisense strand comprising the sequence of any of the sequences in Table 2, Table 3, or Table 10. Also described herein are compositions for use in such methods.

The described RAGE RNAi agents and/or compositions that include RAGE RNAi agents can be used in methods for therapeutic treatment of disease or conditions caused by enhanced or elevated RAGE receptor activity levels. Such methods include administration of a RAGE RNAi agent as described herein to a subject, e.g., a human or animal subject.

In another aspect, the disclosure provides methods for the treatment (including prophylactic treatment) of a pathological state (such as a condition or disease) mediated at least in part by RAGE expression, wherein the methods include administering to a subject a therapeutically effective amount of an RNAi agent that includes an antisense strand comprising the sequence of any of the sequences in Table 2, Table 3, or Table 10.

In some embodiments, methods for inhibiting expression of an AGER gene are disclosed herein, wherein the methods include administering to a cell an RNAi agent that includes an antisense strand comprising the sequence of any of the sequences in Table 2, Table 3, or Table 10.

In some embodiments, methods for the treatment (including prophylactic treatment) of a pathological state mediated at least in part by RAGE expression are disclosed herein, wherein the methods include administering to a subject a therapeutically effective amount of an RNAi agent that includes a sense strand comprising the sequence of any of the sequences in Table 2, Table 4, Table 5, Table 6, or Table 10.

In some embodiments, methods for inhibiting expression of an AGER gene are disclosed herein, wherein the methods comprise administering to a cell an RNAi agent that includes a sense strand comprising the sequence of any of the sequences in Table 2, Table 4, Table 5, Table 6, or Table 10.

In some embodiments, methods for the treatment (including prophylactic treatment) of a pathological state mediated at least in part by RAGE expression are disclosed herein, wherein the methods include administering to a subject a therapeutically effective amount of an RNAi agent that includes a sense strand comprising the sequence of any of the sequences in Table 4, Table 5, Table 6, or Table 10, and an antisense strand comprising the sequence of any of the sequences in Table 3 or Table 10.

In some embodiments, methods for inhibiting expression of an AGER (RAGE) gene are disclosed herein, wherein the methods include administering to a cell an RNAi agent that includes a sense strand comprising the sequence of any of the sequences in Table 4, Table 5, Table 6, or Table 10, and an antisense strand comprising the sequence of any of the sequences in Table 3 or Table 10.

In some embodiments, methods of inhibiting expression of an AGER gene are disclosed herein, wherein the methods include administering to a subject a RAGE RNAi agent that includes a sense strand consisting of the nucleobase sequence of any of the sequences in Table 4, Table 5, Table 6, or Table 10, and the antisense strand consisting of the nucleobase sequence of any of the sequences in Table 3 or Table 10. In other embodiments, disclosed herein are methods of inhibiting expression of an AGER gene, wherein the methods include administering to a subject a RAGE RNAi agent that includes a sense strand consisting of the modified sequence of any of the modified sequences in Table 4, Table 5, Table 6, or Table 10, and the antisense strand consisting of the modified sequence of any of the modified sequences in Table 3 or Table 10.

In some embodiments, methods for inhibiting expression of an AGER gene in a cell are disclosed herein, wherein the methods include administering one or more RAGE RNAi agents comprising a duplex structure of one of the duplexes set forth in Tables 7A, 7B, 8, 9A, 9B, and 10.

In some embodiments, the gene expression level and/or mRNA level of an AGER gene in certain epithelial cells of subject to whom a described RAGE RNAi agent is administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99%, relative to the subject prior to being administered the RAGE RNAi agent or to a subject not receiving the RAGE RNAi agent. In some embodiments, the RAGE receptor or RAGE protein levels in certain epithelial cells of a subject to whom a described RAGE RNAi agent is administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99%, relative to the subject prior to being administered the RAGE RNAi agent or to a subject not receiving the RAGE RNAi agent. The gene expression level, protein level, and/or mRNA level in the subject may be reduced in a cell, group of cells, and/or tissue of the subject. In some embodiments, the AGER mRNA levels in certain epithelial cells subject to whom a described RAGE RNAi agent has been administered is reduced by at least about 30%, 35%, 40%, 45%, 50%, 55%, 60%, 65%, 70%, 75%, 80%, 85%, 90%, 95%, or 98% relative to the subject prior to being administered the RAGE RNAi agent or to a subject not receiving the RAGE RNAi agent.

A reduction in gene expression, mRNA, and protein levels can be assessed by any methods known in the art. Reduction or decrease in RAGE receptor activity level and/or RAGE protein levels are collectively referred to herein as a decrease in, reduction of, or inhibition of RAGE expression. The Examples set forth herein illustrate known methods for assessing inhibition of RAGE expression and AGER gene expression.

### Cells, Tissues, Organs, and Non-Human Organisms

Cells, tissues, organs, and non-human organisms that include at least one of the RAGE RNAi agents described herein are contemplated. The cell, tissue, organ, or non-human organism is made by delivering the RNAi agent to the cell, tissue, organ, or non-human organism.

### Additional Illustrative Embodiments

Provided here are certain additional illustrative embodiments of the disclosed technology. These embodiments are illustrative only and do not limit the scope of the present disclosure or of the claims attached hereto.

Embodiment 1. An RNAi agent for inhibiting expression of a receptor for advanced glycation end-products gene, comprising:
an antisense strand comprising at least 15 contiguous nucleotides differing by 0, 1, 2, or 3, nucleotides from any one of the antisense strand sequences disclosed in Table 2 or Table 3; and
a sense strand comprising a nucleotide sequence that is at least partially complementary to the antisense strand.

Embodiment 2. An RNAi agent for inhibiting expression of a receptor for advanced glycation end-products gene, comprising:
a sense strand comprising at least 15 contiguous nucleotides differing by 0, 1, 2, or 3 nucleotides from a stretch of the same length of nucleotides of SEQ ID NO:1; and an antisense strand comprising a nucleotide sequences that is at least partially complementary to the sense strand.

Embodiment 3. An inhibitor of an AGER (RAGE) gene comprising an antisense strand comprising a nucleotide sequence having at least 15 contiguous nucleotides differing by 0, 1, 2, or 3 nucleotides that are complementary to any of the target nucleotide sequences in Table 1.

Embodiment 4. An RNAi agent comprising (i) an antisense strand comprising a nucleotide sequence having at least 15 contiguous nucleotides differing by 0, 1, 2, or 3 nucleotides from any of the nucleotide sequences in Table 2, Table 3 or Table 10, and (ii) a sense strand at least partially complementary to the antisense strand.

Embodiment 5. An RNAi agent comprising (i) an antisense strand comprising, consisting of, or consisting essentially of a nucleotide sequence from any of the antisense strand nucleotide sequences in Table 2, Table 3 or Table 10, and (ii) a sense strand comprising, consisting of, or consisting essentially of a nucleotide sequence from any of the sense strand nucleotide sequences in Table 2, Table 4, Table 5, Table 6, or Table 10.

Embodiment 6. An RNAi agent comprising an antisense strand and sense strand annealed to form a duplex, wherein the duplex has the structure of any of the duplexes set forth in Table 7A, Table 7B, Table 8, Table 9, or Table 10.

Embodiment 7. an RNAi agent capable of inhibiting expression of a Receptor for Advanced Glycation End-products gene comprising:
(i) an antisense strand that is between 18 and 49 nucleotides in length that is at least partially complementary to a Receptor for Advanced Glycation End-products gene (SEQ ID NO:1);
(ii) a sense strand that is at least partially complementary to the antisense strand;
and
(iii) a targeting ligand linked to the sense strand.

Embodiment 8. An RNAi agent for inhibiting expression of a Receptor for Advanced Glycation End-products gene, comprising:
an antisense strand comprising at least 17 contiguous nucleotides differing by 0 or 1 nucleotides from any one of the sequences provided in Table 2 or Table 3; and
a sense strand comprising a nucleotide sequence that is at least partially complementary to the antisense strand.

Embodiment 9. The RNAi agent of any one of embodiments 1-8, wherein the antisense strand comprises nucleotides 2-18 of any one of the sequences provided in Table 2 or Table 3.

Embodiment 10. The RNAi agent of any one of embodiments 1-9, wherein the sense strand comprises a nucleotide sequence of at least 17 contiguous nucleotides differing by 0 or 1 nucleotides from any one of the sequences provided in Table 2 or Table 4, and wherein the sense strand has a region of at least 85% complementarity over the 17 contiguous nucleotides to the antisense strand.

Embodiment 11. The RNAi agent of any one of embodiments 1-10, wherein at least one nucleotide of the RNAi agent is a modified nucleotide or includes a modified internucleoside linkage.

Embodiment 12. The RNAi agent of any one of embodiments 1-11, wherein all or substantially all of the nucleotides are modified nucleotides.

Embodiment 13. The RNAi agent of any one of embodiments 11-12, wherein the modified nucleotide is selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.

Embodiment 14. The RNAi agent of embodiment 12, wherein all or substantially all of the nucleotides are modified with 2′-O-methyl nucleotides, 2′-fluoro nucleotides, or combinations thereof.

Embodiment 15. The RNAi agent of any one of embodiments 1-14, wherein the antisense strand comprises the nucleotide sequence of any one of the modified sequences provided in Table 3.

Embodiment 16. The RNAi agent of any one of embodiments 1-15, wherein the sense strand comprises the nucleotide sequence of any one of the modified sequences provided in Table 4.

Embodiment 17. The RNAi agent of embodiment 1, wherein the antisense strand comprises the nucleotide sequence of any one of the modified sequences provided in Table 3 and the sense strand comprises the nucleotide sequence of any one of the modified sequences provided in Table 4.

Embodiment 18. The RNAi agent of any one of embodiments 1-17, wherein the sense strand is between 18 and 30 nucleotides in length, and the antisense strand is between 18 and 30 nucleotides in length.

Embodiment 19. The RNAi agent of embodiment 18, wherein the sense strand and the antisense strand are each between 18 and 27 nucleotides in length.

Embodiment 20. The RNAi agent of embodiment 19, wherein the sense strand and the antisense strand are each between 18 and 24 nucleotides in length.

Embodiment 21. The RNAi agent of embodiment 20, wherein the sense strand and the antisense strand are each 21 nucleotides in length.

Embodiment 22. The RNAi agent of embodiment 21, wherein the RNAi agent has two blunt ends.

Embodiment 23. The RNAi agent of any one of embodiments 1-22, wherein the sense strand comprises one or two terminal caps.

Embodiment 24. The RNAi agent of any one of embodiments 1-23, wherein the sense strand comprises one or two inverted abasic residues.

Embodiment 25. The RNAi agent of embodiment 8, wherein the RNAi agent is comprised of a sense strand and an antisense strand that form a duplex having the structure of any one of the duplexes in Table 7A, Table 7B, Table 8, Table 9A, Table 9B, or Table 10.

Embodiment 26. The RNAi agent of embodiment 25, wherein all or substantially all of the nucleotides are modified nucleotides.

Embodiment 27. The RNAi agent of embodiment 1, comprising an antisense strand that consists of, consists essentially of, or comprises a nucleotide sequence that differs by 0 or 1 nucleotides from one of the following nucleotide sequences (5′→3′):

(SEQ ID NO: 55)
UUGUGUUCAGUUUCCAUUC;
(SEQ ID NO: 65)
UGAUGUUUUGAGCACCUAC;
(SEQ ID NO: 73)
UUCCAUUCCUGUUCAUUGC;
(SEQ ID NO: 7)
UUGUGUUCAGUUUCCAUUCCG;
(SEQ ID NO: 9)
UGAUGUUUUGAGCACCUACUC;
or
(SEQ ID NO: 8)
UUCCAUUCCUGUUCAUUGCCU.

Embodiment 28. The RNAi agent of embodiment 27, wherein the sense strand consists of, consists essentially of, or comprises a nucleotide sequence that differs by 0 or 1 nucleotides from one of the following nucleotide sequences (5′→3′):

(SEQ ID NO: 298)
GAAUGGAAACUGAACACAA;
(SEQ ID NO: 308)
GUAGGUGCUCAAAACAUCA;
(SEQ ID NO: 316)
GCAAUGAACAGGAAUIGAA;
(SEQ ID NO: 19)
CGGAAUGGAAACUGAACACAA;
(SEQ ID NO: 20)
GAGUAGGUGCUCAAAACAUCA;
or
(SEQ ID NO: 21)
AGGCAAUGAACAGGAAUIGAA.

Embodiment 29. The RNAi agent of embodiment 27 or 28, wherein all or substantially all of the nucleotides are modified nucleotides.

Embodiment 30. The RNAi agent of embodiment 1, comprising an antisense strand that comprises, consists of, or consists essentially of a modified nucleotide sequence that differs by 0 or 1 nucleotides from one of the following nucleotide sequences (5′→3′):

(SEQ ID NO: 2)
usUfsgsUfgUfuCfaGfuUfuCfcAfuUfcCfsg;
(SEQ ID NO: 3)
cPrpusUfsgsUfgUfuCfaGfuUfuCfcAfuUfcCfsg;
(SEQ ID NO: 5)
usGfsasuguuuugaGfcAfcCfuacusc;
(SEQ ID NO: 6)
cPrpusGfsasuguuuugaGfcAfcCfuacusc;
(SEQ ID NO: 4)
usUfscsCfaUfuCfcUfgUfuCfaUfuGfcCfsu;

wherein a, c, g, and u represent 2′-O-methyl adenosine, 2′-O-methyl cytidine, 2′-O-methyl guanosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; cPrpu represents a 5′-cyclopropyl phosphonate-2′-O-methyl uridine; s represents a phosphorothioate linkage; and wherein all or substantially all of the nucleotides on the sense strand are modified nucleotides.

Embodiment 31. The RNAi agent of embodiment 1, wherein the sense strand comprises, consists of, or consists essentially of a modified nucleotide sequence that differs by 0 or 1 nucleotides from one of the following nucleotide sequences (5′→3′):

(SEQ ID NO: 14)
gsaguagGfuGfcUfcaaaacauca;
(SEQ ID NO: 15)
asggcaaugAfAfCfaggaauigaa;
(SEQ ID NO: 13)
csggaauggAfAfAfcugaacacaa;

wherein a, c, g, i, and u represent 2′-O-methyl adenosine, 2′-O-methyl cytidine, 2′-O-methyl guanosine, 2′-O-methyl inosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; and s represents a phosphorothioate linkage; and wherein all or substantially all of the nucleotides on the antisense strand are modified nucleotides.

Embodiment 32. The RNAi agent of any one of embodiments 27-31, wherein the sense strand further includes inverted abasic residues at the 3′ terminal end of the nucleotide sequence, at the 5′ end of the nucleotide sequence, or at both.

Embodiment 33. The RNAi agent of any one of embodiments 1-32, wherein the RNAi agent is linked to a targeting ligand.

Embodiment 34. The RNAi agent of embodiment 33, wherein the targeting ligand has affinity for a cell receptor expressed on an epithelial cell.

Embodiment 35. The RNAi agent of embodiment 34, wherein the targeting ligand comprises an integrin targeting ligand.

Embodiment 36. The RNAi agent of embodiment 35, wherein the integrin targeting ligand is an αvβ6 integrin targeting ligand.

Embodiment 37. The RNAi agent of embodiment 36, wherein the targeting ligand comprises the structure:

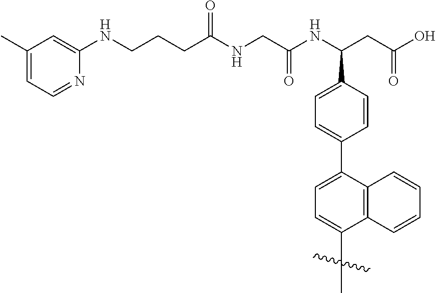 [SMILES: *c1ccc(-c2ccc([C@H](CC(=O)O)NC(=O)CNC(=O)CCCNc3cc(C)ccn3)cc2)c2ccccc12]

or a pharmaceutically acceptable salt thereof, or

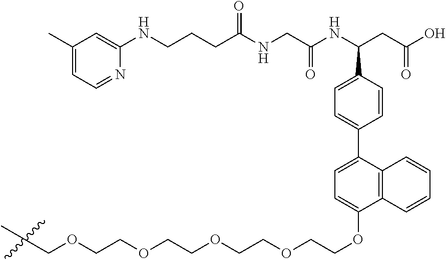 [SMILES: *COCCOCCOCCOCCOc1ccc(-c2ccc([C@H](CC(=O)O)NC(=O)CNC(=O)CCCNc3cc(C)ccn3)cc2)c2ccccc12]

or a pharmaceutically acceptable salt thereof, wherein [symbol] indicates the point of connection to the RNAi agent.

Embodiment 38. The RNAi agent of any one of embodiments 33-36, wherein the targeting ligand has a structure selected from the group consisting of:

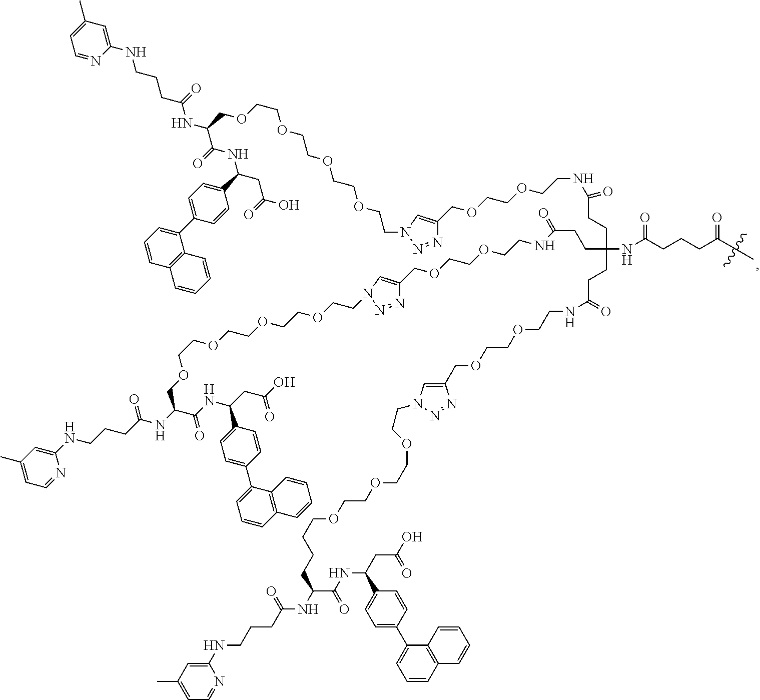 [SMILES: *C(=O)CCCC(=O)NC(CCC(=O)NCCOCCOCc1cn(CCOCCOCCOCCCC[C@H](NC(=O)CCCNc2cc(C)ccn2)C(=O)N[C@@H](CC(=O)O)c2ccc(-c3cccc4ccccc34)cc2)nn1)(CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1)CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1]

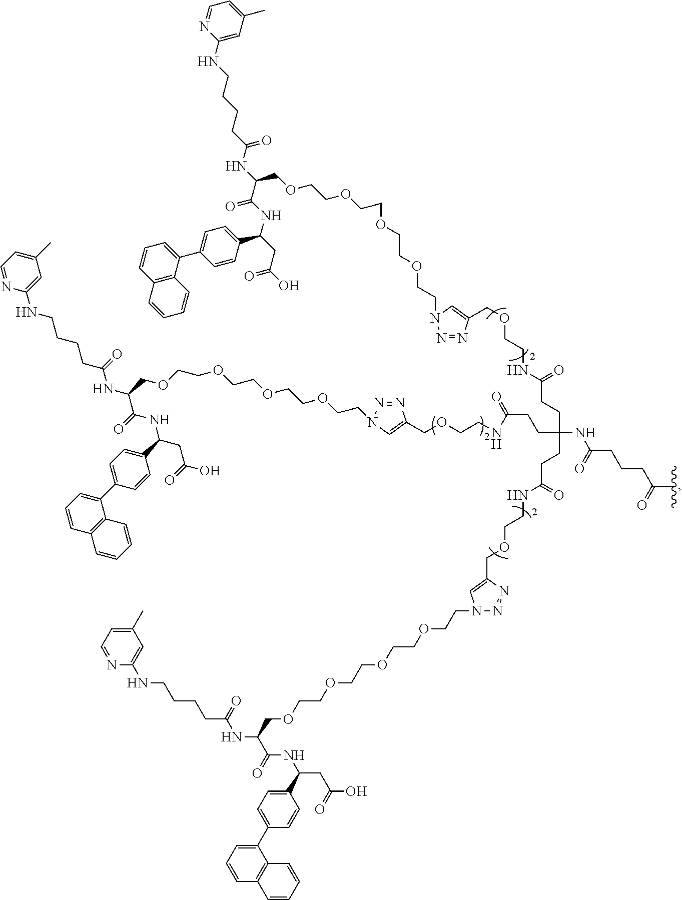

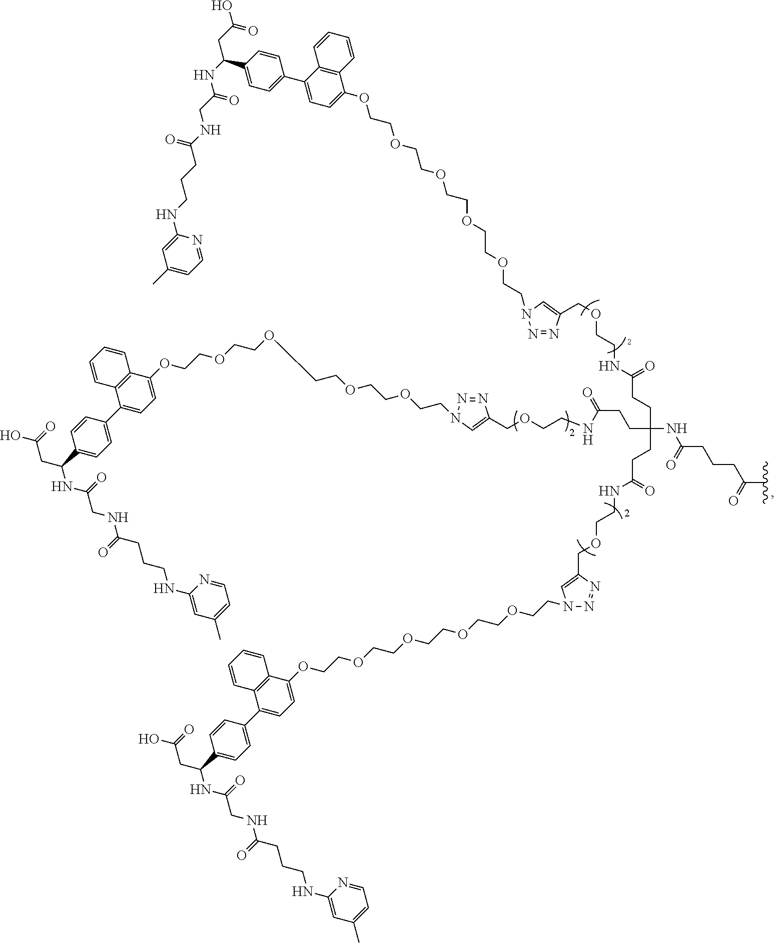

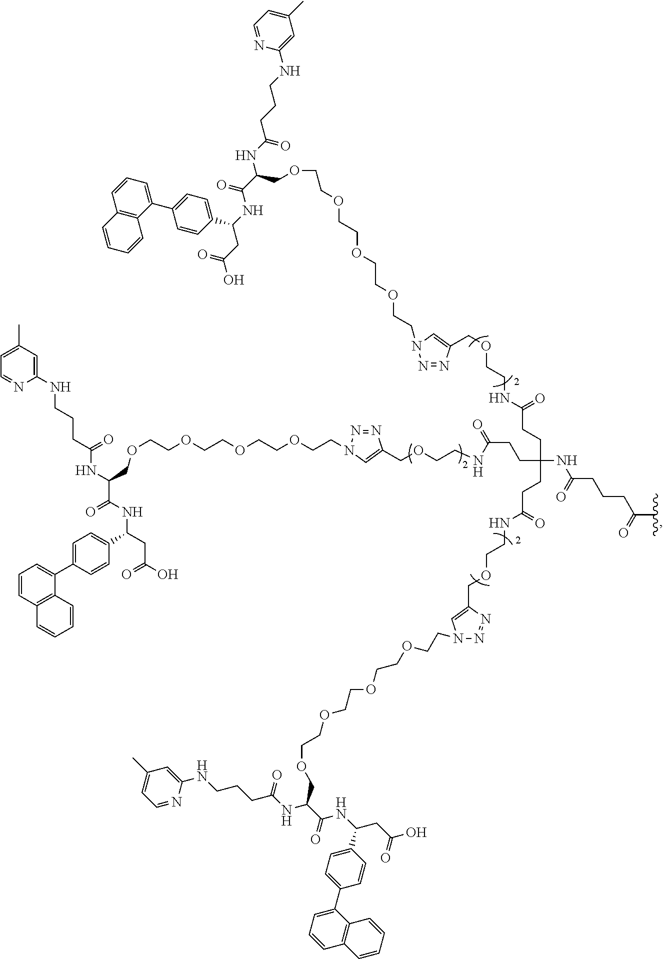

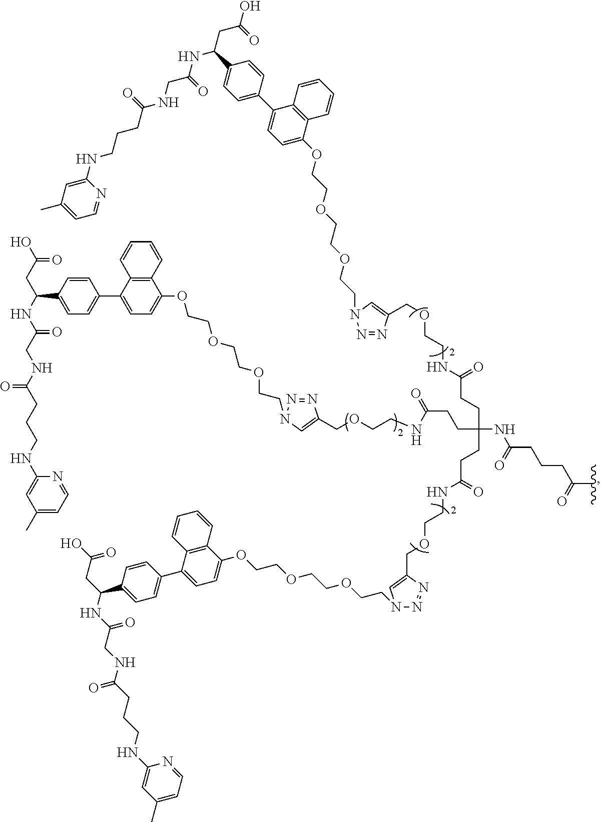

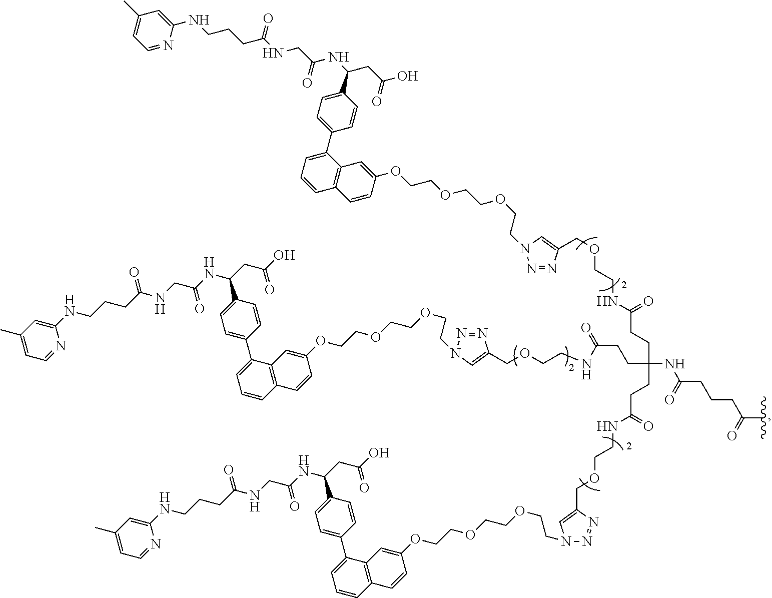

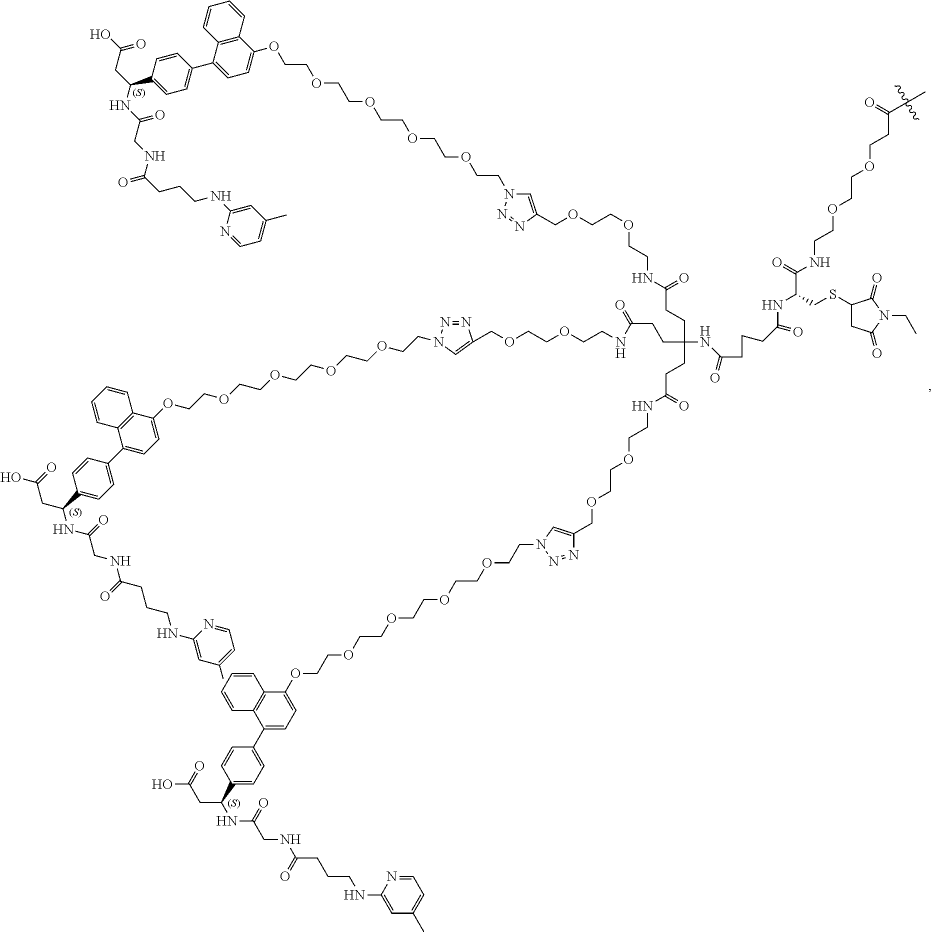

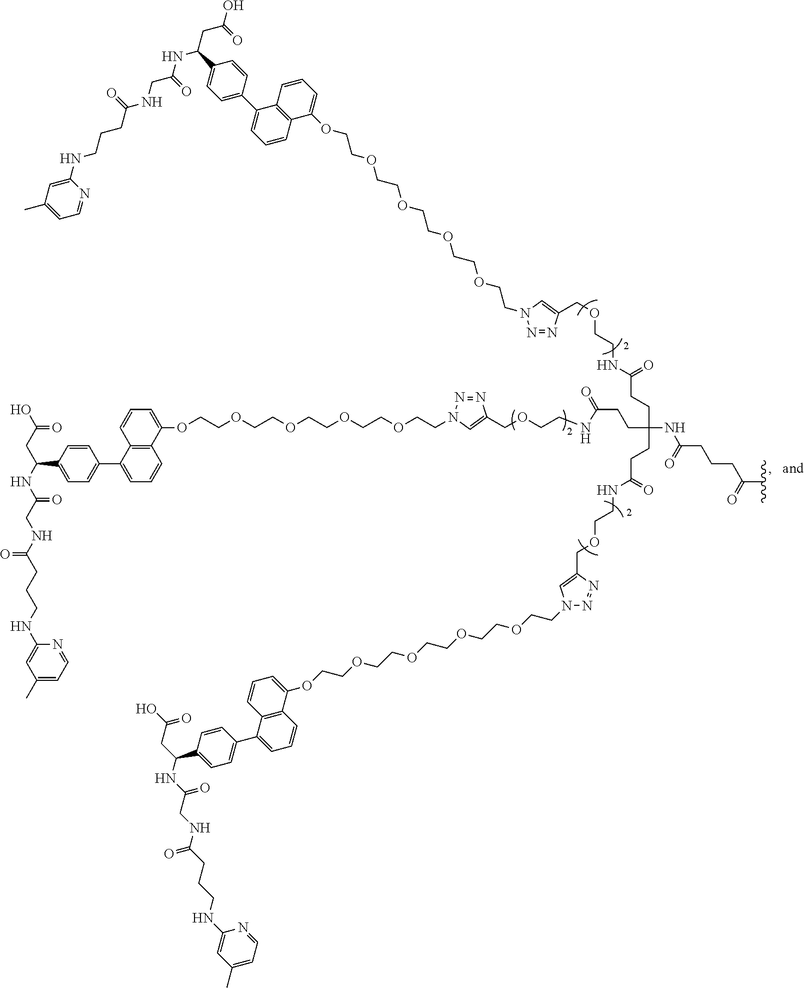

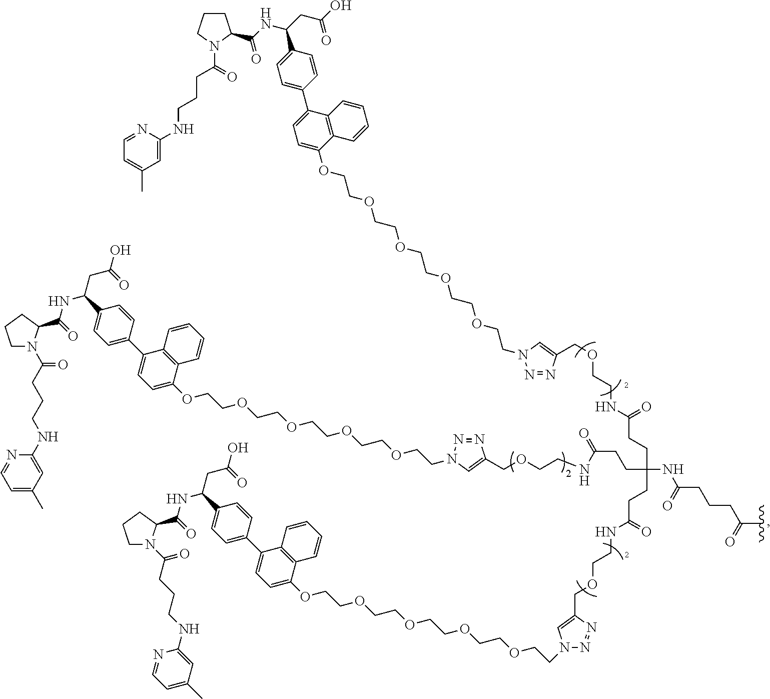

wherein [symbol] indicates the point of connection to the RNAi agent.

Embodiment 39. The RNAi agent of embodiment 38, wherein RNAi agent is conjugated to a targeting ligand having the following structure:

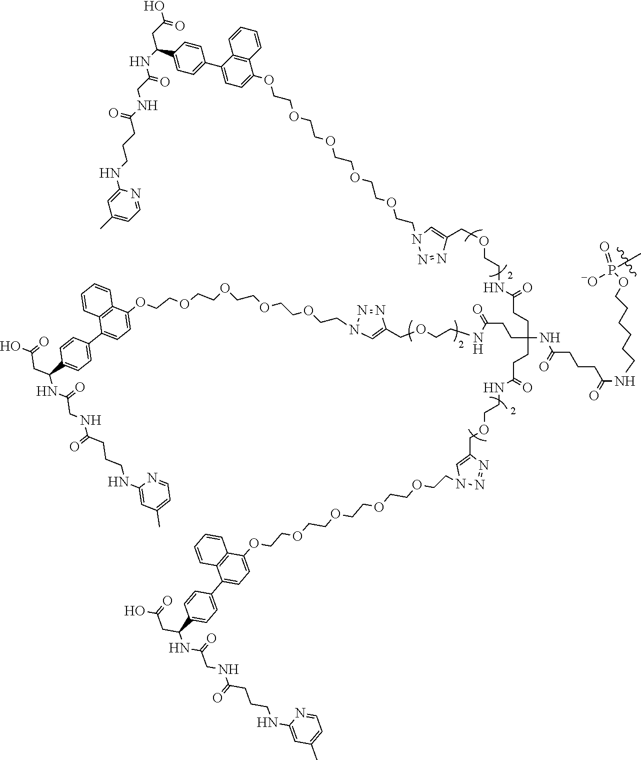

Embodiment 40. The RNAi agent of any one of embodiments 33-36, wherein the targeting ligand has the following structure:

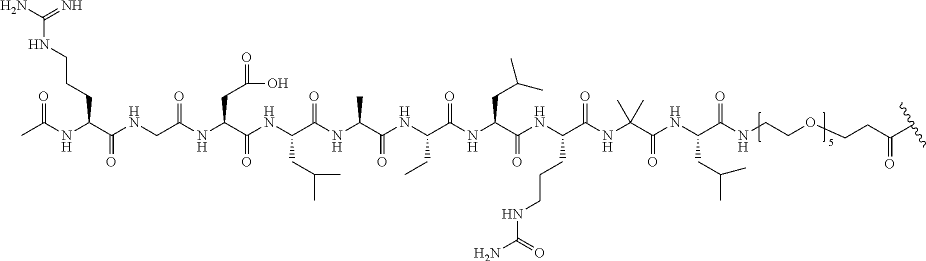

Embodiment 41. The RNAi agent of any one of embodiments 33-36, wherein the targeting ligand is conjugated to the sense strand.

Embodiment 42. The RNAi agent of embodiment 41, wherein the targeting ligand is conjugated to the 5′ terminal end of the sense strand.

Embodiment 43. The RNAi agent of any of the preceding embodiments, wherein the RNAi agent is conjugated to a targeting ligand and has the duplex structure of AC000292, AC001266, AC001267, or AC001268.

Embodiment 44. A composition comprising the RNAi agent of any one of embodiments 1-43, wherein the composition further comprises a pharmaceutically acceptable excipient.

Embodiment 45. The composition of embodiment 44, further comprising a second RNAi agent capable of inhibiting the expression of Receptor for Advanced Glycation End-products gene expression.

Embodiment 46. The composition of any one of embodiments 44-45, further comprising one or more additional therapeutics.

Embodiment 47. The composition of any one of embodiments 44-46, wherein the composition is formulated for administration by inhalation.

Embodiment 48. The composition of embodiment 47, wherein the composition is delivered by a metered-dose inhaler, jet nebulizer, vibrating mesh nebulizer, or soft mist inhaler.

Embodiment 49. The composition of any of embodiments 44-48, wherein the RNAi agent is a sodium salt.

Embodiment 50. The composition of any of embodiments 44-49, wherein the pharmaceutically acceptable excipient is water for injection.

Embodiment 51. The composition of any of embodiments 44-49, wherein the pharmaceutically acceptable excipient is a buffered saline solution.

Embodiment 52. A method for inhibiting expression of a Receptor for Advanced Glycation End-products gene in a cell, the method comprising introducing into a cell an effective amount of an RNAi agent of any one of embodiments 1-43 or the composition of any one of embodiments 44-51.

Embodiment 53. The method of embodiment 52, wherein the cell is within a subject.

Embodiment 54. The method of embodiment 53, wherein the subject is a human subject.

Embodiment 55. The method of any one of embodiments 52-54, wherein following the administration of the RNAi agent the Receptor for Advanced Glycation End-products gene expression is inhibited by at least about 30%.

Embodiment 56. A method of treating one or more symptoms or diseases associated with enhanced or elevated membrane RAGE activity levels, the method comprising administering to a human subject in need thereof a therapeutically effective amount of the composition of any one of embodiments 44-51.

Embodiment 57. The method of embodiment 56, wherein the disease is a respiratory disease.

Embodiment 58. The method of embodiment 57, wherein the respiratory disease is cystic fibrosis, pneumonia, chronic bronchitis, non-cystic fibrosis bronchiectasis, chronic obstructive pulmonary disease (COPD), asthma, respiratory tract infections, primary ciliary dyskinesia, or lung carcinoma cystic fibrosis.

Embodiment 59. The method of embodiment 58, wherein the disease is chronic obstructive pulmonary disease (COPD).

Embodiment 60. The method of embodiment 56, wherein the disease is an ocular disease.

Embodiment 61. The method of embodiment 60, wherein the ocular disease is dry eye syndrome.

Embodiment 62. The method of any one of embodiments 52-61, wherein the RNAi agent is administered at a deposited dose of about 0.01 mg/kg to about 5.0 mg/kg of body weight of the subject.

Embodiment 63. The method of any one of embodiments 52-61, wherein the RNAi agent is administered at a deposited dose of about 0.03 mg/kg to about 2.0 mg/kg of body weight of the subject.

Embodiment 64. The method of any of embodiments 52-61, wherein the RNAi agent is administered in two or more doses.

Embodiment 65. Use of the RNAi agent of any one of embodiments 1-43, for the treatment of a disease, disorder, or symptom that is mediated at least in part by membrane RAGE activity and/or AGER gene expression.

Embodiment 66. Use of the composition according to any one of embodiments 44-51, for the treatment of a disease, disorder, or symptom that is mediated at least in part by Receptor for Advanced Glycation End-products receptor activity and/or Receptor for Advanced Glycation End-products gene expression.

Embodiment 67. Use of the composition according to any one of embodiments 44-51, for the manufacture of a medicament for treatment of a disease, disorder, or symptom that is mediated at least in part by Receptor for Advanced Glycation End-products receptor activity and/or Receptor for Advanced Glycation End-products gene expression.

Embodiment 68. The use of any one of embodiments 65-67, wherein the disease is pulmonary inflammation.

Embodiment 69. A method of making an RNAi agent of any one of embodiments 1-43, comprising annealing a sense strand and an antisense strand to form a double-stranded ribonucleic acid molecule.

Embodiment 70. The method of embodiment 69, wherein the sense strand comprises a targeting ligand.

Embodiment 71. The method of embodiment 70, comprising conjugating a targeting ligand to the sense strand.

The above provided embodiments and items are now illustrated with the following, non-limiting examples.

### EXAMPLES

### Example 1. Synthesis of RAGE RNAi Agents

RAGE RNAi agent duplexes disclosed herein were synthesized in accordance with the following:

A. Synthesis. The sense and antisense strands of the RAGE RNAi agents were synthesized according to phosphoramidite technology on solid phase used in oligonucleotide synthesis. Depending on the scale, a MerMade96ER (Bioautomation), a MerMade12® (Bioautomation), or an OP Pilot 100 (GE Healthcare) was used. Syntheses were performed on a solid support made of controlled pore glass (CPG, 500 Å or 600 Å, obtained from Prime Synthesis, Aston, PA, USA). The monomer positioned at the 3′ end of the respective strand was attached to the solid support as a starting point for synthesis. All RNA and 2′-modified RNA phosphoramidites were purchased from Thermo Fisher Scientific (Milwaukee, WI, USA). Specifically, the 2′-O-methyl phosphoramidites that were used included the following: (5′-O-dimethoxytrityl-N⁶-(benzoyl)-2′-O-methyl-adenosine-3′-O-(2-cyanoethyl-N,N-diisopropylamino)phosphoramidite, 5′-O-dimethoxy-trityl-N⁴-(acetyl)-2′-O-methyl-cytidine-3′-O-(2-cyanoethyl-N,N-diisopropyl-amino)phosphoramidite, (5′-O-dimethoxytrityl-N²-(isobutyryl)-2′-O-methyl-guanosine-3′-O-(2-cyanoethyl-N,N-diisopropylamino) phosphoramidite, and 5′-O-dimethoxytrityl-2′-O-methyl-uridine-3′-O-(2-cyanoethyl-N,N-diisopropylamino)phosphoramidite. The 2′-deoxy-2′-fluoro-phosphoramidites carried the same protecting groups as the 2′-O-methyl RNA amidites. 5′-dimethoxytrityl-2′-O-methyl-inosine-3′-O-(2-cyanoethyl-N,N-diisopropylamino)phosphoramidites were purchased from Glen Research (Virginia). The inverted abasic (3′-O-dimethoxytrityl-2′-deoxyribose-5′-O-(2-cyanoethyl-N,N-diisopropylamino)phosphoramidites were purchased from ChemGenes (Wilmington, MA, USA). The following UNA phosphoramidites were used: 5′-(4,4′-Dimethoxytrityl)-N6-(benzoyl)-2′,3′-seco-adenosine, 2′-benzoyl-3′-[(2-cyanoethyl)-(N,N-diisopropyl)]-phosphoramidite, 5′-(4,4′-Dimethoxytrityl)-N-acetyl-2′,3′-seco-cytosine, 2′-benzoyl-3′-[(2-cyanoethyl)-(N,N-diiso-propyl)]-phosphoramidite, 5′-(4,4′-Dimethoxytrityl)-N-isobutyryl-2′,3′-seco-guanosine, 2′-benzoyl-3′-[(2-cyanoethyl)-(N,N-diisopropyl)]-phosphoramidite, and 5′-(4,4′-Dimethoxy-trityl)-2′,3′-seco-uridine, 2′-benzoyl-3′-[(2-cyanoethyl)-(N,N-diiso-propyl)]-phosphoramidite. TFA aminolink phosphoramidites were also commercially purchased (ThermoFisher). Linker L6 was purchased as propargyl-PEG5-NHS from BroadPharm (catalog #BP-20907) and coupled to the NH₂-C₆ group from an aminolink phosphoramidite to form -L6-C6-, using standard coupling conditions. The linker Alk-cyHex was similarly commercially purchased from Lumiprobe (alkyne phosphoramidite, 5′-terminal) as a propargyl-containing compound phosphoramidite compound to form the linker -Alk-cyHex-. In each case, phosphorothioate linkages were introduced as specified using the conditions set forth herein. The cyclopropyl phosphonate phosphoramidites were synthesized in accordance with International Patent Application Publication No. WO 2017/214112.

Tri-alkyne-containing phosphoramidites were dissolved in anhydrous dichloromethane or anhydrous acetonitrile (50 mM), while all other amidites were dissolved in anhydrous acetonitrile (50 mM) and molecular sieves (3 Å) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 minutes (RNA), 90 seconds (2′ O-Me), and 60 seconds (2′ F). In order to introduce phosphorothioate linkages, a 100 mM solution of 3-phenyl 1,2,4-dithiazoline-5-one (POS, obtained from PolyOrg, Inc., Leominster, MA, USA) in anhydrous acetonitrile was employed.

Alternatively, tri-alkyne moieties were introduced post-synthetically (see section E, below). For this route, the sense strand was functionalized with a 5′ and/or 3′ terminal nucleotide containing a primary amine. TFA aminolink phosphoramidite was dissolved in anhydrous acetonitrile (50 mM) and molecular sieves (3 Å) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 minutes (RNA), 90 seconds (2′ O-Me), and 60 seconds (2′ F). In order to introduce phosphorothioate linkages, a 100 mM solution of 3-phenyl 1,2,4-dithiazoline-5-one (POS, obtained from PolyOrg, Inc., Leominster, MA, USA) in anhydrous acetonitrile was employed.

B. Cleavage and deprotection of support bound oligomer. After finalization of the solid phase synthesis, the dried solid support was treated with a 1:1 volume solution of 40 wt. % methylamine in water and 28% to 31% ammonium hydroxide solution (Aldrich) for 1.5 hours at 30° C. The solution was evaporated and the solid residue was reconstituted in water (see below).

C. Purification. Crude oligomers were purified by anionic exchange HPLC using a TSKgel SuperQ-5 PW 13 μm column and Shimadzu LC-8 system. Buffer A was 20 mM Tris, 5 mM EDTA, pH 9.0 and contained 20% Acetonitrile and buffer B was the same as buffer A with the addition of 1.5 M sodium chloride. UV traces at 260 nm were recorded. Appropriate fractions were pooled then run on size exclusion HPLC using a GE Healthcare XK 16/40 column packed with Sephadex G-25 fine with a running buffer of 100 mM ammonium bicarbonate, pH 6.7 and 20% Acetonitrile or filtered water. Alternatively, pooled fractions were desalted and exchanged into an appropriate buffer or solvent system via tangential flow filtration.

D. Annealing. Complementary strands were mixed by combining equimolar RNA solutions (sense and antisense) in 1×PBS (Phosphate-Buffered Saline, 1×, Corning, Cellgro) to form the RNAi agents. Some RNAi agents were lyophilized and stored at −15 to −25° C. Duplex concentration was determined by measuring the solution absorbance on a UV-Vis spectrometer in 1×PBS. The solution absorbance at 260 nm was then multiplied by a conversion factor (0.050 mg/(mL·cm)) and the dilution factor to determine the duplex concentration.

E. Conjugation of Tri-alkyne linker. In some embodiments a tri-alkyne linker is conjugated to the sense strand of the RNAi agent on resin as a phosphoramidite (see Example 1G for the synthesis of an example tri-alkyne linker phosphoramidite and Example 1A for the conjugation of the phosphoramidite.). In other embodiments, a tri-alkyne linker may be conjugated to the sense strand following cleavage from the resin, described as follows: either prior to or after annealing, in some embodiments, the 5′ or 3′ amine functionalized sense strand is conjugated to a tri-alkyne linker. An example tri-alkyne linker structure that can be used in forming the constructs disclosed herein is as follows:

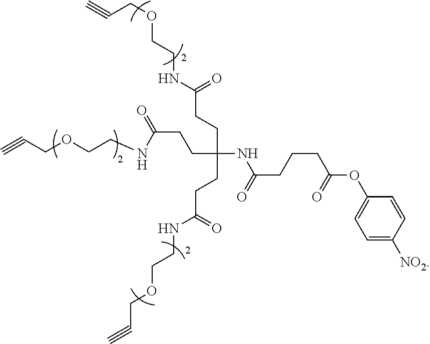

To conjugate the tri-alkyne linker to the annealed duplex, amine-functionalized duplex was dissolved in 90% DMSO/10% H₂O, at ~50-70 mg/mL. 40 equivalents triethylamine was added, followed by 3 equivalents tri-alkyne-PNP. Once complete, the conjugate was precipitated twice in a solvent system of 1× phosphate buffered saline/acetonitrile (1:14 ratio), and dried.

F. Synthesis of Targeting Ligand SM6.1

### ((S)-3-(4-(4-((14-azido-3,6,9,12-tetraoxatetradecyl)oxy)naphthalen-1-yl)phenyl)-3-(2-(4-((4-methylpyridin-2-yl)amino)butanamido)acetamido)propanoic acid)

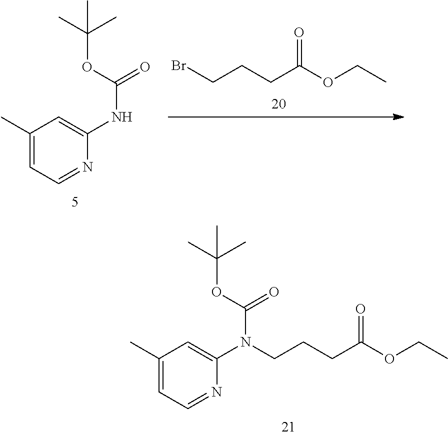

Compound 5 (tert-Butyl (4-methylpyridin-2-yl) carbamate) (0.501 g, 2.406 mmol, 1 equiv.) was dissolved in DMF (17 mL). To the mixture was added NaH (0.116 mg, 3.01 mmol, 1.25 eq, 60% dispersion in oil) The mixture stirred for 10 min before adding Compound 20 (Ethyl 4-Bromobutyrate (0.745 g, 3.82 mmol, 0.547 mL)) (Sigma 167118). After 3 hours the reaction was quenched with ethanol (18 mL) and concentrated. The concentrate was dissolved in DCM (50 mL) and washed with saturated aq. NaCl solution (1×50 mL), dried over Na₂SO₄, filtered and concentrated. The product was purified on silica column, gradient 0-5% Methanol in DCM.

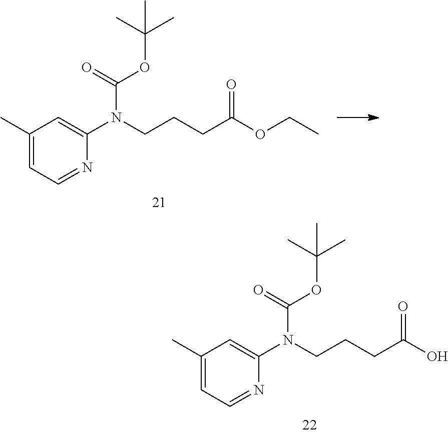

Compound 21 was dissolved (0.80 g, 2.378 mmol) in 100 mL of Acetone: 0.1 M NaOH [1:1]. The reaction was monitored by TLC (5% ethyl acetate in hexane). The organics were concentrated away, and the residue was acidified to pH 3-4 with 0.3 M Citric Acid (40 mL). The product was extracted with DCM (3×75 mL). The organics were pooled, dried over Na₂SO₄, filtered and concentrated. The product was used without further purification.

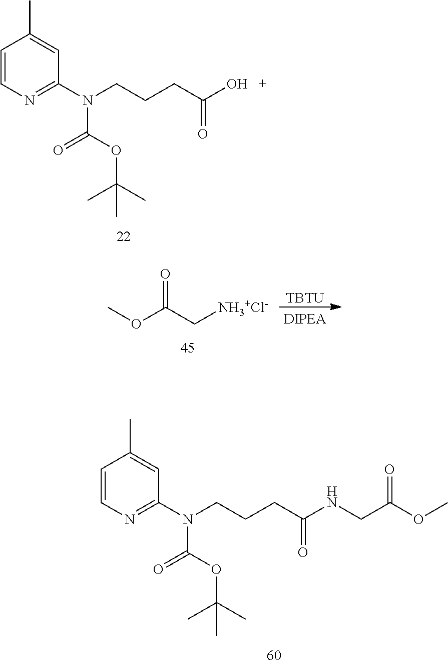

To a solution of Compound 22 (1.1 g, 3.95 mmol, 1 equiv.), Compound 45 (595 mg, 4.74 mmol, 1.2 equiv.), and TBTU (1.52 g, 4.74 mmol, 1.2 equiv.) in anhydrous DMF (10 mL) was added diisopropylethylamine (2.06 mL, 11.85 mmol, 3 equiv.) at 0° C. The reaction mixture was warmed to room temperature and stirred 3 hours. The reaction was quenched by saturated NaHCO₃ solution (10 mL). The aqueous phase was extracted with ethyl acetate (3×10 mL) and the organic phase was combined, dried over anhydrous Na₂SO₄, and concentrated. The product was separated by CombiFlash® using silica gel as the stationary phase. LC-MS: calculated [M+H]+366.20, found 367.

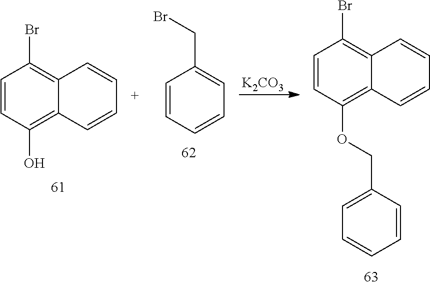

To a solution of compound 61 (2 g, 8.96 mmol, 1 equiv.), and compound 62 (2.13 mL, 17.93 mmol, 2 equiv.) in anhydrous DMF (10 mL) was added K₂CO₃ (2.48 g, 17.93 mmol, 2 equiv.) at 0° C. The reaction mixture was warmed to room temperature and stirred overnight. The reaction was quenched by water (10 mL). The aqueous phase was extracted with ethyl acetate (3×10 mL) and the organic phase was combined, dried over anhydrous Na₂SO₄, and concentrated. The product was separated by CombiFlash® using silica gel as the stationary phase.

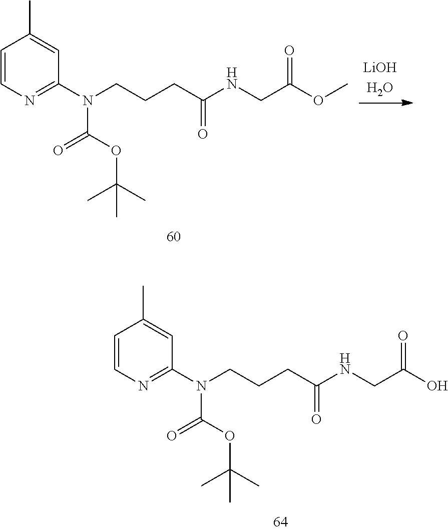 [SMILES: COC(=O)CNC(=O)CCCN(C(=O)OC(C)(C)C)c1cc(C)ccn1 ; Cc1ccnc(N(CCCC(=O)NCC(=O)O)C(=O)OC(C)(C)C)c1]

To a solution of compound 60 (1.77 g, 4.84 mmol, 1 equiv.) in THF (5 mL) and H₂O (5 mL) was added lithium hydroxide monohydrate (0.61 g, 14.53 mmol, 3 equiv.) portion-wise at 0° C. The reaction mixture was warmed to room temperature. After stirring at room temperature for 3 hours, the reaction mixture was acidified by HCl (6 N) to pH 3.0. The aqueous phase was extracted with ethyl acetate (3×20 mL) and the organic layer was combined, dried over Na₂SO₄, and concentrated. LC-MS: calculated [M+H]+352.18, found 352.

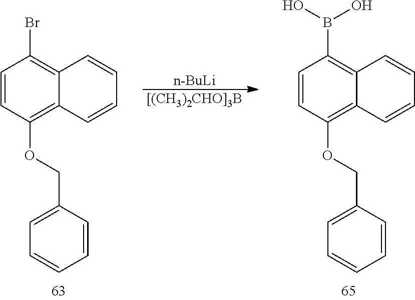

To a solution of compound 63 (1.88 g, 6.0 mmol, 1.0 equiv.) in anhydrous THF (20 mL) was added n-BuLi in hexane (3.6 mL, 9.0 mmol, 1.5 equiv.) drop-wise at −78° C. The reaction was kept at −78° C. for another 1 hour. Triisopropylborate (2.08 mL, 9.0 mmol, 1.5 equiv.) was then added into the mixture at −78° C. The reaction was then warmed up to room temperature and stirred for another 1 hour. The reaction was quenched by saturated NH₄Cl solution (20 mL) and the pH was adjusted to 3. The aqueous phase was extracted with EtOAc (3×20 mL) and the organic phase was combined, dried over Na₂SO₄, and concentrated.

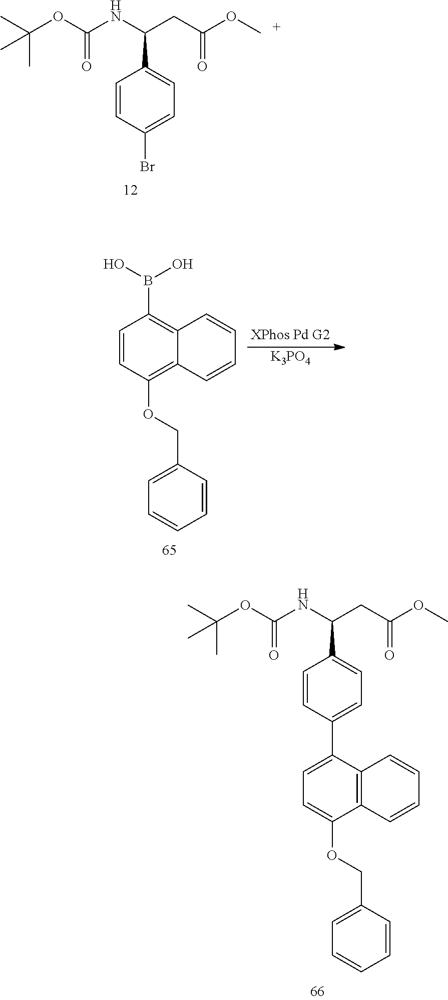

Compound 12 (300 mg, 0.837 mmol, 1.0 equiv.), Compound 65 (349 mg, 1.256 mmol, 1.5 equiv.), XPhos Pd G2 (13 mg, 0.0167 mmol, 0.02 equiv.), and K₃PO₄ (355 mg, 1.675 mmol, 2.0 equiv.) were mixed in a round-bottom flask. The flask was sealed with a screw-cap septum, and then evacuated and backfilled with nitrogen (this process was repeated a total of 3 times). Then, THF (8 mL) and water (2 mL) were added via syringe. The mixture was bubbled with nitrogen for 20 min and the reaction was kept at room temperature for overnight. The reaction was quenched with water (10 mL), and the aqueous phase was extracted with ethyl acetate (3×10 mL). The organic phase was dried over Na₂SO₄, concentrated, and purified via CombiFlash® using silica gel as the stationary phase and was eluted with 15% EtOAc in hexane. LC-MS: calculated [M+H]+512.24, found 512.56.

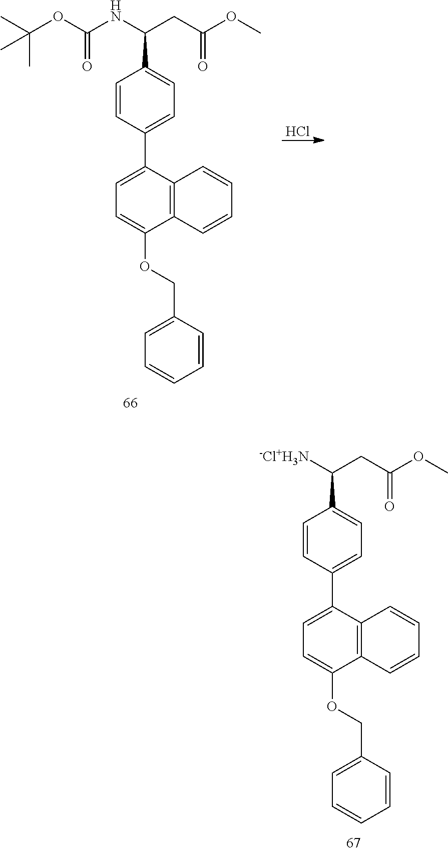

Compound 66 (858 mg, 1.677 mmol, 1.0 equiv.) was cooled by ice bath. HCl in dioxane (8.4 mL, 33.54 mmol, 20 equiv.) was added into the flask. The reaction was warmed to room temperature and stirred for another 1 hr. The solvent was removed by rotary evaporator and the product was directly used without further purification. LC-MS: calculated [M+H]+412.18, found 412.46.

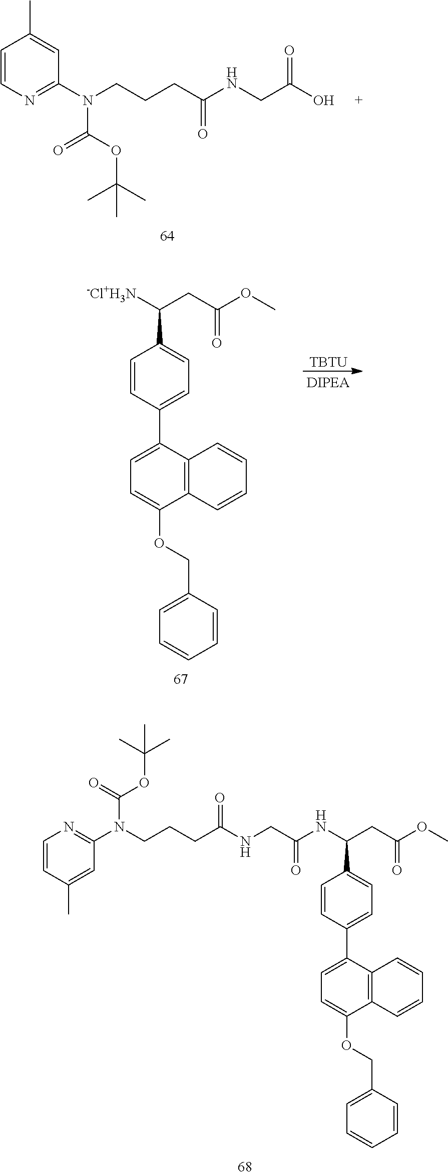

To a solution of compound 64 (500 mg, 1.423 mmol, 1 equiv.), compound 67 (669 mg, 1.494 mmol, 1.05 equiv.), and TBTU (548 mg, 0.492 mmol, 1.2 equiv.) in anhydrous DMF (15 mL) was added diisopropylethylamine (0.744 mL, 4.268 mmol, 3 equiv.) at 0° C. The reaction mixture was warmed to room temperature and stirred for another 1 hr. The reaction was quenched by saturated NaHCO₃ aqueous solution (10 mL) and the product was extracted with ethyl acetate (3×20 mL). The organic phase was combined, dried over Na₂SO₄, and concentrated. The product was purified by CombiFlash® using silica gel as the stationary phase and was eluted with 3-4% methanol in DCM. The yield was 96.23%. LC-MS: calculated [M+H]+745.35, found 746.08.

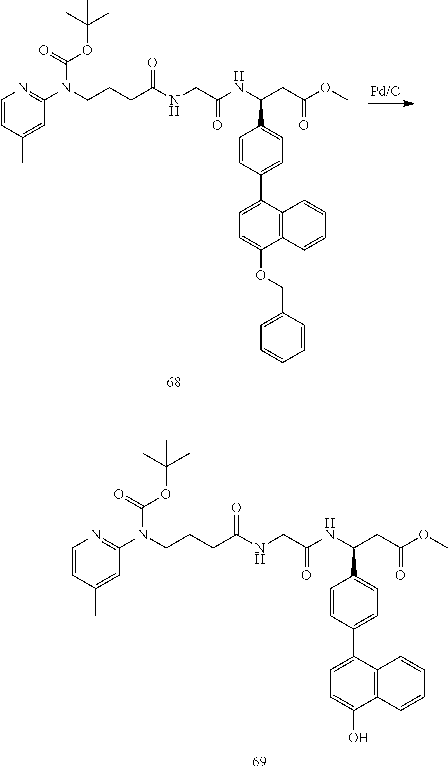 [SMILES: COC(=O)C[C@H](NC(=O)CNC(=O)CCCN(C(=O)OC(C)(C)C)c1cc(C)ccn1)c1ccc(-c2ccc(OCc3ccccc3)c3ccccc23)cc1 ; COC(=O)C[C@H](NC(=O)CNC(=O)CCCN(C(=O)OC(C)(C)C)c1cc(C)ccn1)c1ccc(-c2ccc(O)c3ccccc23)cc1]

To a solution of compound 68 (1.02 g, 1.369 mmol, 1 equiv.) in ethyl acetate (10 mL) was added 10% Pd/C (0.15 g, 50% H₂O) at room temperature. The reaction mixture was warmed to room temperature and the reaction was monitored by LC-MS. The reaction was kept at room temperature overnight. The solids were filtered through Celite® and the solvent was removed by rotary evaporator. The product was directly used without further purification. LC-MS: [M+H]+655.31, found 655.87.

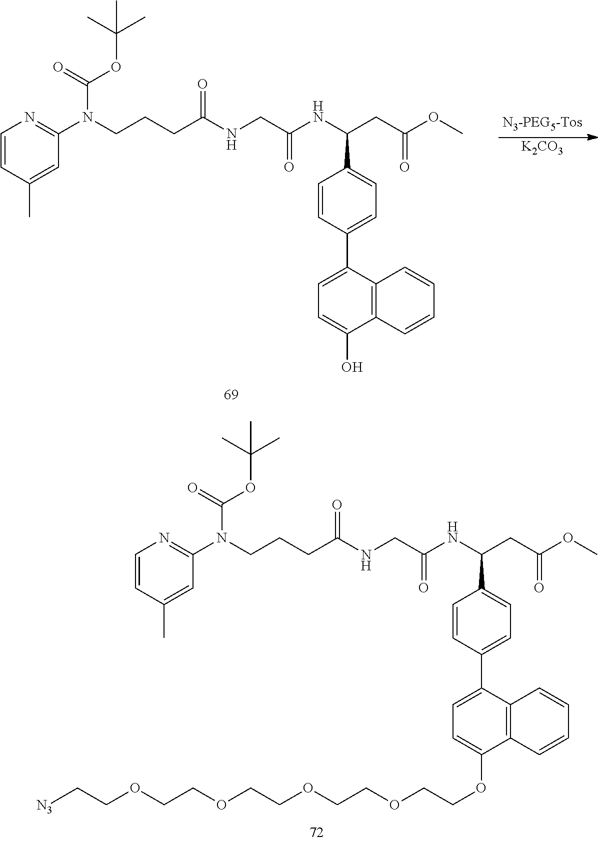

To a solution of compound 69 (100 mg, 0.152 mmol, 1 equiv.) and azido-PEG₅-OTs (128 mg, 0.305 mmol, 2 equiv.) in anhydrous DMF (2 mL) was added K₂CO₃ (42 mg, 0.305 mmol, 2 equiv.) at 0° C. The reaction mixture was stirred for 6 hours at 80° C. The reaction was quenched by saturated NaHCO₃ solution and the aqueous layer was extracted with ethyl acetate (3×10 mL). The organic phase was combined, dried over Na₂SO₄, and concentrated. LC-MS: calculated [M+H]+900.40, found 901.46.

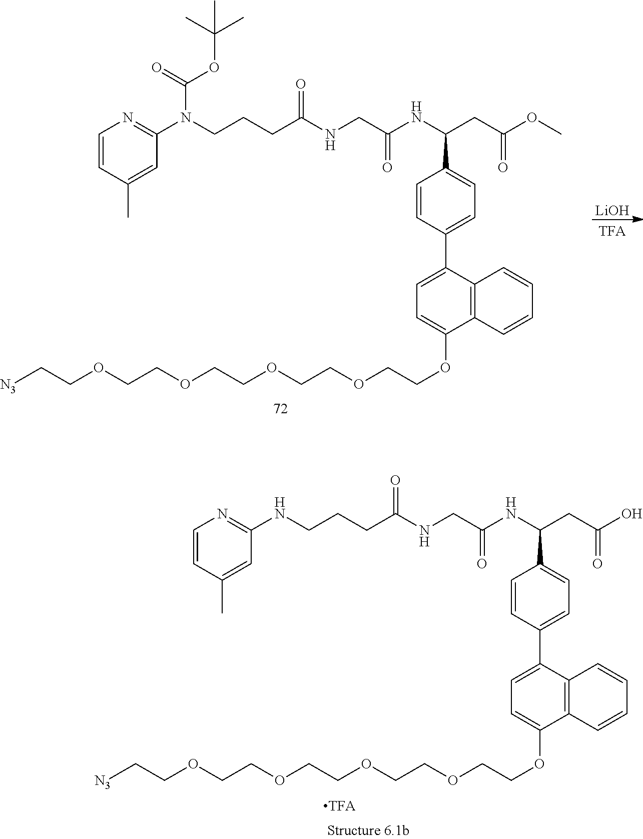 [SMILES: COC(=O)C[C@H](NC(=O)CNC(=O)CCCN(C(=O)OC(C)(C)C)c1cc(C)ccn1)c1ccc(-c2ccc(OCCOCCOCCOCCOCCN=[N+]=[N-])c3ccccc23)cc1 ; "LiOH" ; Cc1ccnc(NCCCC(=O)NCC(=O)N[C@@H](CC(=O)O)c2ccc(-c3ccc(OCCOCCOCCOCCOCCN=[N+]=[N-])c4ccccc34)cc2)c1]

To a solution of compound 72 (59 mg, 0.0656 mmol, 1.0 equiv.) in THF (2 mL) and water (2 mL) was added lithium hydroxide (5 mg, 0.197 mmol, 3.0 equiv.) at room temperature. The mixture was stirred at room temperature for another 1 hr. The pH was adjusted to 3.0 by HCl (6N) and the aqueous phase was extracted with EtOAc (3×10 mL). The organic phase was combined, dried over Na₂SO₄, and concentrated. TFA (0.5 mL) and DCM (0.5 mL) was added into the residue and the mixture was stirred at room temperature for another 3 hr. The solvent was removed by rotary evaporator. LC-MS: calculated [M+H]+786.37, found 786.95.

G. Synthesis of TriAlk 14

TriAlk14 and (TriAlk14) s as shown in Table 11, above, may be synthesized using the synthetic route shown below. Compound 14 may be added to the sense strand as a phosphoramidite using standard oligonucleotide synthesis techniques, or compound 22 may be conjugated to the sense strand comprising an amine in an amide coupling reaction.

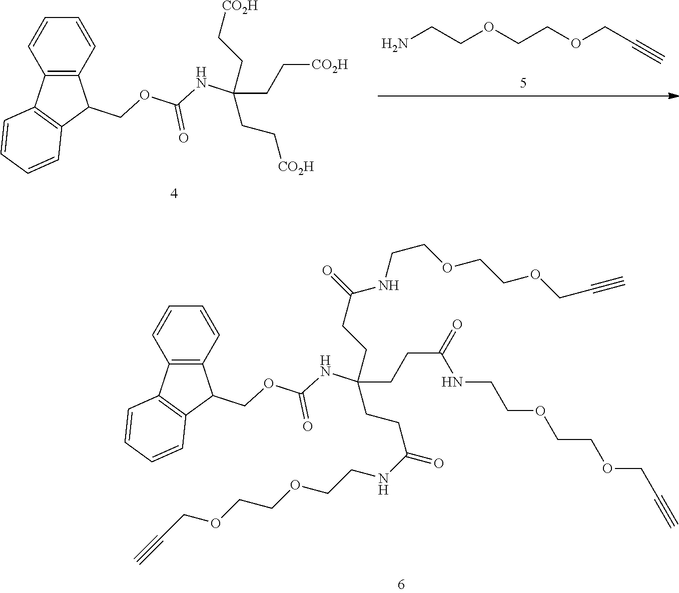

To a 3-L jacketed reactor was added 500 mL DCM and 4 (75.0 g, 0.16 mol). The internal temperature of the reaction was cooled to 0° C. and TBTU (170.0 g, 0.53 mol) was added. The suspension was then treated with the amine 5 (75.5 g, 0.53 mol) dropwise keeping the internal temperature less than 5° C. The reaction was then treated with DIPEA (72.3 g, 0.56 mol) slowly, keeping the internal temperature less than 5° C. After the addition was complete, the reaction was warmed up to 23° C. over 1 hour, and allowed to stir for 3 hours. A 10% kicker charge of all three reagents were added and allowed to stir an additional 3 hours. The reaction was deemed complete when <1% of 4 remained. The reaction mixture was washed with saturated ammonium chloride solution (2×500 mL) and once with saturated sodium bicarbonate solution (500 mL). The organic layer was then dried over sodium sulfate and concentrated to an oil. The mass of the crude oil was 188 g which contained 72% 6 by QNMR. The crude oil was carried to the next step. Calculated mass for C₄₆H₆₀N₄O₁₁=845.0 m/z. Found [M+H]=846.0.

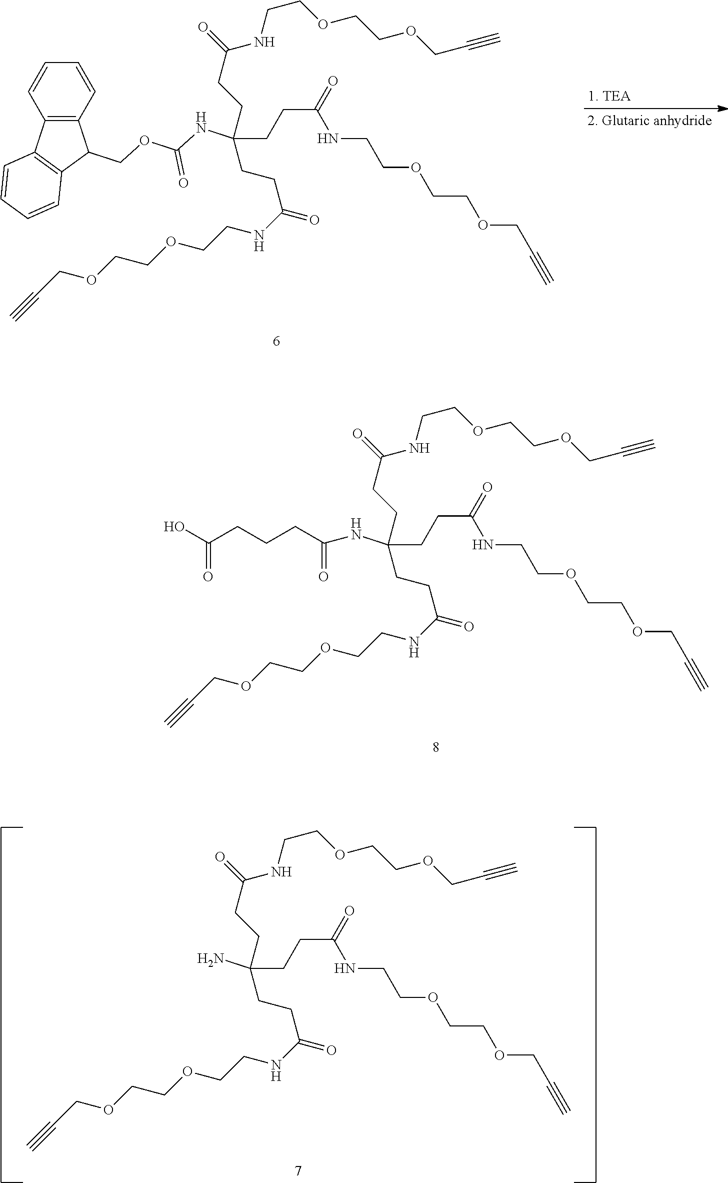

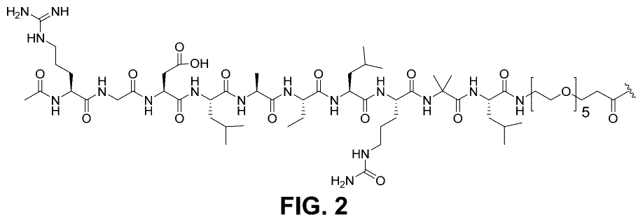

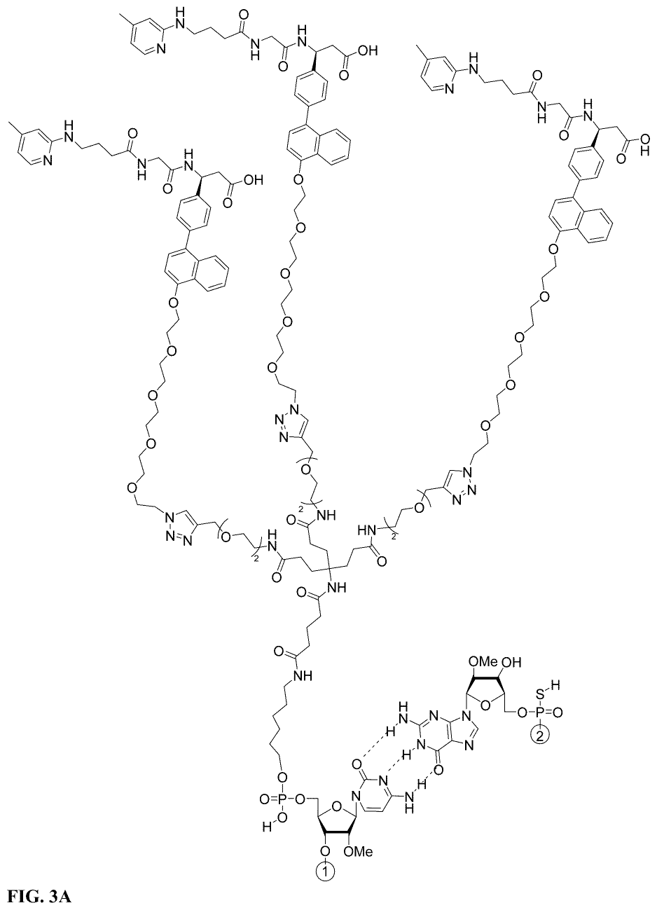

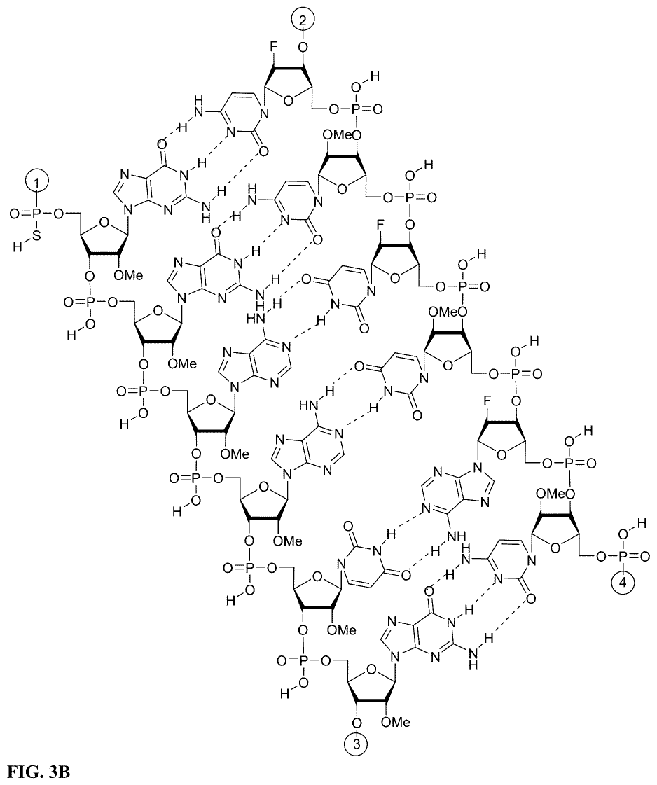

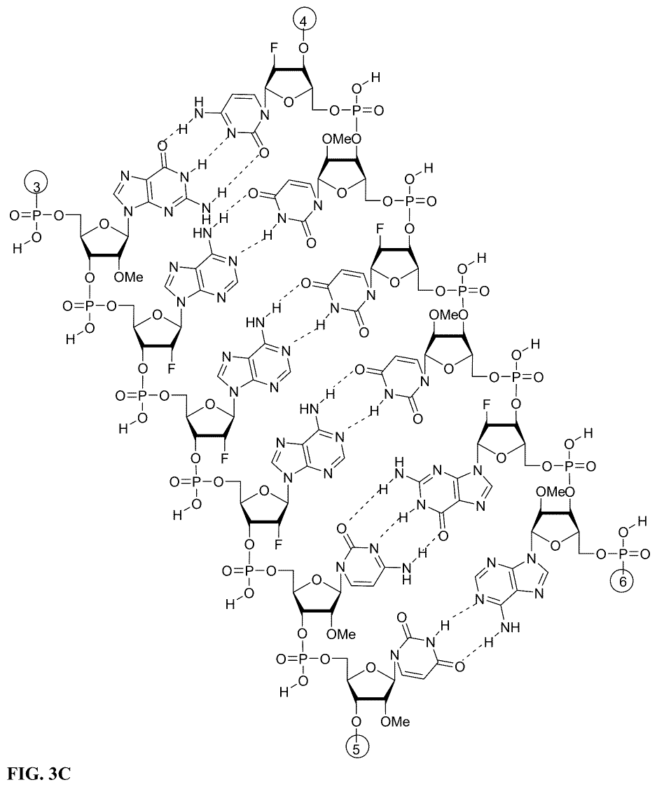

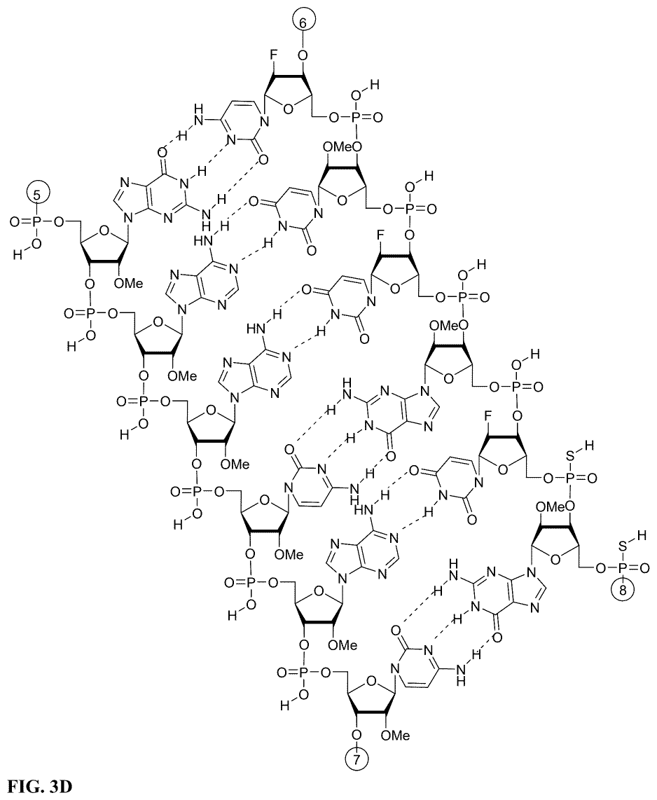

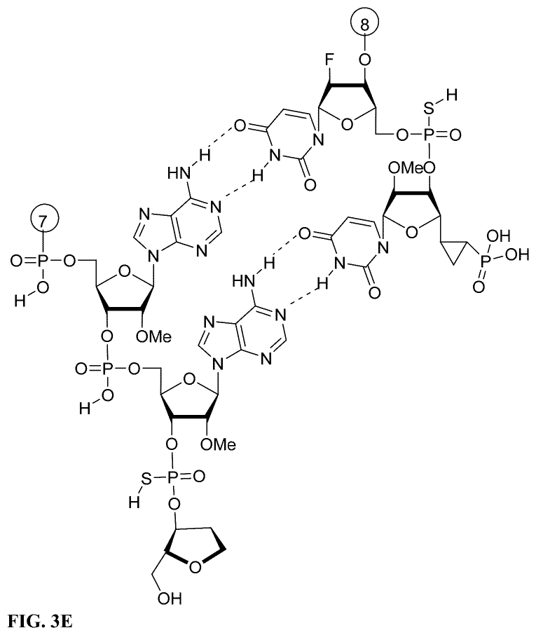

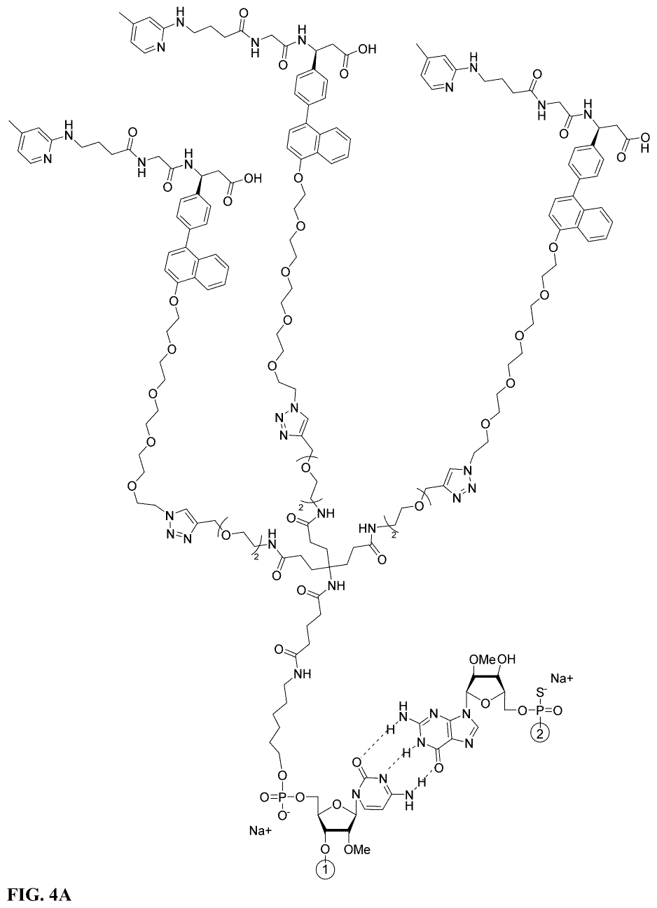

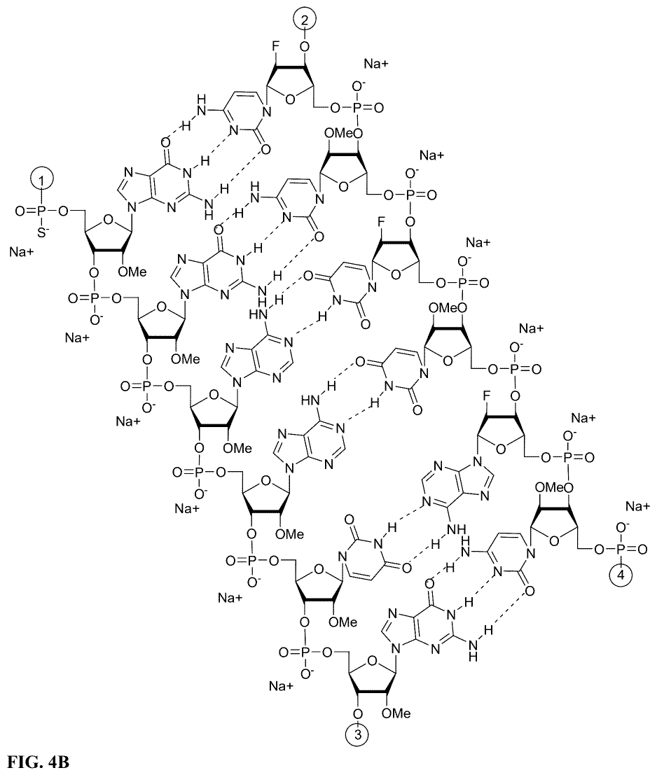

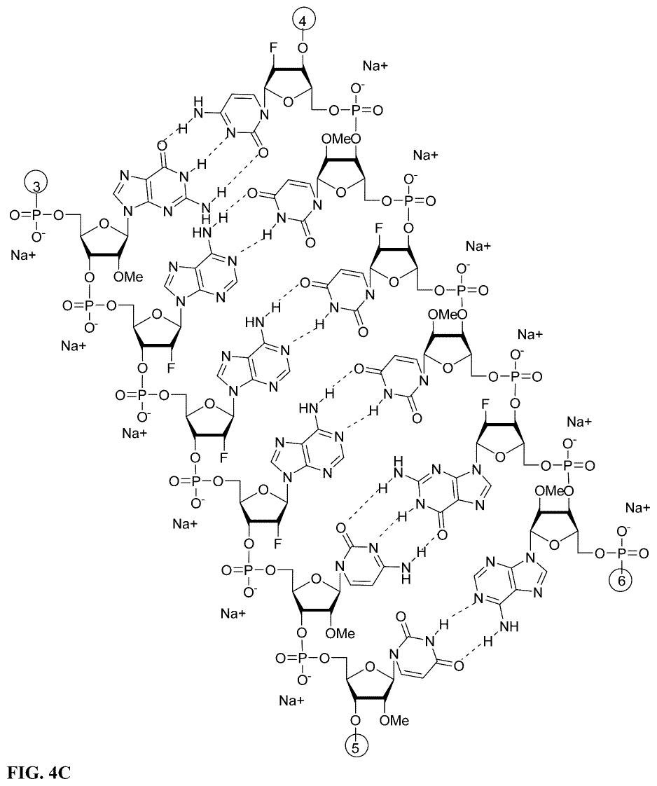

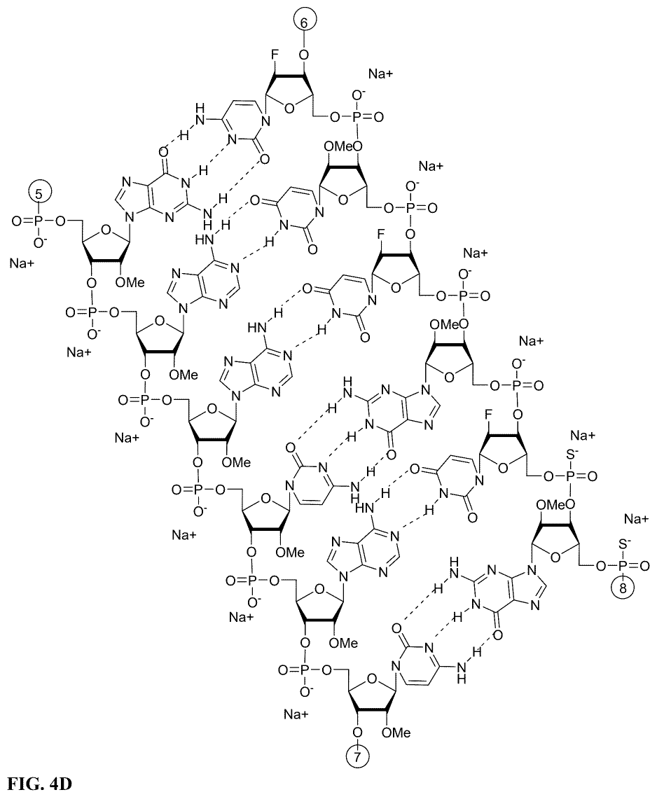

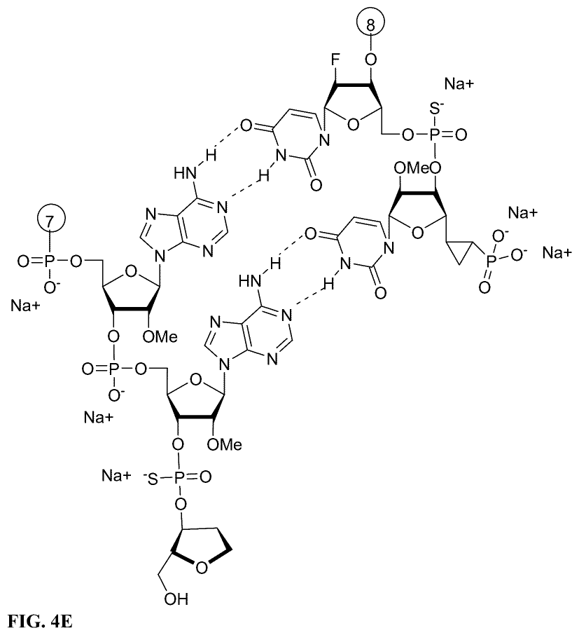

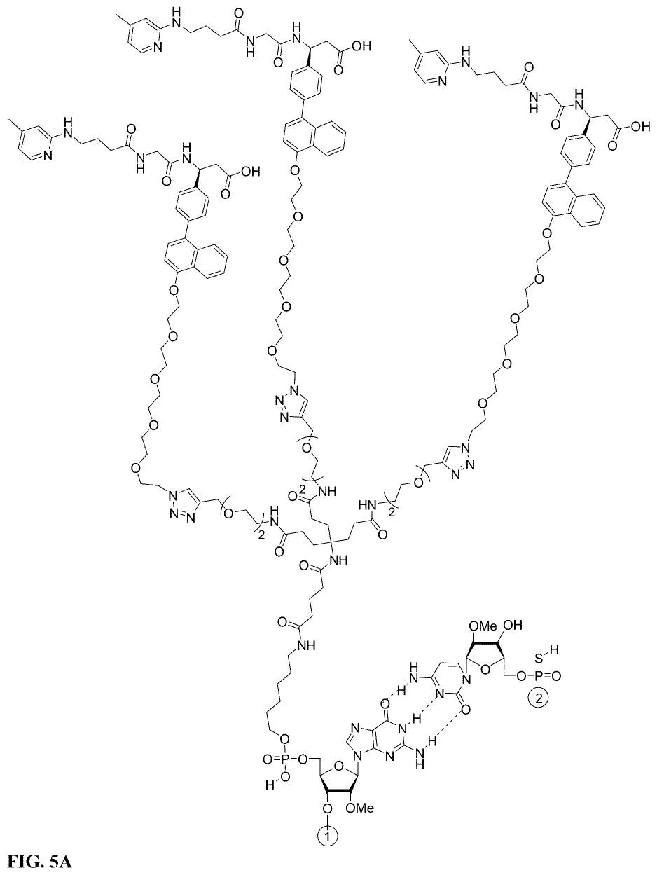

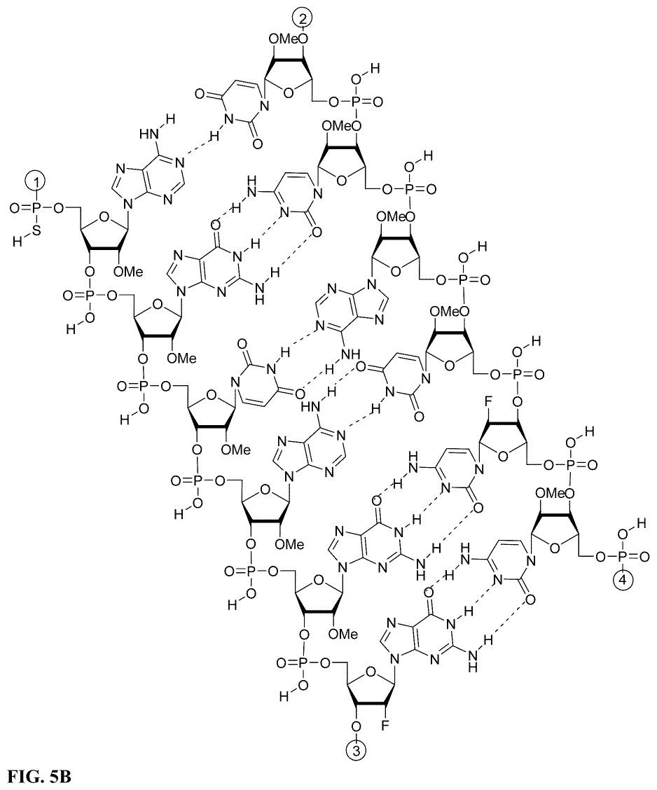

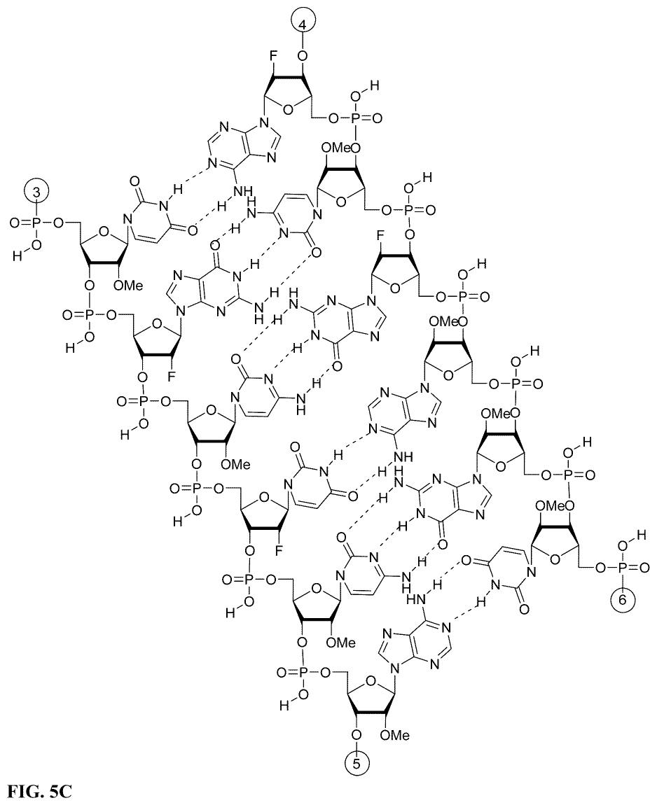

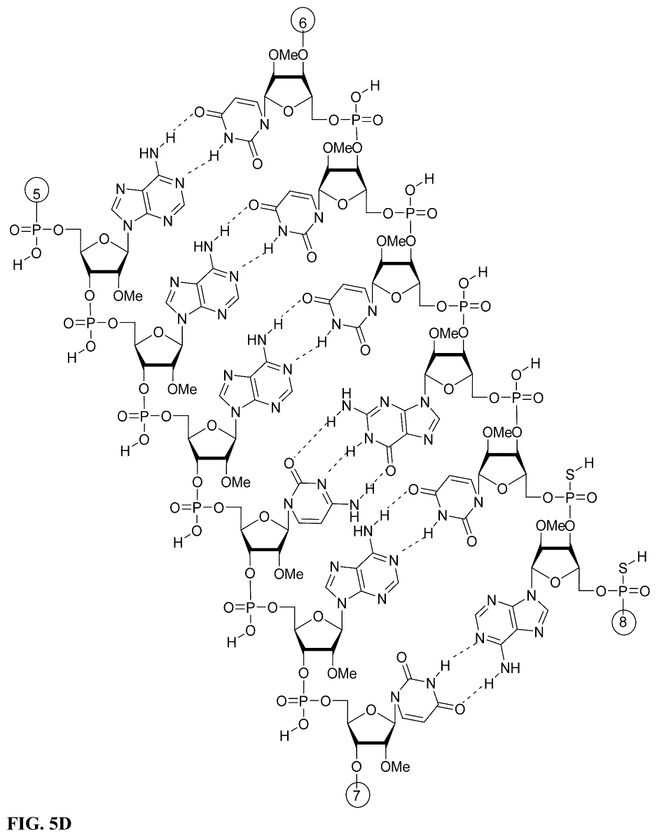

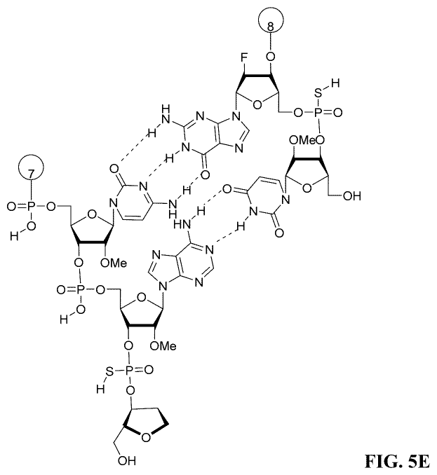

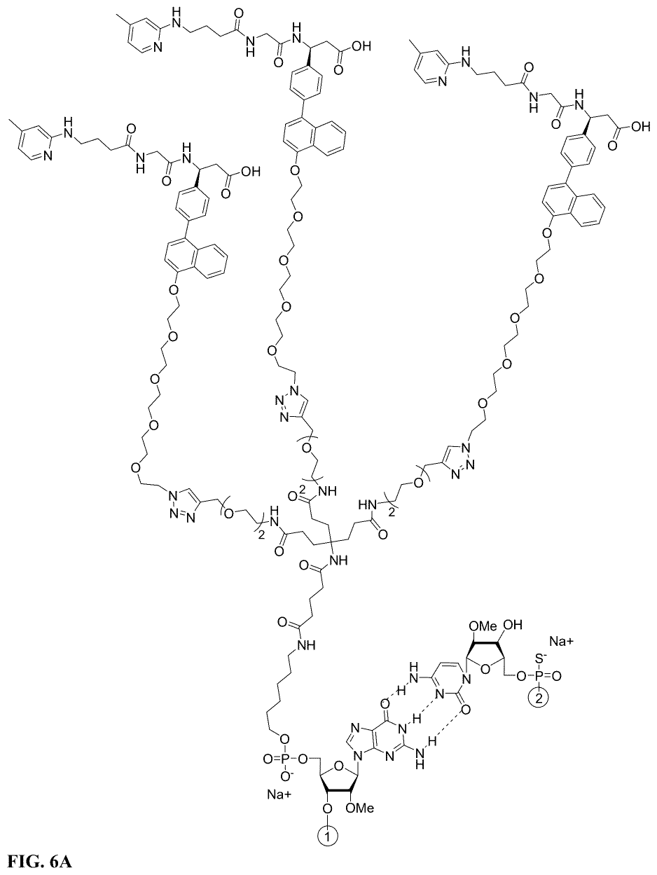

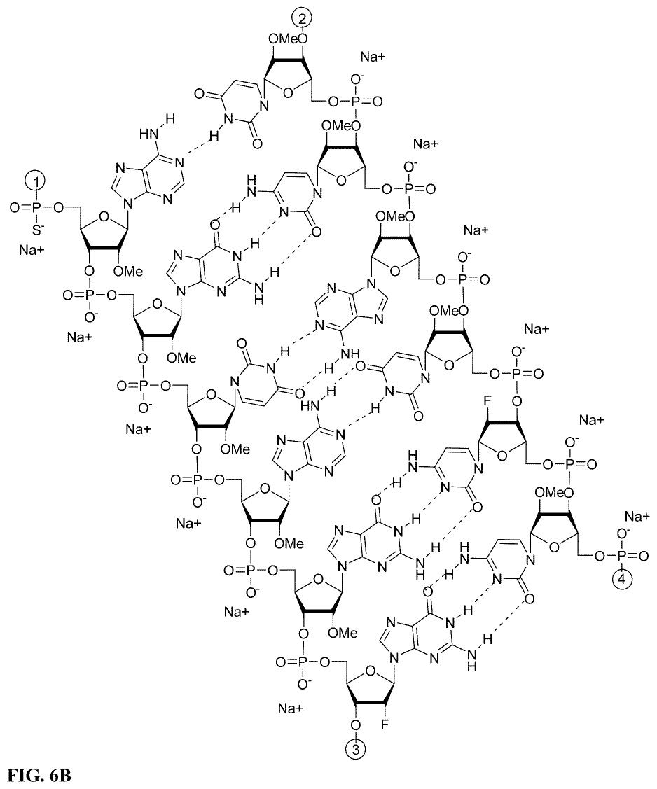

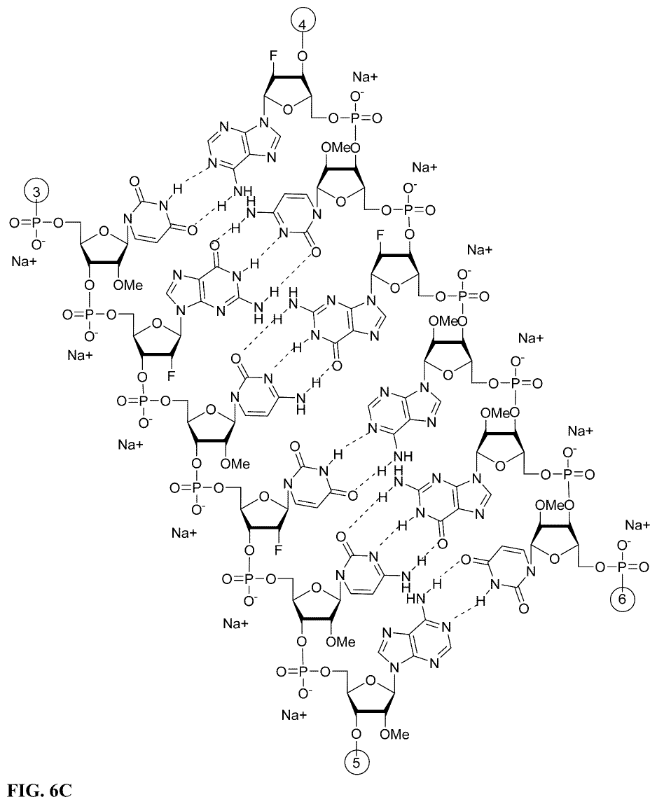

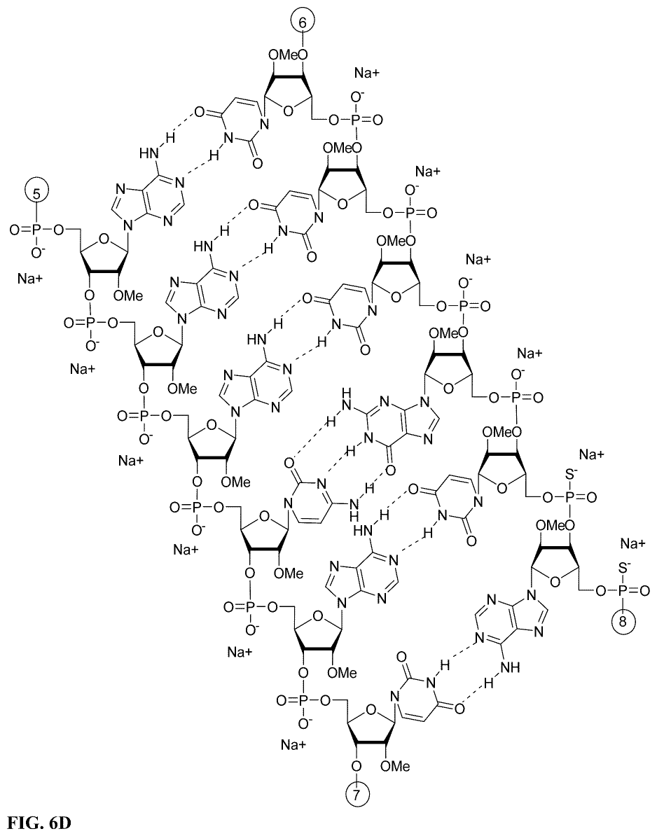

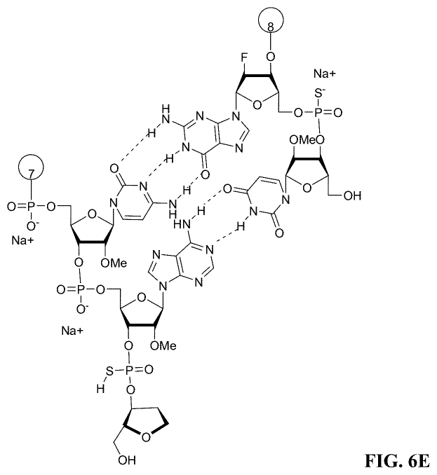

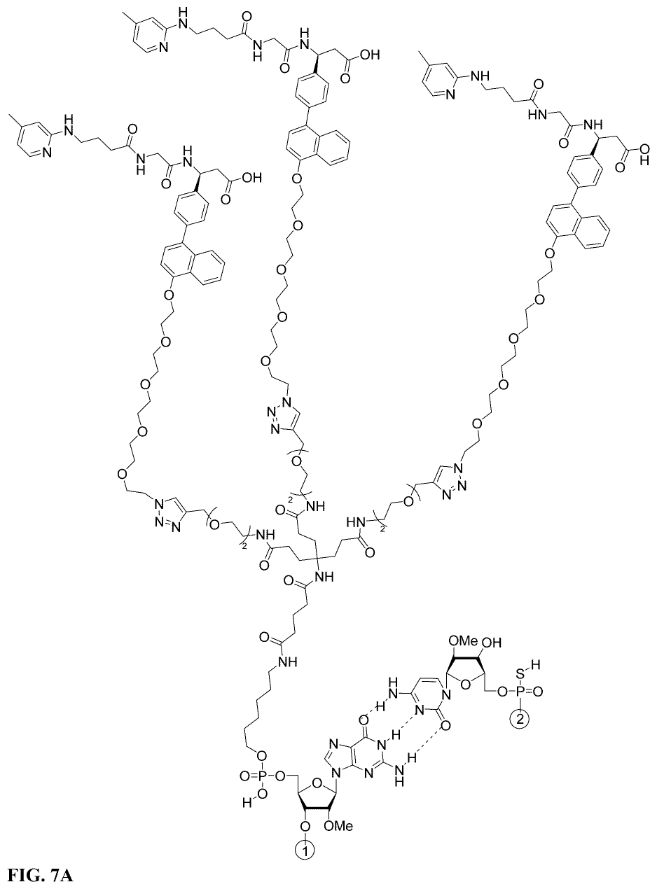

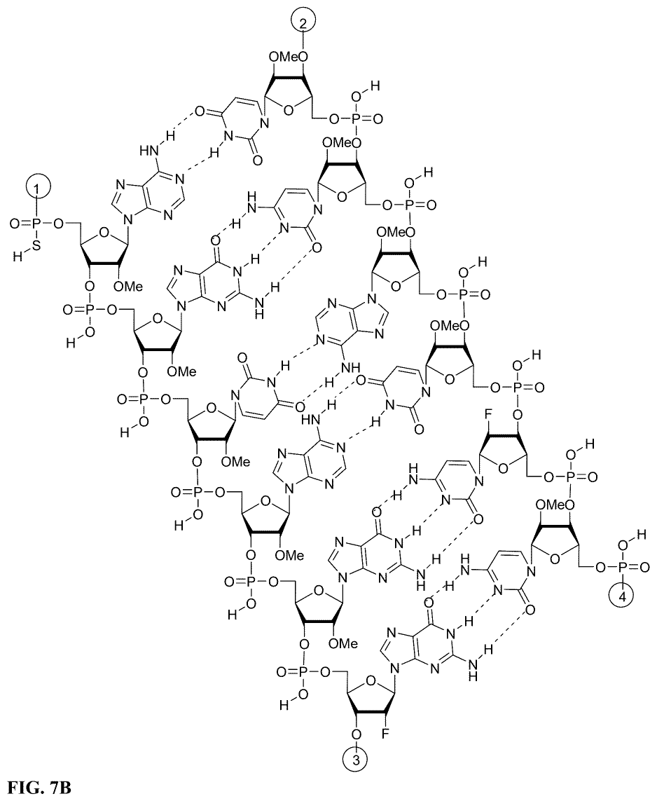

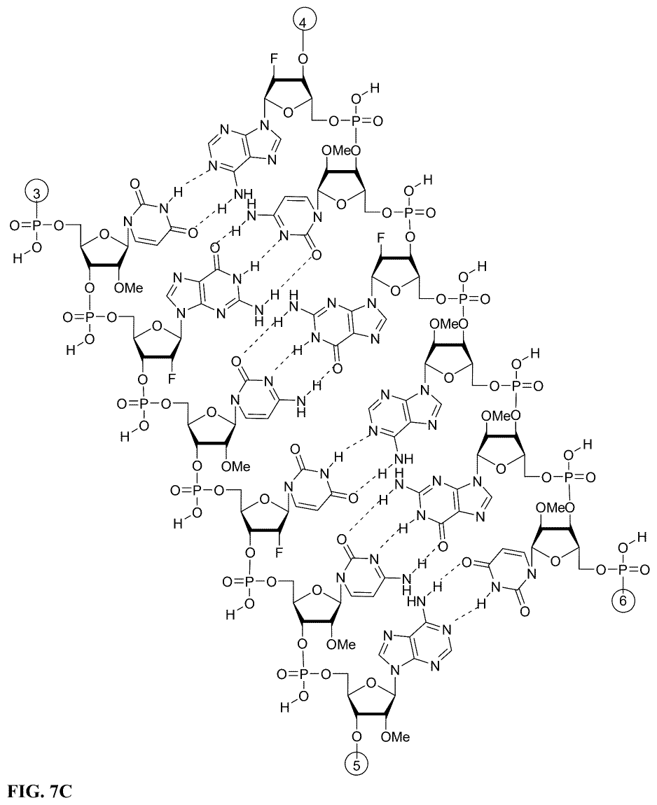

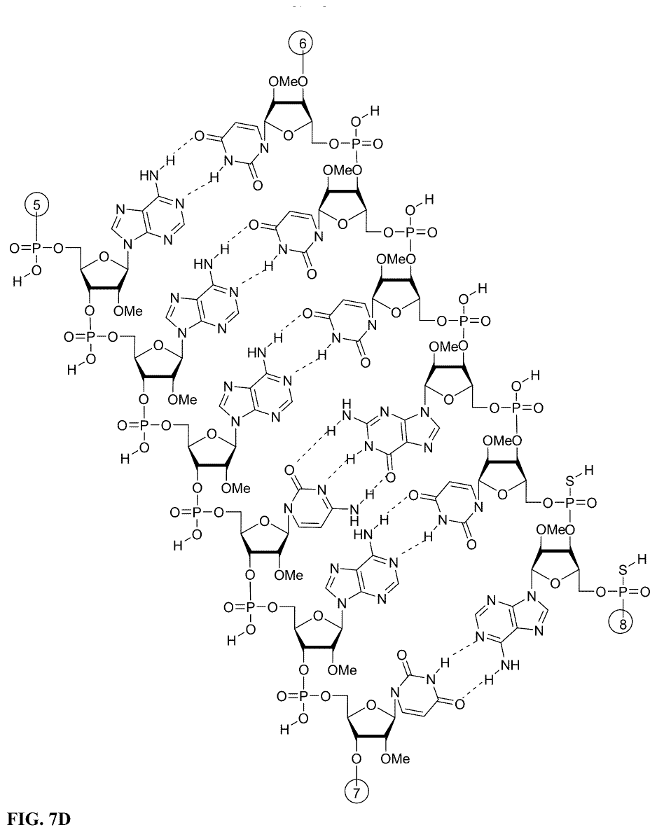

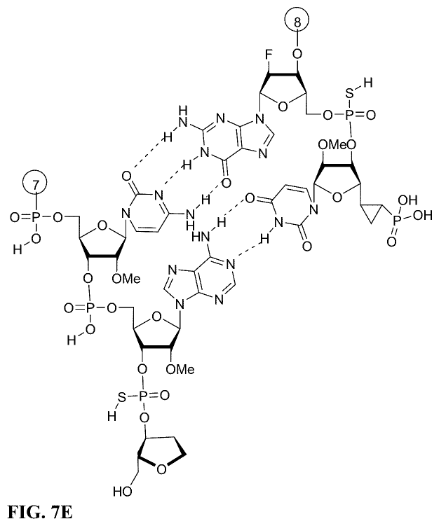

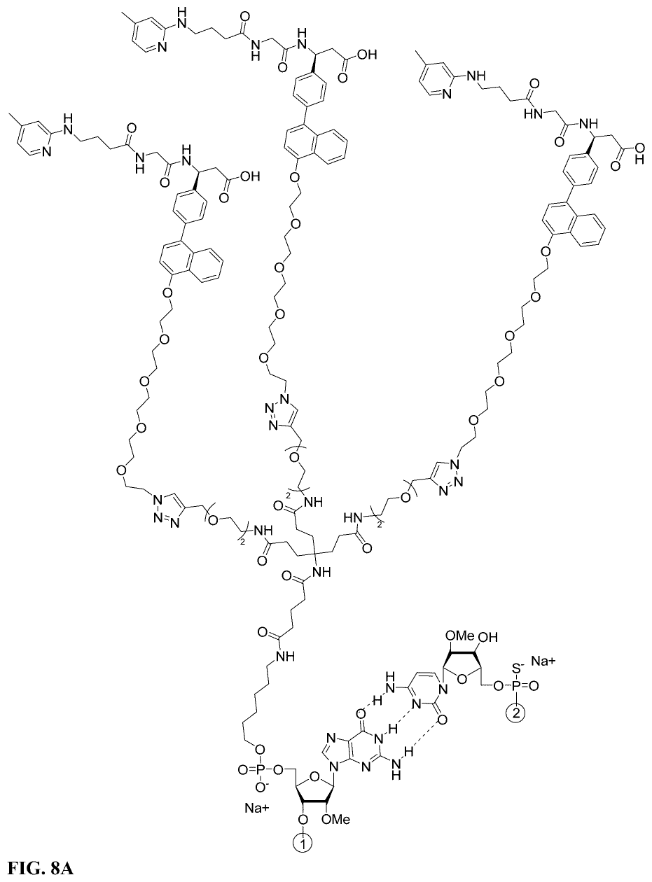

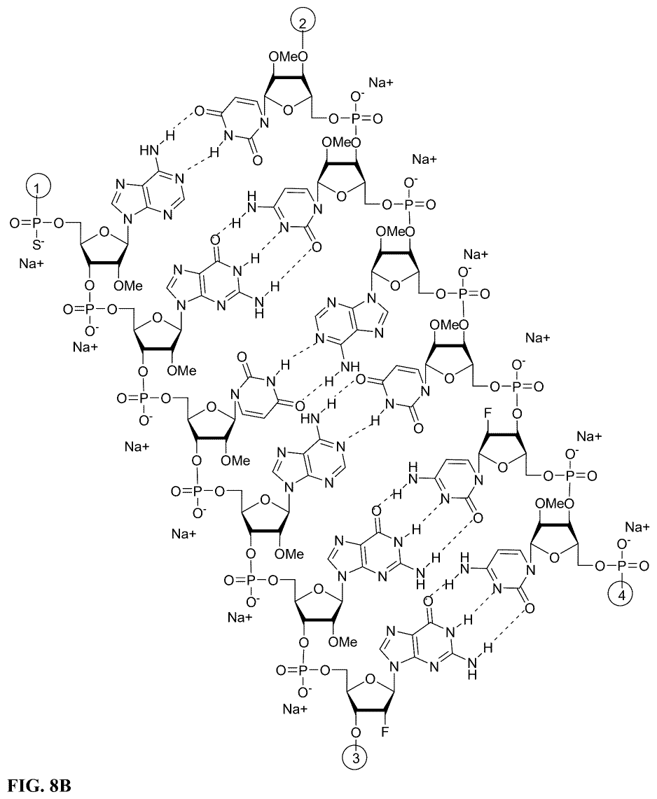

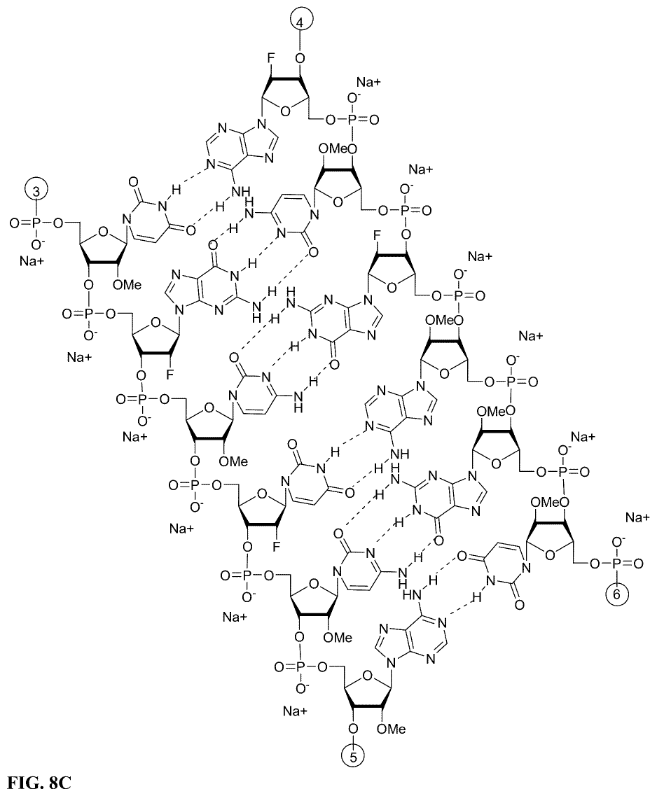

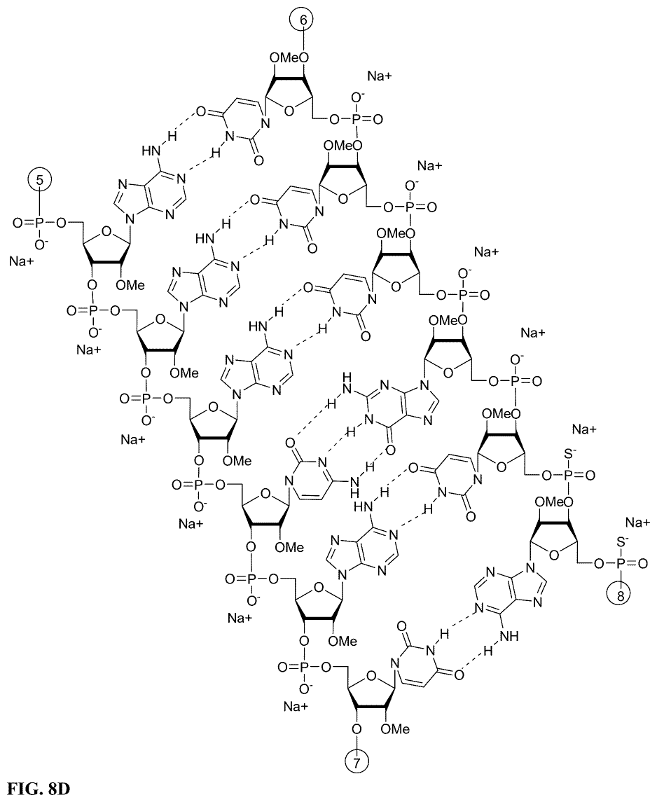

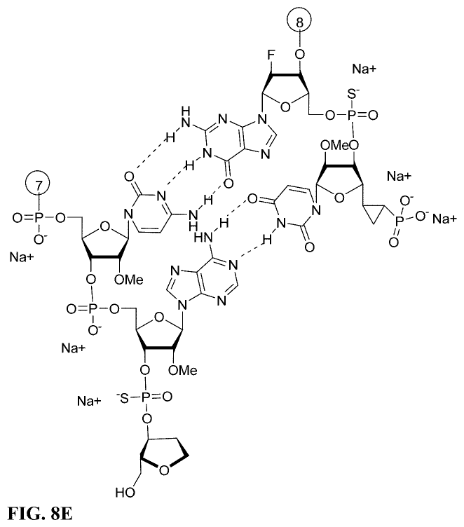

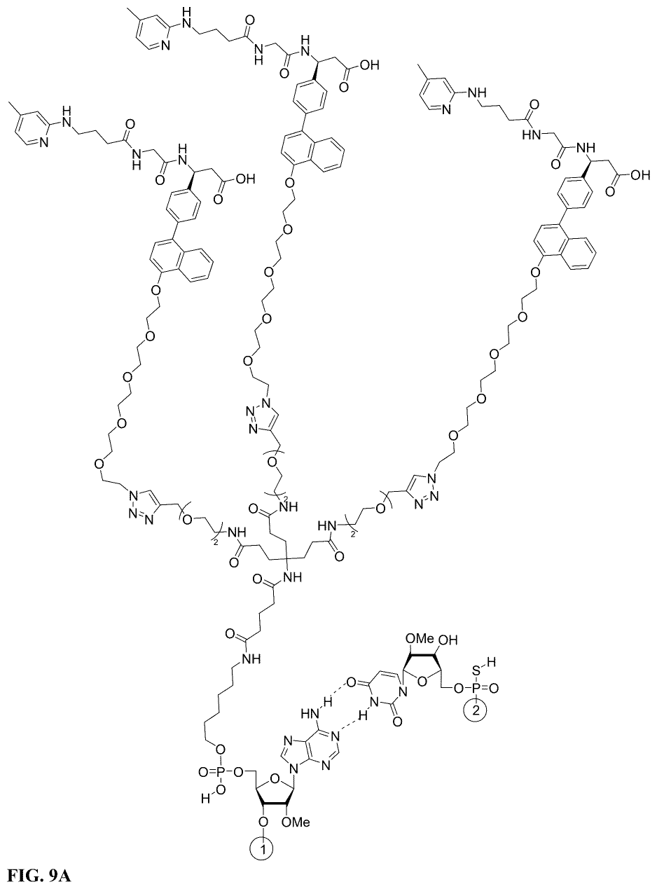

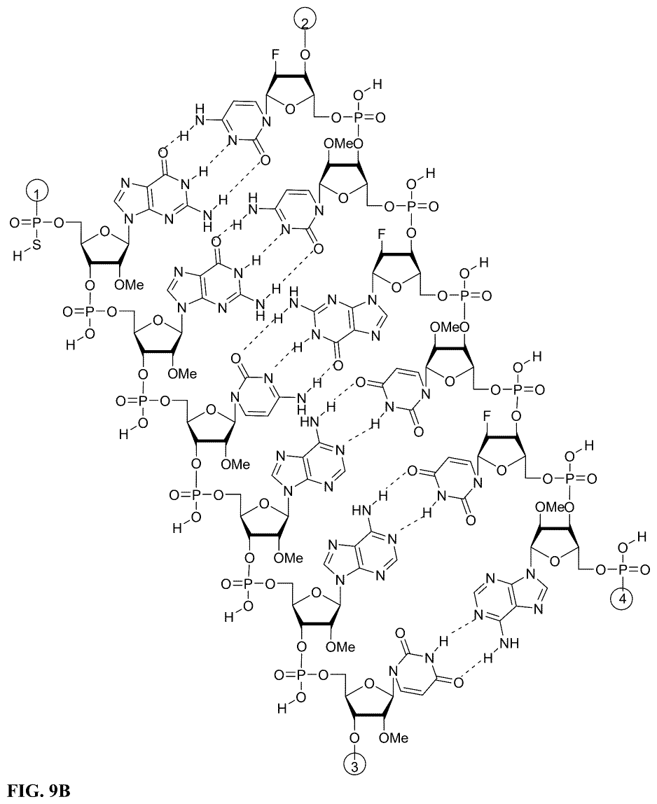

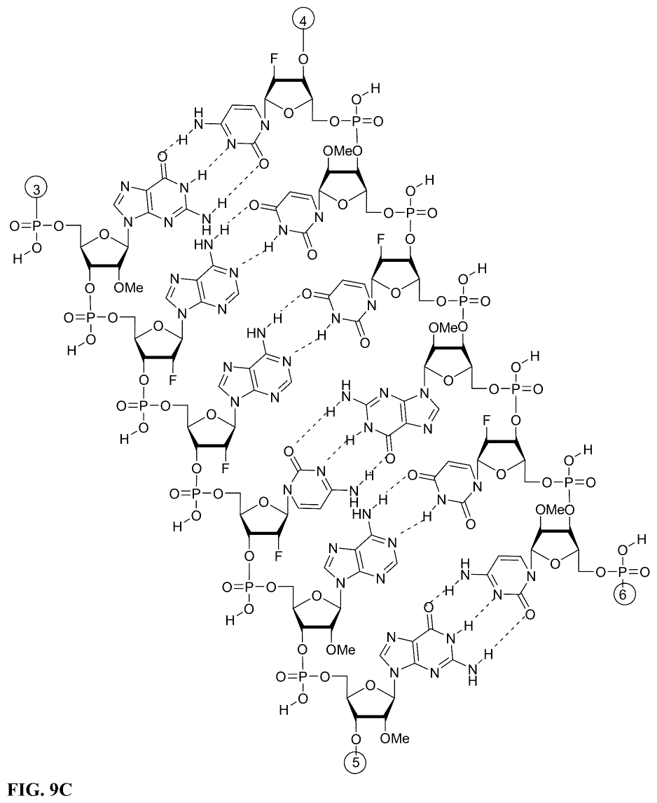

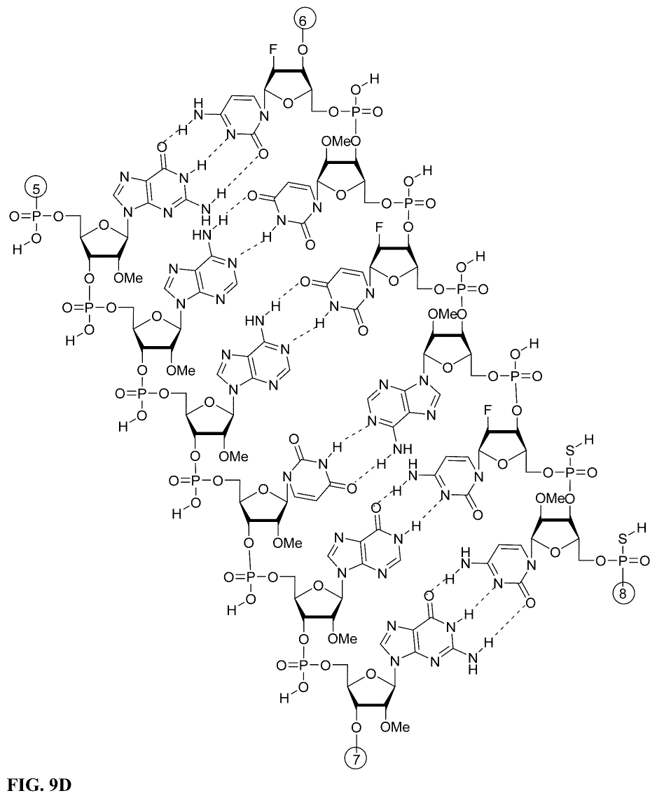

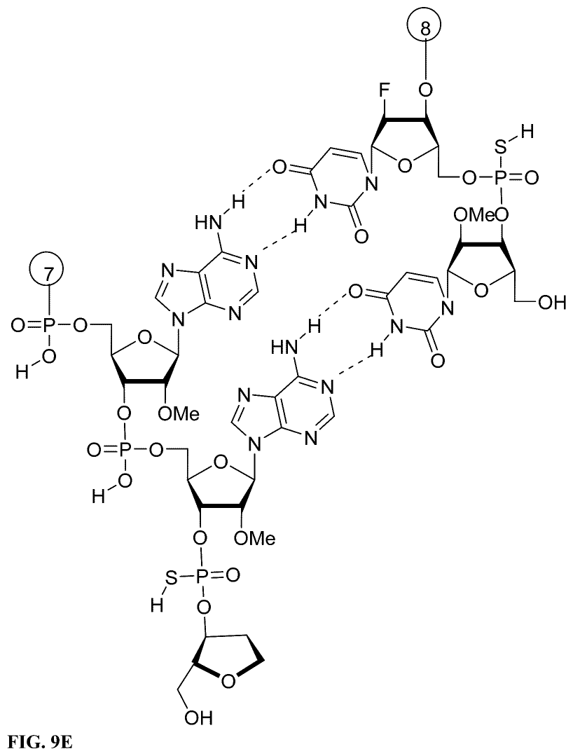

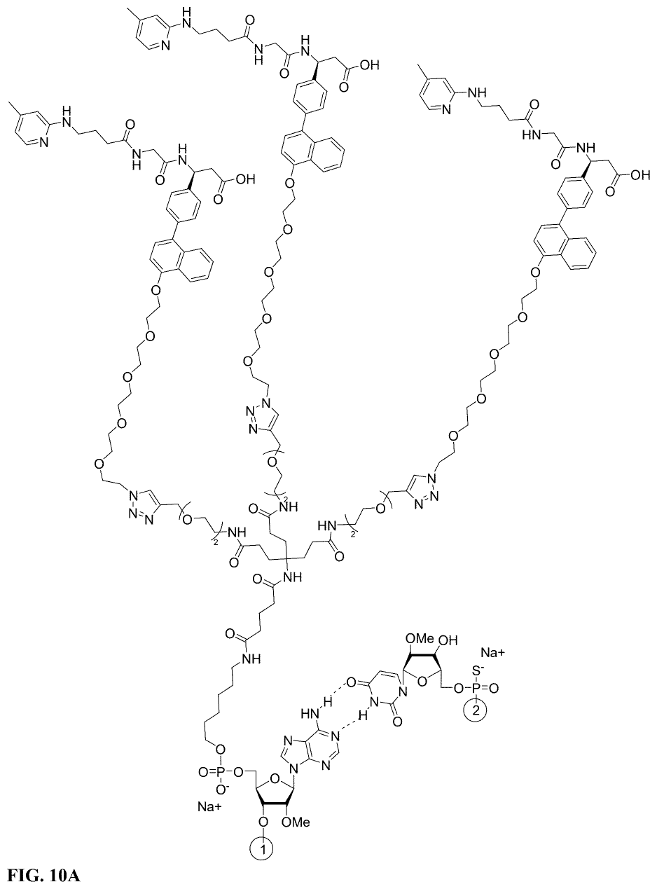

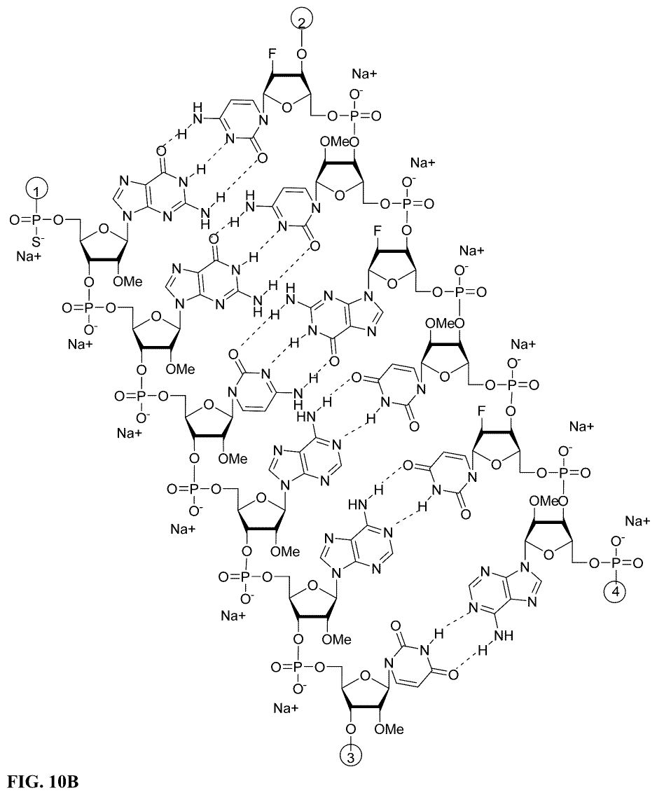

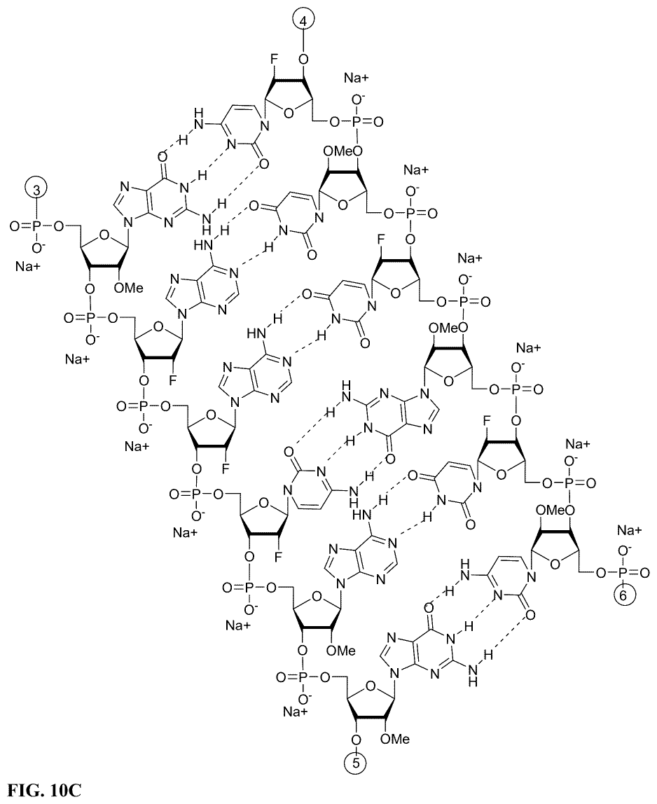

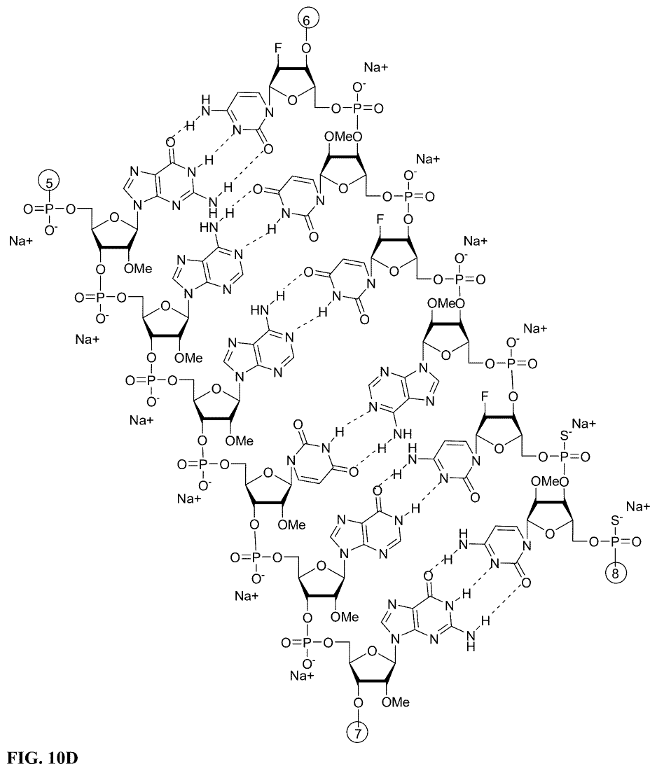

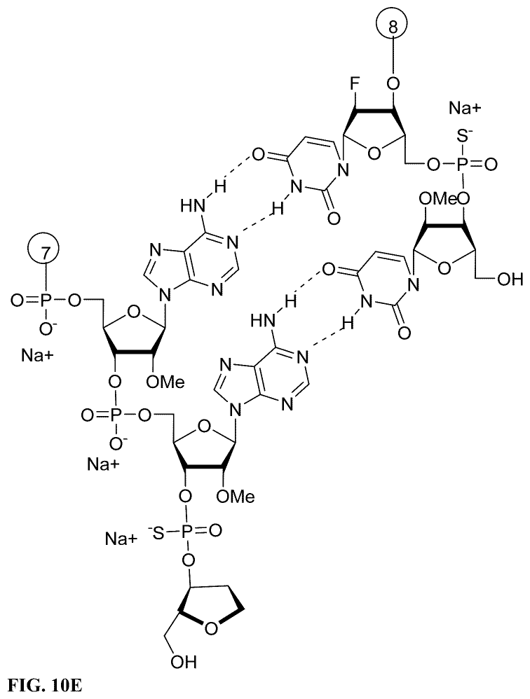

The 121.2 g of crude oil containing 72 wt % compound 6 (86.0 g, 0.10 mol) was dissolved in DMF (344 mL) and treated with TEA (86 mL, 20 v/v %), keeping the internal temperature below 23° C. The formation of dibenzofulvene (DBF) relative to the consumption of Fmoc-amine 6 was monitored via HPLC method 1 (FIG. 2) and the reaction was complete within 10 hours. To the solution was added glutaric anhydride (12.8 g, 0.11 mol) and the intermediate amine 7 was converted to compound 8 within 2 hours. Upon completion, the DMF and TEA were removed at 30° C. under reduced pressure resulting in 100 g of a crude oil. Due to the high solubility of compound 7 in water, an aqueous workup could not be used, and chromatography is the only way to remove DBF, TMU, and glutaric anhydride. The crude oil (75 g) was purified on a Teledyne ISCO Combi-Flash® purification system in three portions. The crude oil (25 g) was loaded onto a 330 g silica column and eluted from 0-20% methanol/DCM over 30 minutes resulting in 42 g of compound 8 (54% yield over 3 steps). Calculated mass for C₃₆H₅₅N₄O₁₂=736.4 m/z. Found [M+H]=737.0.

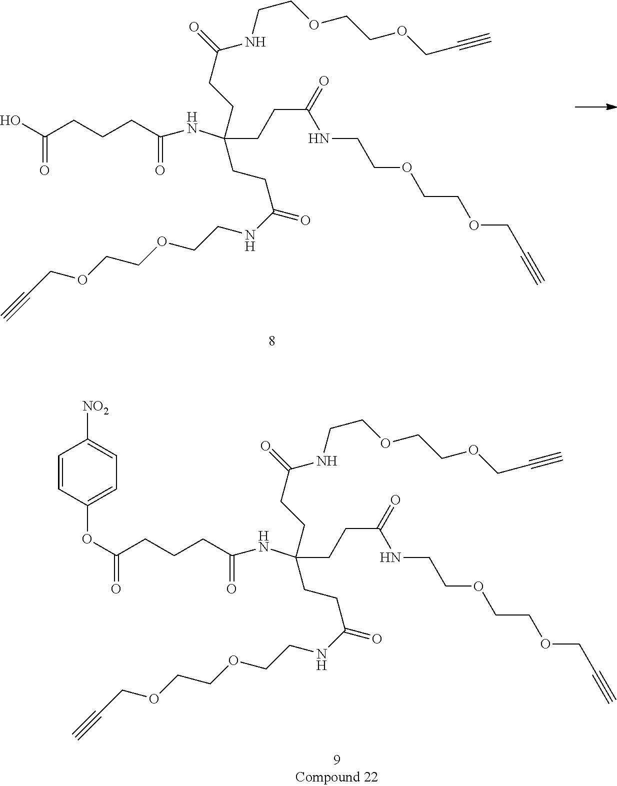 [SMILES: C#CCOCCOCCNC(=O)CCC(CCC(=O)NCCOCCOCC#C)(CCC(=O)NCCOCCOCC#C)NC(=O)CCCC(=O)O ; C#CCOCCOCCNC(=O)CCC(CCC(=O)NCCOCCOCC#C)(CCC(=O)NCCOCCOCC#C)NC(=O)CCCC(=O)Oc1ccc([N+](=O)[O-])cc1]

Compound 8 (42.0 g, 0.057 mol) was co-stripped with 10 volumes of acetonitrile prior to use to remove any residual methanol from chromatography solvents. The oil was redissolved in DMF (210 mL) and cooled to 0° C. The solution was treated with 4-nitrophenol (8.7 g, 0.063 moL) followed by EDC-hydrochloride (12.0 g, 0.063 mol) and found to reach completion within 10 hours. The solution was cooled to 0° C. and 10 volumes ethyl acetate was added followed by 10 volumes saturated ammonium chloride solution, keeping the internal temperature below 15° C. The layers were allowed to separate and the ethyl acetate layer was washed with brine. The combined aqueous layers were extracted twice with 5 volumes ethyl acetate. The combined organic layers were dried over sodium sulfate and concentrated to an oil. The crude oil (55 g) was purified on a Teledyne ISCO Combi-Flash® purification system in three portions. The crude oil (25 g) was loaded onto a 330 g silica column and eluted from 0-10% methanol/DCM over 30 minutes resulting in 22 g of pure 9 (Compound 22) (50% yield). Calculated mass for C₄₂H₅₉N₅O₁₄=857.4 m/z. Found [M+H]=858.0.

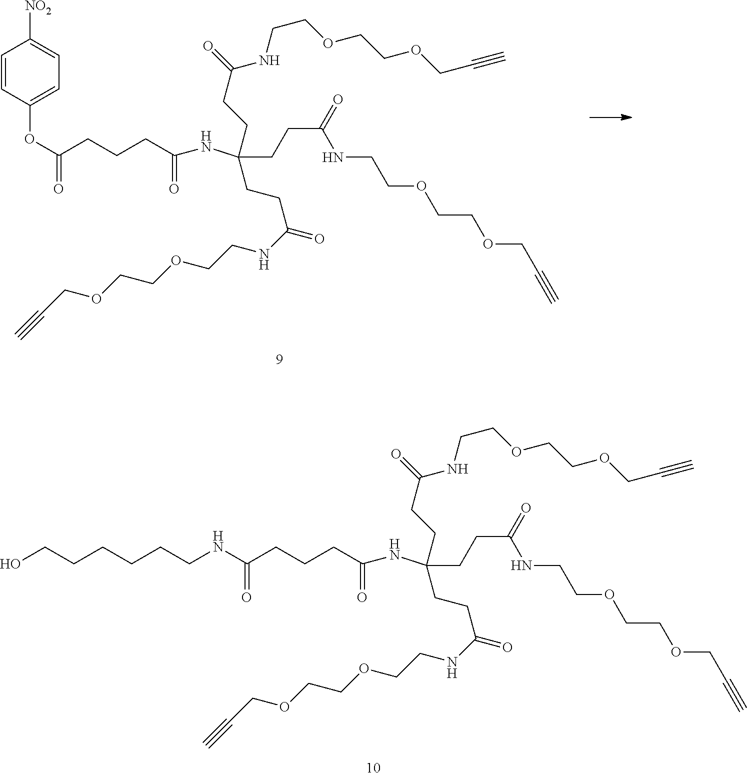 [SMILES: C#CCOCCOCCNC(=O)CCC(CCC(=O)NCCOCCOCC#C)(CCC(=O)NCCOCCOCC#C)NC(=O)CCCC(=O)Oc1ccc([N+](=O)[O-])cc1 ; C#CCOCCOCCNC(=O)CCC(CCC(=O)NCCOCCOCC#C)(CCC(=O)NCCOCCOCC#C)NC(=O)CCCC(=O)NCCCCCCO]

A solution of ester 9 (49.0 g, 57.1 mmol) and 6-amino-1-hexanol (7.36 g, 6.28 mmol) in dichloromethane (3 volumes) was treated with triethylamine (11.56 g, 111.4 mmol) dropwise. The reaction was monitored by observing the disappearance of compound 9 on HPLC Method 1 and was found to be complete in 10 minutes. The crude reaction mixture was diluted with 5 volumes dichloromethane and washed with saturated ammonium chloride (5 volumes) and brine (5 volumes). The organic layer was dried over sodium sulfate and concentrated to an oil. The crude oil was purified on a Teledyne ISCO Combi-Flash® purification system using a 330 g silica column. The 4-nitrophenol was eluted with 100% ethyl acetate and 10 was flushed from the column using 20% methanol/DCM resulting in a colorless oil (39 g, 81% yield). Calculated mass for C₄₂H₆₉N₅O₁₂=836.0 m/z. Found [M+H]=837.0.

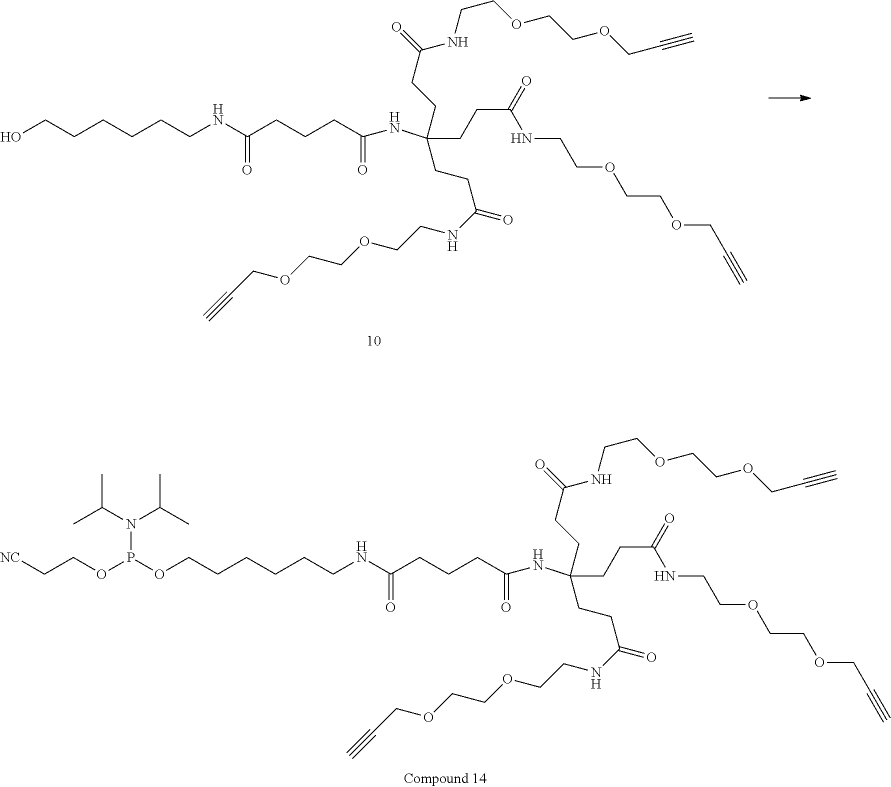 [SMILES: C#CCOCCOCCNC(=O)CCC(CCC(=O)NCCOCCOCC#C)(CCC(=O)NCCOCCOCC#C)NC(=O)CCCC(=O)NCCCCCCO ; C#CCOCCOCCNC(=O)CCC(CCC(=O)NCCOCCOCC#C)(CCC(=O)NCCOCCOCC#C)NC(=O)CCCC(=O)NCCCCCCOP(OCCC#N)N(C(C)C)C(C)C]

Alcohol 10 was co-stripped twice with 10 volumes of acetonitrile to remove any residual methanol from chromatography solvents and once more with dry dichloromethane (KF<60 ppm) to remove trace water. The alcohol 10 (2.30 g, 2.8 mmol) was dissolved in 5 volumes dry dichloromethane (KF<50 ppm) and treated with diisopropylammonium tetrazolide (188 mg, 1.1 mmol). The solution was cooled to 0° C. and treated with 2-cyanoethyl N,N,N′,N′-tetraisopropylphosphoramidite (1.00 g, 3.3 mmol) dropwise. The solution was removed from ice-bath and stirred at 20° C. The reaction was found to be complete within 3-6 hours. The reaction mixture was cooled to 0° C. and treated with 10 volumes of a 1:1 solution of saturated ammonium bicarbonate/brine and then warmed to ambient over 1 minute and allowed to stir an additional 3 minutes at 20° C. The biphasic mixture was transferred to a separatory funnel and 10 volumes of dichloromethane was added. The organic layer was separated and washed with 10 volumes of saturated sodium bicarbonate solution to hydrolyze unreacted bis-phosphorous reagent. The organic layer was dried over sodium sulfate and concentrated to an oil resulting in 3.08 g of 94 wt % Compound 14. Calculated mass for C₅₁H₈₆N₇O₁₃P=1035.6 m/z. Found [M+H]=1036.

H. Conjugation of Targeting Ligands. Either prior to or after annealing, the 5′ or 3′ tridentate alkyne functionalized sense strand is conjugated to targeting ligands. The following example describes the conjugation of targeting ligands to the annealed duplex: Stock solutions of 0.5M Tris(3-hydroxypropyltriazolylmethyl)amine (THPTA), 0.5M of Cu(II) sulfate pentahydrate (Cu(II)SO₄·5H₂O) and 2M solution of sodium ascorbate were prepared in deionized water. A 75 mg/mL solution in DMSO of targeting ligand was made. In a 1.5 mL centrifuge tube containing tri-alkyne functionalized duplex (3 mg, 75 μL, 40 mg/mL in deionized water, ~15,000 g/mol), 25 μL of 1M Hepes pH 8.5 buffer is added. After vortexing, 35 μL of DMSO was added and the solution is vortexed. Targeting ligand was added to the reaction (6 equivalents/duplex, 2 equivalents/alkyne, ~15 μL) and the solution is vortexed. Using pH paper, pH was checked and confirmed to be PH ~8. In a separate 1.5 mL centrifuge tube, 50 μL of 0.5M THPTA was mixed with 10 μL of 0.5M Cu(II)SO₄·5H₂O, vortexed, and incubated at room temp for 5 min. After 5 min, THPTA/Cu solution (7.2 μL, 6 equivalents 5:1 THPTA:Cu) was added to the reaction vial, and vortexed. Immediately afterwards, 2M ascorbate (5 μL, 50 equivalents per duplex, 16.7 per alkyne) was added to the reaction vial and vortexed. Once the reaction was complete (typically complete in 0.5-1 h), the reaction was immediately purified by non-denaturing anion exchange chromatography.

### Example 2. In Vivo Intratracheal Administration of RAGE RNAi Agents in Rats

On study day 1, male Sprague Dawley rats were administered 200 microliters via a microsprayer device (Penn Century, Philadelphia, PA) suitable for intratracheal (IT) administration of isotonic saline or 1.0 mg/kg of one of the following RAGE RNAi agents:

TABLE 12
RAGE RNAi Agent and Dosing for Example 2

| Group ID | AC Duplex Number |
|---|---|
| Group 1 (isotonic saline) | N/A |
| Group 2 (1.0 mg/kg Tri-SM6.1-αvβ6-AD06949) | AC000614 |
| Group 3 (1.0 mg/kg Tri-SM6.1-αvβ6-AD07256) | AC000186 |
| Group 4 (1.0 mg/kg Tri-SM6.1-αvβ6-AD07474) | AC000286 |
| Group 5 (1.0 mg/kg Tri-SM6.1-αvβ6-AD07475) | AC000292 |
| Group 6 (1.0 mg/kg Tri-SM6.1-αvβ6-AD07476) | AC000818 |
| Group 7 (1.0 mg/kg Tri-SM6.1-αvβ6-AD07477) | AC000819 |

As noted in Table 12, each of the RAGE RNAi agents were conjugated to a tridentate small molecule αvβ6 epithelial cell targeting ligand (Tri-SM6.1, see FIG. 1) at the 5′ terminal end of the sense strand, formulated in isotonic saline.

The chemically modified sequences for RAGE RNAi agents AD07474, AD07475, AD07476, and AD07477 are shown in Table 7B (showing duplex), Table 3 (showing respective antisense strand), and Table 5 (showing respective sense strand with linker but without tridentate small molecule αvβ6 epithelial cell targeting ligand (Tri-SM6.1).

AD06949 and AD07256 are rat and mouse-specific sequences that do not have homology with the human AGER gene, and were chemically modified as follows:

Tri-SM6.1-avβ6-AD06949
Modified Sense Strand (5’→3′):
(SEQ ID NO: 888)
Tri-SM6.1-avβ6-(TA14)cscacaugaUfCfCfaugcuiaguas(invAb)
Modified Antisense Strand (5’→3′):
(SEQ ID NO: 889)
usAfscsUfcAfgCfaUfgGfaUfcAfuGfuGfsg
Tri-SM6.1-avβ6-AD07256
Modified Sense Strand (5’→3′):
(SEQ ID NO: 890)
Tri-SM6.1-avβ6-(TA14)cscacaugaUfCfCfaugcuiaguas(invAb)
Modified Antisense Strand (5’→3′):
(SEQ ID NO: 891)
cPrpusAfscsUfcAfgCfaUfgGfaUfcAfuGfuGfsg

Five (5) rats were dosed per group. Rats were sacrificed on study day 8, and total RNA was isolated from both lungs following collection and homogenization. Rat AGER mRNA expression was quantitated by probe-based quantitative PCR, normalized to rat GAPDH expression, and expressed as fraction of vehicle control group (geometric mean, +/−95% confidence interval).

TABLE 13
Average Relative Rat RAGE mRNA
Expression at Sacrifice (Day 8) in Example 2

| | Average Relative | | |
|---|---|---|---|
| | rAGER mRNA | | |
| | Expression | Low | High |
| Group ID | (n = 5) | (error) | (error) |
| Group 1 (isotonic saline) | 1.000 | 0.060 | 0.065 |
| Group 2 (1.0 mg/kg | 0.306 | 0.019 | 0.017 |
| Tri-SM6.1-αvβ6-AD06949) | | | |
| Group 3 (1.0 mg/kg | 0.142 | 0.019 | 0.010 |
| Tri-SM6.1-αvβ6-AD07256) | | | |
| Group 4 (1.0 mg/kg | 0.195 | 0.031 | 0.067 |
| Tri-SM6.1-αvβ6-AD07474) | | | |
| Group 5 (1.0 mg/kg | 0.125 | 0.037 | 0.038 |
| Tri-SM6.1-αvβ6-AD07475) | | | |
| Group 6 (1.0 mg/kg | 0.294 | 0.039 | 0.057 |
| Tri-SM6.1-αvβ6-AD07476) | | | |
| Group 7 (1.0 mg/kg | 0.230 | 0.037 | 0.038 |
| Tri-SM6.1-αvβ6-AD07477) | | | |

As shown in the data in Table 13 above, each of the RAGE RNAi agents showed significant AGER gene inhibition, with the RAGE RNAi agents targeted to inhibit expression at position 177 (Group 4 (80.5% inhibition) and Group 5 (87.5% inhibition)) provided slightly better inhibition compared to the RAGE RNAi agents targeting position 178 of the AGER gene (Group 6 (70.6% inhibition) and Group 7 (77% inhibition)).

### Example 3. In Vivo Intratracheal Administration of RAGE RNAi Agents in Mice

On study day 1, male c57bl/6 mice were administered 50 microliters via a microsprayer device (Penn Century, Philadelphia, PA) suitable for intratracheal (IT) administration of isotonic saline of isotonic saline or 3.0 mg/kg of one of the following RAGE RNAi agents:

TABLE 14
RAGE RNAi Agent and Dosing for Example 3

| Group ID | AC Duplex Number |
|---|---|
| Group 1 (isotonic saline) | N/A |
| Group 2 (1.0 mg/kg Tri-SM6.1-αvβ6-AD06949) | AC000614 |
| Group 3 (1.0 mg/kg Tri-SM6.1-αvβ6-AD07256) | AC000186 |
| Group 4 (1.0 mg/kg Tri-SM6.1-αvβ6-AD07478) | AC000820 |
| Group 5 (1.0 mg/kg Tri-SM6.1-αvβ6-AD07479) | AC000821 |
| Group 6 (1.0 mg/kg Tri-SM6.1-αvβ6-AD07480) | AC000822 |
| Group 7 (1.0 mg/kg Tri-SM6.1-αvβ6-AD07481) | AC000822 |

As noted in Table 14, each of the RAGE RNAi agents were conjugated to a tridentate small molecule αvβ6 epithelial cell targeting ligand (Tri-SM6.1, see FIG. 1) at the 5′ terminal end of the sense strand, formulated in isotonic saline.

The chemically modified sequences for RAGE RNAi agents AD07478, AD07479, AD07480, and AD07481 are shown in Table 7B (showing duplex), Table 3 (showing respective antisense strand), and Table 5 (showing respective sense strand with linker but without tridentate small molecule αvβ6 epithelial cell targeting ligand (Tri-SM6.1)).

The chemically modified sequences for RAGE RNAi agents AD06949 and AD07256 are shown in Example 2, above.

Five (5) mice were dosed per group. Mice were sacrificed on study day 8, and total RNA was isolated from both lungs following collection and homogenization. Murine AGER mRNA expression was quantitated by probe-based quantitative PCR, normalized to rat GAPDH expression, and expressed as fraction of vehicle control group (geometric mean, +/−95% confidence interval).

TABLE 15
Average Relative Murine RAGE mRNA
Expression at Sacrifice (Day 8) in Example 3.

| | Average Relative | | |
|---|---|---|---|
| | mAGER mRNA | | |
| | Expression | Low | High |
| Group ID | (n = 5) | (error) | (error) |
| Group 1 (isotonic saline) | 1.000 | 0.225 | 0.290 |
| Group 2 (1.0 mg/kg | 0.403 | 0.116 | 0.164 |
| Tri-SM6.1-αvβ6-AD06949) | | | |
| Group 3 (1.0 mg/kg | 0.358 | 0.153 | 0.268 |
| Tri-SM6.1-αvβ6-AD07256) | | | |
| Group 4 (1.0 mg/kg | 1.016 | 0.205 | 0.257 |
| Tri-SM6.1-αvβ6-AD07478) | | | |
| Group 5 (1.0 mg/kg | 1.063 | 0.132 | 0.150 |
| Tri-SM6.1-αvβ6-AD07479) | | | |
| Group 6 (1.0 mg/kg | 0.718 | 0.115 | 0.137 |
| Tri-SM6.1-αvβ6-AD07480) | | | |
| Group 7 (1.0 mg/kg | 0.681 | 0.077 | 0.086 |
| Tri-SM6.1-αvβ6-AD07481) | | | |

As shown in the data in Table 15 above, the RAGE RNAi agents of Groups 4 and 5 (targeting position 384) showed no inhibition and were completely inactive in vivo. The RAGE RNAi agents of Groups 6 and 7 (targeting position 391) showed relatively limited inhibition in vivo and appear insufficiently active to be considered as viable therapeutic candidates for treatment of humans.

### Example 4. In Vivo Intratracheal Administration of RAGE RNAi Agents in Rats

On study day 1, male Sprague Dawley rats were administered 200 microliters via a microsprayer device (Penn Century, Philadelphia, PA) suitable for intratracheal (IT) administration of isotonic saline or 0.5 mg/kg of one of the following RAGE RNAi agents:

TABLE 16
RAGE RNAi Agent and Dosing for Example 4

| Group ID | AC Duplex Number |
|---|---|
| Group 1 (isotonic saline) | N/A |
| Group 2 (0.5 mg/kg Tri-SM6.1-αvβ6-AD07474) | AC000286 |
| Group 3 (0.5 mg/kg Tri-SM6.1-αvβ6-AD07475) | AC000292 |
| Group 4 (0.5 mg/kg Tri-SM6.1-αvβ6-AD07700) | AC000790 |
| Group 5 (0.5 mg/kg Tri-SM6.1-αvβ6-AD07701) | AC000791 |
| Group 6 (0.5 mg/kg Tri-SM6.1-αvβ6-AD07702) | AC000792 |
| Group 7 (0.5 mg/kg Tri-SM6.1-αvβ6-AD07703) | AC000793 |
| Group 8 (0.5 mg/kg Tri-SM6.1-αvβ6-AD07704) | AC000438 |
| Group 9 (0.5 mg/kg Tri-SM6.1-αvβ6-AD07705) | AC000794 |
| Group 10 (0.5 mg/kg Tri-SM6.1-αvβ6-AD07706) | AC000795 |
| Group 11 (0.5 mg/kg Tri-SM6.1-αvβ6-AD07707) | AC000796 |
| Group 12 (0.5 mg/kg Tri-SM6.1-αvβ6-AD07708) | AC000439 |

As noted in Table 16, each of the RAGE RNAi agents were conjugated to a tridentate small molecule αvβ6 epithelial cell targeting ligand (Tri-SM6.1, see FIG. 1) at the 5′ terminal end of the sense strand, formulated in isotonic saline.

The chemically modified sequences for RAGE RNAi agents AD07474, AD07475, AD07700, AD07701, AD07702, AD07703, AD07704, AD07705, AD07706, AD07707, and AD07708 are shown in Table 7B (showing duplex), Table 3 (showing respective antisense strand), and Table 5 (showing respective sense strand with linker but without tridentate small molecule αvβ6 epithelial cell targeting ligand (Tri-SM6.1).

Five (5) rats were dosed per group. Rats were sacrificed on study day 8, and total RNA was isolated from both lungs following collection and homogenization. Rat AGER mRNA expression was quantitated by probe-based quantitative PCR, normalized to rat GAPDH expression, and expressed as fraction of vehicle control group (geometric mean, +/−95% confidence interval).

TABLE 17
Average Relative Rat RAGE mRNA Expression at
Sacrifice (Day 3) in Example 4

| | Average Relative | | |
|---|---|---|---|
| | rAGER mRNA | | |
| | Expression | Low | High |
| Group ID | (n = 5) | (error) | (error) |
| Group 1 (isotonic saline) | 1.000 | 0.075 | 0.081 |
| Group 2 (0.5 mg/kg | 0.355 | 0.147 | 0.250 |
| Tri-SM6.1-αvβ6-AD07474) | | | |
| Group 3 (0.5 mg/kg | 0.193 | 0.110 | 0.254 |
| Tri-SM6.1-αvβ6-AD07475) | | | |
| Group 4 (0.5 mg/kg | 0.233 | 0.060 | 0.080 |
| Tri-SM6.1-αvβ6-AD07700) | | | |
| Group 5 (0.5 mg/kg | 0.344 | 0.135 | 0.221 |
| Tri-SM6.1-αvβ6-AD07701) | | | |
| Group 6 (0.5 mg/kg | 0.194 | 0.036 | 0.044 |
| Tri-SM6.1-αvβ6-AD07702) | | | |
| Group 7 (0.5 mg/kg | 0.265 | 0.024 | 0.026 |
| Tri-SM6.1-αvβ6-AD07703) | | | |
| Group 8 (0.5 mg/kg | 0.174 | 0.030 | 0.036 |
| Tri-SM6.1-αvβ6-AD07704) | | | |
| Group 9 (0.5 mg/kg | 0.188 | 0.052 | 0.071 |
| Tri-SM6.1-αvβ6-AD07705) | | | |
| Group 10 (0.5 mg/kg | 0.215 | 0.077 | 0.119 |
| Tri-SM6.1-αvβ6-AD07706) | | | |
| Group 11 (0.5 mg/kg | 0.182 | 0.059 | 0.087 |
| Tri-SM6.1-αvβ6-AD07707) | | | |

As shown in the data in Table 17 above, each of the RAGE RNAi agents showed significant AGER gene inhibition at a dose of only 0.5 mg/kg. Each of the RAGE RNAi agents tested including different chemical modifications but all included underlying nucleotide sequences targeting position 177 of the AGER gene.

### Example 5. In Vivo Inhaled Aerosolized Administration of RAGE RNAi Agents in Rats

On study day 1, male Sprague Dawley rats were administered a single pulmonary deposited dose (PDD) of 0.5 mg/kg of the RAGE RNAi agent Tri-SM6.1-αvβ6-AD07475. Using a jet nebulizer (Misty Max 10), aerosol was delivered to a rodent single-tier flow-past nose-only inhalation exposure chamber (CH Technologies). One of the ports was equipped with a filter housing so that RNAi agent aerosol concentration could be assessed. Using an assumed respiratory minute volume allometrically scaled to rodent body weight, along with aerosol concentration determined from filter collection and RNAi agent quantification, exposure times were adjusted to target the reported PDD of 0.5 mg/kg. As noted, the RAGE RNAi agent was conjugated to a tridentate small molecule αvβ6 epithelial cell targeting ligand (Tri-SM6.1, see FIG. 1) at the 5′ terminal end of the sense strand, formulated in isotonic saline. The chemically modified sequences for RAGE RNAi agent AD07475 are shown in Table 7B (showing duplex), Table 3 (showing respective antisense strand), and Table 5 (showing respective sense strand with linker but without tridentate small molecule αvβ6 epithelial cell targeting ligand (Tri-SM6.1).

Five (5) rats were dosed per group. Rats were sacrificed on various study days after administration in accordance with the following schedule:

TABLE 18
RAGE RNAi Agent and Dosing for Example 5

| | Date Of |
|---|---|
| | Sacrifice After |
| Group ID | Administartion |
| Group 1 (isotonic saline) | Day 8 |
| Group 2 (Tri-SM6.1-αvβ6-AD07475) | Day 2 |
| Group 3 (Tri-SM6.1-αvβ6-AD07475) | Day 3 |
| Group 4 (Tri-SM6.1-αvβ6-AD07475) | Day 4 |
| Group 5 (Tri-SM6.1-αvβ6-AD07475) | Day 5 |
| Group 6 (Tri-SM6.1-αvβ6-AD07475) | Day 8 |
| Group 7 (Tri-SM6.1-αvβ6-AD07475) | Day 15 |
| Group 8 (Tri-SM6.1-αvβ6-AD07475) | Day 22 |
| Group 9 (Tri-SM6.1-αvβ6-AD07475) | Day 29 |
| Group 10 (Tri-SM6.1-αvβ6-AD07475) | Day 43 |
| Group 11 (Tri-SM6.1-αvβ6-AD07475) | Day 57 |

Upon sacrifice on the respective date, total RNA was isolated from both lungs following collection and homogenization. Rat AGER mRNA expression was quantitated by probe-based quantitative PCR, normalized to rat GAPDH expression, and expressed as fraction of vehicle control group (geometric mean, +/−95% confidence interval).

TABLE 19
Average Relative Rat RAGE mRNA Expression at Sacrifice in Example 5

| | Average Relative | | |
|---|---|---|---|
| | rAGER mRNA Expression | Low | High |
| Group ID | (n = 5) | (error) | (error) |
| Group 1 (isotonic saline; day 8 sacrifice) | 1.000 | 0.261 | 0.352 |
| Group 2 (0.5 mg/kg deposited dose Tri- | 0.428 | 0.074 | 0.090 |
| SM6.1-αvβ6-AD07475; day 2 sacrifice) | | | |
| Group 3 (0.5 mg/kg deposited dose Tri- | 0.179 | 0.025 | 0.028 |
| SM6.1-αvβ6-AD07475; day 3 sacrifice) | | | |
| Group 4 (0.5 mg/kg deposited dose Tri- | 0.077 | 0.014 | 0.017 |
| SM6.1-αvβ6-AD07475; day 4 sacrifice) | | | |
| Group 5 (0.5 mg/kg deposited dose Tri- | 0.061 | 0.005 | 0.005 |
| SM6.1-αvβ6-AD07475; day 5 sacrifice) | | | |
| Group 6 (0.5 mg/kg deposited dose Tri- | 0.042 | 0.005 | 0.006 |
| SM6.1-αvβ6-AD07475; day 8 sacrifice) | | | |
| Group 7 (0.5 mg/kg deposited dose Tri- | 0.042 | 0.007 | 0.009 |
| SM6.1-αvβ6-AD07475; day 15 sacrifice) | | | |
| Group 8 (0.5 mg/kg deposited dose Tri- | 0.049 | 0.010 | 0.012 |
| SM6.1-αvβ6-AD07475; day 22 sacrifice) | | | |
| Group 9 (0.5 mg/kg deposited dose Tri- | 0.097 | 0.014 | 0.016 |
| SM6.1-αvβ6-AD07475; day 29 sacrifice) | | | |
| Group 10 (0.5 mg/kg deposited dose Tri- | 0.137 | 0.015 | 0.017 |
| SM6.1-αvβ6-AD07475; day 43 sacrifice) | | | |
| Group 11 (0.5 mg/kg deposited dose Tri- | 0.123 | 0.013 | 0.015 |
| SM6.1-αvβP6-AD07475; day 57 sacrifice) | | | |

As shown in the data in Table 19 above, the RAGE RNAi agent AD07475 showed significant AGER gene inhibition at a dose of only 0.5 mg/kg, and the duration of knockdown was maintained through at least day 22 before slowly beginning to return to baseline levels, suggesting that monthly dosing (e.g., administration every 28 days) may be feasible.

### Example 6. In Vivo Inhaled Aerosolized Administration of RAGE RNAi Agents in Rats and Targeting Ligand Effect

On study day 1, male Sprague Dawley rats were administered a single dose at varying concentrations of the RAGE RNAi agent Tri-SM6.1-αvβ6-AD07475, or the RAGE RNAi agent AD7475 without the αvβ6 epithelial cell targeting ligand attached. Using a jet nebulizer (Misty Max 10), aerosol was delivered to a rodent single-tier flow-past nose-only inhalation exposure chamber (CH Technologies). One of the ports was equipped with a filter housing so that RNAi agent aerosol concentration could be assessed. Using an assumed respiratory minute volume allometrically scaled to rodent body weight, along with aerosol concentration determined from filter collection and RNAi agent quantification, exposure times were adjusted to target the reported PDD listed in Table 20. For the Groups that included the targeting ligand, the RAGE RNAi agent was conjugated to a tridentate small molecule αvβ6 epithelial cell targeting ligand (Tri-SM6.1, see FIG. 1) at the 5′ terminal end of the sense strand, formulated in isotonic saline. The chemically modified sequences for RAGE RNAi agent AD07475 are shown in Table 7B (showing duplex), Table 3 (showing respective antisense strand), and Table 5 (showing respective sense strand with linker but without tridentate small molecule αvβ6 epithelial cell targeting ligand (Tri-SM6.1). The dosing groups were as follows:

TABLE 20
RAGE RNAi Agent and Dosing for Example 6

| | AC Duplex |
|---|---|
| Group ID | Number |
| Group 1 (isotonic saline) | N/A |
| Group 2 (0.24 mg/kg deposited dose Tri-SM6.1-αvβ6-AD07475) | AC000292 |
| Group 3 (0.12 mg/kg deposited dose Tri-SM6.1-αvβ6-AD07475) | AC000292 |
| Group 4 (0.06 mg/kg deposited dose Tri-SM6.1-αvβ6-AD07475) | AC000292 |
| Group 5 (0.03 mg/kg deposited dose Tri-SM6.1-αvβ6-AD07475) | AC000292 |
| Group 6 (0.015 mg/kg deposited dose | AC000292 |
| Tri-SM6.1-αvβ6-AD07475) | |
| Group 7 (0.24 mg/kg deposited dose AD07475) | N/A |
| Group 8 (0.12 mg/kg deposited dose AD07475) | N/A |
| Group 9 (0.06 mg/kg deposited dose AD07475) | N/A |
| Group 10 (0.03 mg/kg deposited dose AD07475) | N/A |
| Group 11 (0.015 mg/kg deposited dose AD07475) | N/A |

Five (5) rats were dosed per group. Rats were sacrificed on study day 8, and total RNA was isolated from both lungs following collection and homogenization. Rat AGER mRNA expression was quantitated by probe-based quantitative PCR, normalized to rat GAPDH expression, and expressed as fraction of vehicle control group (geometric mean, +/−95% confidence interval).

TABLE 21
Average Relative Rat RAGE mRNA
Expression at Sacrifice in Example 6

| | Average | | |
|---|---|---|---|
| | Relative | | |
| | rAGER | | |
| | mRNA | | |
| | Expression | Low | High |
| Group ID | (n = 5) | (error) | (error) |
| Group 1 (isotonic saline) | 1.000 | 0.143 | 0.168 |
| Group 2 (0.24 mg/kg deposited | 0.081 | 0.010 | 0.011 |
| dose Tri-SM6.1-αvβ6-AD07475) | | | |
| Group 3 (0.12 mg/kg deposited | 0.081 | 0.019 | 0.025 |
| dose Tri-SM6.1-αvβ6-AD07475) | | | |
| Group 4 (0.06 mg/kg deposited | 0.130 | 0.033 | 0.043 |
| dose Tri-SM6.1-αvβ6-AD07475) | | | |
| Group 5 (0.03 mg/kg deposited | 0.342 | 0.112 | 0.165 |
| dose Tri-SM6.1-αvβ6-AD07475) | | | |
| Group 6 (0.015 mg/kg deposited | 0.436 | 0.088 | 0.110 |
| dose Tri-SM6.1-αvβ6-AD07475) | | | |
| Group 7 (0.24 mg/kg deposited | 0.107 | 0.032 | 0.046 |
| dose AD07475) | | | |
| Group 8 (0.12 mg/kg deposited | 0.157 | 0.041 | 0.055 |
| dose AD07475) | | | |
| Group 9 (0.06 mg/kg deposited | 0.309 | 0.067 | 0.086 |
| dose AD07475) | | | |
| Group 10 (0.03 mg/kg deposited | 0.436 | 0.120 | 0.165 |
| dose AD07475) | | | |
| Group 11 (0.015 mg/kg deposited | 0.537 | 0.052 | 0.058 |
| dose AD07475) | | | |

As shown in the data in Table 21 above, at each of the time points measured the RAGE RNAi agent AD07475 showed substantial inhibition compared to control both with and without a targeting ligand. Indeed, even at the lowest dose tested of 0.015 mg/kg deposited dose, inhibition levels approaching 50% (see Group 6 (with targeting ligand Tri-SM6.1-αvβ6; 56.4% gene inhibition) and Group 11 (no targeting ligand; 46.3% gene inhibition)). Further, at each of the respective dose levels measured, the RNAi agent conjugated to the targeting ligand numerically outperformed the RNAi agent without the targeting ligand (with the differences generally becoming more pronounced at lower dose levels), indicating the existence of a ligand effect that can provide increased inhibitory activity in vivo.

### Example 7. In Vivo Intratracheal Administration of RAGE RNAi Agents in Rats

On study day 1, male Sprague Dawley rats were administered 200 microliters via a microsprayer device (Penn Century, Philadelphia, PA) suitable for intratracheal (IT) administration of isotonic saline or 0.25 mg/kg of one of the following RAGE RNAi agents:

TABLE 22
RAGE RNAi Agent and Dosing for Example 7

| Group ID | AC Duplex Number |
|---|---|
| Group 1 (isotonic saline) | AC000286 |
| Group 2 (0.25 mg/kg Tri-SM6.1-αvβ36-AD07474) | AC000292 |
| Group 3 (0.25 mg/kg Tri-SM6.1-αvβ36-AD07475) | AC000293 |
| Group 4 (0.25 mg/kg Tri-SM6.1-αvβ36-AD07972) | AC000294 |
| Group 5 (0.25 mg/kg Tri-SM6.1-αvβ36-AD07973) | AC000290 |
| Group 6 (0.25 mg/kg Tri-SM6.1-αvβ36-AD07974) | AC000291 |
| Group 7 (0.25 mg/kg Tri-SM6.1-αvβ36-AD07975) | AC000312 |
| Group 8 (0.25 mg/kg Tri-SM6.1-αvβ36-AD07976) | AC000286 |

As noted in Table 22, each of the RAGE RNAi agents were conjugated to a tridentate small molecule αvβ6 epithelial cell targeting ligand (Tri-SM6.1, see FIG. 1) at the 5′ terminal end of the sense strand, formulated in isotonic saline.

The chemically modified sequences for RAGE RNAi agents AD07474, AD07475, AD07972, AD07973, AD07974, AD07975, and AD07976 are shown in Table 7B (showing duplex), Table 3 (showing respective antisense strand), and Table 5 (showing respective sense strand with linker but without tridentate small molecule αvβ6 epithelial cell targeting ligand (Tri-SM6.1).

Five (5) rats were dosed per group. Rats were sacrificed on study day 8, and total RNA was isolated from both lungs following collection and homogenization. Rat AGER mRNA expression was quantitated by probe-based quantitative PCR, normalized to rat GAPDH expression, and expressed as fraction of vehicle control group (geometric mean, +/−95% confidence interval).

TABLE 23
Average Relative Rat RAGE mRNA Expression at Sacrifice
(Day 8) in Example 7

| | Average Relative | | |
|---|---|---|---|
| | rAGER mRNA | | |
| | Expression | Low | High |
| Group ID | (n = 5) | (error) | (error) |
| Group 1 (isotonic saline) | 1.000 | 0.132 | 0.152 |
| Group 2 (0.25 mg/kg | 0.210 | 0.038 | 0.047 |
| Tri-SM6.1-αvβ-AD07474) | | | |
| Group 3 (0.25 mg/kg | 0.150 | 0.038 | 0.052 |
| Tri-SM6.1-αvβ-AD07475) | | | |
| Group 4 (0.25 mg/kg | 0.118 | 0.020 | 0.024 |
| Tri-SM6.1-αvβ-AD07972) | | | |
| Group 5 (0.25 mg/kg | 0.145 | 0.038 | 0.052 |
| Tri-SM6.1-αvβ-AD07973) | | | |
| Group 6 (0.25 mg/kg | 0.338 | 0.041 | 0.046 |
| Tri-SM6.1-αvβ-AD07974) | | | |
| Group 7 (0.25 mg/kg | 0.194 | 0.051 | 0.070 |
| Tri-SM6.1-αvβ-AD07975) | | | |
| Group 8 (0.25 mg/kg | 0.244 | 0.046 | 0.057 |
| Tri-SM6.1-αvβ-AD07976) | | | |

As shown in the data in Table 23 above, each of the RAGE RNAi agents showed significant AGER gene inhibition at a dose of only 0.25 mg/kg. Each of the RAGE RNAi agents tested including different chemical modifications but all included underlying nucleotide sequences targeting position 177 of the AGER gene.

### Example 8. In Vivo Intratracheal Administration of RAGE RNAi Agents in Rats

On study day 1, male Sprague Dawley rats were administered 200 microliters via a microsprayer device (Penn Century, Philadelphia, PA) suitable for intratracheal (IT) administration of isotonic saline or 0.25 mg/kg of one of the following RAGE RNAi agents:

TABLE 24
RAGE RNAi Agent and Dosing for Example 8

| Group ID | AC Duplex Number |
|---|---|
| Group 1 (isotonic saline) | N/A |
| Group 2 (0.1 mg/kg Tri-SM6.1-αvβ6-AD07475) | AC000292 |
| Group 3 (0.1 mg/kg Tri-SM6.1-αvβ6-AD07704) | AC000438 |
| Group 4 (0.1 mg/kg Tri-SM6.1-αvβ6-AD07708) | AC000439 |
| Group 5 (0.1 mg/kg Tri-SM6.1-αvβ6-AD08030) | AC000440 |
| Group 6 (0.1 mg/kg Tri-SM6.1-αvβ6-AD08031) | AC000441 |
| Group 7 (0.1 mg/kg Tri-SM6.1-αvβ6-AD08032) | AC000442 |
| Group 8 (0.1 mg/kg Tri-SM6.1-αvβ6-AD08033) | AC000414 |
| Group 9 (0.1 mg/kg Tri-SM6.1-αvβ6-AD08034) | AC000415 |
| Group 10 (0.1 mg/kg Tri-SM6.1-αvβ6-AD08035) | AC000416 |
| Group 11 (0.1 mg/kg Tri-SM6.1-αvβ6-AD08036) | AC000417 |
| Group 12 (0.1 mg/kg Tri-SM6.1-αvβ6-AD08037) | AC000418 |
| Group 13 (0.1 mg/kg Tri-SM6.1-αvβ6-AD08038) | AC000419 |
| Group 14 (0.1 mg/kg Tri-SM6.1-αvβ6-AD08039) | AC000420 |

As noted in Table 24, each of the RAGE RNAi agents were conjugated to a tridentate small molecule αvβ6 epithelial cell targeting ligand (Tri-SM6.1, see FIG. 1) at the 5′ terminal end of the sense strand, formulated in isotonic saline.

The chemically modified sequences for the RAGE RNAi agents in Example 8 are shown in Table 7B (showing duplex), Table 3 (showing respective antisense strand), and Table 5 (showing respective sense strand with linker but without tridentate small molecule αvβ6 epithelial cell targeting ligand (Tri-SM6.1).

Five (5) rats were dosed per group. Rats were sacrificed on study day 8, and total RNA was isolated from both lungs following collection and homogenization. Rat AGER mRNA expression was quantitated by probe-based quantitative PCR, normalized to rat GAPDH expression, and expressed as fraction of vehicle control group (geometric mean, +/−95% confidence interval).

TABLE 25
Average Relative Rat RAGE mRNA Expression at
Sacrifice (Day 8) in Example 8

| | Average Relative | | |
|---|---|---|---|
| | rAGER mRNA | | |
| | Expression | Low | High |
| Group ID | (n = 5) | (error) | (error) |
| Group 1 (isotonic saline) | 1.000 | 0.118 | 0.134 |
| Group 2 (0.1 mg/kg | 0.232 | 0.030 | 0.034 |
| Tri-SM6.1-αvβ6-AD07475) | | | |
| Group 3 (0.1 mg/kg | 0.372 | 0.049 | 0.057 |
| Tri-SM6.1-αvβ6-AD07704) | | | |
| Group 4 (0.1 mg/kg | 0.382 | 0.102 | 0.139 |
| Tri-SM6.1-αvβ6-AD07708) | | | |
| Group 5 (0.1 mg/kg | 0.268 | 0.054 | 0.067 |
| Tri-SM6.1-αvβ6-AD08030) | | | |
| Group 6 (0.1 mg/kg | 0.215 | 0.065 | 0.093 |
| Tri-SM6.1-αvβ6-AD08031) | | | |
| Group 7 (0.1 mg/kg | 0.314 | 0.034 | 0.038 |
| Tri-SM6.1-αvβ6-AD08032) | | | |
| Group 8 (0.1 mg/kg | 0.244 | 0.075 | 0.108 |
| Tri-SM6.1-αvβ6-AD08033) | | | |
| Group 9 (0.1 mg/kg | 0.723 | 0.078 | 0.088 |
| Tri-SM6.1-αvβ6-AD08034) | | | |
| Group 10 (0.1 mg/kg | 0.281 | 0.076 | 0.104 |
| Tri-SM6.1-αvβ6-AD08035) | | | |
| Group 11 (0.1 mg/kg | 0.288 | 0.029 | 0.033 |
| Tri-SM6.1-αvβ6-AD08036) | | | |
| Group 12 (0.1 mg/kg | 0.253 | 0.056 | 0.071 |
| Tri-SM6.1-αvβ6-AD08037) | | | |
| Group 13 (0.1 mg/kg | 0.359 | 0.105 | 0.149 |
| Tri-SM6.1-αvβ6-AD08038) | | | |
| Group 14 (0.1 mg/kg | 0.317 | 0.125 | 0.206 |
| Tri-SM6.1-αvβ6-AD08039) | | | |

As shown in the data in Table 25 above, each of the RAGE RNAi agents showed significant AGER gene inhibition at a dose of only 0.1 mg/kg. Each of the RAGE RNAi agents tested including different chemical modifications but all included underlying nucleotide sequences targeting position 177 of the AGER gene.

### Example 9. In Vivo Inhaled Aerosolized Administration of RAGE RNAi Agents in Cynomolgus Monkeys

On study day 1, female cynomolgus monkeys were administered a single dose at 1 mg/kg PDD of the RAGE RNAi agent Tri-SM6.1-αvβ6-AD09150, Tri-SM6.1-αvβ6-AD09151, or Tri-SM6.1-αvβ6-AD09152. Using a vibrating mesh nebulizer (Aeroneb® Solo), aerosol was delivered to restrained, anesthetized monkeys fitted with a primate inhalation helmet. One of the helmets was equipped with a filter housing so that RNAi agent aerosol concentration could be assessed. Using an assumed respiratory minute volume allometrically scaled to rodent body weight, along with aerosol concentration determined from filter collection and RNAi agent quantification, exposure times were adjusted to target the reported PDD listed in Table 26. The RAGE RNAi agent was conjugated to a tridentate small molecule αvβ6 integrin receptor targeting ligand (Tri-SM6.1, see FIG. 1) at the 5′ terminal end of the sense strand, formulated in isotonic saline. The chemically modified sequences for RAGE RNAi agents are shown in Table 7B (showing duplex), Table 3 (showing respective antisense strand), and Table 5 (showing respective sense strand with linker but without tridentate small molecule αvβ6 integrin receptor targeting ligand (Tri-SM6.1). The dosing groups were as follows:

TABLE 26
RAGE RNAi Agent and Dosing for Example 9
(calculated with 25% deposited fraction).

| | AC Duplex |
|---|---|
| Group ID | Number |
| Group 1 (isotonic saline) | N/A |
| Group 2 (0.98 mg/kg deposited dose | AC001266 |
| Tri-SM6.1-αvβ6-AD09150) | |
| Group 3 (1.05 mg/kg deposited dose | AC001267 |
| Tri-SM6.1-αvβ6-AD09151) | |
| Group 4 (1.04 mg/kg deposited dose | AC001268 |
| Tri-SM6.1-αvβ6-AD09152) | |

Three (3) monkeys were dosed per group. Monkeys were sacrificed on study day 15, and total RNA was isolated from lung samples following collection and homogenization. The data in the following Table 27 shows mRNA expression sampled from the distal left caudal lobe. The data in the following Table 28 shows mRNA expression sampled from the distal right caudal lobe. Cynomolgus monkey AGER mRNA expression was quantitated by probe-based quantitative PCR, normalized to Cynomolgus monkey beta-actin expression, and expressed as fraction of vehicle control group (geometric mean, +/−95% confidence interval).

TABLE 27
Average Relative Cynomolgus Monkey RAGE
mRNA Expression, Distal Left
Caudal Lobe, at Sacrifice in Example 9.

| | Average Relative | | |
|---|---|---|---|
| | cAGER mRNA | Low | High |
| Group ID | Expression (n = 3) | (error) | (error) |
| Group 1 (isotonic saline) | 1.000 | 0.342 | 0.520 |
| Group 2 (0.98 mg/kg | 0.113 | 0.069 | 0.178 |
| deposited dose Tri- | | | |
| SM6.1-αvβ6-AD09150) | | | |
| Group 3 (1.05 mg/kg | 0.081 | 0.040 | 0.079 |
| deposited dose Tri- | | | |
| SM6.1-αvβ6-AD09151) | | | |
| Group 4 (1.04 mg/kg | 0.708 | 0.437 | 1.142 |
| deposited dose Tri- | | | |
| SM6.1-αvβ6-AD09152) | | | |

TABLE 28
Average Relative Cynomolgus Monkey RAGE
mRNA Expression, Distal
Right Caudal Lobe, at Sacrifice in Example 9.

| | Average Relative | | |
|---|---|---|---|
| | cAGER mRNA | Low | High |
| Group ID | Expression (n = 3) | (error) | (error) |
| Group 1 (isotonic saline) | 1.000 | 0.330 | 0.492 |
| Group 2 (0.98 mg/kg | 0.069 | 0.032 | 0.059 |
| deposited dose Tri- | | | |
| SM6.1-αvβ6-AD09150) | | | |
| Group 3 (1.05 mg/kg | 0.069 | 0.028 | 0.049 |
| deposited dose Tri- | | | |
| SM6.1-αvβ6-AD09151) | | | |
| Group 4 (1.04 mg/kg | 0.630 | 0.406 | 1.141 |
| deposited dose Tri- | | | |
| SM6.1-αvβ6-AD09152) | | | |

As shown in the data in Table 28 above, RNAi agent AD09150 and AD09151 showed substantial inhibition (93%) compared to control, demonstrating the ability to robustly silence AGER expression in non-human primates.

In a separate qPCR assay, cynomolgus monkey AGER mRNA expression was quantified. The data in the following Tables 29 through 42 show mRNA expression sampled from the cynomolgus lung samples. Cynomolgus monkey AGER mRNA expression was quantitated by probe-based quantitative PCR, normalized to Cynomolgus monkey beta-actin expression, and expressed as fraction of vehicle control group (geometric mean, +/−95% confidence interval). Some lung tissue was repeated with different tissue samples, when available, for example, Distal Left Caudal Lobe and Distal Left Cranial Lobe.

TABLE 29
Average Relative Cynomolgus Monkey
RAGE mRNA Expression, Distal Left
Caudal Lobe, at Sacrifice in Example 9.

| | Average Relative | | |
|---|---|---|---|
| | cAGER mRNA | Low | High |
| Group ID | Expression (n = 3) | (error) | (error) |
| Group 1 (isotonic saline) | 1.000 | 0.272 | 0.373 |
| Group 2 (0.98 mg/kg | 0.138 | 0.097 | 0.330 |
| deposited dose Tri- | | | |
| SM6.1-αvβ6-AD09150) | | | |
| Group 3 (1.05 mg/kg | 0.065 | 0.031 | 0.059 |
| deposited dose Tri- | | | |
| SM6.1-αvβ6-AD09151) | | | |
| Group 4 (1.04 mg/kg | 0.850 | 0.435 | 0.890 |
| deposited dose Tri- | | | |
| SM6.1-αvβ6-AD09152) | | | |

TABLE 30
Average Relative Cynomolgus Monkey
RAGE mRNA Expression, Medial
Left Caudal Lobe, at Sacrifice in Example 9.

| | Average Relative | | |
|---|---|---|---|
| | cAGER mRNA | Low | High |
| Group ID | Expression (n = 3) | (error) | (error) |
| Group 1 (isotonic saline) | 1.000 | 0.433 | 0.762 |
| Group 2 (0.98 mg/kg | 0.122 | 0.086 | 0.298 |
| deposited dose Tri- | | | |
| SM6.1-αvβ6-AD09150) | | | |
| Group 3 (1.05 mg/kg | 0.133 | 0.039 | 0.054 |
| deposited dose Tri- | | | |
| SM6.1-αvβ6-AD09151) | | | |
| Group 4 (1.04 mg/kg | 0.877 | 0.528 | 1.329 |
| deposited dose Tri- | | | |
| SM6.1-αvβ6-AD09152) | | | |

TABLE 31
Average Relative Cynomolgus Monkey
RAGE mRNA Expression, Proximal
Left Caudal Lobe, at Sacrifice in Example 9.

| | Average Relative | | |
|---|---|---|---|
| | cAGER mRNA | Low | High |
| Group ID | Expression (n = 3) | (error) | (error) |
| Group 1 (isotonic saline) | 1.000 | 0.297 | 0.422 |
| Group 2 (0.98 mg/kg | 0.152 | 0.094 | 0.246 |
| deposited dose Tri- | | | |
| SM6.1-αvβ6-AD09150) | | | |
| Group 3 (1.05 mg/kg | 0.084 | 0.032 | 0.053 |
| deposited dose Tri- | | | |
| SM6.1-αvβ6-AD09151) | | | |
| Group 4 (1.04 mg/kg | 0.781 | 0.446 | 1.038 |
| deposited dose Tri- | | | |
| SM6.1-αvβ6-AD09152) | | | |

TABLE 32
Average Relative Cynomolgus Monkey
RAGE mRNA Expression, Distal Left
Cranial Lobe, at Sacrifice in Example 9.

| | Average Relative | | |
|---|---|---|---|
| | cAGER mRNA | Low | High |
| Group ID | Expression (n = 3) | (error) | (error) |
| Group 1 (isotonic saline) | 1.000 | 0.231 | 0.300 |
| Group 2 (0.98 mg/kg | 0.126 | 0.072 | 0.166 |
| deposited dose Tri- | | | |
| SM6.1-αvβ6-AD09150) | | | |
| Group 3 (1.05 mg/kg | 0.104 | 0.056 | 0.119 |
| deposited dose Tri- | | | |
| SM6.1-αvβ6-AD09151) | | | |
| Group 4 (1.04 mg/kg | 0.549 | 0.370 | 1.134 |
| deposited dose Tri- | | | |
| SM6.1-αvβ6-AD09152) | | | |

TABLE 33
Average Relative Cynomolgus Monkey
RAGE mRNA Expression, Distal Left
Cranial Lobe, at Sacrifice in Example 9.

| | Average Relative | | |
|---|---|---|---|
| | cAGER mRNA | Low | High |
| Group ID | Expression (n = 3) | (error) | (error) |
| Group 1 (isotonic saline) | 1.000 | 0.306 | 0.440 |
| Group 2 (0.98 mg/kg | 0.076 | 0.045 | 0.113 |
| deposited dose Tri- | | | |
| SM6.1-αvβ6-AD09150) | | | |
| Group 3 (1.05 mg/kg | 0.048 | 0.019 | 0.031 |
| deposited dose Tri- | | | |
| SM6.1-αvβ6-AD09151) | | | |
| Group 4 (1.04 mg/kg | 0.548 | 0.355 | 1.008 |
| deposited dose Tri- | | | |
| SM6.1-αvβ6-AD09152) | | | |

TABLE 34
Average Relative Cynomolgus
Monkey RAGE mRNA Expression, Medial
Left Cranial Lobe, at Sacrifice in Example 9.

| | Average Relative | | |
|---|---|---|---|
| | cAGER mRNA | Low | High |
| Group ID | Expression (n = 3) | (error) | (error) |
| Group 1 (isotonic saline) | 1.000 | 0.383 | 0.620 |
| Group 2 (0.98 mg/kg | 0.157 | 0.116 | 0.447 |
| deposited dose Tri- | | | |
| SM6.1-αvβ6-AD09150) | | | |
| Group 3 (1.05 mg/kg | 0.054 | 0.028 | 0.059 |
| deposited dose Tri- | | | |
| SM6.1-αvβ6-AD09151) | | | |
| Group 4 (1.04 mg/kg | 0.826 | 0.485 | 1.175 |
| deposited dose Tri- | | | |
| SM6.1-αvβ6-AD09152) | | | |

TABLE 35
Average Relative Cynomolgus
Monkey RAGE mRNA Expression, Proximal
Left Cranial Lobe, at Sacrifice in Example 9.

| | Average Relative | | |
|---|---|---|---|
| | cAGER mRNA | Low | High |
| Group ID | Expression (n = 3) | (error) | (error) |
| Group 1 (isotonic saline) | 1.000 | 0.174 | 0.211 |
| Group 2 (0.98 mg/kg | 0.106 | 0.074 | 0.242 |
| deposited dose Tri- | | | |
| SM6.1-αvβ6-AD09150) | | | |
| Group 3 (1.05 mg/kg | 0.106 | 0.053 | 0.104 |
| deposited dose Tri- | | | |
| SM6.1-αvβ6-AD09151) | | | |
| Group 4 (1.04 mg/kg | 0.404 | 0.275 | 0.864 |
| deposited dose Tri- | | | |
| SM6.1-αvβ6-AD09152) | | | |

TABLE 36
Average Relative Cynomolgus Monkey
RAGE mRNA Expression, Distal
Right Caudal Lobe, at Sacrifice in Example 9.

| | Average Relative | | |
|---|---|---|---|
| | cAGER mRNA | Low | High |
| Group ID | Expression (n = 3) | (error) | (error) |
| Group 1 (isotonic saline) | 1.000 | 0.070 | 0.075 |
| Group 2 (0.98 mg/kg | 0.116 | 0.081 | 0.267 |
| deposited dose Tri- | | | |
| SM6.1-αvβ6-AD09150) | | | |
| Group 3 (1.05 mg/kg | 0.050 | 0.027 | 0.059 |
| deposited dose Tri- | | | |
| SM6.1-αvβ6-AD09151) | | | |
| Group 4 (1.04 mg/kg | 0.690 | 0.222 | 0.328 |
| deposited dose Tri- | | | |
| SM6.1-αvβ6-AD09152) | | | |

TABLE 37
Average Relative Cynomolgus Monkey
RAGE mRNA Expression, Medial
Right Caudal Lobe, at Sacrifice in Example 9.

| | Average Relative | | |
|---|---|---|---|
| | cAGER mRNA | Low | High |
| Group ID | Expression (n = 3) | (error) | (error) |
| Group 1 (isotonic saline) | 1.000 | 0.493 | 0.972 |
| Group 2 (0.98 mg/kg | 0.243 | 0.145 | 0.359 |
| deposited dose Tri- | | | |
| SM6.1-αvβ6-AD09150) | | | |
| Group 3 (1.05 mg/kg | 0.124 | 0.057 | 0.105 |
| deposited dose Tri- | | | |
| SM6.1-αvβ6-AD09151) | | | |
| Group 4 (1.04 mg/kg | 0.549 | 0.184 | 0.277 |
| deposited dose Tri- | | | |
| SM6.1-αvβ6-AD09152) | | | |

TABLE 38
Average Relative Cynomolgus Monkey
RAGE mRNA Expression, Proximal
Right Caudal Lobe, at Sacrifice in Example 9.

| | Average Relative | | |
|---|---|---|---|
| | cAGER mRNA | Low | High |
| Group ID | Expression (n = 3) | (error) | (error) |
| Group 1 (isotonic saline) | 1.000 | 0.451 | 0.821 |
| Group 2 (0.98 mg/kg | 0.160 | 0.088 | 0.198 |
| deposited dose Tri- | | | |
| SM6.1-αvβ6-AD09150) | | | |
| Group 3 (1.05 mg/kg | 0.122 | 0.078 | 0.216 |
| deposited dose Tri- | | | |
| SM6.1-αvβ6-AD09151) | | | |
| Group 4 (1.04 mg/kg | 0.262 | 0.175 | 0.522 |
| deposited dose Tri- | | | |
| SM6.1-αvβ6-AD09152) | | | |

TABLE 39
Average Relative Cynomolgus Monkey
RAGE mRNA Expression, Distal
Right Cranial Lobe, at Sacrifice in Example 9.

| | Average Relative | | |
|---|---|---|---|
| | cAGER mRNA | Low | High |
| Group ID | Expression (n = 3) | (error) | (error) |
| Group 1 (isotonic saline) | 1.000 | 0.161 | 0.192 |
| Group 2 (0.98 mg/kg | 0.268 | 0.142 | 0.300 |
| deposited dose Tri- | | | |
| SM6.1-αvβ6-AD09150) | | | |
| Group 3 (1.05 mg/kg | 0.097 | 0.039 | 0.066 |
| deposited dose Tri- | | | |
| SM6.1-αvβ6-AD09151) | | | |
| Group 4 (1.04 mg/kg | 0.395 | 0.243 | 0.634 |
| deposited dose Tri- | | | |
| SM6.1-αvβ6-AD09152) | | | |

TABLE 40
Average Relative Cynomolgus Monkey
RAGE mRNA Expression, Distal
Right Cranial Lobe, at Sacrifice in Example 9.

| | Average Relative | | |
|---|---|---|---|
| | cAGER mRNA | Low | High |
| Group ID | Expression (n = 3) | (error) | (error) |
| Group 1 (isotonic saline) | 1.000 | 0.362 | 0.568 |
| Group 2 (0.98 mg/kg | 0.172 | 0.112 | 0.320 |
| deposited dose Tri- | | | |
| SM6.1-αvβ6-AD09150) | | | |
| Group 3 (1.05 mg/kg | 0.156 | 0.034 | 0.044 |
| deposited dose Tri- | | | |
| SM6.1-αvβ6-AD09151) | | | |
| Group 4 (1.04 mg/kg | 0.923 | 0.613 | 1.820 |
| deposited dose Tri- | | | |
| SM6.1-αvβ6-AD09152) | | | |

TABLE 41
Average Relative Cynomolgus Monkey
RAGE mRNA Expression, Medial
Right Cranial Lobe, at Sacrifice in Example 9.

| | Average Relative | | |
|---|---|---|---|
| | cAGER mRNA | Low | High |
| Group ID | Expression (n = 3) | (error) | (error) |
| Group 1 (isotonic saline) | 1.000 | 0.496 | 0.984 |
| Group 2 (0.98 mg/kg | 0.301 | 0.154 | 0.315 |
| deposited dose Tri- | | | |
| SM6.1-αvβ6-AD09150) | | | |
| Group 3 (1.05 mg/kg | 0.098 | 0.050 | 0.100 |
| deposited dose Tri- | | | |
| SM6.1-αvβ6-AD09151) | | | |
| Group 4 (1.04 mg/kg | 0.303 | 0.233 | 1.007 |
| deposited dose Tri- | | | |
| SM6.1-αvβ6-AD09152) | | | |

TABLE 42
Average Relative Cynomolgus Monkey
RAGE mRNA Expression, Proximal
Right Cranial Lobe, at Sacrifice in Example 9.

| | Average Relative | | |
|---|---|---|---|
| | cAGER mRNA | Low | High |
| Group ID | Expression (n = 3) | (error) | (error) |
| Group 1 (isotonic saline) | 1.000 | 0.098 | 0.108 |
| Group 2 (0.98 mg/kg | 0.156 | 0.075 | 0.146 |
| deposited dose Tri- | | | |
| SM6.1-αvβ6-AD09150) | | | |
| Group 3 (1.05 mg/kg | 0.080 | 0.047 | 0.114 |
| deposited dose Tri- | | | |
| SM6.1-αvβ6-AD09151) | | | |
| Group 4 (1.04 mg/kg | 0.445 | 0.198 | 0.358 |
| deposited dose Tri- | | | |
| SM6.1-αvβ6-AD09152) | | | |

As shown in the data in Tables 29 through 42 above, RNAi agent AD09150 and AD09151 showed substantial inhibition (up to 92%) compared to control, demonstrating the ability to robustly silence AGER expression in non-human primates.

### Example 10. In Vivo Anti-Inflammatory Effect of RAGE Knock-Down in Rat Model of Airway Inflammation, Delivery Via Aerosol

On study day 1 and again on study day 15, male Sprague Dawley rats were administered a 0.5 mg/kg deposited dose of the RAGE RNAi agent Tri-SM6.1-αvβ6-AD07475, or a vehicle control without an RNAi agent, in accordance with Table 43 below. Using a jet nebulizer (Misty Max 10), aerosol was delivered to a rodent single-tier flow-past nose-only inhalation exposure chamber (CH Technologies). One of the ports was equipped with a filter housing so that RNAi agent aerosol concentration could be assessed. Using an assumed respiratory minute volume allometrically scaled to rodent body weight, along with aerosol concentration determined from filter collection and RNAi agent quantification.

On day 40, rats in Group 4 and Group 5 were challenged with a single intra-tracheal dose of 400 ug/rat of Alternaria alternata (Alt) prepared in saline, and rats in Group 2 and 3 where administered a 400 ug/rat dose of saline control. Rats in Group N-1 were naïve and had no treatment administered.

TABLE 43
RAGE RNAi Agent and Dosing for Example 10.

| | AC Duplex | Animals |
|---|---|---|
| Group ID | Number | per Group |
| Group 1 (naïve: no treatment) | N/A | 3 |
| Group 2 (saline aerosol days 1 and 15)/ | N/A | 5 |
| (saline IT day 40) | | |
| Group 3 (0.5 mg/kg deposited dose Tri-SM6.1- | AC000292 | 5 |
| αvβ6-AD07475 days 1 and 15)/(saline IT day 40) | | |
| Group 4 (saline aerosol days 1 and 15)/(Alternaria | N/A | 7 |
| IT day 40) | | |
| Group 5 (0.5 mg/kg deposited dose Tri-SM6.1- | AC000292 | 7 |
| αvβ6-AD07475)/(Alternaria IT day 40) | | |

After 48 hours post-administration of the Alternaria (i.e., Day 42), rats were anesthetized with isoflurane/O₂, had blood drawn, and were euthanized by exsanguination. Trachea was canulated and bronchoalveolar lavage (BAL) was collected after washing with 2×10 mL of ice-cold PBS. BAL samples were spun down, cells resuspended with 1 mL of ice-cold PBS, and aliquot was mixed with Turk's solution (ratio 1:1), and total cell counted via hemocytomers. Cytospins were prepared, stained and differential cell counting performed. Supernatant was used for soluble RAGE (sRAGE) and cytokines measurements. One lobe was used to determine AGER mRNA expression and tissue samples were analyzed to assess concentration of RAGE protein.

Granulocytes, both eosinophils and neutrophils, are well known markers for cellular inflammation. For the BAL samples, the total and differential cells were counted and the number of inflammatory cells were derived. Group 5 (in which RAGE RNAi agent was administered and the tissues were challenged by Alternaria) showed a reduction of inflammatory cells as compared to Group 4 (in which no RNAi agent was administered).

Further, VEGF is known to cause vascular remodeling and is induced by inflammation. The groups administered with RAGE RNAi agent (Groups 3 and 5) showed reductions in VEGF levels in the BAL samples examined.

### Example 11. In Vivo Anti-Inflammatory Effect of RAGE Knock-Down in Rat Model of Airway Inflammation, Delivery Via Intra-Tracheal Microsprayer

On study day 1, day 8, and day 29, male Brown-Norway rats were administered a dose of 3 mg/kg (1.5 mL/kg) of the RAGE RNAi agent Tri-SM6.1-αvβ6-AD07475 or vehicle. Volume calculated at 1.5 mL/kg was loaded into a syringe that was connected to a microsprayer device (Penn Century, Philadelphia, PA).

On day 43, rats in Group 3 and Group 4 were challenged with a single intra-tracheal dose of 400 ug/rat of Alternaria alternata prepared in saline, and rats in Group 1 and 2 were administered a 400 ug/rat dose of saline control. Rats in Group N-1 were naïve and had no treatment administered.

TABLE 44
RAGE RNAi Agent and Dosing for Example 11.

| | AC Duplex | Animals |
|---|---|---|
| Group ID | Number | per Group |
| Group N-1 (naïve: no treatment) | N/A | 4 |
| Group 1 (saline IT days 1, 8, and 29)/(saline | N/A | 6 |
| IT day 43) | | |
| Group 2 (IT dose 3.0 mg/kg Tri-SM6.1-αvβ6- | AC000292 | 4 |
| AD07475 on days 1, 8, and 29)/(Alternaria | | |
| IT day 43) | | |
| Group 3 (saline IT days 1 and 15)/(saline day 43) | N/A | 8 |
| Group 4 (IT dose 3.0 mg/kg Tri-SM6.1-αvβ6- | AC000292 | 8 |
| AD07475 on days 1, 8, and 29)/(Alternaria | | |
| IT day 43) | | |

After 48 hours post-administration of the Alternaria (i.e. day 45), rats were anesthetized with isoflurane/O₂, blood was drawn, and were euthanized by exsanguination. Trachea was canulated and bronchoalveolar lavage (BAL) collected after washing with 2×10 mL of ice-cold PBS. BAL samples were spun down, cells resuspended with 1 mL of ice-cold PBS, and aliquot was mixed with Turk's solution (ratio 1:1), and total cell counted via hemocytomers. Cytospins were prepared, stained and differential cell counting performed. Supernatant was used for soluble RAGE (sRAGE) and cytokines measurements. One lobe was used to determine AGER mRNA expression and part of tissue was used to determine tissue concentration of RAGE protein.

Soluble RAGE (sRAGE) was measured in the serum and bronchoalveolar lavage fluid (BALF) samples by ELISA. sRAGE was nearly completely reduced by administration of the RAGE RNAi agents (see Groups 2 and 4).

Further, as noted in the prior Example, granulocytes (both eosinophils and neutrophils) are well known markers for cellular inflammation. For the BAL samples, the total and differential cells were counted and the number of inflammatory cells were derived. The impact of RAGE inhibition by the RAGE RNAi agents disclosed herein on eosinophilic inflammation induced by Alternaria extract was assessed. Group 4 (treated with RAGE RNAi agent) showed a reduction in total granulocytes compared to Group 3 (no RAGE RNAi agent administered).

Other biomarkers, such as MIP1a, IL-13, IP-10 and VEGF, are also indicative of cellular inflammation. For the Alternaria challenged groups, administration of the RAGE RNAi agent (Group 4) resulted in a reductions of each of these pro-inflammatory biomarkers compared to the group in which no RAGE RNAi agent was administered (Group 3).

### Example 12. Dose Response of In Vivo Inhaled Aerosolized Administration of RAGE RNAi Agents in Cynomolgus Monkeys

Fifteen (15) female cynomolgus animals were randomly assigned to five (5) treatment groups. The animals were dosed according to as summarized in the following Table 45. Animals were fasted the night before study days requiring anesthesia, but given water ad libitum. Animals were anesthetized with ketamine hydrochloride (5-10 mg/kg, IM), followed by isoflurane inhalation. Animals were initially anesthetized with isoflurane (4-5%) using a mask until a properly sized cuffed endotracheal tube was inserted, just proximal to the carina to allow the insertion of a pediatric fiberoptic bronchoscope to perform bronchoalveolar lavage (BAL) and to allow exposure through the endotracheal tube. Anesthetized animals were moved to the exposure system and connected to the ventilator. Anaesthetized and ventilated animals received a single inhalation exposure with either isotonic saline or RAGE RNAi agent Tri-SM6.1-αvβ6-AD09151 (AC001267). Animals were ventilated during the duration of the exposure using a Harvard pump set to 10 to 15 mL/kg tidal volume, 30 breaths per minute and inspiratory volume of 35:65. After finishing the exposure, the anesthesia was removed and animal then covered with blankets or Bair Hugger at a recovery station and connected to a monitor device for continuous capture of heart rate and O₂ saturation. The RAGE RNAi agent was conjugated to a tridentate small molecule αvβ6 epithelial cell targeting ligand (Tri-SM6.1, see FIG. 1) at the 5′ terminal end of the sense strand, formulated in isotonic saline. The chemically modified sequences for RAGE RNAi agents are shown in Table 7B (showing duplex), Table 3 (showing respective antisense strand), and Table 5 (showing respective sense strand with linker but without tridentate small molecule αvβ6 epithelial cell targeting ligand (Tri-SM6.1).

TABLE 45
RAGE RNAi Agent and Dosing for Example 12.

| | | Animals | Targeted | Calculated |
|---|---|---|---|---|
| | AC Duplex | per | Deposited | Deposited |
| Group ID | Number | Group | Dose/Animal | Dose |
| Group 1 (isotonic | N/A | 3 | (same exposure | N/A |
| saline) | | | time as Groups 2 | |
| | | | through 5) | |
| Group 2 (Tri-SM6.1- | AC001267 | 3 | 0.0625 mg/kg | 0.13 mg/kg |
| αvβ6-AD09151) | | | | |
| Group 3 (Tri-SM6.1- | AC001267 | 3 | 0.125 mg/kg | 0.20 mg/kg |
| αvβ6-AD09151) | | | | |
| Group 4 (Tri-SM6.1- | AC001267 | 3 | 0.25 mg/kg | 0.31 mg/kg |
| αvβ6-AD09151) | | | | |
| Group 5 (Tri-SM6.1- | AC001267 | 3 | 0.5 mg/kg | 0.47 mg/kg |
| αvβ6-AD09151) | | | | |

Bronchoalveolar lavage (BAL) and blood (serum) were collected at −7 days prior to exposure and 2-4 weeks after inhalation exposure (Days −7, 15, 29). All animals were euthanized just before sample collection on Day 29 to collect lung tissue.

The left lung of each animal was harvested for histology. The right lung was obtained, lobes separated, individually weighted and snap frozen. Separate lobes were sectioned in three (3) approximately equal pieces sliced at a longitudinal plane, perpendicular to the airway. Namely, separate lobes were sectioned in three (3) approximately equal pieces at a longitudinal plane, perpendicular to the airway, namely the proximal, medial, and distal. For cranial and medial lobes, two to three equally divided samples were collected from proximal slice, and three from the other two slices. Approximately equal size tissue cubes were obtained with presence and absence of visible airways.

The data in the following Tables 47 through 71 show mRNA expression sampled from the cynomolgus lung samples. Cynomolgus monkey AGER mRNA expression was quantitated by probe-based quantitative PCR, normalized to Cynomolgus monkey GAPDH expression, and expressed as fraction of vehicle control group (geometric mean, +/−95% confidence interval).

TABLE 47
Average Relative Cynomolgus Monkey RAGE mRNA Expression,
Right Distal Cranial Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.111 | 0.125 |
| Group 2 (0.13 mg/kg deposited | 0.406 | 0.113 | 0.156 |
| dose Tri-SM6.1-αvβ6-AD09151) | | | |
| Group 3 (0.20 mg/kg deposited | 0.589 | 0.165 | 0.229 |
| dose Tri-SM6.1-αvβ6-AD09151) | | | |
| Group 4 (0.31 mg/kg deposited | 0.353 | 0.076 | 0.096 |
| dose Tri-SM6.1-αvβ6-AD09151) | | | |
| Group 5 (0.47 mg/kg deposited | 0.358 | 0.139 | 0.227 |
| dose Tri-SM6.1-αvβ6-AD09151) | | | |

TABLE 48
Average Relative Cynomolgus Monkey RAGE mRNA Expression, Right
Distal Middle Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.156 | 0.185 |
| Group 2 (0.13 mg/kg deposited dose Tri- | 0.373 | 0.106 | 0.149 |
| SM6.1-αvβ6-AD09151) | | | |
| Group 3 (0.20 mg/kg deposited dose Tri- | 0.468 | 0.126 | 0.172 |
| SM6.1-αvβ6-AD09151) | | | |
| Group 4 (0.31 mg/kg deposited dose Tri- | 0.226 | 0.047 | 0.061 |
| SM6.1-αvβ6-AD09151) | | | |
| Group 5 (0.47 mg/kg deposited dose Tri- | 0.208 | 0.068 | 0.101 |
| SM6.1-αvβ6-AD09151) | | | |

TABLE 49
Average Relative Cynomolgus Monkey RAGE mRNA Expression, Right
Distal Caudal Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.070 | 0.075 |
| Group 2 (0.13 mg/kg deposited dose Tri- | 0.327 | 0.053 | 0.063 |
| SM6.1-αvβ6-AD09151) | | | |
| Group 3 (0.20 mg/kg deposited dose Tri- | 0.432 | 0.041 | 0.045 |
| SM6.1-αvβ6-AD09151) | | | |
| Group 4 (0.31 mg/kg deposited dose Tri- | 0.338 | 0.146 | 0.278 |
| SM6.1-αvβ6-AD09151) | | | |
| Group 5 (0.47 mg/kg deposited dose Tri- | 0.155 | 0.032 | 0.041 |
| SM6.1-αvβ6-AD09151) | | | |

TABLE 50
Average Relative Cynomolgus Monkey RAGE mRNA Expression, Right
Proximal Caudal Hilar Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.200 | 0.250 |
| Group 2 (0.13 mg/kg deposited dose Tri- | 0.436 | 0.113 | 0.153 |
| SM6.1-αvβ6-AD09151) | | | |
| Group 3 (0.20 mg/kg deposited dose Tri- | 0.449 | 0.073 | 0.087 |
| SM6.1-αvβ6-AD09151) | | | |
| Group 4 (0.31 mg/kg deposited dose Tri- | 0.371 | 0.055 | 0.064 |
| SM6.1-αvβ6-AD09151) | | | |
| Group 5 (0.47 mg/kg deposited dose Tri- | 0.337 | 0.099 | 0.140 |
| SM6.1-αvβ6-AD09151) | | | |

TABLE 51
Average Relative Cynomolgus Monkey RAGE mRNA Expression, Right
Proximal Middle Hilar Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.090 | 0.098 |
| Group 2 (0.13 mg/kg deposited dose Tri- | 0.491 | 0.128 | 0.172 |
| SM6.1-αvβ6-AD09151) | | | |
| Group 3 (0.20 mg/kg deposited dose Tri- | 0.597 | 0.090 | 0.106 |
| SM6.1-αvβ6-AD09151) | | | |
| Group 4 (0.31 mg/kg deposited dose Tri- | 0.450 | 0.034 | 0.033 |
| SM6.1-αvβ6-AD09151) | | | |
| Group 5 (0.47 mg/kg deposited dose Tri- | 0.345 | 0.038 | 0.043 |
| SM6.1-αvβ6-AD09151) | | | |

TABLE 52
Average Relative Cynomolgus Monkey RAGE mRNA Expression, Right
Proximal Caudal Hilar Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.083 | 0.090 |
| Group 2 (0.13 mg/kg deposited dose Tri- | 0.419 | 0.074 | 0.090 |
| SM6.1-αvβ6-AD09151) | | | |
| Group 3 (0.20 mg/kg deposited dose Tri- | 0.569 | 0.093 | 0.111 |
| SM6.1-αvβ6-AD09151) | | | |
| Group 4 (0.31 mg/kg deposited dose Tri- | 0.273 | 0.056 | 0.078 |
| SM6.1-αvβ6-AD09151) | | | |
| Group 5 (0.47 mg/kg deposited dose Tri- | 0.308 | 0.080 | 0.108 |
| SM6.1-αvβ6-AD09151) | | | |

TABLE 53
Average Relative Cynomolgus Monkey RAGE mRNA Expression, Right
Distal Cranial Hilar Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.074 | 0.080 |
| Group 2 (0.13 mg/kg deposited dose Tri- | 0.265 | 0.072 | 0.099 |
| SM6.1-αvβ6-AD09151) | | | |
| Group 3 (0.20 mg/kg deposited dose Tri- | 0.555 | 0.055 | 0.061 |
| SM6.1-αvβ6-AD09151) | | | |
| Group 4 (0.31 mg/kg deposited dose Tri- | 0.286 | 0.017 | 0.018 |
| SM6.1-αvβ6-AD09151) | | | |
| Group 5 (0.47 mg/kg deposited dose Tri- | 0.296 | 0.057 | 0.070 |
| SM6.1-αvβ6-AD09151) | | | |

TABLE 54
Average Relative Cynomolgus Monkey RAGE mRNA Expression, Right
Distal Middle Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.053 | 0.056 |
| Group 2 (0.13 mg/kg deposited dose Tri- | 0.395 | 0.129 | 0.191 |
| SM6.1-αvβ6-AD09151) | | | |
| Group 3 (0.20 mg/kg deposited dose Tri- | 0.569 | 0.109 | 0.135 |
| SM6.1-αvβ6-AD09151) | | | |
| Group 4 (0.31 mg/kg deposited dose Tri- | 0.169 | 0.030 | 0.042 |
| SM6.1-αvβ6-AD09151) | | | |
| Group 5 (0.47 mg/kg deposited dose Tri- | 0.333 | 0.132 | 0.220 |
| SM6.1-αvβ6-AD09151) | | | |

TABLE 55
Average Relative Cynomolgus Monkey RAGE mRNA Expression, Right
Distal Caudal Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.395 | 0.652 |
| Group 2 (0.13 mg/kg deposited dose Tri- | 0.567 | 0.070 | 0.080 |
| SM6.1-αvβ6-AD09151) | | | |
| Group 3 (0.20 mg/kg deposited dose Tri- | 0.713 | 0.096 | 0.111 |
| SM6.1-αvβ6-AD09151) | | | |
| Group 4 (0.31 mg/kg deposited dose Tri- | 0.442 | 0.133 | 0.204 |
| SM6.1-αvβ6-AD09151) | | | |
| Group 5 (0.47 mg/kg deposited dose Tri- | 0.285 | 0.084 | 0.120 |
| SM6.1-αvβ6-AD09151) | | | |

TABLE 56
Average Relative Cynomolgus Monkey RAGE mRNA Expression, Right
Proximal Cranial Hilar Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.269 | 0.368 |
| Group 2 (0.13 mg/kg deposited dose Tri- | 0.451 | 0.127 | 0.176 |
| SM6.1-αvβ6-AD09151) | | | |
| Group 3 (0.20 mg/kg deposited dose Tri- | 0.513 | 0.134 | 0.181 |
| SM6.1-αvβ6-AD09151) | | | |
| Group 4 (0.31 mg/kg deposited dose Tri- | 0.355 | 0.053 | 0.062 |
| SM6.1-αvβ6-AD09151) | | | |
| Group 5 (0.47 mg/kg deposited dose Tri- | 0.307 | 0.106 | 0.161 |
| SM6.1-αvβ6-AD09151) | | | |

TABLE 57
Average Relative Cynomolgus Monkey RAGE mRNA Expression, Right
Medial Cranial Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.082 | 0.090 |
| Group 2 (0.13 mg/kg deposited dose Tri- | 0.397 | 0.103 | 0.139 |
| SM6.1-αvβ6-AD09151) | | | |
| Group 3 (0.20 mg/kg deposited dose Tri- | 0.549 | 0.049 | 0.053 |
| SM6.1-αvβ6-AD09151) | | | |
| Group 4 (0.31 mg/kg deposited dose Tri- | 0.278 | 0.071 | 0.095 |
| SM6.1-αvβ6-AD09151) | | | |
| Group 5 (0.47 mg/kg deposited dose Tri- | 0.280 | 0.091 | 0.135 |
| SM6.1-αvβ6-AD09151) | | | |

TABLE 58
Average Relative Cynomolgus Monkey RAGE mRNA Expression, Right
Medial Middle Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.058 | 0.061 |
| Group 2 (0.13 mg/kg deposited dose Tri- | 0.396 | 0.072 | 0.088 |
| SM6.1-αvβ6-AD09151) | | | |
| Group 3 (0.20 mg/kg deposited dose Tri- | 0.577 | 0.124 | 0.157 |
| SM6.1-αvβ6-AD09151) | | | |
| Group 4 (0.31 mg/kg deposited dose Tri- | 0.375 | 0.074 | 0.093 |
| SM6.1-αvβ6-AD09151) | | | |
| Group 5 (0.47 mg/kg deposited dose Tri- | 0.307 | 0.039 | 0.045 |
| SM6.1-αvβ6-AD09151) | | | |

TABLE 59
Average Relative Cynomolgus Monkey RAGE mRNA Expression, Right
Medial Caudal Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.082 | 0.089 |
| Group 2 (0.13 mg/kg deposited | 0.408 | 0.122 | 0.174 |
| dose Tri-SM6.1-αvβ6-AD09151) | | | |
| Group 3 (0.20 mg/kg deposited | 0.507 | 0.052 | 0.058 |
| dose Tri-SM6.1-αvβ6-AD09151) | | | |
| Group 4 (0.31 mg/kg deposited | 0.280 | 0.036 | 0.046 |
| dose Tri-SM6.1-αvβ6-AD09151) | | | |
| Group 5 (0.47 mg/kg deposited | 0.283 | 0.048 | 0.058 |
| dose Tri-SM6.1-αvβ6-AD09151) | | | |

TABLE 60
Average Relative Cynomolgus Monkey RAGE mRNA Expression, Right
Distal Cranial Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.088 | 0.096 |
| Group 2 (0.13 mg/kg deposited | 0.351 | 0.066 | 0.082 |
| dose Tri-SM6.1-αvβ6-AD09151) | | | |
| Group 3 (0.20 mg/kg deposited | 0.509 | 0.077 | 0.090 |
| dose Tri-SM6.1-αvβ6-AD09151) | | | |
| Group 4 (0.31 mg/kg deposited | 0.316 | 0.064 | 0.081 |
| dose Tri-SM6.1-αvβ6-AD09151) | | | |
| Group 5 (0.47 mg/kg deposited | 0.311 | 0.085 | 0.117 |
| dose Tri-SM6.1-αvβ6-AD09151) | | | |

TABLE 61
Average Relative Cynomolgus Monkey RAGE mRNA Expression, Right
Distal Middle Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.227 | 0.294 |
| Group 2 (0.13 mg/kg deposited | 0.195 | 0.086 | 0.154 |
| dose Tri-SM6.1-αvβ6-AD09151) | | | |
| Group 3 (0.20 mg/kg deposited | 0.429 | 0.119 | 0.165 |
| dose Tri-SM6.1-αvβ6-AD09151) | | | |
| Group 4 (0.31 mg/kg deposited | 0.289 | 0.028 | 0.040 |
| dose Tri-SM6.1-αvβ6-AD09151) | | | |
| Group 5 (0.47 mg/kg deposited | 0.242 | 0.069 | 0.096 |
| dose Tri-SM6.1-αvβ6-AD09151) | | | |

TABLE 62
Average Relative Cynomolgus Monkey RAGE mRNA Expression, Right
Distal Caudal Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.077 | 0.083 |
| Group 2 (0.13 mg/kg deposited | 0.434 | 0.114 | 0.155 |
| dose Tri-SM6.1-αvβ6-AD09151) | | | |
| Group 3 (0.20 mg/kg deposited | 0.554 | 0.131 | 0.171 |
| dose Tri-SM6.1-αvβ6-AD09151) | | | |
| Group 4 (0.31 mg/kg deposited | 0.540 | 0.240 | 0.405 |
| dose Tri-SM6.1-αvβ6-AD09151) | | | |
| Group 5 (0.47 mg/kg deposited | 0.314 | 0.087 | 0.121 |
| dose Tri-SM6.1-αvβ6-AD09151) | | | |

TABLE 63
Average Relative Cynomolgus Monkey RAGE mRNA Expression, Right
Medial Cranial Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.131 | 0.151 |
| Group 2 (0.13 mg/kg deposited | 0.499 | 0.169 | 0.256 |
| dose Tri-SM6.1-αvβ6-AD09151) | | | |
| Group 3 (0.20 mg/kg deposited | 0.545 | 0.116 | 0.147 |
| dose Tri-SM6.1-αvβ6-AD09151) | | | |
| Group 4 (0.31 mg/kg deposited | 0.332 | 0.020 | 0.021 |
| dose Tri-SM6.1-αvβ6-AD09151) | | | |
| Group 5 (0.47 mg/kg deposited | 0.360 | 0.100 | 0.138 |
| dose Tri-SM6.1-αvβ6-AD09151) | | | |

TABLE 64
Average Relative Cynomolgus Monkey RAGE mRNA Expression, Right
Medial Middle Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.071 | 0.076 |
| Group 2 (0.13 mg/kg deposited | 0.571 | 0.130 | 0.169 |
| dose Tri-SM6.1-αvβ6-AD09151) | | | |
| Group 3 (0.20 mg/kg deposited | 0.708 | 0.131 | 0.161 |
| dose Tri-SM6.1-αvβ6-AD09151) | | | |
| Group 4 (0.31 mg/kg deposited | 0.306 | 0.035 | 0.039 |
| dose Tri-SM6.1-αvβ6-AD09151) | | | |
| Group 5 (0.47 mg/kg deposited | 0.258 | 0.074 | 0.103 |
| dose Tri-SM6.1-αvβ6-AD09151) | | | |

TABLE 65
Average Relative Cynomolgus Monkey RAGE mRNA Expression, Right
Medial Caudal Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.180 | 0.219 |
| Group 2 (0.13 mg/kg deposited | 0.381 | 0.080 | 0.101 |
| dose Tri-SM6.1-αvβ6-AD09151) | | | |
| Group 3 (0.20 mg/kg deposited | 0.580 | 0.109 | 0.135 |
| dose Tri-SM6.1-αvβ6-AD09151) | | | |
| Group 4 (0.31 mg/kg deposited | 0.304 | 0.041 | 0.052 |
| dose Tri-SM6.1-αvβ6-AD09151) | | | |
| Group 5 (0.47 mg/kg deposited | 0.235 | 0.039 | 0.046 |
| dose Tri-SM6.1-αvβ6-AD09151) | | | |

TABLE 66
Average Relative Cynomolgus Monkey RAGE mRNA Expression, Right
Proximal Cranial Hilar Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.153 | 0.180 |
| Group 2 (0.13 mg/kg deposited | 0.492 | 0.152 | 0.220 |
| dose Tri-SM6.1-αvβ6-AD09151) | | | |
| Group 3 (0.20 mg/kg deposited | 0.486 | 0.115 | 0.151 |
| dose Tri-SM6.1-αvβ6-AD09151) | | | |
| Group 4 (0.31 mg/kg deposited | 0.265 | 0.026 | 0.029 |
| dose Tri-SM6.1-αvβ6-AD09151) | | | |
| Group 5 (0.47 mg/kg deposited | 0.272 | 0.053 | 0.065 |
| dose Tri-SM6.1-αvβ6-AD09151) | | | |

TABLE 67
Average Relative Cynomolgus Monkey RAGE mRNA Expression, Right
Proximal Middle Hilar Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.124 | 0.141 |
| Group 2 (0.13 mg/kg deposited | 0.537 | 0.121 | 0.157 |
| dose Tri-SM6.1-αvβ6-AD09151) | | | |
| Group 3 (0.20 mg/kg deposited | 0.509 | 0.156 | 0.226 |
| dose Tri-SM6.1-αvβ6-AD09151) | | | |
| Group 4 (0.31 mg/kg deposited | 0.247 | 0.064 | 0.077 |
| dose Tri-SM6.1-αvβ6-AD09151) | | | |
| Group 5 (0.47 mg/kg deposited | 0.322 | 0.090 | 0.125 |
| dose Tri-SM6.1-αvβ6-AD09151) | | | |

TABLE 68
Average Relative Cynomolgus Monkey RAGE mRNA Expression, Right
Proximal Caudal Hilar Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.088 | 0.097 |
| Group 2 (0.13 mg/kg deposited | 0.310 | 0.067 | 0.086 |
| dose Tri-SM6.1-αvβ6-AD09151) | | | |
| Group 3 (0.20 mg/kg deposited | 0.502 | 0.105 | 0.133 |
| dose Tri-SM6.1-αvβ6-AD09151) | | | |
| Group 4 (0.31 mg/kg deposited | 0.248 | 0.043 | 0.054 |
| dose Tri-SM6.1-αvβ6-AD09151) | | | |
| Group 5 (0.47 mg/kg deposited | 0.219 | 0.050 | 0.065 |
| dose Tri-SM6.1-αvβ6-AD09151) | | | |

TABLE 69
Average Relative Cynomolgus Monkey RAGE mRNA Expression, Right
Medial Caudal Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.112 | 0.126 |
| Group 2 (0.13 mg/kg deposited | 0.422 | 0.097 | 0.127 |
| dose Tri-SM6.1-αvβ6-AD09151) | | | |
| Group 3 (0.20 mg/kg deposited | 0.492 | 0.049 | 0.054 |
| dose Tri-SM6.1-αvβ6-AD09151) | | | |
| Group 4 (0.31 mg/kg deposited | 0.222 | 0.018 | 0.019 |
| dose Tri-SM6.1-αvβ6-AD09151) | | | |
| Group 5 (0.47 mg/kg deposited | 0.221 | 0.088 | 0.147 |
| dose Tri-SM6.1-αvβ6-AD09151) | | | |

TABLE 70
Average Relative Cynomolgus Monkey RAGE mRNA Expression, Right
Medial Middle Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.086 | 0.094 |
| Group 2 (0.13 mg/kg deposited | 0.434 | 0.144 | 0.216 |
| dose Tri-SM6.1-αvβ6-AD09151) | | | |
| Group 3 (0.20 mg/kg deposited | 0.693 | 0.134 | 0.167 |
| dose Tri-SM6.1-αvβ6-AD09151) | | | |
| Group 4 (0.31 mg/kg deposited | 0.291 | 0.057 | 0.065 |
| dose Tri-SM6.1-αvβ6-AD09151) | | | |
| Group 5 (0.47 mg/kg deposited | 0.277 | 0.064 | 0.084 |
| dose Tri-SM6.1-αvβ6-AD09151) | | | |

TABLE 71
Average Relative Cynomolgus Monkey RAGE mRNA Expression, Right
Medial Caudal Lobe, at Sacrifice in Example 12.

| | Average Relative | | |
|---|---|---|---|
| | cAGER mRNA | Low | High |
| Group ID | Expression (n = 3) | (error) | (error) |
| Group 1 (isotonic saline) | 1.000 | 0.159 | 0.189 |
| Group 2 (0.13 mg/kg deposited | 0.338 | 0.080 | 0.106 |
| dose Tri-SM6.1-αvβ6-AD09151) | | | |
| Group 3 (0.20 mg/kg deposited | 0.438 | 0.058 | 0.067 |
| dose Tri-SM6.1-αvβ6-AD09151) | | | |
| Group 4 (0.31 mg/kg deposited | 0.297 | 0.090 | 0.113 |
| dose Tri-SM6.1-αvβ6-AD09151) | | | |
| Group 5 (0.47 mg/kg deposited | 0.215 | 0.072 | 0.108 |
| dose Tri-SM6.1-αvβ6-AD09151) | | | |

As shown in the data in Tables 47 through 71 above, RNAi agent AD09151 showed substantial inhibition (>70% inhibition observed with single inhaled dose at 0.31 mg/kg and 0.47 mg/kg) compared to control, demonstrating the ability to robustly silence AGER expression in non-human primates.

Tissue RAGE protein was analyzed via Western Blot. Pulverized lung tissue aliquot, based on availability of tissues, which included caudal distal lung tissue, and middle lobe tissue, was homogenized in radioimmunoprecipitation assay buffer (RIPA) using Qiagen bead-based homogenizer. Protein concentration was determined via bicinchoninic acid assay (BCA assay). The samples were prepared in Laemelli buffer and boiled. The gel was loaded with 10 μg total protein, and semi-dry transferred into a polyvinylidene difluoride (PVDF) membrane. The blot was assayed via Pierce Fast-western kit (30 min. primary antibody (ab216329) 1:1000 in proprietary blocking buffer, 15 min. HRP, 3×5 min. washes, 5 min. detection substrate West Pico). The resulting blot was imaged on iBright for RAGE and total protein stain, with the membrane treated with total protein normalization reagent. Densitometry of both bands of interest and for total protein minus background signal was quantified using iBright software.

Dose response of cynomolgus monkeys after administration of RAGE RNAi agent shows progressively higher RAGE protein inhibition in higher RAGE RNAi agent dose concentrations. This can be seen in both the middle and caudal lobes, with up to ~80-88% RAGE protein inhibition at 0.47 mg/kg.

Serum samples from cynomolgus monkeys at Day 15 and 29 after dosing with RAGE RNAi agent were collected, and Serum sRAGE levels were quantified via Gyros Protein Technologies assay. The following Table 72 shows the serum sRAGE levels at Day 15, normalized to baseline, along with standard deviation. The following Table 73 shows the serum sRAGE levels at Day 29, normalized to baseline, along with standard deviation.

TABLE 72
Serum sRAGE levels of cynomolgus monkeys after exposure to
RAGE RNAi agent, Day 15.

| | | Serum | |
|---|---|---|---|
| | | sRAGE | Standard |
| Group ID | Dose | (% BSL) | Deviation |
| Group 1 (isotonic saline) | N/A | 92.710 | 0.014 |
| Group 2 Tri-SM6.1-αvβ6-AD09151 | 0.13 mg/kg | 63.293 | 16.806 |
| Group 3 Tri-SM6.1-αvβ6-AD09151 | 0.20 mg/kg | 57.873 | 7.367 |
| Group 4 Tri-SM6.1-αvβ6-AD09151 | 0.31 mg/kg | 47.710 | 15.971 |
| Group 5 Tri-SM6.1-αvβ6-AD09151 | 0.47 mg/kg | 43.377 | 4.487 |

TABLE 73
Serum sRAGE levels of cynomolgus monkeys after exposure to
RAGE RNAi agent, Day 29.

| | | Serum | |
|---|---|---|---|
| | | sRAGE | Standard |
| Group ID | Dose | (% BSL) | Deviation |
| Group 1 (isotonic saline) | N/A | 100.470 | 28.206 |
| Group 2 Tri-SM6.1-αvβ6-AD09151 | 0.13 mg/kg | 79.750 | 17.336 |
| Group 3 Tri-SM6.1-αvβ6-AD09151 | 0.20 mg/kg | 66.523 | 5.243 |
| Group 4 Tri-SM6.1-αvβ6-AD09151 | 0.31 mg/kg | 46.873 | 25.249 |
| Group 5 Tri-SM6.1-αvβ6-AD09151 | 0.47 mg/kg | 39.833 | 13.811 |

After dosing with RAGE RNAi agents, cynomolgus monkeys showed significantly reduced serum sRAGE levels, compared to baseline levels. As shown in Tables 72 and 73, the dose response showed progressively more reduced sRAGE levels (higher sRAGE inhibition) at higher RAGE RNAi agent dosing concentrations, showing up to ~39-43% serum sRAGE levels (~57-61% inhibition) at Days 15 and 29 after exposure to RAGE RNAi agent.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An RNAi agent for inhibiting expression of a Receptor for Advanced Glycation End-products gene, comprising:
an antisense strand comprising at least 17 contiguous nucleotides differing by 0 or 1 nucleotides from any one of the sequences provided in Table 2 or Table 3; and
a sense strand comprising a nucleotide sequence that is at least partially complementary to the antisense strand.

2.-3. (canceled)

4. The RNAi agent of claim 1, wherein at least one nucleotide of the RNAi agent is a modified nucleotide or includes a modified internucleoside linkage.

5. (canceled)

6. The RNAi agent of claim 4, wherein the modified nucleotide is selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.

7.-10. (canceled)

11. The RNAi agent of claim 1, wherein the sense strand is between 18 and 30 nucleotides in length, and the antisense strand is between 18 and 30 nucleotides in length.

12.-16. (canceled)

17. The RNAi agent of claim 1, wherein the sense strand comprises one or two inverted abasic residues.

18. The RNAi agent of claim 1, wherein the RNAi agent is comprised of a sense strand and an antisense strand that form a duplex having the structure of any one of the duplexes in Table 7A, Table 7B, Table 8, Table 9A, Table 9B, or Table 10.

19. (canceled)

20. The RNAi agent of claim 1, comprising an antisense strand that consists of, consists essentially of, or comprises a nucleotide sequence that differs by 0 or 1 nucleotides from one of the following nucleotide sequences (5′→3′):
(SEQ ID NO: 55)
UUGUGUUCAGUUUCCAUUC;
(SEQ ID NO: 65)
UGAUGUUUUGAGCACCUAC;
(SEQ ID NO: 73)
UUCCAUUCCUGUUCAUUGC;
(SEQ ID NO: 7)
UUGUGUUCAGUUUCCAUUCCG;
(SEQ ID NO: 9)
UGAUGUUUUGAGCACCUACUC;
or
(SEQ ID NO: 8)
UUCCAUUCCUGUUCAUUGCCU.

21. The RNAi agent of claim 20, wherein the sense strand consists of, consists essentially of, or comprises a nucleotide sequence that differs by 0 or 1 nucleotides from one of the following nucleotide sequences (5′→3′):
(SEQ ID NO: 298)
GAAUGGAAACUGAACACAA;
(SEQ ID NO: 308)
GUAGGUGCUCAAAACAUCA;
(SEQ ID NO: 316)
GCAAUGAACAGGAAUIGAA;
(SEQ ID NO: 19)
CGGAAUGGAAACUGAACACAA;
(SEQ ID NO: 20)
GAGUAGGUGCUCAAAACAUCA;
or
(SEQ ID NO: 21)
AGGCAAUGAACAGGAAUIGAA.

22. (canceled)

23. The RNAi agent of claim 1, comprising an antisense strand that comprises, consists of, or consists essentially of a modified nucleotide sequence that differs by 0 or 1 nucleotides from one of the following nucleotide sequences (5′→3′):
(SEQ ID NO: 2)
usUfsgsUfgUfuCfaGfuUfuCfcAfuUfcCfsg;
(SEQ ID NO: 3)
cPrpusUfsgsUfgUfuCfaGfuUfuCfcAfuUfcCfsg;
(SEQ ID NO: 5)
usGfsasuguuuugaGfcAfcCfuacusc;
(SEQ ID NO: 6)
cPrpusGfsasuguuuugaGfcAfcCfuacusc;
(SEQ ID NO: 4)
usUfscsCfaUfuCfcUfgUfuCfaUfuGfcCfsu;
wherein a represents 2′-O-methyl adenosine, c represents 2′-O-methyl cytidine, g represents 2′-O-methyl guanosine, u represents 2′-O-methyl uridine; Af represents 2′-fluoro adenosine, Cf represents 2′-fluoro cytidine, Gf represents 2′-fluoro guanosine, Uf represents 2′-fluoro uridine; cPrpu represents 5′-cyclopropyl phosphonate-2′-O-methyl uridine; s represents a phosphorothioate linkage; and wherein all or substantially all of the nucleotides on the sense strand are modified nucleotides.

24. The RNAi agent of claim 1, wherein the sense strand comprises, consists of, or consists essentially of a modified nucleotide sequence that differs by 0 or 1 nucleotides from one of the following nucleotide sequences (5′→3′):
(SEQ ID NO: 14)
gsaguagGfuGfcUfcaaaacauca;
(SEQ ID NO: 15)
asggcaaugAfAfCfaggaauigaa;
(SEQ ID NO: 13)
csggaauggAfAfAfcugaacacaa;
wherein a represents 2′-O-methyl adenosine, c represents 2′-O-methyl cytidine, g represents 2′-O-methyl guanosine, i represents 2′-O-methyl inosine, u represents 2′-O-methyl uridine; Af represents 2′-fluoro adenosine, Cf represents 2′-fluoro cytidine, Gf represents 2′-fluoro guanosine, Uf represents 2′-fluoro uridine; and s represents a phosphorothioate linkage; and wherein all or substantially all of the nucleotides on the antisense strand are modified nucleotides.

25. (canceled)

26. The RNAi agent of claim 1, wherein the RNAi agent is linked to a targeting ligand.

27.-29. (canceled)

30. The RNAi agent of claim 26, wherein the targeting ligand comprises the structure: 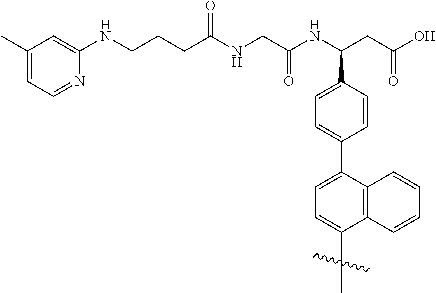 [SMILES: *c1ccc(-c2ccc([C@H](CC(=O)O)NC(=O)CNC(=O)CCCNc3cc(C)ccn3)cc2)c2ccccc12]
or a pharmaceutically acceptable salt thereof, or 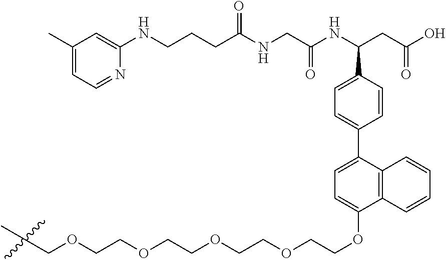 [SMILES: *COCCOCCOCCOCCOc1ccc(-c2ccc([C@H](CC(=O)O)NC(=O)CNC(=O)CCCNc3cc(C)ccn3)cc2)c2ccccc12]
or a pharmaceutically acceptable salt thereof,
wherein [symbol] indicates the point of connection to the RNAi agent.

31. The RNAi agent of claim 26, wherein the targeting ligand has a structure selected from the group consisting of: 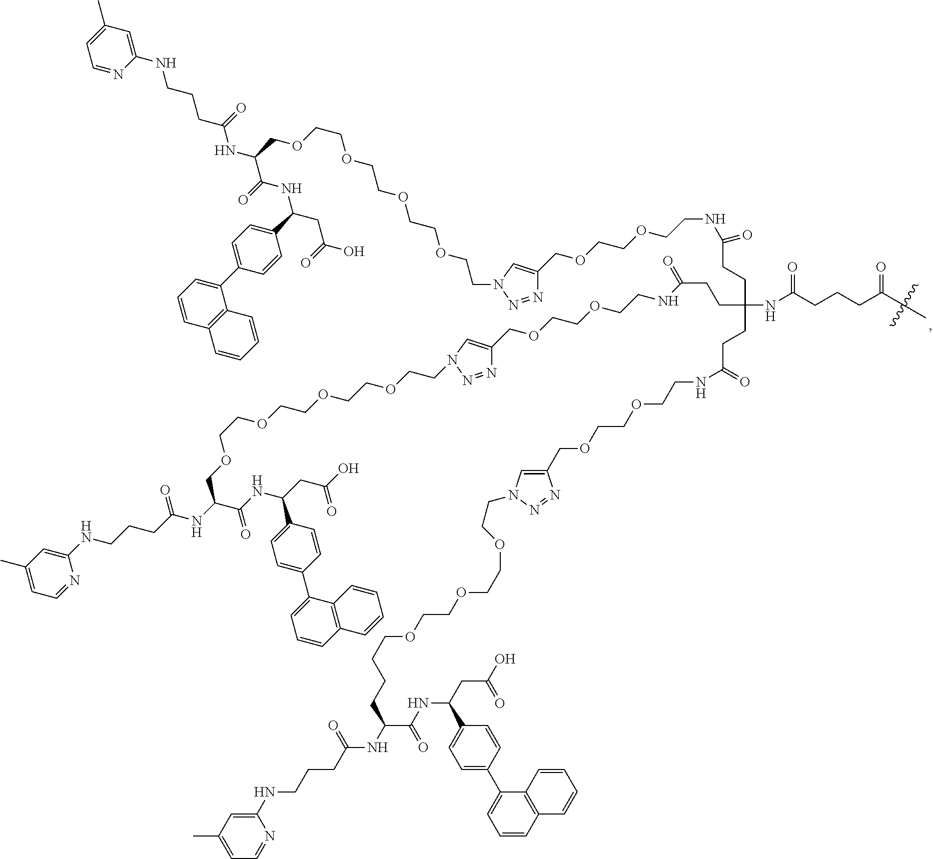 [SMILES: *C(=O)CCCC(=O)NC(CCC(=O)NCCOCCOCc1cn(CCOCCOCCOCCCC[C@H](NC(=O)CCCNc2cc(C)ccn2)C(=O)N[C@@H](CC(=O)O)c2ccc(-c3cccc4ccccc34)cc2)nn1)(CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1)CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1] 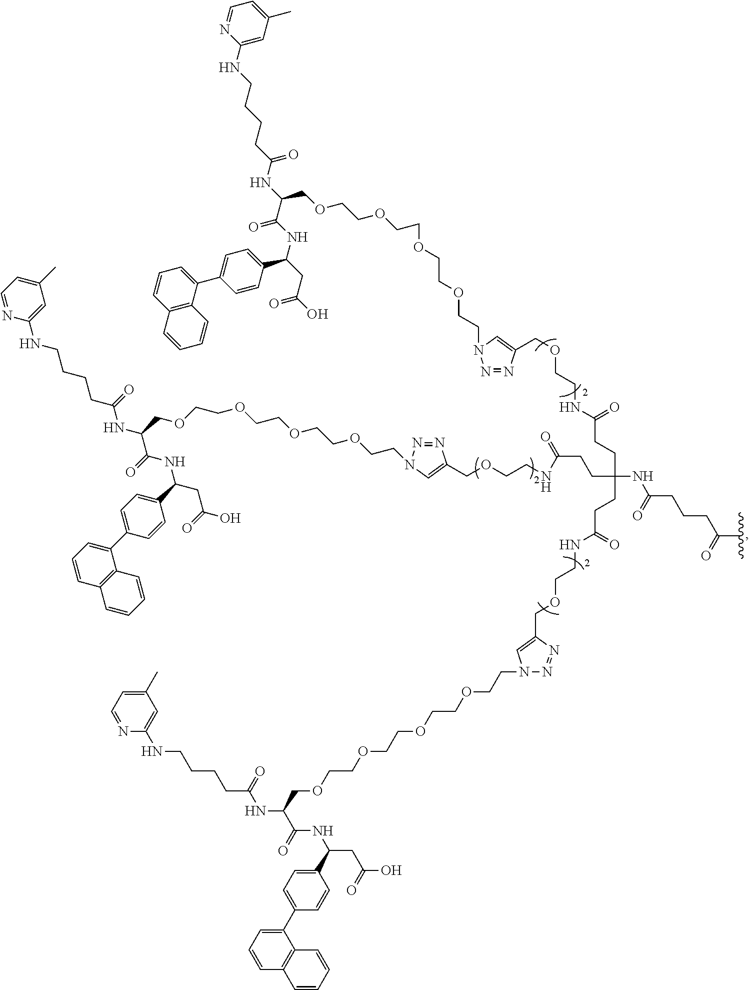 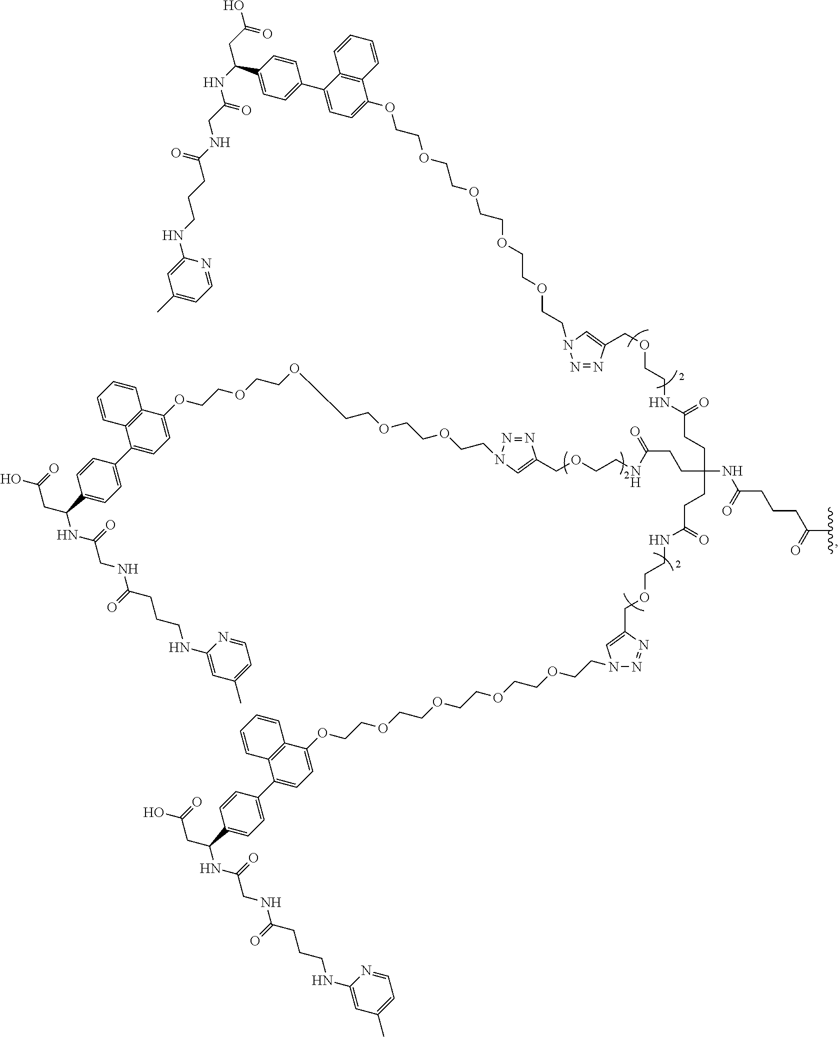 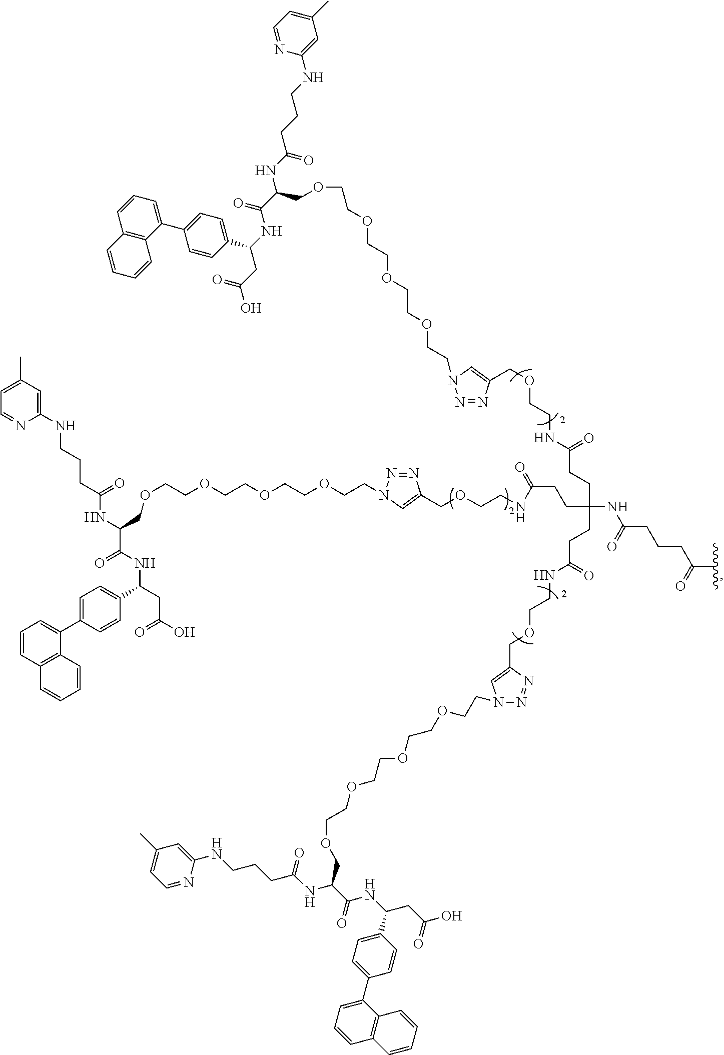 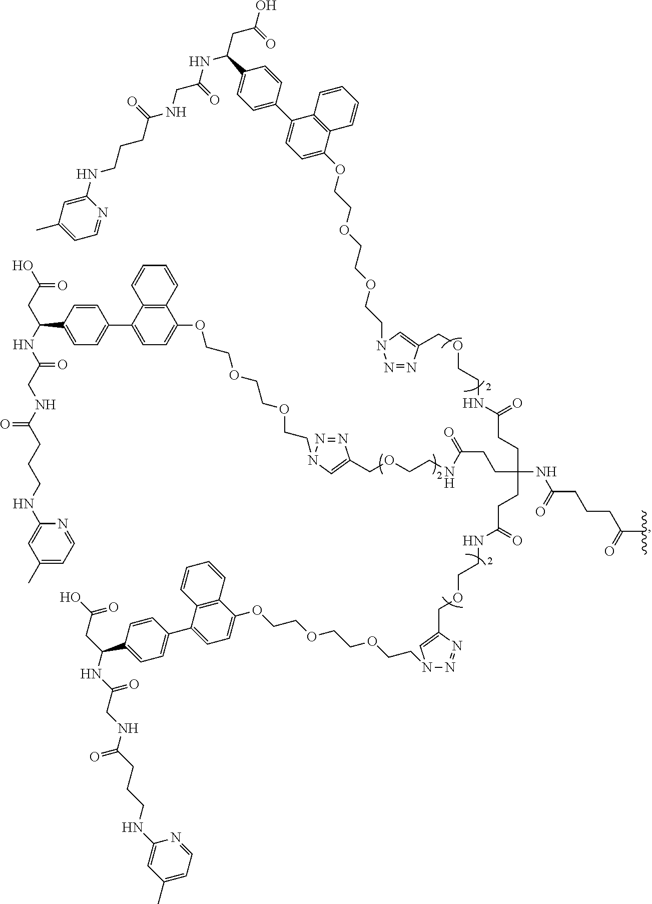 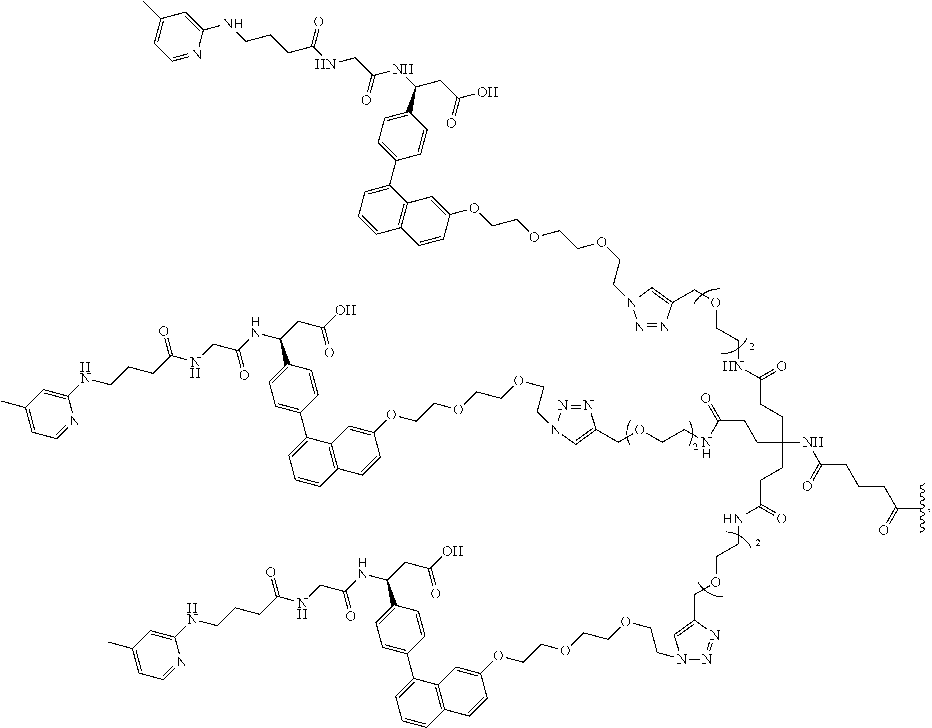 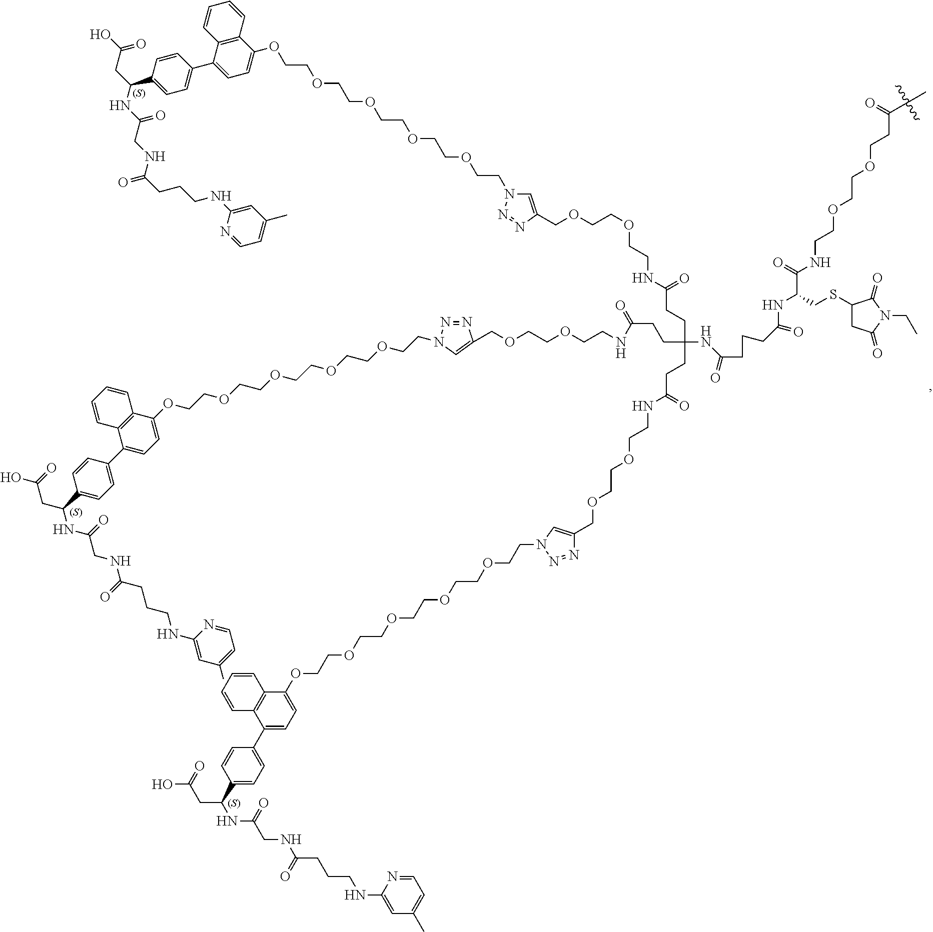 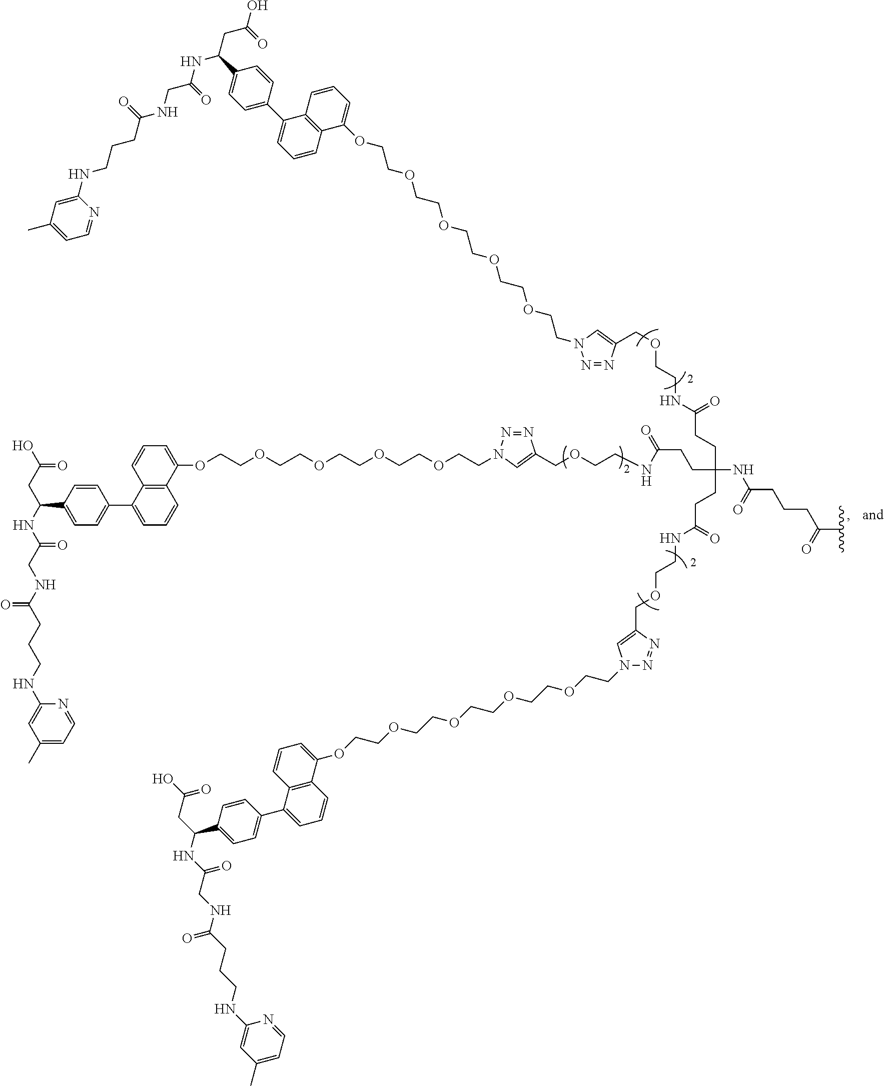 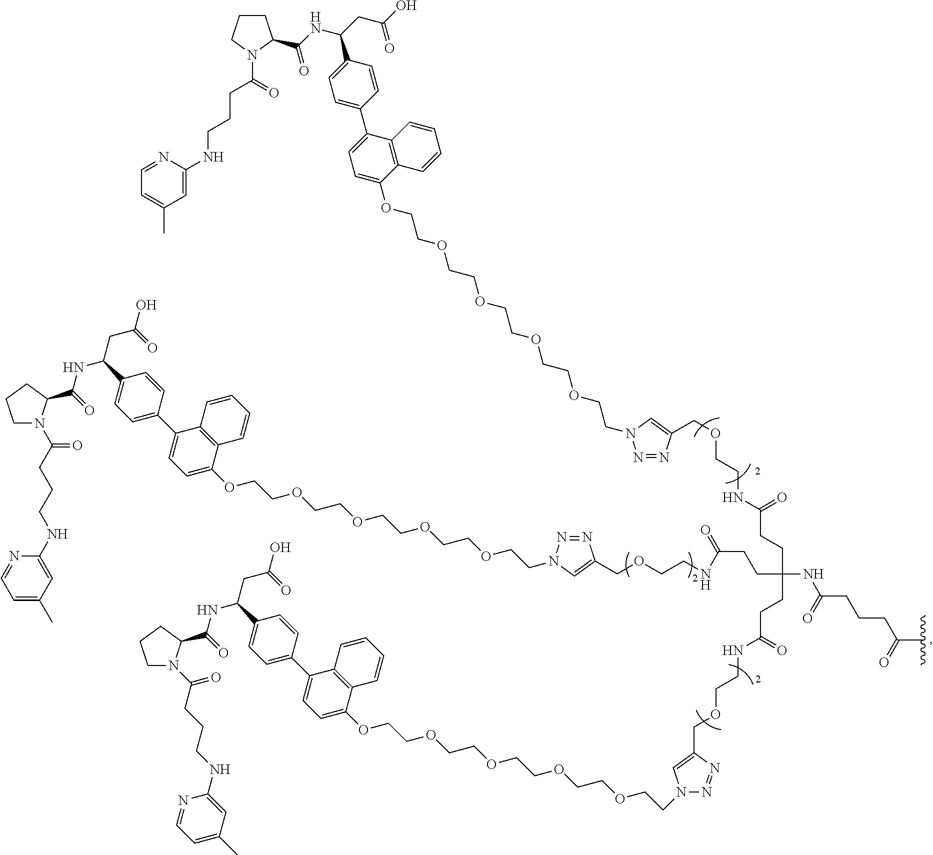
wherein [symbol] indicates the point of connection to the RNAi agent.

32. The RNAi agent of claim 31, wherein RNAi agent is conjugated to a targeting ligand having the following structure: 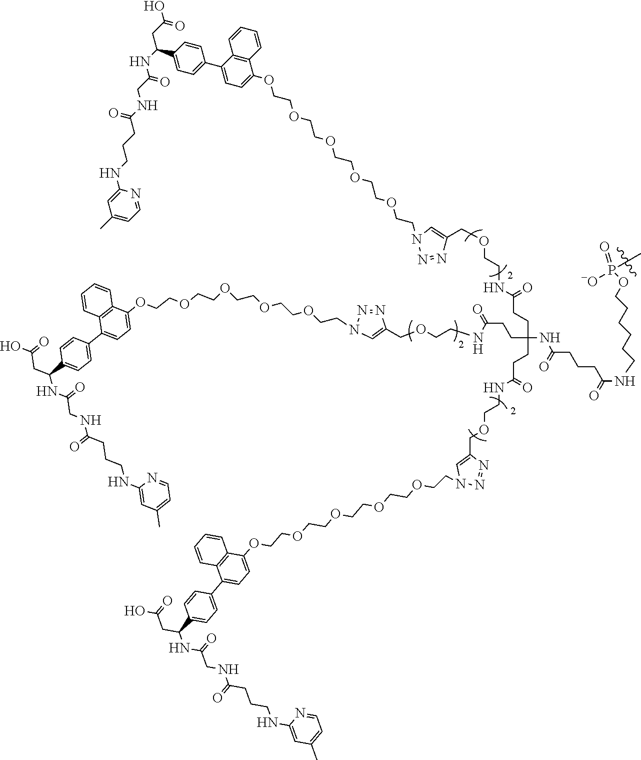
wherein [symbol] indicates the point of connection to the RNAi agent.

33.-36. (canceled)

37. A composition comprising the RNAi agent of claim 1, wherein the composition further comprises a pharmaceutically acceptable excipient.

38.-44. (canceled)

45. A method for inhibiting expression of a Receptor for Advanced Glycation End-products gene in a cell, the method comprising introducing into a cell an effective amount of an RNAi agent of claim 1.

46. The method of claim 45, wherein the cell is within a subject.

47.-48. (canceled)

49. A method of treating one or more symptoms or diseases associated with enhanced or elevated membrane RAGE activity levels, the method comprising administering to a human subject in need thereof a therapeutically effective amount of the composition of claim 37.

50. The method of claim 49, wherein the disease is a respiratory disease.

51.-61. (canceled)

62. A method of making an RNAi agent of claim 1, comprising annealing a sense strand and an antisense strand to form a double-stranded ribonucleic acid molecule.

63.-64. (canceled)
